(19) United States
(12) Patent Application Publication
(10) Pub. No.: US 2025/0213604 A1
(43) Pub. Date: Jul. 3, 2025

(54) TARGETED DELIVERY

(71) Applicant: Judo Bio Inc., Cambridge, MA (US)

(72) Inventors: Steven L. Robinette, Cambridge, MA (US); Chelsea Schwartz Place Johnson, Cambridge, MA (US); Brian R. Bettencourt, Cambridge, MA (US); Andrew W. Fraley, Cambridge, MA (US); Jonathan Lawrence, Cambridge, MA (US); Haojing Rong, Cambridge, MA (US)

(21) Appl. No.: 18/850,312

(22) PCT Filed: Mar. 24, 2023
(86) PCT No.: PCT/US23/16319

§ 371 Date: Sep. 24, 2024

Related U.S. Application Data
(60) Provisional application No. 63/323,633, filed on Mar. 25, 2022.
(60) Provisional application No. 63/323,635, filed on Mar. 25, 2022.

(51) Int. Cl. A61K 31/713; A61K 47/61; A61K 47/64; C12N 15/87

(52) U.S. Cl. CPC A61K 31/713; A61K 47/61; A61K 47/64; C12N 15/87

(57) ABSTRACT

Disclosed herein are conjugate agents comprising a targeting moiety, directly or indirectly conjugated with a payload moiety, compositions comprising the same as well as methods of making and using the same.

### CROSS-REFERENCE TO RELATED APPLICATIONS

This application claims priority to U.S. Provisional Patent Applications 63/323,633 filed on Mar. 25, 2023, and 63/323,635 filed on Mar. 25, 2023, the entire contents of each of which is hereby incorporated by reference.

### BACKGROUND

Targeted delivery of therapeutic modalities can be challenging, for example due to unwanted effects in cells or tissues that do not represent the intended site of therapeutic action.

### SUMMARY

The present disclosure encompasses a recognition that there is a need for effective delivery of therapeutic agents, including particularly to kidney cells. The present disclosure also encompasses a recognition that there is a particular need for effective delivery of nucleic acid therapeutic agents (e.g., oligonucleotide agents). Among other things, the present disclosure teaches that targeting an internalized receptor can provide a particularly useful approach for delivery of certain agents, in particular nucleic acid agents (e.g., oligonucleotide agents) into cells, specifically including kidney cells. The present disclosure further encompasses a recognition that nucleic acid agents may represent a particularly useful therapeutic modality for treatment of certain kidney diseases, disorders or conditions.

The present disclosure provides, among other things, technologies that achieve targeted delivery of therapeutic agents, and/or of nucleic acid agents. In some embodiments, provided compositions and technologies achieve delivery by targeting a cell surface factor (e.g., a cell surface receptor) that is internalized when bound by a targeting moiety (e.g., a megalin targeting moiety). In some embodiments, targeted delivery (e.g., megalin targeted delivery) in accordance with the present disclosure may be to kidney cells. In some embodiments, the present disclosure provides technologies particularly useful for delivery, for example to proximal tubule epithelial cells and/or to podocytes.

Among other things, the present disclosure appreciates that some of the challenges often associated with targeted delivery (e.g., megalin targeted delivery) is inefficient and/or insufficiently specific delivery; unwanted off-target effects; or effects in cells or tissues that do not represent the intended site of action which can be particularly problematic.

The present disclosure provides, among other things, conjugate agents comprising a targeting moiety (e.g., a megalin targeting moiety); directly or indirectly conjugated with a payload moiety. A targeting moiety as described herein binds specifically to a factor present on the surface of target cell(s) of interest—e.g., kidney-associated cells. In some embodiments, provided technologies achieve targeted delivery of payload moieties to a target cell, tissue, organ or organism of interest, for example with minimal off-target effects. In some embodiments, a targeting moiety as described herein (e.g., a megalin targeting moiety) binds specifically to a factor that is preferentially present on the surface of target cell(s) or tissue(s) of interest—e.g., relative to one or more non-target cell(s) or tissue(s). In some embodiments, a targeting moiety as described herein (e.g., a megalin targeting moiety) binds specifically to a factor that is specific to target cell(s) or tissue(s) of interest.

Among other things, the present disclosure provides an insight that targeting megalin and/or cubilin represents a particularly useful strategy for delivering certain agents, and specifically for delivering nucleic acid agents, into cells. The present disclosure provides a particular insight that targeting megalin and/or cubilin represents a particularly useful strategy for delivering certain agents, and specifically for delivery nucleic acid agents, into kidney-associated cells (e.g., kidney cells).

Moreover, the present specification specifically teaches that conjugate agents as described herein that include a megalin-binding moiety conjugated (optionally by way of a linker) with a nucleic acid agent are particularly useful for delivering such nucleic acid agent into megalin-expressing cells. The present specification particularly establishes usefulness of such conjugate agents in delivering nucleic acid agents to kidney cells.

In some embodiments, conjugate agents disclosed herein are characterized in that, for example, when they are provided to a relevant system (e.g., comprising one or more cell(s), tissue(s), organ(s), or organism(s)) they impact expression and/or activity of one or more targets or form(s) thereof, significantly more as compared to when the system is contacted with an unconjugated payload under otherwise comparable conditions.

This disclosure provides, among other things, a conjugate agent comprising: (i) a targeting moiety, directly or indirectly conjugated with (ii) a payload moiety. In some embodiments, a targeting moiety and payload moiety are indirectly conjugated by way of a linker. In some embodiments, a targeting moiety specifically binds a cell surface factor, e.g., a kidney cell surface factor. In some embodiments, a kidney cell surface factor is a receptor, e.g., Megalin and/or Cubilin.

In some embodiments, a kidney cell surface factor is internalized when bound by a targeting moiety.

In some embodiments, a targeting moiety is chosen from: a polypeptide, an aminoglycoside, an endogenous ligand (e.g., a ligand disclosed in Table 1), a xenobiotic, an antibody or a fragment thereof, an aptamer, a small molecule, or a combination thereof.

In some embodiments, a targeting moiety is or comprises an endogenous ligand, e.g., a ligand disclosed in Table 1. In some embodiments, a targeting moiety is or comprises a vitamin. In some embodiments, a vitamin is or comprises a vitamin provided in Table 1. In some embodiments, a vitamin is or comprises vitamin B12.

In some embodiments, a targeting moiety is or comprises a polypeptide. In some embodiments, a polypeptide is chosen from: a peptide having a KKEEE motif; a fragment of receptor associated protein (RAP), a peptide derived from a radiopharmaceutical conjugates such as ocreotide, ocreotate, exendin, minigastrin, and/or neurotensin; or a combination thereof.

In some embodiments, a polypeptide is or comprises a KKEEE motif. In some embodiments, a polypeptide comprises the sequence of SEQ ID NO:1.

In some embodiments, a polypeptide comprises a RAP fragment, or a variant thereof. In some embodiments, a RAP fragment comprises a polypeptide comprising residues 219-323 of RAP.

In some embodiments, a polypeptide is or comprises a peptide derived from a radiopharmaceutical conjugates such as ocreotide, ocreotate, exendin, minigastrin, and/or neurotensin.

In some embodiments, a polypeptide is or comprises a knotted peptide.

In some embodiments, a targeting moiety is or comprises an aminoglycoside. In some embodiments, a aminoglycoside is chosen from one or more, or all of: streptomycin, neomycin, kanamycin, paromomycin, gentamicin, G-418 (geneticin) ELX-202, tobramycin, amikacin, netilmicin, spectinomycin, sisomicin, dibekacin, isepamicin, framycetin, paromomycin, apramycin, fradiomycin, arbekacin, plazomicin, or a derivative, or a fragment, or a variant thereof.

In some embodiments, a targeting moiety is or comprises a xenobiotic. In some embodiments, a xenobiotic is or comprises polymixin, aprotinin, trichosanthin, or a combination thereof.

In some embodiments, a targeting moiety is or comprises an antibody of a fragment thereof. In some embodiments, an antibody or fragment thereof selectively binds Megalin, Cubilin, or both.

In some embodiments, an antibody or fragment thereof specifically binds Megalin.

In some embodiments, an antibody or fragment thereof specifically binds Cubilin.

In some embodiments, an antibody of fragment thereof is a bispecific antibody or a multi-specific antibody.

In some embodiments, an antibody comprises one or more modifications of an Fc domain, e.g., an Fc variant.

In some embodiments, a targeting moiety binds a cell surface receptor at one or more extracellular domains on a receptor. In some embodiments, a targeting moiety binds a receptor at or near one or more complement type repeat domains.

In some embodiments, a targeting moiety binds a cell surface receptor (e.g., a kidney cell surface factor) at a nephron apical membrane. In some embodiments, a targeting moiety binds a cell surface receptor (e.g., a kidney cell surface factor) at a nephron basolateral membrane.

In some embodiments, a payload moiety acts on a target chosen from a target provided in any one of Tables 2-5, or a combination thereof. In some embodiments, a payload moiety is or comprises a nucleic acid agent. In some embodiments, a nucleic acid agent is or comprises an antisense sequence element. In some embodiments, an antisense sequence element is complementary to at least a portion of one or more of: an exon, an intron, an untranslated region, a splice junction, a promoter region, an enhancer region, or a non-coding region in a target sequence.

In some embodiments, a nucleic acid agent comprises a sequence element that is at least 80% complementary to a target sequence in a sense strand. In some embodiments, a nucleic acid agent comprises a sequence element that is at least 80% complementary to a target sequence in an antisense strand.

In some embodiments, a nucleic acid agent comprises at least one sequence element with at least 3 contiguous nucleotides having at least 80% complementarity to a portion of a target sequence.

In some embodiments, a nucleic acid agent is single stranded. In some embodiments, a nucleic acid agent is double stranded.

In some embodiments, a nucleic acid agent has a length within a range of about 10-50 nucleotides, about 10-49 nucleotides, about 10-48 nucleotides, about 10-47 nucleotides, about 10-46 nucleotides, about 10-45 nucleotides, about 10-44 nucleotides, about 10-43 nucleotides, about 10-42 nucleotides, about 10-41 nucleotides, about 10-40 nucleotides, about 10-39 nucleotides, about 10-38 nucleotides, about 10-37 nucleotides, about 10-36 nucleotides, about 10-35 nucleotides, about 10-34 nucleotides, about 10-33 nucleotides, about 10-32 nucleotides, about 10-31 nucleotides, about 10-30 nucleotides, about 10-29 nucleotides, about 10-28 nucleotides, about 10-27 nucleotides, about 10-26 nucleotides, about 10-25 nucleotides, about 10-24 nucleotides, about 10-23 nucleotides, about 10-22 nucleotides, about 10-21 nucleotides, about 10-20 nucleotides, about 10-19 nucleotides, about 10-18 nucleotides, about 10-17 nucleotides, about 10-16 nucleotides, about 10-15 nucleotides, about 10-14 nucleotides, about 10-13 nucleotides, about 10-12 nucleotides, about 10-11 nucleotides. In some embodiments, a nucleic acid has a length within a range of about 11-50 nucleotides, about 12-50 nucleotides, about 13-50 nucleotides, about 14-50 nucleotides, about 15-50 nucleotides, about 16-50 nucleotides, about 17-50 nucleotides, about 18-50 nucleotides, about 19-50 nucleotides, about 20-50 nucleotides, about 21-50 nucleotides, about 22-50 nucleotides, about 23-50 nucleotides, about 24-50 nucleotides, about 25-50 nucleotides, about 26-50 nucleotides, about 27-50 nucleotides, about 28-50 nucleotides, about 29-50 nucleotides, about 30-50 nucleotides, about 31-50 nucleotides, about 32-50 nucleotides, about 33-50 nucleotides, about 34-50 nucleotides, about 35-50 nucleotides, about 36-50 nucleotides, about 37-50 nucleotides, about 38-50 nucleotides, about 39-50 nucleotides, about 40-50 nucleotides, about 41-50 nucleotides, about 42-50 nucleotides, about 43-50 nucleotides, about 44-50 nucleotides, about 45-50 nucleotides, about 46-50 nucleotides, about 47-50 nucleotides, about 48-50 nucleotides, about 49-50 nucleotides.

In some embodiments, a nucleic acid agent is about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides in length.

In some embodiments, a nucleic acid agent is or comprises RNA.

In some embodiments, a nucleic acid agent is or comprises DNA.

In some embodiments, a nucleic acid agent comprises DNA residues and/or RNA residues.

In some embodiments, a nucleic acid agent comprises DNA analogs and/or RNA analogs. In some embodiments, a nucleic acid agent comprises one or more morpholino subunits linked together by phosphorus-containing linkage.

In some embodiments, a nucleic acid agent is or comprises an interfering RNA (RNAi) agent. In some embodiments, an RNA is or comprises a short interfering RNA (siRNA) agent. In some embodiments, an RNA is or comprises a micro RNA (miRNA) agent. In some embodiments, a nucleic acid agent is or comprises a guide RNA (gRNA) agent.

In some embodiments, a nucleic acid agent is or comprises an exon skipping agent, an exon inclusion agent, or other splicing modulator.

In some embodiments, a nucleic acid agent is or comprises an aptamer agent.

In some embodiments, a nucleic acid agent is or comprises an antisense oligo (ASO). In some embodiments, an ASO modulates gene expression via RNase H mediated mechanisms. In some embodiments, an ASO modulates gene expression via steric hindrance.

In some embodiments, a nucleic acid agent is or comprises a phosphorodiamidate morpholino oligonucleotide (PMO).

In some embodiments, a nucleic acid agent is or comprises a peptide-nucleic acid (PNA).

In some embodiments, a nucleic acid agent comprises one or more modifications. In some embodiments, a nucleic acid agent comprises a modification comprising: a modified backbone, a modified nucleobase, a modified ribose, a modified deoxyribose, or a combination thereof. In some embodiments, a modification is chosen from: a 2′-O-methyl modified nucleotide, a 5-methylcytidine, a 5-methyluridine, a nucleotide comprising a 5′-phosphorothioate group, a morpholino nucleotide (e.g., a PMO), or a terminal nucleotide linked to a cholesteryl derivative or a dodecanoic acid bisdecylamide group, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide (e.g., PMO), a phosphoramidate, a phosphoryl guanidine (PN), or a non-natural base comprising nucleotide, or a combination thereof. In some embodiments, a modification is chosen from: a C7-modified deaza-adenine, a C7-modified deaza-guanosine, a C5-modified cytosine, a C5-modified uridine, N1-methyl-pseudouridine (mlw), 1-ethyl-pseudouridine (elw), 5-methoxy-uridine (mo5U), 5-methyl-cytidine (m5C), pseudouridine (w), 5-methoxymethyl uridine, 5-methylthio uridine, 1-methoxymethyl pseudouridine, 5-methyl cytidine, 5-methoxy cytidine, or a combination thereof. In some embodiments, a modification is chosen from: a 2′fluoro modification, a 2′-O-methyl (2′OMe) modification, a locked nucleic acid (LNA), a 2′-fluoro arabinose nucleic acid (FANA), a hexitol nucleic acid (HNA), a 2′O-methoxyethyl (2′MOE) modification, or a combination thereof.

In some embodiments, a modification is chosen from: a phosphorothioate (PS) modification, a borano-phosphate modification, an alkyl phosphonate nucleic acid (phNA), a peptide nucleic acid (PNA), a phosphoryl guanidine (PN) modification, or a combination thereof.

In some embodiments, a nucleic acid agent comprises one or more modification to a 5′ end of a nucleic acid. In some embodiments, a nucleic acid agent comprise a 5′ amino modification.

In some embodiments, a nucleic acid agent or a conjugate agent comprising the same is characterized in that when delivered to a cell expressing the target, reduced expression and/or activity of a target is observed as compared to a cell which has not been delivered a nucleic acid agent or a cell which does not express a target.

In some embodiments of a conjugate disclosed herein, a linker is a cleavable linker. In some embodiments, a linker becomes cleaved when exposed to a cell-internal environment.

In some embodiments, a targeting moiety and a payload moiety are conjugated by a linker comprising the structure:

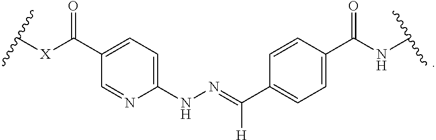

wherein X is NH or O.

In some embodiments, a targeting moiety and a payload moiety are conjugated by a linker comprising the structure:

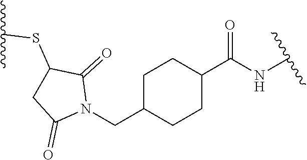 [SMILES: *NC(=O)C1CCC(CN2C(=O)CC(S*)C2=O)CC1]

Also disclosed herein is a conjugate agent comprising: (i) a targeting moiety; directly or indirectly conjugated with (ii) a payload moiety comprising a nucleic acid agent that targets a target which is present: in a cell in which a cell surface factor is present. In some embodiments, a targeting moiety comprises a kidney-specific targeting moiety.

In some embodiments, a conjugate agent is characterized in that when delivered to a cell, tissue or organism, a payload moiety is delivered to, and/or expressed in, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, more target cells compared to an otherwise similar cell, tissue or organism delivered an unconjugated payload moiety.

In some embodiments, a conjugate agent is characterized in that when delivered to a tissue or organism, a payload moiety is delivered to, and/or expressed in, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, more target cells compared to non-target cells.

In some embodiments, a conjugate agent is characterized in that when delivered to a cell, tissue or organism, expression and/or activity of a target of a payload moiety is modulated, e.g., reduced, by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, compared to an otherwise similar cell, tissue or organism delivered an unconjugated payload moiety.

In some embodiments, this disclosure provides a conjugate agent comprising: (i) a targeting moiety specific for an internalizing cell surface factor; and (ii) a payload moiety comprising a nucleic acid agent, wherein the binding moiety and nucleic acid agent are conjugated to one another by way of a cleavable linker so that the conjugate agent is in a first, associated state, when extracellular to a kidney cell and a second, disassociated state, when internal to a cell in which a cell surface factor is present.

This disclosure provides, among other things, a conjugate comprising the structure of Formula I:

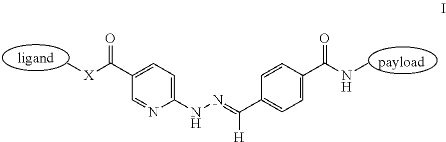

wherein X is NH or O; the ligand is a targeting moiety; and the payload is a payload moiety.

Also provided herein is a conjugate comprising the structure of Formula II:

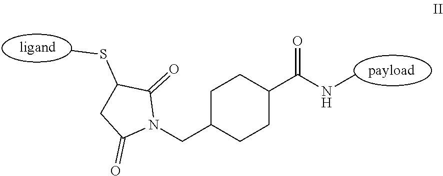

wherein the ligand is a targeting moiety; and the payload is a payload moiety.

Further provided herein is a conjugate comprising the structure of Formula III:

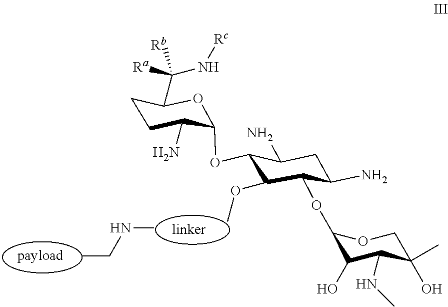

wherein each of R^{a}, R^{b}, and R^{c} is selected from H and CH3;
wherein the linker is a bivalent linker; and a payload is a payload moiety.

In some embodiments, provided herein is a conjugate comprising the structure of Formula IV:

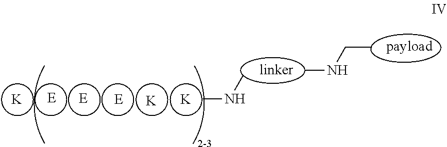

wherein the linker is a bivalent linker; and the payload is a payload moiety.

Also provided herein is a conjugate comprising the structure of Formula VI:

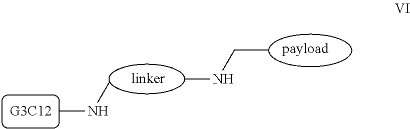

wherein the linker is a bivalent linker; and the payload is a payload moiety.

This disclosure also provides a pharmaceutical composition that comprises or delivers a conjugate agent disclosed herein. In some embodiments, a pharmaceutical composition is formulated for intravenous, subcutaneous, intramuscular, parenteral, or oral delivery. In some embodiments, a pharmaceutical composition comprises one or more pharmaceutically or physiologically acceptable carriers, diluents, or excipients. In some embodiments, a pharmaceutical composition comprises less than 5% of an impurity. In some embodiments, an impurity comprises one or more of: an endotoxin, a cellular component, or an aggregate.

In some embodiments, provided herein is a cell comprising a conjugate agent disclosed herein. In some embodiments, a cell is in a tissue, an organ, or an organism.

This disclosure provides a payload moiety comprising a nucleic acid agent recognizing a target, linked to a cleaved first portion of a linker. In some embodiments, a payload moiety is in a cell in which a cell surface factor is present. In some embodiments, a cell further comprises a targeting moiety linked to a cleaved second portion of the linker.

Provided herein, is a method of delivering a conjugate agent to a subject, the method comprising a step of: administering to a subject, a conjugate agent comprising a targeting moiety directly or indirectly linked with a payload moiety, or a pharmaceutical composition comprising the same.

Also disclosed herein is a method of treating a disease or disorder, the method comprising a step of: administering to a subject, a conjugate agent comprising a targeting moiety directly or indirectly linked with a payload moiety, or a pharmaceutical composition comprising the same.

Further disclosed herein is a method of treating a disease with a nucleic acid agent, the improvement comprising a step of: administering a nucleic acid agent as a conjugate with a targeting moiety, e.g., as disclosed herein.

In some embodiments, the disclosure provides improving delivery of an agent to a cell, the method comprising contacting a system or subject comprising at least one cell with a conjugate agent disclosed herein or a pharmaceutical composition comprising the same.

In some embodiments of any of the methods disclosed herein, a conjugate agent is delivered to a cell expressing a cell surface factor. In some embodiments, a cell surface factor is a kidney cell surface factor. In some embodiments, a kidney cell surface factor is chosen from megalin and/or cubilin.

In some embodiments of any of the methods disclosed herein, a conjugate agent is delivered to a tissue, organ, or fluid compartment.

In some embodiments of any of the methods, conjugate agents, compositions, or

cells disclosed herein, a conjugate agent is internalized upon binding to a cell surface factor. In some embodiments, internalization of a conjugate agent delivers a payload moiety into an internal compartment of, or a vesicle in a cell.

In some embodiments of any of the methods, conjugate agents, compositions, or cells disclosed herein, a payload reduces expression and/or activity of a target provided in any one of Tables 2-5, or a combination thereof.

In some embodiments of any of the methods disclosed herein, contacting comprises administering a conjugate agent to: a cell; a tissue comprising a cell; or an organism comprising a cell.

In some embodiments of any of the methods disclosed herein, administering a conjugate agent to a cell, tissue or organism, delivers a payload moiety to at least 5% more, at least 10% more, 15% more, at least 20% more, at least 25% more, at least 30% more, at least 35% more, at least 40% more, at least 45% more, at least 50% more, at least 55% more, at least 60% more, at least 65% more, at least 70% more, at least 75% more, at least 80% more, at least 85% more, at least 90% more, at least 95% more, or at least 99% more target cells compared to an otherwise similar cell, tissue or organism delivered an unconjugated payload moiety.

In some embodiments of any of the methods disclosed herein, administering a conjugate agent to a cell, tissue or organism, delivers a payload moiety to at least 5% more, at least 10% more, 15% more, at least 20% more, at least 25% more, at least 30% more, at least 35% more, at least 40% more, at least 45% more, at least 50% more, at least 55% more, at least 60% more, at least 65% more, at least 70% more, at least 75% more, at least 80% more, at least 85% more, at least 90% more, at least 95% more, or at least 99% more target cells compared to non-target cells.

In some embodiments of any of the methods, conjugate agents, compositions or cells disclosed herein, a target cell is or comprises a kidney cell.

In some embodiments of any of the methods, conjugate agents, compositions or cells disclosed herein, a target cell is or comprises a cell that has expression of (e.g., detectable expression of) a cell surface factor. In some embodiments, a cell surface factor is or comprises a kidney cell surface factor. In some embodiments, a kidney cell surface factor is Megalin, or a variant or a fragment thereof. In some embodiments, a kidney cell surface factor is Cubilin, or a variant or a fragment thereof.

In some embodiments of any of the methods, conjugate agents, compositions or cells disclosed herein, a target cell is or comprises expresses of one or more targets chosen from: a target provided in any one of Tables 2-5.

In some embodiments of any of the methods, conjugate agents, compositions or cells disclosed herein, a non-target cell is or comprises a cell that has no expression of (e.g., no detectable expression of) a cell surface factor. In some embodiments, a non-target cell is or comprises a cell that does not express (e.g., has no detectable expression of) a kidney cell surface factor (e.g., Megalin and/or Cubilin).

In some embodiments of any of the methods disclosed herein, administering a conjugate agent to a cell, tissue or organism, reduces expression and/or activity of a target of the a moiety by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, compared to an otherwise similar cell, tissue or organism delivered an unconjugated payload moiety.

In some embodiments of any of the methods disclosed herein, a conjugate agent is delivered to a cell expressing a cell surface factor, e.g., as described herein. In some embodiments, a cell surface factor is chosen from: Megalin and/or Cubilin.

In some embodiments, a cell is chosen from: immune cells; nervous system cells; muscle cells; small intestine cells; colon cells; adipocytes; kidney cells; liver cells; lung cells; splenic cells; stomach cells; esophagus cells; bladder cells; pancreas cells; thyroid cells; salivary gland cells; adrenal gland cells; pituitary gland cells; breast cells; skin cells; ovary cells; uterus cells; placenta cells; prostate cells; or testis cells, or a combination thereof.

In some embodiments, a cell is chosen from: renal cells, thyroid cells, parathyroid cells, cells of the inner ear, or nervous system cells, or a combination thereof.

In some embodiments, a cell is chosen from: proximal tubular epithelial cell and/or a podocyte.

In some embodiments of any of the methods disclosed herein, a disease is a disease associated with expression of a cell surface receptor. In some embodiments, disease is a disease comprising a cell in which both a cell surface receptor and a target recognized by the payload moiety are present.

In some embodiments of any of the methods disclosed herein, a disease or disorder is chosen from: a glomerular disorder, a renal tubular disorder, other renal disorders, an inborn error of metabolism, a systemic metabolic disorder, a disorder of the thyroid, a disorder of the parathyroid, a disorder of the inner ear, a neurological disorder, a viral infection, or a combination thereof.

In some embodiments of any of the methods disclosed herein, a conjugate agent is delivered intravenously, subcutaneously, intramuscularly, parenterally or orally.

In some embodiments of any of the methods disclosed herein, a conjugate agent is delivered in one or more doses.

In some embodiments of any of the methods disclosed herein, a conjugate agent is delivered in combination with one or more additional conjugate agents. In some embodiments, one or more additional conjugate agents comprises a different payload moiety, a different linker, a different targeting moiety, or a combination thereof.

In some embodiments of any of the methods disclosed herein, a conjugate agent is delivered in combination with one or more additional therapeutic modalities.

### BRIEF DESCRIPTION OF THE DRAWING

FIGs: 1A-1B show endogenous levels of Megalin in HEK293 cells, HK2 cells and Primary Renal Proximal Tubule Epithelial Cells (RPTEC). FIG. 1A is a graph depicting RT-qPCR of HEK293, HK2 and RPTEC cells (n=3). HMBS is used as housekeeping gene control.

FIG. 1B shows protein expression of Megalin by immunoblotting using β-actin as a loading control.

FIGS. 2A-2C show GTTR internalization is temperature, time and concentration dependent. FIG. 2A is a panel of immunofluorescence of GTTR at 37° C. or at 4° C. FIG. 2B is a panel of immunofluorescence of GTTR at 1, 4, 24 and 28 hours for HEK293 cells incubated with 0, 0.125, 0.25, 0.5, 1 μg/ml of GTTR per well. FIG. 2C is a graph quantifying the data from FIG. 2B. The quantified data was plotted using GraphPad Prism.

FIGS. 3A-3B show GTTR internalization is Megalin-dependent. FIG. 3A is an immunoblot confirming siRNA mediated knockdown of Megalin in HEK293 cells using three concentrations of Megalin or control siRNA: 0.5 uM, 1 uM and 3 uM. FIG. 3B is a panel of immunofluorescence demonstrating the dependence of GTTR uptake on expression of Megalin protein, n=3 (representative images shown). The panel on the left is a panel of cells treated with a control siRNA, the panel in the middle shows cells treated with 0.5 uM Megalin siRNA and the panel on the right shows cells treated with 1 uM Megalin siRNA. All cells were subsequently incubated with GTTR and imaged.

FIG. 4 shows a panel of images from a Western blot showing expression of LRP2 (Megalin) in Megalin knockout cell line clones #45, #49 and #50 compared to controls, as described in Example 6.

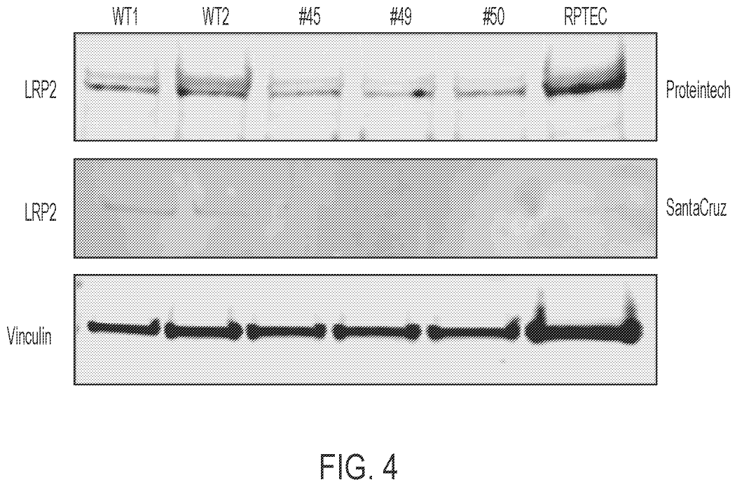

FIGS. 5A-5B depict GTTR update in Megalin knockout (KO) cell line clones. FIG. 5A is a graph showing GTTR uptake at 37C in Megalin KO cell line clone #45 compared to a control cell line. FIG. 5B are representative images of GTTR internalization.

FIG. 6 depicts the effects of Endoporter, and endosomal release agent, in increasing the potency of HPRT PMO in reducing the HPRT expression.

FIGS. 7A-7B depict the effects of conjugated PMOs on expression of HPRT.

FIG. 8 is a graph showing HPRT expression in cells treated with conjugated HPRT siRNA or with control siRNA. Cells were transfected with HPRT siRNA in the presence or absence of RNAiMax

FIG. 9 is a graph showing HPRT expression in cells treated with conjugated HPRT siRNA, or unconjugated control siRNA. Cells were transfected with siRNA in the presence or absence of RNAiMax or chloroquine (CQ).

FIG. 10 is a graph showing concentration in the kidney of gentamicin-conjugated or unconjugated siRNA targeting an exemplary mouse target (muExemplary Target 1) at 0, 0,25, 0,5, 1, 2, 4, 8, 16, 24, 48 or 72 hours after administration.

FIGS. 11A-11B are bar graphs showing expression of the target gene and control genes (Actb, GAPDH, and PPIA) in kidney cells of mice administered gentamicin conjugated siRNA (FIG. 11A) or unconjugated siRNA (FIG. 11B).

FIG. 12 is a graph showing concentration in the kidney of gentamicin-conjugated or unconjugated siRNA targeting an exemplary mouse target (muExemplary Target 1) at 0,25, 0,5, 4, 8, or 24 hours after administration.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Aminoglycoside. The term “aminoglycoside” as used herein refers to a compound having a core structure that comprises 2-deoxystreptamine:

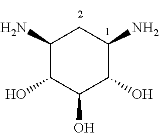 [SMILES: N[C@@H]1C[C@H](N)[C@@H](O)[C@H](O)[C@H]1O]

It will be understood that 2-deoxystreptamine can be attached to other moieties via any available position, as valency rules permit. For example, gentamicin is a compound that comprises a 2-deoxystreptamine core.

Megalin. The term “Megalin,” as used herein refers to a receptor which is a member of the low-density lipoprotein receptor (LDLR) family. Megalin is encoded by the LRP2 gene. Amino acid sequences for full length Megalin, and/or for nucleic acids that encode it can be found in a public database such as GenBank, UniProt and Swiss-Prot. For example, the amino acid sequence of human Megalin (SEQ ID NO:3, for which residues 27-4411 represent an extracellular domain comprising LDL Receptor Class A domains, LDL Receptor Class B domains, and EGF-like domains; residues 4589-4602 represent a DAB2 interaction domain; and residues 4453-4622 represent a cytoplasmic domain comprising NPXY motifs, SH2 binding domains, SH3 binding domains, and proline-rich domains) can be found as UniProt/Swiss-Prot Accession No. P98164 and the nucleic acid sequence (SEQ ID NO: 4) encoding human Megalin can be found at Accession No. NM_004525.3. Megalin is also known, for example, as as Low-density lipoprotein receptor-related protein 2 (LRP2), Glycoprotein 330 (Gp330), Calcium Sensor Protein, or Heymann Nephritis Antigen Homolog. Those skilled in the art will appreciate that sequences presented in SEQ ID NOsNumbers: 3 and 4 are exemplary, and certain variations (including, for example, conservative substitutions in SEQ ID NO:3, codon-optimized variants of SEQ ID NO:4, etc) are understood to also be or encode human Megalin; additionally, those skilled in the art will appreciate that homologs and orthologs of human Megalin are known and/or knowable through the exercise or ordinary skill, for example, based on degree of sequence identity, presence of one or more characteristic sequence elements, and/or one or more shared activities. In some embodiments, Megalin comprises full-length Megalin, or a variant or a fragment thereof. In some embodiments, Megalin that is targeted in accordance with the present disclosure is a Megalin expressed by particular target cell(s) and/or tissue(s) of interest (e.g., in an organism of interest). In some embodiments, a Megalin that is targeted in accordance with the present disclosure is an engineered Megalin. In many embodiments, a Megalin that is targeted in accordance with the present disclosure is present on the surface of target cell(s) of interest (e.g., in target tissue(s) of interest) and that becomes internalized by such cell upon binding of a Megalin binding moiety as described herein. Megalin has been reported to be expressed in one or more of the following tissues and/or cells:immune cells (e.g., bone marrow cells, lymph node cells, thymic cells, peripheral blood mononuclear cells [e.g., myeloid and/or lymphoid cells], erythrocytes, eosinophils, neutrophils, and/or platelets); nervous system cells (e.g., brain tissue, cortex, cerebellum, retinal cells, spinal cord cells, nerve cells, neurons, and/or supporting cells); endothelial cells; muscle (e.g., heart muscle, smooth muscle, and/or skeletal muscle); small intestine; colon; adipocytes; kidney; liver; lung; spleen; stomach; esophagus; bladder; pancreas; thyroid; salivary gland; adrenal gland; pituitary gland; breast; skin; ovary; uterus; placenta; prostate; and testis. Megalin expression is reported to be enriched (e.g., high relative to one or more other tissues) in the following tissues and/or cells in particular: renal tissue, thyroid tissue, parathyroid tissue, cells of the inner ear, and nervous system tissue. Megalin has been specifically reported to be expressed (e.g., at relatively high level(s)) on surfaces of kidney cells such a proximal tubular epithelial cells and podocytes. See Nielsen R. et al. (2016), Kidney Int. 89 (1): 58-67.

Megalin-binding moiety. The term “Megalin-binding moiety” as used herein refers to a moiety that binds to Megalin when contacted therewith. Typically, a Megalin-binding moiety useful in accordance with the present disclosure binds specifically to Megalin under the circumstances of the contacting. In some embodiments, a Megalin-binding moiety is or comprises: a peptide, an aminoglycoside, an endogenous ligand (e.g., a ligand disclosed in Table 1 or an analog or variant thereof), a xenobiotic, an antibody or a fragment thereof, or a combination thereof. In some embodiments, a Megalin-binding moiety is internalized upon binding to Megalin on a cell surface.

Cubilin. The term “Cubilin,” as used herein refers to a receptor encoded by the CUBN gene. Amino acid sequences for full length Cubilin, and/or for nucleic acids that encode it can be found in a public database such as GenBank, UniProt and Swiss-Prot. For example, the amino acid sequence of human Cubilin (SEQ ID NO:5, for which residues 1-23 represent a signal peptide; residues 24-35 represent a propeptide which can be removed in the mature form, and residues 36-3623 represent a mature Cubilin polypeptide) can be found as UniProt/Swiss-Prot Accession No. 060494 and the nucleic acid sequence (SEQ ID NO: 6) encoding human Cubilin can be found at Accession No. NM_001081.3. Cubilin is also known, for example, known as IFCR, Gp280, Intrinsic Factor-Vitamin B12 Receptor, MGA1, or IGS1. Those skilled in the art will appreciate that sequences presented in SEQ ID Numbers: 5 and 6 are exemplary, and certain variations (including, for example, conservative substitutions in SEQ ID NO:5, codon-optimized variants of SEQ ID NO:6, etc) are understood to also be or encode human Cubilin; additionally, those skilled in the art will appreciate that homologs and orthologs of human Cubilin are known and/or knowable through the exercise or ordinary skill, for example, based on degree of sequence identity, presence of one or more characteristic sequence elements, and/or one or more shared activities. In some embodiments, Cubilin comprises full-length Cubilin, or a variant or a fragment thereof. In some embodiments, Cubilin that is targeted in accordance with the present disclosure is a Cubilin expressed by particular target cell(s) and/or tissue(s) of interest (e.g., in an organism of interest). In some embodiments, a Cubilin that is targeted in accordance with the present disclosure is an engineered Cubilin. In many embodiments, a Cubilin that is targeted in accordance with the present disclosure is present on the surface of target cell(s) of interest (e.g., in target tissue(s) of interest) and that becomes internalized by such cell upon binding of a Cubilin binding moiety as described herein. Cubilin has been reported to be expressed in one or more of the following tissues and/or cells:immune cells (e.g., bone marrow cells, lymph node cells, thymic cells, peripheral blood mononuclear cells [e.g., myeloid and/or lymphoid cells], erythrocytes, eosinophils, neutrophils, and/or platelets); nervous system (e.g., brain tissue, cortex, cerebellum, retinal cells, spinal cord cells, nerve cells, neurons, and/or supporting cells; endothelial cells; muscle (e.g., heart muscle, smooth muscle, and/or skeletal muscle); small intestine; colon; adipocytes; kidney; liver; lung; spleen; stomach; esophagus; bladder; pancreas; thyroid; salivary gland; adrenal gland; pituitary gland; breast; skin; ovary; uterus; placenta; prostate; and testis. Cubilin expression is reported to be enriched (e.g., high relative to one or more other tissues) in the following tissues and/or cells in particular: renal tissue, thyroid tissue, parathyroid tissue, cells of the inner ear, and nervous system tissue. Cubilin has been specifically reported to be expressed (e.g., at relatively high level(s)) on surfaces of kidney cells such a proximal tubular epithelial cells and podocytes. See Nielsen R. et al. (2016), Kidney Int. 89 (1): 58-67.

Cubilin-binding moiety. The term “Cubilin-binding moiety” as used herein refers to a moiety that binds to Cubilin when contacted therewith. Typically, a Cubilin-binding moiety useful in accordance with the present disclosure binds specifically to Cubilin under the circumstances of the contacting. In some embodiments, a Cubilin-binding moiety is or comprises a ligand provided in Table 6. In some embodiments, a Cubilin-binding moiety is internalized upon binding to Cubilin on a cell surface.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” refers to the administration of a composition (e.g., a compound [e.g., a conjugate] as described herein or a preparation that includes or otherwise delivers such compound) to a subject or system, or to a cell or tissue thereof. Administration to an animal subject (e.g., to a human) can be by an appropriate route, such as one described herein. In some embodiments, administration may be local. In some embodiments, administration may be systemic. In some embodiments, administration may be enteral. In many embodiments, administration may be parenteral. In some particular embodiments, parenteral administration may be intravenous, subcutaneous, intramuscular, intradermal, etc.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological-setting). In some embodiments, affinity is assessed relative to a reference (e.g., that has a known affinity above a particular threshold [a “positive control” reference] or that has a known affinity below a particular threshold [a “negative control” reference “]. In some embodiments, affinity may be assessed relative to a contemporaneous reference; in some embodiments, affinity may be assessed relative to a historical reference. Typically, when affinity is assessed relative to a reference, it is assessed under comparable conditions.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: in its broadest sense, as used herein, refers to any compound and/or substance that can be incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. In some embodiments, antibodies produced and/or utilized in accordance with the present disclosure include one or more modifications on an Fc domain. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of dog, cat, mouse, rabbit, primate, or human antibodies. In some embodiments, antibody sequence elements are human, humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies, alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies, etc. In some embodiments, relevant formats may be or include: Adnectins®; Affibodies®; Affilins®; Anticalins®; Avimers®; BiTERs; cameloid antibodies; Centyrins®, ankyrin repeat proteins or DARPINS®; dual-affinity re-targeting (DART) agents; Fynomers®; shark single domain antibodies such as IgNAR; immune mobilixing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™ ”); single chain or Tandem diabodies (TandAb®); TCR-like antibodies;, Trans-bodies®; TrimerX®; VHHs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated: Two events or entities are “associated” with one another, as that term is used herein, if the presence, level, degree, type and/or form of one is correlated with that of the other. For example, a particular entity (e.g., polypeptide, etc) is considered to be associated with a particular cell type (e.g., kidney cell) or a particular disease, disorder, or condition, if its presence, level and/or form correlates with identity of such cell type or with incidence of, susceptibility to, severity of, stage of, etc such disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: Those skilled in the art will appreciate that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell). Binding between two entities may be considered “specific” if, under the conditions assessed, the relevant entities are more likely to associate with one another than with other available binding partners.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

“Cell associated with a kidney” or “kidney cell”. The phrase “cell associated with a kidney” as used herein refers to a cell that is or can be found in a kidney (e.g., during development, during tissue homeostasis, or in the course of a disease or disorder). In some embodiments, a cell associated with a kidney is also referred to as a kidney cell herein. In some embodiments, a cell associated with a kidney includes any one or all of the following cell types: a proximal tubule epithelial cell, a podocyte, a parietal epithelial cell, a mesangial cell, a renal stem cell, an epithelial progenitor cell, a fibroblast, a myo-fibroblast, a pericyte, an ascending loop of Henle cell, a descending loop of Henle cell, a distal tubule cell, a connecting tubule cell, an intercalated cell, a principal cell. Exemplary renal cell populations are provided in Schumacher A. et a., (2021) npj Regen Med 6, 45, the entire contents of which are hereby incorporated by reference. In some embodiments, a kidney cell is or comprises a cell derived from a kidney, e.g., a kidney tumor cell and/or a metastatic kidney tumor cell.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Conservative: As used herein, the term “conservative” refers to instances describing a conservative amino acid substitution, including a substitution of an amino acid residue by another amino acid residue having a side chain R group with similar structural, chemical (e.g., charge or hydrophobicity), and/or functional properties. In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet et al., Science 256:1443, 1992, which is incorporated herein by reference in its entirety. In some embodiments, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein. In some embodiments, a “conservative” substitution is considered a “homologous” residue for purposes of calculating percent homology between amino acid sequences.

EXEMPLARY CONSERVATIVE AMINO ACID SUBSTITUTIONS

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, Met, Ile, D-Met, |
| | | D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, Met, D-Met |
| Leucine | L | D-Leu, Val, D-Val, AdaA, AdaG, Leu, D-Leu, Met, D-Met |
| Lysine | K | D-Lys, Arg, D-Arg, homo-Arg, D-homo-Arg, Met, D-Met, Ile, |
| | | D-Ile, Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, Trans-3,4 or |
| | | 5-phenylproline, AdaA, AdaG, cis-3,4 or 5-phenylproline, Bpa, |
| | | D-Bpa |
| Proline | P | D-Pro, L-I-thioazolidine-4-carboxylic acid, D-or-L-1-oxazolidine- |
| | | 4-carboxylic acid (Kauer, U.S. Pat. No. 4,511,390) |
| Serine | S | D-Ser, Thr, D-Thr, allo-Thr, Met, D-Met, Met (O), D-Met (O), L- |
| | | Cys, D-Cys |
| Threonine | T | D-Thr, Ser, D-Ser, allo-Thr, Met, D-Met, Met (O), D-Met (O), |
| | | Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, AdaA, AdaG |

Conjugate agent. The term “conjugate agent” as used herein refers to an agent that is or comprises a targeting moiety directly or indirectly conjugated with a payload moiety. In some embodiments, a targeting moiety is a kidney-specific binding moiety. In some embodiments, a conjugate agent has a structure represented by the following formula: (Xn1-Yn2-Zn3), wherein X is a targeting moiety and n1 is an integer (i.e. 1 or greater, typically less than 5); Y is a linker and n2 is 0 or an integer (i.e., 1 or greater, typically less than 5); and Z is a payload moiety and n3 is an integer (i.e. 1 or greater, typically less than 5); in many embodiments, n2=n1 and/or n2=n3. In many embodiments, n1 and/or n3 is/are 1. In many embodiments, a conjugate agent has a structure represented by the formula (X—Y—Z). In some embodiments, a conjugate agent has a structure represented by a formula of: (X—Y)n—Z, wherein n is an integer greater than 1, and a conjugate agent comprises more than one targeting moiety. In some embodiments, a conjugate agent has structure represented by a formula of: X—(Y—Z)n, wherein n is an integer greater than 1, and a conjugate agent comprises more than one payload moiety.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Designed: As used herein, the term “designed” refers to an agent (i) whose structure is or was selected by the hand of man; (ii) that is produced by a process requiring the hand of man; and/or (iii) that is distinct from natural substances and other known agents.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature. For example, in some embodiments described abd.ir utilized herein, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Functional: As used herein, a “functional” biological molecule is a biological molecule in a form in which it exhibits a property and/or activity by which it is characterized.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Human: In some embodiments, a human is an embryo, a fetus, an infant, a child, a teenager, an adult, or a senior citizen.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

K_{D}: as used herein, refers to the dissociation constant of a binding agent from a complex with its partner.

Targeting moiety: The term “targeting moiety” as used herein, refers to a moiety that, when contacted with a system that includes one or more target cells of interest cells (e.g., in culture, in a tissue, and/or in an organism) binds specifically with such target cells. In many embodiments, a targeting moiety binds to a cell surface factor (e.g., to a factor that is preferentially or specifically found on surface(s) of such target cells of interest). In some embodiments, binding of a targeting moiety to a cell surface factor results in internalization of a targeting moiety. Typically, a targeting moiety useful in accordance with the present disclosure retains its specific binding character when included in a conjugate agent as described herein; in some embodiments, binding of such a conjugate agent to a relevant cell surface factor results in internalization of a conjugate agent. In some embodiments, a targeting moiety binds specifically to a factor on the surface of kidney cells. In some embodiments, a targeting moiety binds specifically to cubilin. In some embodiments, a targeting moiety binds specifically to megalin.

Cell surface factor. The term “cell surface factor” as used herein, refers to a factor (e.g., that is or comprises a polypeptide) that is present on the surface of cell(s) of interest (e.g., of target cell(s) as described herein which, in many embodiments, may be kidney cells). In some embodiments, a cell surface factor is preferentially present on the surface of target cell(s) (e.g., kidney cells) as compared with cells of one or more other tissues. In some embodiments, a cell surface factor is present on certain non-target cells in addition to target cells. In some embodiments, a cell surface factor is not preferentially or specifically present on relevant target cells of interest. In some embodiments, a cell surface factor is or comprises a receptor. In some embodiments, a cell surface factor is internalized when bound by one or more particular ligands (e.g., with a targeting moiety as described herein). In some embodiments, a cell surface factor may interact with (e.g., bind to, form a complex with, etc) one or more other components of a cell (e.g., with one or more cell membrane components and/or one or more cell surface components and/or one or more cell-internal components) on whose surface it is found. In some embodiments, a cell surface factor, and/or a particular form or variant thereof, and/or a cell surface factor of any of the foregoing, may be associated with a particular cell state or condition (e.g., stage of development, disease state, etc).

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in a particular form (e.g., in a solid form or a liquid form), and/or may be specifically adapted for, for example: oral administration (for example, as a drenche [aqueous or non-aqueous solutions or suspensions], tablet, capsule, bolus, powder, granule, paste, etc, which may be formulated specifically for example for buccal, sublingual, or systemic absorption); parenteral administration (for example, by subcutaneous, intramuscular, intravenous or epidural injection as, for example, a sterile solution or suspension, or sustained-release formulation, etc); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specificity: As is known in the art, “specificity” is a measure of the ability of a particular ligand to distinguish its binding partner from other potential binding partners.

Subject As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject is suffering from a disease, disorder or condition, e.g., a disease, disorder or condition that can be treated as provided herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g,. clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial sequence identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215 (3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Treat: As used herein, the terms “treat,” “treated,” and “treating” refer to delaying onset of and/or reducing severity and/or frequency of one or more undesired physiological events or states (e.g., which may be indicative of a particular condition, disorder, or disease) and/or achieving a particular beneficial or desired physiological or result(s) and/or administration of a regimen or therapy demonstrated or reasonably expected to accomplish such delaying, reducing or achieving. In some embodiments, a beneficial or desired clinical results include, but are not limited to, alleviation of one or more symptoms; diminishment of the extent of a condition, disorder, or disease; stabilization (e.g.,., not worsening) of a state of a condition, disorder, or disease; delay in onset or slowing of progression of a condition, disorder, or disease; amelioration of the condition, disorder, or disease state, remission (whether partial or total), whether detectable or undetectable; an amelioration of at least one measurable physical parameter, not necessarily discernible by the patient; enhancement or improvement of condition, disorder, or disease, etc. In some embodiments, treatment may involve eliciting a clinically significant response without excessive side effects. In some embodiments, treatment may be or comprise prolonging survival as compared to an expected survival if not receiving treatment.

Variant: The term “variant”, as used herein, refers to a molecule or entity (e.g., that are or comprise a nucleic acid, protein, or small molecule) that shows significant structural identity with a reference molecule or entity but differs structurally from the reference molecule or entity, e.g., in the presence or absence or in the level of one or more chemical moieties as compared to the reference molecule or entity. In some embodiments, a “variant” may be referred to as a “derivative”. In some embodiments, a variant differs functionally from its reference molecule or entity. In many embodiments, whether a particular molecule or entity is properly considered to be a “variant” of a reference is based on its degree of structural identity with the reference molecule. As will be appreciated by those skilled in the art, a biological or chemical reference molecule in typically characterized by certain characteristic structural elements. A variant, by definition, is a distinct molecule or entity that shares one or more such characteristic structural elements but differs in at least one aspect from the reference molecule or entity. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1)number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### Detailed Description of Certain Embodiments

Disclosed herein, inter alia, are conjugate agents comprising a targeting moiety, directly or indirectly conjugated with a payload moiety. In some embodiments, the targeting moiety specifically binds to a surface factor on target cells of interest (e.g., on kidney cells). In some embodiments, the payload moiety is or comprises a nucleic acid agent. In some embodiments, a payload moiety is or comprises a therapeutic agent (e.g., a therapeutic oligonucleotide).

Among other things, the present disclosure provides an insight that conjugate agents as described herein may be particularly useful or effective for the delivery of nucleic acid agents to kidney cells and/or to other cells that express or otherwise comprise a surface factor (e.g., megalin or cubilin) specifically bound by a targeting moiety as described herein.

### Targeting Moieties

A conjugate agent of the present disclosure comprises a targeting moiety. A targeting moiety for use as disclosed herein can bind to, e.g., selectively bind to, a surface factor (e.g., to a moiety or portion thereof, and/or to a particular form, such as a disease-associated form thereof) present on surfaces of target cell(s) of interest (e.g., of kidney cells) as disclosed herein.

Without wishing to be bound by theory, the present disclosure proposes that binding of a targeting moiety to a cell surface factor present on the surface of a relevant (e.g., kidney) cell, e.g., of a tissue, can achieve internalization of the cell surface factor, along with the bound targeting moiety (which may, for example, be part of a conjugate agent as described herein). In some embodiments, such internalization may mean that the relevant cell surface factor is no longer (at least for a period of time) available at the surface of the cell, e.g., of a tissue, for, e.g., signaling and/or binding to a ligand.

Among other things, the present disclosure provides an insight that triggering internalization of a surface factor may usefully achieve delivery of a targeting moiety (and/or an agent, such as a conjugate agent as described herein, that includes it), e.g., into an internal compartment such as a vesicle and/or an organelle, and/or the cytoplasm of the cell. The present disclosure further provides an insight that such internalization may be particularly useful for delivering a conjugate agent as described herein, and/or a portion thereof (e.g., a payload moiety thereof), into the cell. The present disclosure provides a specific insight that such internalization may be particularly useful for delivery of nucleic acid agents as described herein, including specifically in the context of a conjugate agent (e.g., as a payload moiety thereof) as described herein.

In some embodiments, at least 5% of a cell surface factor (for example, at least 10% of a cell surface factor, at least 20% of a cell surface factor, at least 30% of a cell surface factor, at least 40% of a cell surface factor, at least 50% of a cell surface factor, at least 60% of a cell surface factor, at least 75% of a cell surface factor, at least 90% of a cell surface factor, or at least 95% of a cell surface factor) is internalized upon binding to a targeting moiety. In some embodiments, substantially all or all of a cell surface factor is internalized upon binding to a targeting moiety.

In some embodiments, binding of a targeting moiety to a cell surface factor on the surface of a cell, e.g., of a tissue, does not internalize the cell surface factor.

In some embodiments, a conjugate agent described herein comprises one or more payload moieties and/or one or more targeting moieties.

In some embodiments, a conjugate agent described herein comprises one payload moiety and one or more targeting moieties.

In some embodiments, a conjugate agent described herein comprises one or more payload moieties and one targeting moiety.

In some embodiments, a cell surface factor is or comprises a polypeptide which is present (e.g., can be detected on) on a surface of a cell, e.g., of a tissue. In some embodiments, a cell surface factor is present on (e.g., can be detected on) a surface of a cell expressing Megalin, e.g., as described herein. In some embodiments, a cell surface factor comprises a receptor.

In some embodiments, a cell surface factor is or comprises a kidney cell surface factor. In some embodiments, a kidney cell surface factor is present on (e.g., can be detected on) a surface of a cell associated with a kidney, e.g., a cell that is or can be found in a kidney, e.g., during development, during tissue homeostasis, or in the course of a disease or disorder. In some embodiments, a kidney cell surface factor is present on, e.g., can be detected on, a proximal tubule epithelial cell and/or a podocyte.

In some embodiments, a kidney cell surface factor is present on, e.g., can be detected on, a surface of a tissue associated with a kidney, e.g., a tissue that is part of or can be found in a kidney, e.g., during development, during tissue homeostasis, and/or in the course of a disease or disorder.

In some embodiments, a kidney cell surface factor is or comprises a receptor which is present, e.g., can be detected on, a surface of a cell, e.g., a cell associated with a kidney as described herein, or a tissue associated with a kidney as described herein. In some embodiments, a kidney cell surface factor can bind to one or more co-receptors on the surface of a cell, e.g., of a tissue. In some embodiments, a kidney cell surface factor can be internalized upon binding of a kidney-specific binding moiety in a conjugate agent to a kidney cell surface factor. In some embodiments, internalization of a kidney cell surface factor as a result of binding to a kidney-specific binding moiety in a conjugate agent, also internalizes a conjugate agent (e.g., a portion thereof, e.g., a payload moiety), into a cell. In some embodiments, an internalized conjugate agent (e.g., a portion thereof, e.g., a payload moiety), is delivered to a vesicle in a cell (e.g., a lysosome, an endosome, a clathrin coated pit, or an intracellular membranous organelle, or a combination thereof). In some embodiments, an internalized conjugate agent (e.g., a portion thereof, e.g., a payload moiety), is delivered to a compartment in a cell, e.g., a cytoplasm, a mitochondria, a ribosome, a nucleus, a nucleolus, or any other compartment in a cell, or a combination thereof.

In some embodiments, an internalized conjugate agent (e.g., a portion thereof, e.g., a payload moiety), in a cell (e.g., in a vesicle or a compartment in a cell) can reduce the expression and/or activity of a target of a payload moiety.

In some embodiments, internalization of a conjugate agent (e.g., a portion thereof, e.g., a payload moiety) into a cell (e.g., into a vesicle or a compartment in a cell) uncouples, e.g., separates, a targeting moiety from a payload moiety. In some embodiments, a targeting moiety is uncoupled, e.g., separated, from a payload moiety by a chemical reaction and/or mechanical separation. In some embodiments, a chemical reaction comprises an enzymatic reaction to cleave a linker linking a targeting moiety to a payload moiety.

In some embodiments, internalization of a conjugate agent (e.g., a portion thereof, e.g., a payload moiety) into a cell (e.g., into a vesicle or a compartment in a cell) uncouples a targeting moiety from a payload moiety.

In some embodiments, a conjugate agent disclosed herein can be filtered by a glomerular capillary, e.g., into a Bowman's capsule. In some embodiments, a conjugate agent disclosed herein has a size, charge, conformation, and/or other properties that allows it to be filtered by a glomerular capillary. In some embodiments, a threshold for glomerular filtration is in the range of 30-50 kDa.

In some embodiments, a cell surface factor (e.g., a kidney cell surface factor) is or comprises a receptor chosen from Megalin, Cubilin, or both.

### Megalin-Binding Moieties

Megalin is a receptor of about 600 kDa (about 4655 amino acids) and belongs to the low-density lipoprotein receptor family (as disclosed in Nielsen R. et al. (2016), Kidney Int. 89 (1): 58-67). Megalin is also known as LDL Receptor Related Protein 2 (LRP2), Glycoprotein 330 (Gp330), Calcium Sensor Protein, or Heymann Nephritis Antigen Homolog.

| Human Megalin protein sequence: | |
|---|---|
| SEQ ID NO: 3 | |
| MDRGPAAVACTLLLALVACLAPASGQECDSAHFRCGSGHCIPADWRCDGTKDCSDDADEI | |
| GCAVVTCQQGYFKCQSEGQCIPNSWVCDQDQDCDDGSDERQDCSQSTCSSHQITCSNGQC | |
| IPSEYRCDHVRDCPDGADENDCQYPTCEQLTCDNGACYNTSQKCDWKVDCRDSSDEINCT | |
| EICLHNEFSCGNGECIPRAYVCDHDNDCQDGSDEHACNYPTCGGYQFTCPSGRCIYQNWV | |
| CDGEDDCKDNGDEDGCESGPHDVHKCSPREWSCPESGRCISIYKVCDGILDCPGREDENN | |
| TSTGKYCSMTLCSALNCQYQCHETPYGGACFCPPGYIINHNDSRTCVEFDDCQIWGICDQ | |
| KCESRPGRHLCHCEEGYILERGQYCKANDSFGEASIIFSNGRDLLIGDIHGRSFRILVES | |
| QNRGVAVGVAFHYHLQRVFWTDTVQNKVFSVDINGLNIQEVLNVSVETPENLAVDWVNNK | |
| IYLVETKVNRIDMVNLDGSYRVTLITENLGHPRGIAVDPTVGYLFFSDWESLSGEPKLER | |
| AFMDGSNRKDLVKTKLGWPAGVTLDMISKRVYWVDSRFDYIETVTYDGIQRKTVVHGGSL | |
| IPHPFGVSLFEGQVFFTDWTKMAVLKANKFTETNPQVYYQASLRPYGVTVYHSLRQPYAT | |
| NPCKDNNGGCEQVCVLSHRTDNDGLGFRCKCTFGFQLDTDERHCIAVQNFLIESSQVAIR | |
| GIPFTLSTQEDVMVPVSGNPSFFVGIDFDAQDSTIFFSDMSKHMIFKQKIDGTGREILAA | |
| NRVENVESLAFDWISKNLYWTDSHYKSISVMRLADKTRRTVVQYLNNPRSVVVHPFAGYL | |
| FFTDWFRPAKIMRAWSDGSHLLPVINTTLGWPNGLAIDWAASRLYWVDAYFDKIEHSTED | |
| GLDRRRLGHIEQMTHPFGLAIFGEHLFFTDWRLGAIIRVRKADGGEMTVIRSGIAYILHL | |
| KSYDVNIQTGSNACNQPTHPNGDCSHFCFPVPNFQRVCGCPYGMRLASNHLTCEGDPTNE | |
| PPTEQCGLFSFPCKNGRCVPNYYLCDGVDDCHDNSDEQLCGTLNNTCSSSAFTCGHGECI | |
| PAHWRCDKRNDCVDGSDEHNCPTHAPASCLDTQYTCDNHQCISKNWVCDTDNDCGDGSDE | |
| KNCNSTETCQPSQFNCPNHRCIDLSFVCDGDKDCVDGSDEVGCVLNCTASQFKCASGDKC | |
| IGVTNRCDGVFDCSDNSDEAGCPTRPPGMCHSDEFQCQEDGICIPNFWECDGHPDCLYGS | |
| DEHNACVPKTCPSSYFHCDNGNCIHRAWLCDRDNDCGDMSDEKDCPTQPFRCPSWQWQCL | |
| GHNICVNLSVVCDGIFDCPNGTDESPLCNGNSCSDFNGGCTHECVQEPFGAKCLCPLGFL | |
| LANDSKTCEDIDECDILGSCSQHCYNMRGSFRCSCDTGYMLESDGRTCKVTASESLLLLV | |
| ASQNKIIADSVTSQVHNIYSLVENGSYIVAVDFDSISGRIFWSDATQGKTWSAFQNGTDR | |
| RVVFDSSIILTETIAIDWVGRNLYWTDYALETIEVSKIDGSHRTVLISKNLTNPRGLALD | |
| PRMNEHLLFWSDWGHHPRIERASMDGSMRTVIVQDKIFWPCGLTIDYPNRLLYFMDSYLD | |
| YMDFCDYNGHHRRQVIASDLIIRHPYALTLFEDSVYWTDRATRRVMRANKWHGGNQSVVM | |
| YNIQWPLGIVAVHPSKQPNSVNPCAFSRCSHLCLLSSQGPHFYSCVCPSGWSLSPDLLNC | |
| LRDDQPFLITVRQHIIFGISLNPEVKSNDAMVPIAGIQNGLDVEFDDAEQYIYWVENPGE | |
| IHRVKTDGTNRTVFASISMVGPSMNLALDWISRNLYSTNPRTQSIEVLTLHGDIRYRKTL | |
| IANDGTALGVGFPIGITVDPARGKLYWSDQGTDSGVPAKIASANMDGTSVKTLFTGNLEH | |
| LECVTLDIEEQKLYWAVTGRGVIERGNVDGTDRMILVHQLSHPWGIAVHDSFLYYTDEQY | |
| EVIERVDKATGANKIVLRDNVPNLRGLQVYHRRNAAESSNGCSNNMNACQQICLPVPGGL | |
| FSCACATGFKLNPDNRSCSPYNSFIVVSMLSAIRGFSLELSDHSETMVPVAGQGRNALHV | |
| DVDVSSGFIYWCDFSSSVASDNAIRRIKPDGSSLMNIVTHGIGENGVRGIAVDWVAGNLY | |
| FTNAFVSETLIEVLRINTTYRRVLLKVTVDMPRHIVVDPKNRYLFWADYGQRPKIERSFL | |
| DCTNRTVLVSEGIVTPRGLAVDRSDGYVYWVDDSLDIIARIRINGENSEVIRYGSRYPTP | |
| YGITVFENSIIWVDRNLKKIFQASKEPENTEPPTVIRDNINWLRDVTIFDKQVQPRSPAE | |
| VNNNPCLENNGGCSHLCFALPGLHTPKCDCAFGTLQSDGKNCAISTENFLIFALSNSLRS | |
| LHLDPENHSPPFQTINVERTVMSLDYDSVSDRIYFTQNLASGVGQISYATLSSGIHTPTV | |
| IASGIGTADGIAFDWITRRIYYSDYLNQMINSMAEDGSNRTVIARVPKPRAIVLDPCQGY | |
| LYWADWDTHAKIERATLGGNFRVPIVNSSLVMPSGLTLDYEEDLLYWVDASLQRIERSTL | |
| TGVDREVIVNAAVHAFGLTLYGQYIYWTDLYTQRIYRANKYDGSGQIAMTTNLLSQPRGI | |
| NTVVKNQKQQCNNPCEQFNGGCSHICAPGPNGAECQCPHEGNWYLANNRKHCIVDNGERC | |
| GASSFTCSNGRCISEEWKCDNDNDCGDGSDEMESVCALHTCSPTAFTCANGRCVQYSYRC | |
| DYYNDCGDGSDEAGCLFRDCNATTEFMCNNRRCIPREFICNGVDNCHDNNTSDEKNCPDR | |
| TCQSGYTKCHNSNICIPRVYLCDGDNDCGDNSDENPTYCTTHTCSSSEFQCASGRCIPQH | |
| WYCDQETDCFDASDEPASCGHSERTCLADEFKCDGGRCIPSEWICDGDNDCGDMSDEDKR | |
| HQCQNQNCSDSEFLCVNDRPPDRRCIPQSWVCDGDVDCTDGYDENQNCTRRTCSENEFTC | |
| GYGLCIPKIFRCDRHNDCGDYSDERGCLYQTCQQNQFTCQNGRCISKTFVCDEDNDCGDG | |
| SDELMHLCHTPEPTCPPHEFKCDNGRCIEMMKLCNHLDDCLDNSDEKGCGINECHDPSIS | |
| GCDHNCTDTLTSFYCSCRPGYKLMSDKRTCVDIDECTEMPFVCSQKCENVIGSYICKCAP | |
| GYLREPDGKTCRQNSNIEPYLIFSNRYYLRNLTIDGYFYSLILEGLDNVVALDEDRVEKR | |
| LYWIDTQRQVIERMELNKTNKETIINHRLPAAESLAVDWVSRKLYWLDARLDGLFVSDLN | |
| GGHRRMLAQHCVDANNTFCFDNPRGLALHPQYGYLYWADWGHRAYIGRVGMDGTNKSVII | |
| STKLEWPNGITIDYTNDLLYWADAHLGYIEYSDLEGHHRHTVYDGALPHPFAITIFEDTI | |
| YWTDWNTRTVEKGNKYDGSNRQTLVNTTHRPFDIHVYHPYRQPIVSNPCGTNNGGCSHLC | |
| LIKPGGKGFTCECPDDFRTLQLSGSTYCMPMCSSTQFLCANNEKCIPIWWKCDGQKDCSD | |
| GSDELALCPQRFCRLGQFQCSDGNCTSPQTLCNAHQNCPDGSDEDRLLCENHHCDSNEWQ | |
| CANKRCIPESWQCDTENDCEDNSDEDSSHCASRTCRPGQFRCANGRCIPQAWKCDVDNDC | |
| GDHSDEPIEECMSSAHLCDNFTEFSCKTNYRCIPKWAVCNGVDDCRDNSDEQGCEERTCH | |
| PVGDFRCKNHHCIPLRWQCDGQNDCGDNSDEENCAPRECTESEFRCVNQQCIPSRWICDH | |
| YNDCGDNSDERDCEMRTCHPEYFQCTSGHCVHSELKCDGSADCLDASDEADCPTRFPDGA | |
| YCQATMFECKNHVCIPPYWKCDGDDDCGDGSDEELHLCLDVPCNSPNRFRCDNNRCIYSH | |
| EVCNGVDDCGDGTDETEEHCRKPTPKPCTEYEYKCGNGHCIPHDNVCDDADDCGDWSDEL | |
| GCNKGKERTCAENICEQNCTQLNEGGFICSCTAGFETNVFDRTSCLDINECEQFGTCPQH | |
| CRNTKGSYECVCADGFTSMSDRPGKRCAAEGSSPLLLLPDNVRIRKYNLSSERFSEYLQD | |
| EEYIQAVDYDWDPKDIGLSVVYYTVRGEGSRFGAIKRAYIPNFESGRNNLVQEVDLKLKY | |
| VMQPDGIAVDWVGRHIYWSDVKNKRIEVAKLDGRYRKWLISTDLDQPAAIAVNPKLGLMF | |
| WTDWGKEPKIESAWMNGEDRNILVFEDLGWPTGLSIDYLNNDRIYWSDFKEDVIETIKYD | |
| GTDRRVIAKEAMNPYSLDIFEDQLYWISKEKGEVWKQNKFGQGKKEKTLVVNPWLTQVRI | |
| FHQLRYNKSVPNLCKQICSHLCLLRPGGYSCACPQGSSFIEGSTTECDAAIELPINLPPP | |
| CRCMHGGNCYFDETDLPKCKCPSGYTGKYCEMAFSKGISPGTTAVAVLLTILLIVVIGAL | |
| AIAGFFHYRRTGSLLPALPKLPSLSSLVKPSENGNGVTFRSGADLNMDIGVSGFGPETAI | |
| DRSMAMSEDFVMEMGKQPIIFENPMYSARDSAVKVVQPIQVTVSENVDNKNYGSPINPSE | |
| IVPETNPTSPAADGTQVTKWNLFKRKSKQTTNFENPIYAQMENEQKESVAATPPPSPSLP | |
| AKPKPPSRRDPTPTYSATEDTFKDTANLVKEDSEV | |
| Megalin is encoded by the LRP2 gene. A nucleic | |
| acid sequence encoding human Megalin is provided | |
| by SEQ ID NO: 4: | |
| 1 | aggcttttgg ccactaggag ctggcggagg tgcagaccta aaggagcgtt cgctagcaga |
| 61 | ggcgctgccg gtgcggtgtg ctacgcgcgc ccacctcccg gggaaggaac ggcgaggccg |
| 121 | gggaccgtcg cggagatgga tcgcgggccg gcagcagtgg cgtgcacgct gctcctggct |
| 181 | ctcgtcgcct gcctagcgcc ggccagtggc caagaatgtg acagtgcgca ttttcgctgt |
| 241 | ggaagtgggc attgcatccc tgcagactgg aggtgtgatg ggaccaaaga ctgttcagat |
| 301 | gacgcggatg aaattggctg cgctgttgtg acctgccagc agggctattt caagtgccag |
| 361 | agtgagggac aatgcatccc caactcctgg gtgtgtgacc aagatcaaga ctgtgatgat |
| 421 | ggctcagatg aacgtcaaga ttgctcacaa agtacatgct caagtcatca gataacatgc |
| 481 | tccaatggtc agtgtatccc aagtgaatac aggtgcgacc acgtcagaga ctgccccgat |
| 541 | ggagctgatg agaatgactg ccagtaccca acatgtgagc agcttacttg tgacaatggg |
| 601 | gcctgctata acaccagtca gaagtgtgat tggaaagttg attgcaggga ctcctcagat |
| 661 | gaaatcaact gcactgagat atgcttgcac aatgagtttt catgtggcaa tggagagtgt |
| 721 | atccctcgtg cttatgtctg tgaccatgac aatgattgcc aagacggcag tgacgaacat |
| 781 | gcttgcaact atccgacctg cggtggttac cagttcactt gccccagtgg ccgatgcatt |
| 841 | tatcaaaact gggtttgtga tggagaagat gactgtaaag ataatggaga tgaagatgga |
| 901 | tgtgaaagcg gtcctcatga tgttcataaa tgttccccaa gagaatggtc ttgcccagag |
| 961 | tcgggacgat gcatctccat ttataaagtt tgtgatggga ttttagattg cccaggaaga |
| 1021 | gaagatgaaa acaacactag taccggaaaa tactgtagta tgactctgtg ctctgccttg |
| 1081 | aactgccagt accagtgcca tgagacgccg tatggaggag cgtgtttttg tcccccaggt |
| 1141 | tatatcatca accacaatga cagccgtacc tgtgttgagt ttgatgattg ccagatatgg |
| 1201 | ggaatttgtg accagaagtg tgaaagccga cctggccgtc acctgtgcca ctgtgaagaa |
| 1261 | gggtatatct tggagcgtgg acagtattgc aaagctaatg attcctttgg cgaggcctcc |
| 1321 | attatcttct ccaatggtcg ggatttgtta attggtgata ttcatggaag gagcttccgg |
| 1381 | atcctagtgg agtctcagaa tcgtggagtg gccgtgggtg tggctttcca ctatcacctg |
| 1441 | caaagagttt tttggacaga caccgtgcaa aataaggttt tttcagttga cattaatggt |
| 1501 | ttaaatatcc aagaggttct caatgtttct gttgaaaccc cagagaacct ggctgtggac |
| 1561 | tgggttaata ataaaatcta tctagtggaa accaaggtca accgcataga tatggtaaat |
| 1621 | ttggatggaa gctatcgggt tacccttata actgaaaact tggggcatcc tagaggaatt |
| 1681 | gccgtggacc caactgttgg ttatttattt ttctcagatt gggagagcct ttctggggaa |
| 1741 | cctaagctgg aaagggcatt catggatggc agcaaccgta aagacttggt gaaaacaaag |
| 1801 | ctgggatggc ctgctggggt aactctggat atgatatcga agcgtgttta ctgggttgac |
| 1861 | tctcggtttg attacattga aactgtaact tatgatggaa ttcaaaggaa gactgtagtt |
| 1921 | catggaggct ccctcattcc tcatcccttt ggagtaagct tatttgaagg tcaggtgttc |
| 1981 | tttacagatt ggacaaagat ggccgtgctg aaggcaaaca agttcacaga gaccaaccca |
| 2041 | caagtgtact accaggcttc cctgaggccc tatggagtga ctgtttacca ttccctcaga |
| 2101 | cagccctatg ctaccaatcc gtgtaaagat aacaatgggg gctgtgagca ggtctgtgtc |
| 2161 | ctcagccaca gaacagataa tgatggtttg ggtttccgtt gcaagtgcac attcggcttc |
| 2221 | caactggata cagatgagcg ccactgcatt gctgttcaga atttcctcat tttttcatcc |
| 2281 | caagttgcta ttcgtgggat cccgttcacc ttgtctaccc aggaagatgt catggttcca |
| 2341 | gtttcgggga atccttcttt ctttgtcggg attgattttg acgcccagga cagcactatc |
| 2401 | tttttttcag atatgtcaaa acacatgatt tttaagcaaa agattgatgg cacaggaaga |
| 2461 | gaaattctcg cagctaacag ggtggaaaat gttgaaagtt tggcttttga ttggatttca |
| 2521 | aagaatctct attggacaga ctctcattac aagagtatca gtgtcatgag gctagctgat |
| 2581 | aaaacgagac gcacagtagt tcagtattta aataacccac ggtcggtggt agttcatcct |
| 2641 | tttgccgggt atctattctt cactgattgg ttccgtcctg ctaaaattat gagagcatgg |
| 2701 | agtgacggat ctcacctctt gcctgtaata aacactactc ttggatggcc caatggcttg |
| 2761 | gccatcgatt gggctgcttc acgattgtac tgggtagatg cctattttga taaaattgag |
| 2821 | cacagcacct ttgatggttt agacagaaga agactgggcc atatagagca gatgacacat |
| 2881 | ccgtttggac ttgccatctt tggagagcat ttatttttta ctgactggag actgggtgcc |
| 2941 | attattcgag tcaggaaagc agatggtgga gaaatgacag ttatccgaag tggcattgct |
| 3001 | tacatactgc atttgaaatc gtatgatgtc aacatccaga ctggttctaa cgcctgtaat |
| 3061 | caacccacgc atcctaacgg tgactgcagc cacttctgct tcccggtgcc aaatttccag |
| 3121 | cgagtgtgtg ggtgccctta tggaatgagg ctggcttcca atcacttgac atgcgagggg |
| 3181 | gacccaacca atgaaccacc cacagagcag tgtggcttat tttccttccc ctgtaaaaat |
| 3241 | ggcagatgtg tgcccaatta ctatctctgt gatggagtcg atgattgtca tgataacagt |
| 3301 | gatgagcaac tatgtggcac acttaataat acctgttcat cttcggcgtt cacctgtggc |
| 3361 | catggggagt gcattcctgc acactggcgc tgtgacaaac gcaacgactg tgtggatggc |
| 3421 | agtgatgagc acaactgccc cacccacGca cctgcttcct gccttgacac ccaatacacc |
| 3481 | tgtgataatc accagtgtat ctcaaagaac tgggtctgtg acacagacaa tgattgtggg |
| 3541 | gatggatctg atgaaaagaa ctgcaattcg acagagacat gccaacctag tcagtttaat |
| 3601 | tgccccaatc atcgatgtat tgacctatcg tttgtctgtg atggtgacaa ggattgtgtt |
| 3661 | gatggatctg atgaggttgg ttgtgtatta aactgtactg cttctcaatt caagtgtgcc |
| 3721 | agtggggata aatgtattgg cgtcacaaat cgttgtgatg gtgtttttga ttgcagtgac |
| 3781 | aactcggatg aagcaggctg tccaaccagg cctcctggta tgtgccactc agatgaattt |
| 3841 | cagtgccaag aagatggtat ctgcatcccg aacttctggg aatgtgatgg gcatccagac |
| 3901 | tgcctctatg gatctgatga gcacaatgcc tgtgtcccca agacttgccc ttcatcatat |
| 3961 | ttccactgtg acaacggaaa ctgcatccac agggcatggc tctgtgatcg ggacaatgac |
| 4021 | tgcggggata tgagtgatga gaaggactgc cctactcagc cctttcgctg tcctagttgg |
| 4081 | caatggcagt gtcttggcca taacatctgt gtgaatctga gtgtagtgtg tgatggcatc |
| 4141 | tttgactgcc ccaatgggac agatgagtcc ccactttgca atgggaacag ctgctcagat |
| 4201 | ttcaatggtg gttgtactca cgagtgtgtt caagagccct ttggggctaa atgcctatgt |
| 4261 | ccattcggat tcttacttgc caatgattct aagacctgtg aagacataga tgaatgtgat |
| 4321 | attctaggct cttgtagcca gcactgttac aatatgagag gttctttccg gtgctcgtgt |
| 4381 | gatacaggct acatgttaga aagtgatggg aggacttgca aagttacagc atctgagagt |
| 4441 | ctgctgttac ttgtggcaag tcagaacaaa attattgccg acagtgtcac ctcccaggtc |
| 4501 | cacaatatct attcattggt cgagaatggt tcttacattg tagctgttga ttttgattca |
| 4561 | attagtggtc gtatcttttg gtctgatgca actcagggta aaacctggag tgcgtttcaa |
| 4621 | aatggaacgg acagaagagt ggtatttgac agtagcatca tcttgactga aactattgca |
| 4681 | atagattggg taggtcgtaa tctttactgg acagactatg ctctggaaac aattgaagtc |
| 4741 | tccaaaattg atgggagcca caggactgtg ctgattagta aaaacctaac aaatccaaga |
| 4801 | ggactagcat tagatcccag aatgaatgag catctactgt tctggtctga ctggggccac |
| 4861 | caccctcgca tcgagcgagc cagcatggac ggcagcatgc gcactgtcat tgtccaggac |
| 4921 | aagatcttct ggccctgcgg cttaactatt gactacccca acagactgct ctacttcatg |
| 4981 | gactcctatc ttgattacat ggacttttgt gattataatg gacaccatcg gagacaggtg |
| 5041 | atagccagtg atttgattat acggcacccc tatgccctaa ctctctttga agactctgtg |
| 5101 | tactggactg accgtgctac tcgtcgggtt atgcgagcca acaagtggca tggagggaac |
| 5161 | cagtcagttg taatgtataa tattcaatgg ccccttggga ttgttgcggt tcatccttcg |
| 5221 | aaacaaccaa attccgtgaa tccatgtgcc ttttcccgct gcagccatct ctgcctgctt |
| 5281 | tcctcacagg ggcctcattt ttactcctgt gtttgtcctt caggatggag tctgtctcct |
| 5341 | gatctcctga attgcttgag agatgatcaa cctttcttaa taactgtaag gcaacatata |
| 5401 | atttttggaa tctcccttaa tcctgaggtg aagagcaatg atgctatggt ccccatagca |
| 5461 | gggatacaga atggtttaga tgttgaattt gatgatgctg agcaatacat ctattgggtt |
| 5521 | gaaaatccag gtgaaattca cagagtgaag acagatggca ccaacaggac agtatttgct |
| 5581 | tctatatcta tggtggggcc ttctatgaac ctggccttag attggatttc aagaaacctt |
| 5641 | tattctacca atcctagaac tcagtcaatc gaggttttga cactccacgg agatatcaga |
| 5701 | tacagaaaaa cattgattgc caatgatggg acagctcttg gagttggctt tccaattggc |
| 5761 | ataactgttg atcctgctcg tgggaagctg tactggtcag accaaggaac tgacagtggg |
| 5821 | gttcctgcca agatcgccag tgctaacatg gatggcacat ctgtgaaaac tctctttact |
| 5881 | gggaacctcg aacacctgga gtgtgtcact cttgacatcg aagagcagaa actctactgg |
| 5941 | gcagtcactg gaagaggagt gattgaaaga ggaaacgtgg atggaacaga tcgaatgatc |
| 6001 | ctggtacacc agctttccca cccctgggga attgcagtcc atgattcttt cctttattat |
| 6061 | actgatgaac agtatgaggt cattgaaaga gttgataagg ccactggggc caacaaaata |
| 6121 | gtcttgagag ataatgttcc aaatctgagg ggtcttcaag tttatcacag acgcaatgcc |
| 6181 | gccgaatcct caaatggctg tagcaacaac atgaatgcct gtcagcagat ttgcctgcct |
| 6241 | gtaccaggag gattgttttc ctgcgcctgt gccactggat ttaaactcaa tcctgataat |
| 6301 | cggtcctgct ctccatataa ctctttcatt gttgtttcaa tgctgtctgc aatcagaggc |
| 6361 | tttagcttgg aattgtcaga tcattcagaa accatggtgc cggtggcagg ccaaggacga |
| 6421 | aacgcactgc atgtggatgt ggatgtgtcc tctggcttta tttattggtg tgattttagc |
| 6481 | agctcagtgg catctgataa tgcgatccgt agaattaaac cagatggatc ttctctgatg |
| 6541 | aacattgtga cacatggaat aggagaaaat ggagtccggg gtattgcagt ggattgggta |
| 6601 | gcaggaaatc tttatttcac caatgccttt gtttctgaaa cactgataga agttctgcgg |
| 6661 | atcaatacta cttaccgccg tgttcttctt aaagtcacag tggacatgcc taggcatatt |
| 6721 | gttgtagatc ccaagaacag atacctcttc tgggctgact atgggcagag accaaagatt |
| 6781 | gagcgttctt tccttgactg taccaatcga acagtgcttg tgtcagaggg cattgtcaca |
| 6841 | ccacggggct tggcagtgga ccgaagtgat ggctacgttt attgggttga tgattcttta |
| 6901 | gatataattg caaggattcg tatcaatgga gagaactctg aagtgattcg ttatggcagt |
| 6961 | cgttacccaa ctccttatgg catcactgtt tttgaaaatt ctatcatatg ggtagatagg |
| 7021 | aatttgaaaa agatcttcca agccagcaag gaaccagaga acacagagcc acccacagtg |
| 7081 | ataagagaca atatcaactg gctaagagat gtgaccatct ttgacaagca agtccagccc |
| 7141 | cggtcaccag cagaggtcaa caacaaccct tgcttggaaa acaatggtgg gtgctctcat |
| 7201 | ctctgctttg ctctgcctgg attgcacacc ccaaaatgtg actgtgcctt tgggaccctg |
| 7261 | caaagtgatg gcaagaattg tgccatttca acagaaaatt tcctcatctt tgccttgtct |
| 7321 | aattccttga gaagcttaca cttggaccct gaaaaccata gcccaccttt ccaaacaata |
| 7381 | aatgtggaaa gaactgtcat gtctctagac tatgacagtg taagtgatag aatctacttc |
| 7441 | acacaaaatt tagcctctgg agttggacag atttcctatg ccaccctgtc ttcagggatc |
| 7501 | catactccaa ctgtcattgc ttcaggtata gggactgctg atggcattgc ctttgactgg |
| 7561 | attactagaa gaatttatta cagtgactac ctcaaccaga tgattaattc catggctgaa |
| 7621 | gatgggtcta accgcactgt gatagcccgc gttccaaaac caagagcaat tgtgttagat |
| 7681 | ccctgccaag ggtacctgta ctgggctgac tgggatacac atgccaaaat cgagagagcc |
| 7741 | acattgggag gaaacttccg cgtacccatt gtgaacagca gtctggtcat gcccagtggg |
| 7801 | ctgactctgg actatgaaga ggaccttctc tactgggtgg atgctagtct gcagaggatt |
| 7861 | gaacgcagca ctctgacggg cgtggatcgt gaagtcattg tcaatgcagc cgttcatgct |
| 7921 | tttggcttga ctctctatgg ccagtatatt tactggactg acttgtacac acaaagaatt |
| 7981 | taccgagcta acaaatatga cgggtcaggt cagattgcaa tgaccacaaa tttgctctcc |
| 8041 | cagcccaggg gaatcaacac tgttgtgaag aaccagaaac aacagtgtaa caatccttgt |
| 8101 | gaacagttta atgggggctg cagccatatc tgtgcaccag gtccaaatgg tgccgagtgc |
| 8161 | cagtgtccac atgagggcaa ctggtatttg gccaacaaca ggaagcactg cattgtggac |
| 8221 | aatggtgaac gatgtggtgc atcttccttc acctgctcca atgggcgctg catctcggaa |
| 8281 | gagtggaagt gtgataatga caacgactgt ggggatggca gtgatgagat ggaaagtgtc |
| 8341 | tgtgcacttc acacctgctc accgacagcc ttcacctgtg ccaatgggcg atgtgtccaa |
| 8401 | tactcttacc gctgtgatta ctacaatgac tgtggtgatg gcagtgatga ggcagggtgc |
| 8461 | ctgttcaggg actgcaatgc caccacggag tttatgtgca ataacagaag gtgcatacct |
| 8521 | cgtgagttta tctgcaatgg tgtagacaac tgccatgata ataacacttc agatgagaaa |
| 8581 | aattgccctg atcgcacttg ccagtctgga tacacaaaat gtcataattc aaatatttgt |
| 8641 | attcctcgcg tttatttgtg tgacggagac aatgactgtg gagataacag tgatgaaaac |
| 8701 | cctacttatt gcaccactca cacgtgcagc agcagtgagt tccaatgcgc atctgggcgc |
| 8761 | tgtattcctc aacattggta ttgtgatcaa gaaacagatt gttttgatgc ctctgatgaa |
| 8821 | cctgcctctt gtggtcactc tgagcgaaca tgcctagctg atgagttcaa gtgtgatggt |
| 8881 | gggaggtgca tcccaagcga atggatctgt gacggtgata atgactgtgg ggatatgagt |
| 8941 | gacgaggata aaaggcacca gtgtcagaat caaaactgct cggattccga gtttctctgt |
| 9001 | gtaaatgaca gacctccgga caggaggtgc attccccagt cttgggtctg tgatggcgat |
| 9061 | gtggattgta ctgacggcta cgatgagaat cagaattgca ccaggagaac ttgctctgaa |
| 9121 | aatgaattca cctgtggtta cggactgtgt atcccaaaga tattcaggtg tgaccggcac |
| 9181 | aatgactgtg gtgactatag cgacgagagg ggctgcttat accagacttg ccaacagaat |
| 9241 | cagtttacct gtcagaacgg gcgctgcatt agtaaaacct tcgtctgtga tgaggataat |
| 9301 | gactgtggag acggatctga tgagctgatg cacctgtgcc acaccccaga acccacgtgt |
| 9361 | ccacctcacg agttcaagtg tgacaatggg cgctgcatcg agatgatgaa actctgcaac |
| 9421 | cacctagatg actgtttgga caacagcgat gagaaaggct gtggcattaa tgaatgccat |
| 9481 | gacccttcaa tcagtggctg cgatcacaac tgcacagaca ccttaaccag tttctattgt |
| 9541 | tcctgtcgtc ctggttacaa gctcatgtct gacaagcgga cttgtgttga tattgatgaa |
| 9601 | tgcacagaga tgccttttgt ctgtagccag aagtgtgaga atgtaatagg ctcctacatc |
| 9661 | tgtaagtgtg ccccaggcta cctccgagaa ccagatggaa agacctgccg gcaaaacagt |
| 9721 | aacatcgaac cctatctcat ttttagcaac cgttactatt tgagaaattt aactatagat |
| 9781 | ggctattttt actccctcat cttggaagga ctggacaatg ttgtggcatt agattttgac |
| 9841 | cgagtagaga agagattgta ttggattgat acacagaggc aagtcattga gagaatgttt |
| 9901 | ctgaataaga caaacaagga gacaatcata aaccacagac taccagctgc agaaagtctg |
| 9961 | gctgtagact gggtttccag aaagctctac tggttggatg cccgcctgga tggcctcttt |
| 10021 | gtctctgacc tcaatggtgg acaccgccgc atgctggccc agcactgtgt ggatgccaac |
| 10081 | aacaccttct gctttgataa tcccagagga cttgcccttc accctcaata tgggtacctc |
| 10141 | tactgggcag actggggtca ccgcgcatac attgggagag taggcatgga tggaaccaac |
| 10201 | aagtctgtga taatctccac caagttagag tggcctaatg gcatcaccat tgattacacc |
| 10261 | aatgatctac tctactgggc agatgcccac ctgggttaca tagagtactc tgatttggag |
| 10321 | ggccaccatc gacacacggt gtatgatggg gcactgcctc accctttcgc tattaccatt |
| 10381 | tttgaagaca ctatttattg gacagattgg aatacaagga cagtggaaaa gggaaacaaa |
| 10441 | tatgatggat caaatagaca gacactggtg aacacaacac acagaccatt tgacatccat |
| 10501 | gtgtaccatc catataggca gcccattgtg agcaatccct gtggtaccaa caatggtggc |
| 10561 | tgttctcatc tctgcctcat caagccagga ggaaaagggt tcacttgcga gtgtccagat |
| 10621 | gacttccgca cccttcagct gagtggcagc acctactgca tgcccatgtg ctccagcacc |
| 10681 | cagttcctgt gcgctaacaa tgaaaagtgc attcctatct ggtggaaatg tgatggacag |
| 10741 | aaagactgct cagatggctc tgatgaactg gccctttgcc cgcagcgctt ctgccgactg |
| 10801 | ggacagttcc agtgcagtga cggcaactgc accagcccgc agactttatg caatgctcac |
| 10861 | caaaattgcc ctgatgggtc tgatgaagac cgtcttcttt gtgagaatca ccactgtgac |
| 10921 | tccaatgaat ggcagtgcgc caacaaacgt tgcatcccag aatcctggca gtgtgacaca |
| 10981 | tttaacgact gtgaggataa ctcagatgaa gacagttccc actgtgccag caggacctgc |
| 11041 | cggccgggcc agtttcggtg tgctaatggc cgctgcatcc cgcaggcctg gaagtgtgat |
| 11101 | gtggataatg attgtggaga ccactcggat gagcccattg aagaatgcat gagctctgcc |
| 11161 | catctctgtg acaacttcac agaattcagc tgcaaaacaa attaccgctg catcccaaag |
| 11221 | tgggccgtgt gcaatggtgt agatgactgc agggacaaca gtgatgagca aggctgtgag |
| 11281 | gagaggacat gccatcctgt gggggatttc cgctgtaaaa atcaccactg catccctctt |
| 11341 | cgttggcagt gtgatgggca aaatgactgt ggagataact cagatgagga aaactgtgct |
| 11401 | ccccgggagt gcacagagag cgagtttcga tgtgtcaatc agcagtgcat tccctcgcga |
| 11461 | tggatctgtg accattacaa cgactgtggg gacaactcag atgaacggga ctgtgagatg |
| 11521 | aggacctgcc atcctgaata ttttcagtgt acaagtggac attgtgtaca cagtgaactg |
| 11581 | aaatgcgatg gatccgctga ctgtttggat gcgtctgatg aagctgattg tcccacacgc |
| 11641 | tttcctgatg gtgcatactg ccaggctact atgttcgaat gcaaaaacca tgtttgtatc |
| 11701 | ccgccatatt ggaaatgtga tggcgatgat gactgtggcg atggttcaga tgaagaactt |
| 11761 | cacctgtgct tggatgttcc ctgtaattca ccaaaccgtt tccggtgtga caacaatcgc |
| 11821 | tgcatttata gtcatgaggt gtgcaatggt gtggatgact gtggagatgg aactgatgag |
| 11881 | acagaggagc actgtagaaa accgacccct aaaccttgta cagaatatga atataagtgt |
| 11941 | ggcaatgggc attgcattcc acatgacaat gtgtgtgatg atgccgatga ctgtggtgac |
| 12001 | tggtccgatg aactgggttg caataaagga aaagaaagaa catgtgctga aaatatatgc |
| 12061 | gagcaaaatt gtacccaatt aaatgaagga ggatttatct gctcctgtac agctgggttc |
| 12121 | gaaaccaatg tttttgacag aacctcctgt ctagatatca atgaatgtga acaatttggg |
| 12181 | acttgtcccc agcactgcag aaataccaaa ggaagttatg agtgtgtctg tgctgatggc |
| 12241 | ttcacgtcta tgagtgaccg ccctggaaaa cgatgtgcag ctgagggtag ctctcctttg |
| 12301 | ttgctactgc ctgacaatgt ccgaattcga aaatataatc tctcatctga gaggttctca |
| 12361 | gagtatcttc aagatgagga atatatccaa gctgttgatt atgattggga tcccaaggac |
| 12421 | ataggcctca gtgttgtgta ttacactgtg cgaggggagg gctctaggtt tggtgctatc |
| 12481 | aaacgtgcct acatccccaa ctttgaatcc ggccgcaata atcttgtgca ggaagttgac |
| 12541 | ctgaaactga aatacgtaat gcagccagat ggaatagcag tggactgggt tggaaggcat |
| 12601 | atttactggt cagatgtcaa gaataaacgc attgaggtgg ctaaacttga tggaaggtac |
| 12661 | agaaagtggc tgatttccac tgacctggac caaccagctg ctattgctgt gaatcccaaa |
| 12721 | ctagggctta tgttctggac tgactgggga aaggaaccta aaatcgagtc tgcctggatg |
| 12781 | aatggagagg accgcaacat cctggttttc gaggaccttg gttggccaac tggcctttct |
| 12841 | atcgattatt tgaacaatga ccgaatctac tggagtgact tcaaggagga cgttattgaa |
| 12901 | accataaaat atgatgggac tgataggaga gtcattgcaa aggaagcaat gaacccttac |
| 12961 | agcctggaca tctttgaaga ccagttatac tggatatcta aggaaaaggg agaagtatgg |
| 13021 | aaacaaaata aatttgggca aggaaagaaa gagaaaacgc tggtagtgaa cccttggctc |
| 13081 | actcaagttc gaatctttca tcaactcaga tacaataagt cagtgcccaa cctttgcaaa |
| 13141 | cagatctgca gccacctctg ccttctgaga cctggaggat acagctgtgc ctgtccccaa |
| 13201 | ggctccagct ttatagaggg gagcaccact gagtgtgatg cagccatcga actgcctatc |
| 13261 | aacctgcccc ccccatgcag gtgcatgcac ggaggaaatt gctattttga tgagactgac |
| 13321 | ctccccaaat gcaagtgtcc tagcggctac accggaaaat attgtgaaat ggcgttttca |
| 13381 | aaaggcatct ctccaggaac aaccgcagta gctgtgctgt tgacaatcct cttgatcgtc |
| 13441 | gtaattggag ctctggcaat tgcaggattc ttccactata gaaggaccgg ctcccttttg |
| 13501 | cctgctctgc ccaagctgcc aagcttaagc agtctcgtca agccctctga aaatgggaat |
| 13561 | ggggtgacct tcagatcagg ggcagatctt aacatggata ttggagtgtc tggttttgga |
| 13621 | cctgagactg ctattgacag gtcaatggca atgagtgaag actttgtcat ggaaatgggg |
| 13681 | aagcagccca taatatttga aaacccaatg tactcagcca gagacagtgc tgtcaaagtg |
| 13741 | gttcagccaa tccaggtgac tgtatctgaa aatgtggata ataagaatta tggaagtccc |
| 13801 | ataaaccctt ctgagatagt tccagagaca aacccaactt caccagctgc tgatggaact |
| 13861 | caggtgacaa aatggaatct cttcaaacga aaatctaaac aaactaccaa ctttgaaaat |
| 13921 | ccaatctatg cacagatgga gaacgagcaa aaggaaagtg ttgctgcgac accacctcca |
| 13981 | tcaccttcgc tccctgctaa gcctaagcct ccttcgagaa gagacccaac tccaacctat |
| 14041 | tctgcaacag aagacacttt taaagacacc gcaaatcttg ttaaagaaga ctctgaagta |
| 14101 | tagctatacc agctatttag ggaataatta gaaacacact tttgcacata tattttttac |
| 14161 | aaacagatga aaaaagttaa cattcagtac tttatgaaaa aaatatattt ttccctgttt |
| 14221 | gcctatagtt ggaggtatcc tgtgtgtctt tttttactta tgccgtctca tatttttaca |
| 14281 | aataattatc acaatgtact atatgtatat ctttgcactg aagttgtctg aaggtaatac |
| 14341 | tataaatata ttgtatattt gtaaattttg gaaagattat cctgttactg aatttgctaa |
| 14401 | taaagatgtc tgctgatttg gttggtgatc attatagtaa atgatccaac aagaaaagga |
| 14461 | attgactggg gacctttagc cgtgtctaaa gaagaggcac cactcatatt tcctataaaa |
| 14521 | ttatctagga aaggaatcca ggccccgctc ttgggtccat ttttacacat tagcacttaa |
| 14581 | ttaatgttca atattacatg tcaatttgat taatggctat gttgataggg gccactatgt |
| 14641 | gttgtataga catctggact tgactgtaga ctcctcagat aatacagaag gtaggaaaag |
| 14701 | caattcagtt tggcccttct gtgtgttggc attgtctaac cagaactctc tgtttcatgt |
| 14761 | gtgttctctc actagctgcc aagacaacat ttttatttgt gatgtctatg aggaaatccc |
| 14821 | atatcattaa gtgccagtgt cctgcattga gtttgtggtt aattaaatga gctcttctgc |
| 14881 | tgatggaccc tggagcaatt tctcccctca cctgacattc aaggtggtca cctgccctag |
| 14941 | tagttggagc tcagtagctg aatttctgaa accaaatctg tgtcttcata aaataaggtg |
| 15001 | caaaaaaaaa aaataccagt taagtaaagc ctcaactggg tttttgtttc tatgaaaata |
| 15061 | tcattataat cactatttat ttcctaagtt gaacctgaat agaaagggaa accattctta |
| 15121 | ttaagctttt tattaggccc tgtggctaaa tgtgtacatt tatattagaa tgtactgtac |
| 15181 | agtccagatc ttttctttaa ttcttattgg tttttttttt tttttttttt ttagagatgg |
| 15241 | agtcttgcta tattgccaag gctgatcttg aagtcctggg ctcaagtgat cctcccacct |
| 15301 | cagcctcctg agtggttggg gttacgggcg tgagccactg tgcctggctt ccagctctcc |
| 15361 | tcttaaatag tgggtatagt ctgcacaaca ggaaccatgg caggaatata cactttccca |
| 15421 | tagcaaatag catacctgac tctctgtgct aatattgcac atttgttaaa caatgaatga |
| 15481 | atggatggat ggatggatgg atgaatgaat gaaacatata ctactgatta ttttattcca |
| 15541 | gagttctcaa aatatttgtt gctgatattt tgagtgctga ctgtaattac tttgattaga |
| 15601 | taaacaactg gaaataatgc tgctgaaaaa gttctaataa atgtgtattt tatcaga |

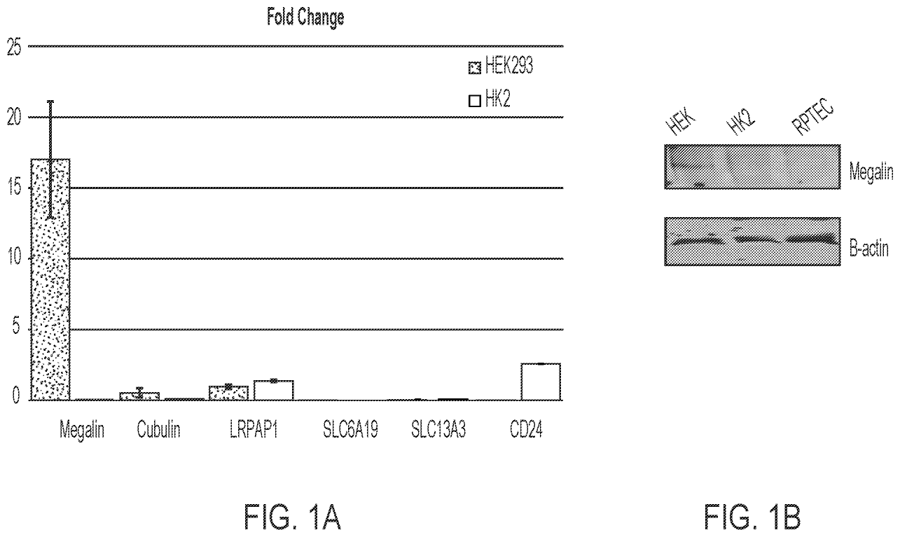

The extracellular domain of Megalin includes clusters of cysteine-rich complement-type repeats. The repeats are separated by beta-propeller domains comprising YWTD motifs and EGF-type repeats. Megalin has one transmembrane domain which positions it in parts of the cell membrane that includes cholesterol and/or glycosphingolipids. Megalin also has an intracellular C-terminal cytoplasmic domain which can regulate receptor trafficking and/or endocytosis. The cytoplasmic domain of Megalin comprises NPXY motifs and several other domains such as proline-rich sequences and PDZ motifs. Megalin's cytoplasmic domain has been linked to receptor internalization. A typical structure of Megalin is disclosed in FIG. 1 of Marzolo and Farfan (2011), Biol Res 44:89-105, the entire contents of which are hereby incorporated by reference. The extracellular domain of Megalin may also include one or more post-translational modifications, such as glycosylation.

In some embodiments, Megalin interacts with a co-receptor, Cubilin.

Megalin has been reported to be found on surfaces of one or more of the following tissues and/or cells:immune cells (e.g., bone marrow cells, lymph node cells, thymic cells, peripheral blood mononuclear cells [e.g., myeloid and/or lymphoid cells], erythrocytes, eosinophils, neutrophils, and/or platelets); nervous system (e.g., brain tissue, cortex, cerebellum, retinal cells, spinal cord cells, nerve cells, neurons, and/or supporting cells; endothelial cells; muscle (e.g., heart muscle, smooth muscle, and/or skeletal muscle); small intestine; colon; adipocytes; kidney; liver; lung; spleen; stomach; esophagus; bladder; pancreas; thyroid; salivary gland; adrenal gland; pituitary gland; breast; skin; ovary; uterus; placenta; prostate; and testis. In kidney tissue, Megalin has been reported to be found on the surface of proximal tubular epithelial cells and podocytes. In proximal tubule epithelia cells of the kidney, Megalin expression has been observed in the brush border, in endocytic vesicles, dense apical tubules and/or lysosomes. Several ligands of Megalin have been identified, some of which are disclosed in Nielsen et al. 2016.

TABLE 1
Exemplary Megalin ligands

| | |
|---|---|
| Vitamin carrier proteins | Transcobalamin-vitamin B12 |
| | Vitamin D-binding protein |
| | Retinol-binding protein |
| | Folate-binding protein |
| Other carrier proteins | Albumin |
| | Myoglobin |
| | Hemoglobin |
| | Lactoferrin |
| | Selenoprotein P |
| | Metallothionein |
| | Neutrophil gelatinase-associated |
| | lipocalin |
| | Odorant-binding protein Transthyretin |
| | Liver-type fatty acid-binding protein |
| | Sex hormone binding globulin |
| Lipoproteins | Apolipoprotein B |
| | Apolipoprotein E |
| | Apolipoprotein J/clusterin |
| | Apolipoprotein H |
| | Apolipoprotein M |
| Hormones and signaling | Parathyroid hormone |
| proteins | Insulin |
| | Epidermal growth factor |
| | Prolactin |
| | Thyroglobulin |
| | Sonic hedgehog protein |
| | Angiotensin II |
| | Leptin |
| | Bone morphogenic protein 4 |
| | Connective tissue growth factor |
| | Insulin-like growth factor |
| | Survivin |
| Enzymes and enzyme | Plasminogen activator |
| inhibitors | inhibitor type I |
| | Plasminogen activator inhibitor |
| | type I-urokinase Plasminogen activator |
| | inhibitor |
| | type I-tissue plasminogen activator |
| | Pro-urokinase |
| | Lipoprotein lipase |
| | Plasminogen |
| | a-Amylase |
| | Lysozyme |
| | Cathepsin B |
| | a-Galactosidase A |
| | Cystatin C |
| | Recombinant activated factor VIIa |
| Immune- and stress-related | Ig light chains |
| proteins | Pancreatitis-associated protein 1 |
| | a1-Microglobulin |
| | b2-Microglobulin |
| | Ig light chains |
| Others | Receptor-associated protein |
| | Ca2b |
| | Cytochrome C |
| | Seminal vesicle secretory protein II |
| | Coagulation factor VII |
| | Coagulation factor VIII |

Additional exemplary Megalin binding moieties or ligands are disclosed in U.S. Pat. Nos. 7,560,431, 8,877,714, 8,795,627; International Patent Application WO 2006/138343, U.S. Pat. Nos. 9,388,418, 10,065,993, International Patent Application WO 2017/100700, and International Patent Application WO 2018/232122, the entire contents of each of which are hereby incorporated by reference.

In some embodiments, a kidney cell surface factor is Megalin, or a fragment, or a variant thereof.

In some embodiments, a targeting moiety is or comprises a megalin-binding moiety. In particular embodiments, a targeting moiety binds an extracellular domain (e.g., to a site on the extracellular domain, e.g., a site that is exposed when megalin is on a cell surface) of megalin. In other particular embodiments, a conjugate comprises a targeting moiety that binds an extracellular domain (e.g., to a site on an extracellular domain, e.g., a site that is exposed when megalin is on a cell surface) of megalin and, upon binding to megalin, causes the internalization of megalin.

In some embodiments, a targeting moiety comprising a megalin-binding moiety binds an extracellular domain of megalin at a nephron apical membrane.

In some embodiments, a targeting moiety comprising a megalin-binding moiety binds an extracellular domain of megalin at a nephron basolateral membrane.

### Cubilin-Binding Moieties

Cubilin is a receptor of about 460 kDa. Cubilin is also known as IFCR, Gp280, Intrinsic Factor-Vitamin B12 Receptor, MGA1, or IGS1. As an extracellular protein, Cubilin can interact with other membrane proteins, e.g., Megalin. One of the functions of Cubilin is as a receptor for intrinsic factor-vitamin B12 complexes.

Human Cubilin protein sequence:
SEQ ID NO: 5
MMNMSLPFLWSLLTLLIFAEVNGEAGELELQRQKRSINLQQPRMATERGNLVELTGSAQN
IEFRTGSLGKIKLNDEDLSECLHQIQKNKEDIIELKGSAIGLPQNISSQIYQLNSKLVDL
ERKFQGLQQTVDKKVCSSNPCQNGGTCLNLHDSFFCICPPQWKGPLCSADVNECEIYSGT
PLSCQNGGTCVNTMGSYSCHCPPETYGPQCASKYDDCEGGSVARCVHGICEDLMREQAGE
PKYSCVCDAGWMFSPNSPACTLDRDECSFQPGPCSTLVQCENTQGSFYCGACPTGWQGNG
YICEDINECEINNGGCSVAPPVECVNTPGSSHCQACPPGYQGDGRVCTLTDICSVSNGGC
HPDASCSSTLGSLPLCTCLPGYTGNGYGPNGCVQLSNICLSHPCLNGQCIDTVSGYFCKC
DSGWTGVNCTENINECLSNPCLNGGTCVDGVDSFSCECTRLWTGALCQVPQQVCGESLSG
INGSFSYRSPDVGYVHDVNCFWVIKTEMGKVLRITFTFFRLESMDNCPHEFLQVYDGDSS
SAFQLGRFCGSSLPHELLSSDNALYFHLYSEHLRNGRGFTVRWETQQPECGGILTGPYGS
IKSPGYPGNYPPGRDCVWIVVTSPDLLVTFTFGTLSLEHHDDCNKDYLEIRDGPLYQDPL
LGKFCTTFSVPPLQTTGPFARIHFHSDSQISDQGFHITYLTSPSDLRCGGNYTDPEGELF
LPELSGPFTHTRQCVYMMKQPQGEQIQINFTHVELQCQSDSSQNYIEVRDGETLLGKVCG
NGTISHIKSITNSVWIRFKIDASVEKASFRAVYQVACGDELTGEGVIRSPFFPNVYPGER
TCRWTIHQPQSQVILLNFTVFEIGSSAHCETDYVEIGSSSILGSPENKKYCGTDIPSFIT
SVYNFLYVTFVKSSSTENHGFMAKFSAEDLACGEILTESTGTIQSPGHPNVYPHGINCTW
HILVQPNHLIHLMFETFHLEFHYNCTNDYLEVYDTDSETSLGRYCGKSIPPSLTSSGNSL
MLVFVTDSDLAYEGFLINYEAISAATACLQDYTDDLGTFTSPNFPNNYPNNWECIYRITV
RTGQLIAVHFTNFSLEEAIGNYYTDFLEIRDGGYEKSPLLGIFYGSNLPPTIISHSNKLW
LKFKSDQIDTRSGFSAYWDGSSTGCGGNLTTSSGTFISPNYPMPYYHSSECYWWLKSSHG
SAFELEFKDFHLEHHPNCTLDYLAVYDGPSSNSHLLTQLCGDEKPPLIRSSGDSMFIKLR
TDEGQQGRGFKAEYRQTCENVVIVNQTYGILESIGYPNPYSENQHCNWTIRATTGNTVNY
TFLAFDLEHHINCSTDYLELYDGPRQMGRYCGVDLPPPGSTTSSKLQVLLLTDGVGRREK
GFQMQWFVYGCGGELSGATGSFSSPGFPNRYPPNKECIWYIRTDPGSSIQLTIHDEDVEY
HSRCNFDVLEIYGGPDFHSPRIAQLCTQRSPENPMQVSSTGNELAIRFKTDLSINGRGEN
ASWQAVTGGCGGIFQAPSGEIHSPNYPSPYRSNTDCSWVIRVDRNHRVLLNFTDEDLEPQ
DSCIMAYDGLSSTMSRLARTCGREQLANPIVSSGNSLFLRFQSGPSRQNRGFRAQFRQAC
GGHILTSSFDTVSSPRFPANYPNNQNCSWIIQAQPPLNHITLSFTHFELERSTTCARDFV
EILDGGHEDAPLRGRYCGTDMPHPITSFSSALTLRFVSDSSISAGGFHTTVTASVSACGG
TFYMAEGIFNSPGYPDIYPPNVECVWNIVSSPGNRLQLSFISFQLEDSQDCSRDEVEIRE
GNATGHLVGRYCGNSFPLNYSSIVGHTLWVRFISDGSGSGTGFQATFMKIFGNDNIVGTH
GKVASPFWPENYPHNSNYQWTVNVNASHVVHGRILEMDIEEIQNCYYDKLRIYDGPSIHA
RLIGAYCGTQTESFSSTGNSLTFHFYSDSSISGKGFLLEWFAVDAPDGVLPTIAPGACGG
FLRTGDAPVFLFSPGWPDSYSNRVDCTWLIQAPDSTVELNILSLDIESHRTCAYDSLVIR
DGDNNLAQQLAVLCGREIPGPIRSTGEYMFIRFTSDSSVTRAGENASFHKSCGGYLHADR
GIITSPKYPETYPSNLNCSWHVLVQSGLTIAVHFEQPFQIPNGDSSCNQGDYLVLRNGPD
ICSPPLGPPGGNGHFCGSHASSTLFTSDNQMFVQFISDHSNEGQGFKIKYEAKSLACGGN
VYIHDADSAGYVTSPNHPHNYPPHADCIWILAAPPETRIQLQFEDREDIEVTPNCTSNYL
ELRDGVDSDAPILSKFCGTSLPSSQWSSGEVMYLRFRSDNSPTHVGFKAKYSIAQCGGRV
PGQSGVVESIGHPTLPYRDNLFCEWHLQGLSGHYLTISFEDENLQNSSGCEKDFVEIWDN
HTSGNILGRYCGNTIPDSIDTSSNTAVVRFVTDGSVTASGFRLRFESSMEECGGDLQGSI
GTFTSPNYPNPNPHGRICEWRITAPEGRRITLMENNLRLATHPSCNNEHVIVENGIRSNS
PQLEKLCSSVNVSNEIKSSGNTMKVIFFTDGSRPYGGFTASYTSSEDAVCGGSLPNTPEG
NFTSPGYDGVRNYSRNLNCEWTLSNPNQGNSSISIHFEDFYLESHQDCQFDVLEFRVGDA
DGPLMWRLCGPSKPTLPLVIPYSQVWIHFVTNERVEHIGFHAKYSFTDCGGIQIGDSGVI
TSPNYPNAYDSLTHCSSLLEAPQGHTITLTFSDFDIEPHTTCAWDSVTVRNGGSPESPII
GQYCGNSNPRTIQSGSNQLVVTFNSDHSLQGGGFYATWNTQTLGCGGIFHSDNGTIRSPH
WPQNFPENSRCSWTAITHKSKHLEISFDNNFLIPSGDGQCQNSFVKVWAGTEEVDKALLA
TGCGNVAPGPVITPSNTFTAVFQSQEAPAQGFSASFVSRCGSNFTGPSGYIISPNYPKQY
DNNMNCTYVIEANPLSVVLLTFVSFHLEARSAVTGSCVNDGVHIIRGYSVMSTPFATVCG
DEMPAPLTIAGPVLLNFYSNEQITDFGFKFSYRIISCGGVFNESSGIITSPAYSYADYPN
DMHCLYTITVSDDKVIELKFSDFDVVPSTSCSHDYLAIYDGANTSDPLLGKFCGSKRPPN
VKSSNNSMLLVFKTDSFQTAKGWKMSFRQTLGPQQGCGGYLTGSNNTFASPDSDSNGMYD
KNLNCVWIIIAPVNKVIHLTENTFALEAASTRQRCLYDYVKLYDGDSENANLAGTFCGST
VPAPFISSGNFLTVQFISDLTLEREGFNATYTIMDMPCGGTYNATWTPQNISSPNSSDPD
VPFSICTWVIDSPPHQQVKITVWALQLTSQDCTQNYLQLQDSPQGHGNSRFQFCGRNASA
VPVFYSSMSTAMVIFKSGVVNRNSRMSFTYQIADCNRDYHKAFGNLRSPGWPDNYDNDKD
CTVTLTAPQNHTISLFFHSLGIENSVECRNDFLEVRNGSNSNSPLLGKYCGTLLPNPVES
QNNELYLRFKSDSVTSDRGYEIIWTSSPSGCGGTLYGDRGSFTSPGYPGTYPNNTYCEWV
LVAPAGRLVTINFYFISIDDPGDCVQNYLTLYDGPNASSPSSGPYCGGDTSIAPFVASSN
QVFIKFHADYARRPSAFRLTWDS
Cubilin is encoded by the CUBN gene. A nucleic acid
sequence encoding human Cibiling is provided by
SEQ ID NO: 6

| | |
|---|---|
| 1 | atgctcagtt ggttggagtg gcctcactct tacctgccaa cctgggaggt tgatgatgaa |
| 61 | catgtcttta ccttttcttt ggagtttgct taccttatta atatttgctg aagtaaatgg |
| 121 | cgaagctgga gaacttgagc tgcagagaca aaaaagaagc atcaatctcc aacagcctcg |
| 181 | aatggctaca gagagaggaa atttggtgtt tcttacgggg tctgctcaaa acattgagtt |
| 241 | tagaaccgga tccctgggaa aaattaaatt aaatgatgaa gatctcagtg agtgtttaca |
| 301 | tcagatccag aaaaacaaag aagatattat agagttaaaa gggagtgcaa ttggtctgcc |
| 361 | tcaaaatata tctagtcaaa tctatcagct taattccaag ctggtggatc ttgagagaaa |
| 421 | attccaaggc ttgcagcaga ctgttgacaa aaaggtttgc agcagcaatc cttgccagaa |
| 481 | tggtggaacc tgcctcaatc tgcatgattc ctttttttgt atctgtcccc cacagtggaa |
| 541 | gggtcctctc tgctcagctg atgttaacga atgtgagatt tactcaggaa cacccttgag |
| 601 | ctgccagaat ggaggcacat gtgttaatac aatgggaagt tacagttgtc actgcccacc |
| 661 | tgagacgtac ggaccccagt gtgcatccaa atatgacgac tgtgaagggg gttctgtggc |
| 721 | acgctgtgtc catggcatct gtgaggattt aatgcgagag caagctggag agcccaagta |
| 781 | cagctgcgtc tgtgatgctg ggtggatgtt ttcacccaac agccctgcct gcacgctgga |
| 841 | cagagacgag tgcagcttcc agcccgggcc ttgctccaca cttgtgcagt gtttcaacac |
| 901 | tcaaggctct ttctactgtg gggcctgtcc aacaggctgg caaggcaatg gatatatttg |
| 961 | cgaagatatc aatgaatgtg agataaataa cggcggctgt tctgtggctc cacccgttga |
| 1021 | gtgtgtgaat acacctgggt cttcccactg ccaggcctgt ccaccagggt accagggtga |
| 1081 | cggaagagtg tgcacactca cagacatctg ctcagtcagt aatggaggct gccacccaga |
| 1141 | tgcctcatgc tcctcaactc taggttcctt acctctctgc acgtgtctcc cgggttatac |
| 1201 | tggaaatggt tatgggccaa atggatgtgt gcagctcagt aatatttgcc taagtcaccc |
| 1261 | ctgtctaaat ggacaatgca tcgacactgt ctctggttat ttttgtaagt gtgactcagg |
| 1321 | ttggacaggt gtcaactgta cagaaaacat caatgagtgt ttgagcaacc cctgtttgaa |
| 1381 | tggaggaact tgtgttgatg gcgttgattc tttcagttgt gaatgcacac gtctctggac |
| 1441 | tggagctctc tgtcaggttc ctcagcaagt ttgtggagag tccctctcag gaataaatgg |
| 1501 | aagcttcagc tacaggagcc cggatgttgg ttatgttcat gatgttaact gcttctgggt |
| 1561 | tatcaaaact gaaatgggaa aggtcctgcg tatcactttc acttttttcc ggttagaatc |
| 1621 | catggacaac tgtccacacg agtttcttca ggtttatgat ggagattcct cttctgcttt |
| 1681 | tcaacttgga agattttgtg gctccagcct ccctcatgaa ctcctcagca gtgacaatgc |
| 1741 | tctctatttt catctctatt ctgaacattt aagaaatggg agaggcttta cagtaagatg |
| 1801 | ggaaacacag caaccagagt gtggaggtat cctgactggt ccttacggtt ctattaagtc |
| 1861 | tccggggtat cctggaaact atcccccagg aagagattgt gtctggattg ttgtaactag |
| 1921 | tcctgacctc ctggtaacat ttacttttgg gaccttgagc ctcgagcacc atgatgactg |
| 1981 | caacaaagat taccttgaga ttcgagatgg tcctttgtat caggaccccc ttcttgggaa |
| 2041 | gttctgcacc actttctctg tcccaccgct ccagactact ggcccctttg ccagaattca |
| 2101 | cttccattca gactcccaga ttagtgacca aggcttccat atcacctact taacatcacc |
| 2161 | ttcggatctg cgttgtggtg ggaactacac ggacccagag ggtgaactct tcttgcctga |
| 2221 | gttgtctggg cctttcactc acaccaggca atgcgtctat atgatgaagc agccccaggg |
| 2281 | agaacaaata caaatcaact tcacccacgt ggagctgcaa tgccagagtg acagttctca |
| 2341 | gaattacatt gaggttcgag atggtgaaac cttacttgga aaagtctgtg gcaacggaac |
| 2401 | catctctcac attaaatcca ttactaatag tgtctggatc aggtttaaaa tagatgcttc |
| 2461 | tgttgaaaaa gctagtttca gagctgttta tcaagtcgct tgcggggatg aattaactgg |
| 2521 | agaaggggtc attcgctcgc ctttttttcc taacgtgtat cctggagaaa gaacctgtag |
| 2581 | gtggaccatc caccagcccc aaagccaagt cattctcctc aacttcactg tctttgaaat |
| 2641 | tggaagttct gcccactgtg aaacagatta tgttgagatt ggtagcagtt ccattttggg |
| 2701 | ttctcctgaa aataaaaagt attgcggtac agacatacct tcatttataa catctgtgta |
| 2761 | caattttctt tatgtcacat tcgtgaaaag ttcttctact gaaaaccatg gtttcatggc |
| 2821 | taagttcagt gctgaggatt tggcatgtgg agaaattctt acagaatcaa cagggaccat |
| 2881 | tcaaagtcct ggccatccaa atgtctaccc ccacggtatc aactgtactt ggcatatatt |
| 2941 | agtccaacct aatcacctga ttcatttaat gttcgaaaca tttcatctgg agtttcatta |
| 3001 | caattgcaca aacgactact tggaagttta tgacaccgac tctgagacat cccttggaag |
| 3061 | atactgtgga aagtcgatcc cgccatctct cacaagcagt ggtaactcat tgatgctggt |
| 3121 | gtttgtgact gactccgacc tcgcttatga aggcttctta ataaactatg aagcaatcag |
| 3181 | tgcagcaaca gcatgtttgc aagactacac agatgatttg gggacattca cttctccaaa |
| 3241 | cttccccaat aattatccca acaactggga atgcatttat cggatcacag tgagaactgg |
| 3301 | ccaactgatt gcagtgcact tcacaaactt ctccttggag gaagccattg gaaactatta |
| 3361 | tacagatttt ctggaaatca gagatggagg ctatgaaaaa tcaccattgc tgggaatatt |
| 3421 | ctatggctca aatctacccc caacaatcat ctctcatagt aacaaactat ggttaaaatt |
| 3481 | taagagtgac caaatagaca caaggtctgg attctcagct tactgggatg ggtcatcaac |
| 3541 | aggttgcggg ggtaatctca ccacttcaag cggcacgttc atatctccca actacccgat |
| 3601 | gccctattac cacagctctg aatgctactg gtggttgaaa tctagccacg gcagcgcatt |
| 3661 | tgaactggaa ttcaaagact ttcacttgga gcatcatcca aactgcactt tagattacct |
| 3721 | ggctgtatat gatggcccaa gtagcaactc tcatctgcta actcagcttt gtggggatga |
| 3781 | gaaaccccct cttattcgtt ctagtggaga cagcatgttt ataaaactga ggacagatga |
| 3841 | aggtcagcaa ggacgtggct tcaaggctga ataccggcag acatgtgaga atgtggtaat |
| 3901 | agtcaatcaa acctatggca tcttagagag tatagggtat ccgaatcctt attctgaaaa |
| 3961 | tcagcattgc aactggacca tccgggcaac aacaggcaac actgtgaact acacattttt |
| 4021 | agcatttgac ttggaacatc acataaactg ctccacagat tatttagagc tctatgatgg |
| 4081 | accacggcag atgggacgct actgtggagt agacctgccc cctccaggga gtactacaag |
| 4141 | ctccaagctt caagtgctgc tccttacaga tggggttggc cgccgtgaga aaggatttca |
| 4201 | gatgcagtgg tttgtttacg gttgtggtgg agagctgtct ggggccacag gctccttcag |
| 4261 | cagccccggg ttccccaaca ggtatccacc aaacaaggag tgtatctggt acattaggac |
| 4321 | ggaccccggg agtagcattc agctcaccat ccatgacttc gatgtggagt atcattcaag |
| 4381 | gtgcaacttt gatgtcttgg agatctatgg aggccccgat ttccactctc ccagaatagc |
| 4441 | ccaactgtgt acccagagat cacctgagaa ccccatgcag gtctccagca ctggaaatga |
| 4501 | gctagcaatt cgattcaaga ccgacttgtc cataaatggg agaggcttca atgcgtcatg |
| 4561 | gcaagcagtc actggaggtt gtggtgggat tttccaggct cccagtggag agattcattc |
| 4621 | tccaaattac cccagtcctt ataggagcaa cacagactgt tcttgggtca ttcgggttga |
| 4681 | cagaaatcat cgtgttctct tgaacttcac tgactttgat cttgaaccac aagactcttg |
| 4741 | tattatggca tacgatggct taagctccac aatgtcccgc cttgccagga cgtgtggaag |
| 4801 | ggagcagctg gctaacccca tcgtctcctc aggaaacagc ctcttcttga gatttcagtc |
| 4861 | tggcccttcc agacagaaca gaggcttccg agctcaattc aggcaagcct gcggaggcca |
| 4921 | catcctcacc agctcatttg atactgtttc ctctccacgg ttccctgcca attatccaaa |
| 4981 | caatcagaac tgcagctgga tcattcaagc gcaacctcca ttaaatcata tcaccctctc |
| 5041 | ttttacccac tttgaacttg aaagaagcac aacgtgtgca cgtgactttg tagaaatttt |
| 5101 | ggatggcggc cacgaagacg cgcccctccg aggccgttac tgtggcaccg acatgcccca |
| 5161 | tcctatcaca tccttcagca gcgccctgac gctgagattc gtctctgatt ctagcatcag |
| 5221 | tgctgggggt ttccacacca cggtcaccgc atcagtgtcg gcttgtggtg gaacgttcta |
| 5281 | catggctgaa ggcatcttca acagccctgg ctacccagac atttatcccc ctaatgtgga |
| 5341 | atgtgtctgg aacatcgtca gttcccctgg caaccggctc cagctgtctt ttatatcttt |
| 5401 | ccagttggaa gactctcagg actgcagcag agattttgtg gagatccgtg aaggaaatgc |
| 5461 | cacgggtcac ttggtgggac gatactgtgg aaactccttc cctctcaatt attcttccat |
| 5521 | cgttggacat accctgtggg tcagatttat ctcagatggt tctggcagcg gcacgggctt |
| 5581 | ccaggccaca tttatgaaga tatttggcaa tgataatatt gtgggaactc atgggaaagt |
| 5641 | cgcctctcct ttctggcctg aaaactaccc acataactcc aattaccaat ggacagtaaa |
| 5701 | tgtgaatgca tctcacgttg tccatggtag aatcttggag atggacatag aagaaataca |
| 5761 | aaactgctat tatgacaaat taaggatcta tgatgggcct agcattcacg cccgcctaat |
| 5821 | tggagcttac tgtggtaccc agactgaatc tttcagctcc actggaaatt ctttgacatt |
| 5881 | tcatttttac tccgactctt caatctcagg gaagggattc cttctggagt ggtttgcagt |
| 5941 | ggatgcacct gatggtgttt tacctaccat tgctccaggt gcttgtggtg gcttcctgag |
| 6001 | gacgggagat gcacccgtgt ttctcttctc cccgggctgg cctgacagtt acagtaatag |
| 6061 | agtggactgt acgtggctca tccaggctcc cgactctacc gtggaactca acattctttc |
| 6121 | cctggacatt gaatctcacc gaacgtgtgc ctatgatagc cttgtgatac gagatggaga |
| 6181 | taataacttg gcccagcagc tagcagttct ctgtggcaga gagatccctg ggcccatccg |
| 6241 | gtctactgga gagtacatgt tcatccgctt cacctcggac tccagtgtaa ccagggcagg |
| 6301 | cttcaatgca tcctttcaca agagctgcgg tggatatttg catgcagaca gagggatcat |
| 6361 | cacgtccccc aagtatccag agacttaccc atccaacctc aactgttctt ggcacgtcct |
| 6421 | ggtccaaagt ggcctgacca ttgctgtcca ttttgaacag cctttccaga ttccaaatgg |
| 6481 | agattcttct tgcaaccagg gggattactt ggtgctaaga aatggtcctg atatctgttc |
| 6541 | tccacccttg ggaccccctg gaggaaatgg tcatttttgt ggcagtcatg cttcatcaac |
| 6601 | tctgttcacc tcggataatc aaatgtttgt tcagtttatt tctgatcaca gtaatgaagg |
| 6661 | gcaaggattt aaaatcaaat atgaggcaaa gagtttagcc tgtgggggca acgtctacat |
| 6721 | ccatgatgct gattctgctg ggtatgtgac ctcccccaac caccctcata attatccccc |
| 6781 | gcacgctgat tgcatttgga tcttagcggc tccaccggaa acacgcatac agctgcaatt |
| 6841 | tgaagatcga ttcgatattg aagtaacacc caactgtact tccaactacc ttgagttgcg |
| 6901 | ggatggagtg gattcggatg caccaatact ttccaaattt tgtgggacat ctttgcccag |
| 6961 | cagtcagtgg tcctcaggag aggttatgta tttgagattt cgatctgaca acagccccac |
| 7021 | acatgtggga ttcaaggcca agtattctat agctcagtgt gggggaagag taccagggca |
| 7081 | aagtggtgtt gttgaaagca ttggacatcc aacacttcca tacagagaca acttattctg |
| 7141 | tgagtggcat ctccaggggc tctctggaca ctatctcacc atctcttttg aagactttaa |
| 7201 | ccttcagaat tcttctggct gtgaaaaaga cttcgtggag atctgggaca atcatacctc |
| 7261 | tggaaacatc ttgggcagat actgtggaaa caccattcct gacagcatag acacttctag |
| 7321 | caatactgct gtggtcaggt ttgtcacaga cggctctgtg actgcctcag gattcagact |
| 7381 | gcgatttgaa tccagtatgg aagagtgtgg tggggatctt cagggctcta ttggaacatt |
| 7441 | tacttctccc aactacccga acccaaatcc tcatggccgg atctgcgagt ggagaatcac |
| 7501 | tgccccggag ggaaggcgga tcaccctaat gtttaacaac ctgaggctgg ccacgcatcc |
| 7561 | gtcctgcaac aatgagcatg tgatagtatt caatggcatt agaagtaact caccccagct |
| 7621 | agagaaactg tgtagtagtg tgaatgtaag caatgagatt aaatcttcag gaaacacaat |
| 7681 | gaaagtcatt tttttcacgg atggatccag gccatatggc ggcttcactg cttcctatac |
| 7741 | ctccagtgaa gatgcagtgt gtggtgggtc tcttccaaat actcctgaag gaaactttac |
| 7801 | ttctcctggc tatgacggag tcaggaatta ctcaagaaac ctgaactgcg aatggactct |
| 7861 | cagcaatcca aatcagggaa attcatccat ttccattcac tttgaagatt tttacctaga |
| 7921 | aagtcaccaa gactgtcaat ttgatgtcct cgagtttcga gtgggtgatg ctgatgggcc |
| 7981 | cctgatgtgg agactttgtg gtccttcaaa gcctacattg ccattggtta taccttattc |
| 8041 | tcaggtatgg attcactttg tcaccaacga acgtgtagaa cacattggat tccatgcaaa |
| 8101 | gtattccttt acagattgtg gcggaataca gataggtgac agtggagtga tcacaagccc |
| 8161 | caactatcca aatgcttatg acagcctgac ccactgctct tcgctgttgg aggccccaca |
| 8221 | agggcacacc atcactctca catttagtga ctttgatatt gaaccccata caacttgtgc |
| 8281 | ttgggactct gtcactgtca ggaatggtgg gtcccctgaa tcacccatca taggacaata |
| 8341 | ctgtggaaat tcaaacccca ggacaataca gtcaggttcc aatcagctgg tcgtgacttt |
| 8401 | taactcagac cattcattgc aaggtggtgg attttatgct acgtggaaca cacaaacttt |
| 8461 | aggttgtggt ggaatatttc attctgataa tggtacaatc agatcccctc actggcctca |
| 8521 | gaattttccc gaaaacagca gatgttcctg gacggccatt actcacaaaa gtaaacactt |
| 8581 | ggagatcagc tttgacaaca acttcctaat ccccagcggt gatggacaat gtcagaatag |
| 8641 | cttcgtgaag gtgtgggcag gaactgagga ggtggacaaa gccctgctag ccactggctg |
| 8701 | tgggaacgtg gctccgggtc ccgttatcac accaagtaac acattcactg ccgtcttcca |
| 8761 | gtctcaggag gcaccagctc agggcttctc cgcgtccttt gttagccgat gtggaagtaa |
| 8821 | tttcactggc ccttcaggtt acatcatttc tccaaattac ccaaaacaat atgacaacaa |
| 8881 | catgaattgc acctatgtca tagaggctaa tcctctgtca gtggtcctct tgacttttgt |
| 8941 | gtccttccac ttagaagctc gttccgctgt gacgggaagc tgtgtcaacg atggcgtgca |
| 9001 | cattatcaga ggttacagcg tcatgtccac cccatttgct actgtgtgtg gggatgagat |
| 9061 | gccagctccc ctcaccatcg ctgggccggt tctgcttaac ttctactcca acgagcaaat |
| 9121 | cacagacttc ggattcaagt tttcctatag gataatctcc tgtggtggtg tgttcaattt |
| 9181 | ctcttctgga atcatcacaa gtcctgccta ttcatacgca gactacccaa atgatatgca |
| 9241 | ctgtctgtat accatcaccg ttagtgacga caaggtgatc gagctcaagt tcagtgattt |
| 9301 | tgatgtggtt ccctccacct cctgctccca tgactacctg gcaatttacg atggtgccaa |
| 9361 | taccagcgat ccccttcttg gcaaattctg cggttccaag cgcccaccaa atgtgaagag |
| 9421 | cagcaataat agtatgctcc tggtgttcaa gacagattca tttcagacag caaaaggctg |
| 9481 | gaagatgtct ttccggcaga cattggggcc tcagcaagga tgtggtggtt atctgacagg |
| 9541 | ctcgaataat acctttgcct ctcctgattc tgattcgaat ggaatgtatg acaagaattt |
| 9601 | aaactgtgta tggatcataa ttgcacctgt aaacaaagta attcacctca ccttcaatac |
| 9661 | atttgctctg gaggcagcaa gtactaggca aagatgcctt tatgattatg taaagttata |
| 9721 | tgatggggat agtgaaaatg cgaacttggc tggaacgttt tgtggttcca cagtacctgc |
| 9781 | tccttttatc tcttctggta acttccttac ggttcaattc atcagtgact taacattaga |
| 9841 | gagggaagga tttaatgcta catacaccat catggacatg ccttgtggtg gaacatacaa |
| 9901 | tgcaacttgg accccacaaa atatttcatc acccaattca tcagacccag atgtcccatt |
| 9961 | ttccatctgt acttgggtca ttgattcccc tccgcatcag caggtcaaga taactgtgtg |
| 10021 | ggcattacag ctgacctcgc aagactgcac gcagaattac ttacagcttc aggactcacc |
| 10081 | gcagggtcac ggaaattcaa gatttcagtt ctgtggcaga aatgcttcgg ctgtgccagt |
| 10141 | gttttattct tctatgagta ctgcaatggt cattttcaaa tctggagttg taaacagaaa |
| 10201 | ctctagaatg agtttcacct atcagattgc agattgcaac agagactatc acaaggcatt |
| 10261 | tggcaacctg agaagccctg gatggccaga taactacgac aatgacaagg attgcaccgt |
| 10321 | tactctcaca gccccccaga accacaccat ttccctcttt tttcattcac ttggcatcga |
| 10381 | gaactcagtt gaatgcagaa acgatttctt ggaggtgaga aatggaagta acagcaattc |
| 10441 | accattactg ggcaagtact gtggaactct gctgccaaac cctgtcttct ctcaaaataa |
| 10501 | tgaactatac ctacgattta agagtgatag tgtaacttct gatcgtggat atgaaatcat |
| 10561 | ctggacttca tcaccctctg gatgtggtgg aactctttat ggagacagag gctcattcac |
| 10621 | cagccccggc tatccaggca catacccaaa caacacgtac tgcgagtggg tccttgttgc |
| 10681 | tcctgctgga aggcttgtca ccatcaactt ctacttcatc agcattgacg atccaggaga |
| 10741 | ctgtgtccag aactatctca cactctatga tgggcccaac gccagctctc catcctctgg |
| 10801 | accatactgc ggaggcgaca ccagcatagc tcccttcgtg gcttcctcaa atcaggtctt |
| 10861 | cataaaattt catgctgatt atgcacggcg tccatccgca ttccgattaa cttgggacag |
| 10921 | ctaagtgggt aacaactcgt gttcactcag cactttccct ctgcagcacg ctggacagca |
| 10981 | ctctgccatc ctgatacatg acccctgctg atgccacaga gaataagctg aacttgtatg |
| 11041 | gtttttcacc aaaccatgga tagaatcaat atttgtaggc caggcgtggt ggctcacccc |
| 11101 | cctgtattct cagcactttg ggaggccgag gcaggttgat cacctgaggt caggagtttg |
| 11161 | agactagcct ggccagcatg gtgaaacctc atctctctaa caatataata attagccagg |
| 11221 | cgtggtggtg ggtgcctgta attccagcca ctcgagaggc tgaggcagga gaattgcttg |
| 11281 | aacccaggag gcagaggttg cagtgagcta agatcacacc actacactcc agcctgggcg |
| 11341 | agacggcaag actccatctc aaaaaaaaaa gaaacaaaaa aaaccagaat caatatttgt |
| 11401 | acattttctc gaacatagaa tatagcttct ttagtcttga gtgtgcattt cattctaata |
| 11461 | ttttgagctg aaatttaaaa aaactttgaa agagttggaa atgattatgg catatgtgac |
| 11521 | atacattttt aaaagttaat aataatagcc aggggcagtg gctcataccc ataatcccag |
| 11581 | cacgctggga ggccatgatg ggaggattgc ttgaacctag gagtttgaga ccagcctggg |
| 11641 | caacaaagtg agacctgatt tttacaaaaa atcaaaaaat tagccaggca tggtggcatg |
| 11701 | cacccgtggt tccagctaca caggaggttg aagcaggagg atcacttgag cccagtaggt |
| 11761 | taaggctgca gtgaaaccct gtgaattaac cactgtactc cagcctgggt gacagactga |
| 11821 | gaccctatct caaaaatgac aacaagaaca acaaaagtta atgataatat agaagcataa |
| 11881 | atttcctgtg aatgttcaat tacacataat aaacattatt gaattgtaca caa. |

The extracellular domain of Cubilin includes repeats of CUB domains (complement Clr/C1s, Uegf [epidermal growth factor-related sea urchin protein], and bone morphogenic protein 1) and EGF-type repeats. A typical structure of Cubilin is disclosed in FIG. 1 of Marzolo and Farfan (2011), Biol Res 44:89-105, the entire contents of which are hereby incorporated by reference. The extracellular domain of Cubilin may also include one or more post-translational modifications, such as glycosylation.

Cubilin has been reported to be found on surfaces of one or more of the following tissues and/or cells:immune cells (e.g., bone marrow cells, lymph node cells, thymic cells, peripheral blood mononuclear cells [e.g., myeloid and/or lymphoid cells], erythrocytes, eosinophils, neutrophils, and/or platelets); nervous system (e.g., brain tissue, cortex, cerebellum, retinal cells, spinal cord cells, nerve cells, neurons, and/or supporting cells; endothelial cells; muscle (e.g., heart muscle, smooth muscle, and/or skeletal muscle); small intestine; colon; adipocytes; kidney; liver; lung; splenic; stomach; esophagus; bladder; pancreas; thyroid; salivary gland; adrenal gland; pituitary gland; breast; skin; ovary; uterus; placenta; prostate; and testis. In kidney tissue, Cubilin has been reported to be found on the surface of proximal tubular epithelial cells and podocytes. Several ligands of Cubilin have been identified, some of which are disclosed in Nielsen et al. 2016.

TABLE 6
Exemplary Cubilin Ligands

| | |
|---|---|
| Vitamin carrier proteins | Intrinsic factor vitamin B12 |
| | Vitamin D-binding protein |
| Other carrier proteins | Albumin |
| | Myoglobin |
| | Hemoglobin |
| | Transferrin |
| Lipoproteins | Apolipoprotein A-I |
| | High density lipoprotein |
| Hormones and signaling | Fibroblast growth factor |
| proteins | |
| Enzymes and enzyme | Recombinant activated factor VIIa |
| inhibitors | |
| Immune- and stress-related | Ig light chains |
| proteins | a1-Microglobulin |
| | Clara cell secretory protein |
| Others | Receptor-associated protein |
| | Coagulation factor VII |

Additional exemplary Cubilin binding moieties or ligands are disclosed in U.S. Pat. No. 10,065,993, International Patent Application WO 2017/100700, International Patent Application WO 2018/232122, and International Patent Application WO 2015/027205, the entire contents of each of which are hereby incorporated by reference.

In some embodiments, a kidney cell surface factor is Cubilin, or a fragment, or a variant thereof.

In some embodiments, a targeting moiety is or comprises a Cubilin-binding moiety.

### Targeting Moieties

In some embodiments, a targeting moiety (e.g., a megalin binding moiety) is chosen from: a peptide, an aminoglycoside, an endogenous ligand (e.g., a ligand disclosed in Table 1 or an analog or variant thereof), a xenobiotic, an antibody or a fragment thereof, or a combination thereof.

In some embodiments, a targeting moiety (e.g., a megalin binding moiety) is or comprises a peptide. In some embodiments, a peptide is chosen from: a KKEEEKKEEEKKEEEK (also referred to as (KKEEE) 3K) peptide; a fragment of receptor associated protein (RAP), e.g., a RAP fragment comprising residues 219-323); a peptide derived from a radiopharmaceutical conjugates such as ocreotide, ocreotate, exendin, minigastrin, and/or neurotensin; or a combination thereof.

In some embodiments, a targeting moiety (e.g., a megalin binding moiety) is or comprises KKEEEKKEEEKKEEEK (SEQ ID NO: 1).

RAP (receptor-associated protein) is a cellular protein comprising about 300 amino acids and is encoded by the LRPAP1 gene. An exemplary RAP sequence is provided by NP_002328.1, and encoded by NM_002337.4. RAP has been shown to bind to Megalin to suppress the interaction of the Megalin receptor with one or more ligands (Willnow et al., EMBO J. 15, 2632-2639, 1996). Studies have shown that a minimal functional domain of RAP comprising about 104 amino acids retains RAP's receptor binding and inhibition.

RAP protein sequence as provided by
NM_002337.4: (SEQ ID NO: 2):
MAPRRVRSFLRGLPALLLLLLFLGPWPAASHGGKYSREKNQPKPSPKRES
GEEFRMEKLNQLWEKAQRLHLPPVRLAELHADLKIQERDELAWKKLKLDG
LDEDGEKEARLIRNLNVILAKYGLDGKKDARQVTSNSLSGTQEDGLDDPR
LEKLWHKAKTSGKFSGEELDKLWREFLHHKEKVHEYNVLLETLSRTEEIH
ENVISPSDLSDIKGSVLHSRHTELKEKLRSINQGLDRLRRVSHQGYSTEA
EFEEPRVIDLWDLAQSANLTDKELEAFREELKHFEAKIEKHNHYQKQLEI
AHEKLRHAESVGDGERVSRSREKHALLEGRTKELGYTVKKHLQDLSGRIS
RARHNEL

In some embodiments, a RAP fragment comprises a fragment of SEQ ID NO: 2, or a sequence with at least 90% identity thereto. In some embodiments, a RAP fragment comprises an LDL receptor binding domain of RAP. In some embodiments, a RAP fragment comprises a fragment of about ˜104 amino acids. In some embodiments, a RAP fragment is or comprises residues 219-323 of RAP.

Exemplary RAP fragments are disclosed in U.S. Patent Application US 2008/0153753A1, the entire contents of which are hereby incorporated by reference.

In some embodiments, a peptide disclosed herein further comprise one or more fragments, domains, and/or residues.

In some embodiments, a peptide disclosed herein comprises one or more modified amino acids.

In some embodiments, a peptide has one or more, or all of the following characteristics: (i) low molecular weight, e.g., 0.5 kDa to 10 kDa; (ii) limited charge at pH 7, e.g., −10 to +10; (iii) binding to a cell surface receptor, e.g., Megalin, Cubilin, or both. Exemplary characteristics of peptides that may be useful in a targeting moiety are disclosed in Vegt, et al. Eur J Nucl Med Mol Imaging. 2011, and Wischnjow et al, Bioconjugate Chem. 2016, 27, 1050-1057, the entire contents of each of which are incorporated by reference herein.

In some embodiments, a targeting moiety (e.g., a megalin binding moiety) is or comprises an aminoglycoside. In some embodiments, an aminoglycoside is chosen from one or more, or all of: streptomycin, neomycin, kanamycin, paromomycin, gentamicin, G-418 (geneticin), ELX-02, tobramycin, amikacin, netilmicin, spectinomycin, sisomicin, dibekacin, isepamicin, framycetin, paromomycin, apramycin, fradiomycin, arbekacin, plazomicin, or a derivative or a variant thereof.

In some embodiments, a targeting moiety disclosed herein comprises an aminoglycoside comprising 2-deoxystreptamine.

In some embodiments, an aminoglycoside disclosed herein has minimal bactericidal activity and/or toxicity, e.g., nephrotoxicity.

In some embodiments, an aminoglycoside comprises a variant having reduced toxicity, e.g., reduced nephrotoxicity as compared to an aminoglycoside without a variant. In some embodiments, an aminoglycoside comprises a variant having reduced bactericidal activity as compared to an aminoglycoside without a variant. In some embodiments, an aminoglycoside comprises a variant which retains activity, e.g., readthrough activity of premature termination codons, as compared to an aminoglycoside without a variant. In some embodiments, a variant of an aminoglycoside has reduced overall cationic charge as compared to an aminoglycoside without a variant. Exemplary aminoglycosides and variants thereof are disclosed in: Popadynec M. et al., (2021) ACS Med. Chem. Lett. 12 (9), 1486-1492; and in Brasell E J et al., (2019), PLOS ONE 14 (12): e0223954; the entire contents of each of which is hereby incorporated by reference.

In some embodiments, an aminoglycoside comprises an analog of an aminoglycoside having reduced antimicrobial activity (e.g., an aminoglycoside produced by resistance mutations in bacteria), and/or reduced endosomal or lysosomal stability, or both.

In some embodiments, an aminoglycoside has one or more, or all of the following characteristics: (i) high potency for binding to a cell surface factor, e.g., Megalin, Cubilin, or both; (ii) low nephrotoxicity; (iii) low ototoxicity; (iv) reduced endosomal or lysosomal stability; (v) reduced antimicrobial activity; or (vi) a combination of any one or all of (i) to (v).

As will be appreciated by those in the field, minimum inhibitory concentration (MIC) can be used to determine susceptibility of microorganisms to antimicrobials such as aminoglycosides disclosed herein. MIC is the lowest concentration of an antimicrobial that inhibits visible growth of a microorganism after incubation (e.g., overnight incubation). MIC50 refers to antimicrobial concentration that inhibits growth of 50% of microorganisms tested, and MIC90 refers to antimicrobial concentration that inhibits growth of 90% of microorganisms tested. MIC can be measured with any assay known in the field, including with susceptibility strips. Microorganisms that can be used to determine MIC include gram negative bacteria or gram positive bacteria. Microorganisms with known susceptibility to a particular antimicrobial can be used as a reference in evaluating MIC for particular anti-microbials. Additional information on determining MICs and exemplary methods for determining MICs are disclosed in Kowalska-Krochmal and Dudek-Wicher (2021) Pathogens, 10 (2): 165; and Sueke H. et al., (2010) Immunology and Microbiology, Vol 51 (5), pp. 2519-2524, the entire contents of each of which are hereby incorporated by reference in their entirety.

In some embodiments, an aminoglycoside that is part of a conjugate agent disclosed herein (a conjugated aminoglycoside) has antimicrobial activity similar to that of an otherwise similar but unconjugated aminoglycoside.

In some embodiments, a conjugated aminoglycoside does not have or has lesser antimicrobial activity as compared to an otherwise similar but unconjugated aminoglycoside. In some embodiments, a conjugated aminoglycoside has at least 1.5-fold, at least 2-fold, at least 5-fold, at least 10-fold, at least 20-fold or more lower MIC compared to an unconjugated aminoglycoside. In some embodiments, MIC is measured with gram negative bacteria. In some embodiments, MIC is measured with gram positive bacteria.

In some embodiments, an aminoglycoside disclosed herein binds to one or more extracellular domains of a cell surface factor (e.g., Megalin, Cubilin, or both). In some embodiments, an aminoglycoside disclosed herein binds a cell surface receptor at or near one or more complement type repeats. Exemplary binding of an aminoglycoside to human Megalin is disclosed in Dagil R et al., (2013) Journal of Biological Chemistry; 288 (6); 4424-4435; the entire contents of which are hereby incorporated by reference.

In some embodiments, a targeting moiety (e.g., a megalin binding moiety) is or comprises an endogenous ligand, e.g., a ligand disclosed in Table 1. In some embodiments, an endogenous ligand is chosen from: vitamin carrier proteins, apolioproteins, peptide hormones, or a combination thereof.

In some embodiments, when a targeting moiety (e.g., a megalin binding moiety) is or comprises a vitamin carrier protein, a vitamin carrier protein comprises a vitamin carried by a vitamin carrier protein. For example, for a vitamin carrier protein that carries Vitamin D, in some embodiments such a vitamin carrier protein comprises both a vitamin carrier protein and Vitamin D when used in a kidney-specific binding moiety described herein.

In some embodiments, a targeting moiety (e.g., a megalin binding moiety) is or comprises a ligand or substrate that binds to or is carried by another protein, e.g., a receptor or a carrier protein.

Exemplary endogenous ligands are disclosed in Nielsen R. et al. (2016), Kidney Int. 89 (1): 58-67 the entire contents of which are hereby incorporated by reference.

In some embodiments, a targeting moiety (e.g., a megalin binding moiety) is or comprises a xenobiotic. In some embodiments, a xenobiotic is chosen from: Polymixins, aprotinin, trichosanthin, or a combination thereof.

In some embodiments, a targeting moiety (e.g., a megalin binding moiety) is or comprises an antibody or a fragment thereof. In some embodiments, a targeting moiety (e.g., a megalin binding moiety) is an antibody that binds to a relevant cell surface factor, e.g., Megalin. In some embodiments, an anti-Megalin antibody is a monoclonal antibody or a fragment thereof. In some embodiments, an anti-Megalin antibody is a polyclonal antibody or a fragment thereof. In some embodiments, an anti-Megalin antibody is a bispecific or multispecific antibody, or a fragment thereof. In some embodiments, a bispecific or multispecific antibody binds to Megalin and one or more additional antigens, e.g., a polypeptide present in podocytes.

Exemplary antibodies that bind Megalin include anti-Megalin autoantibodies found in patients having antibrush border antibodies and renal failure (ABBA disease), see e.g., Larsen C. et al., (2018) J Am Soc Nephrol. 29 (2): 644-653. Anti-Megalin antibodies are also disclosed in: Perez-Gomez M V et al., (2020) Clin Kidney J., 13 (3): 281-286; Dinesh K P et al., (2019) Am J Kidney Dis., 74 (1): 132-137, Larsen C P et al., (2018) J Am Soc Nephrol., (2): 644-653, and Gamayo A et al., (2019) Clin Kidney J., 13 (3): 468-472, the entire contents of each of which is hereby incorporated by reference.

Those skilled in the art would also appreciate various commercially available anti-Megalin antibodies (or fragments thereof) that can be utilized as a targeting moiety in a conjugate agent disclosed herein.

In some embodiments, an antibody used in a conjugate agent disclosed herein include one or more modifications of an Fc domain, e.g., an Fc variant. In some embodiments, an Fc variant comprises an effector null mutation. In some embodiments, an Fc variant has one or more of the following properties: (1) reduced effector function (e.g., reduced ADCC, ADCP and/or CDC); (2) reduced binding to one or more Fc receptors; and/or (3) reduced binding to Clq complement. In some embodiments, the reduction in any one, or all of properties (1)-(3) is compared to an otherwise similar antibody with a wildtype Fc region. In some embodiments, an Activin A antibody agent comprising a variant Fc region has reduced affinity to a human Fc receptor, e.g., FcγR I, FcγR II and/or FcγR III. Exemplary Fc region variants are disclosed in Saunders K. O., (2019) Frontiers in Immunology; vol 10, Article 296, the entire contents of which is hereby incorporated by reference. For example, a Fc region variant is or comprises a modification provided in Table 3 of Saunders KO (2019).

In some embodiments, an antibody used in a conjugate agent disclosed herein is a neutral binder, e.g., an antibody having no antagonism or blockage of binding sites for other substrates.

In some embodiments, an antibody which binds to Megalin and used in a conjugate agent disclosed herein can be trafficked intracellularly with Megalin. An exemplary anti-Megalin antibody with such properties is the 20B monoclonal antibody disclosed in Shah M. et al. (2013), Journal of Cell Biology 202 (1): 113-127, the entire contents of which are herein incorporated by reference.

In some embodiments, an antibody used in a conjugate agent disclosed herein comprises an Fc variant and is a neutral binder.

### Linkers

In some embodiments, a conjugate agent has the structure of Formula V:

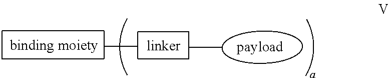

wherein
a is 1-8; and
each of the binding moiety, linker, and payload moiety is as defined above and described herein.

The synthesis and application of conjugate agents (e.g., “bioconjugates”) as tools for life science research, as diagnostic reagents, and as therapeutic agents has exploded in recent years and development of conjugate agents remains an area of intensive activity. In some embodiments, a bioconjugate or conjugate agent comprises a payload moiety that is chemically conjugated or linked covalently to a binding (e.g., targeting) moiety.

In some embodiments, a conjugate agent is prepared by conjugating or covalently linking a payload moiety to a binding moiety. In some embodiments, the payload moiety may be linked to a binding moiety by, for example reaction of the payload moiety in-solution with a binding moiety such as a drug, including a nucleic acid agent, e.g., oligonucleotide. The conjugate agent may also be prepared in a single synthesis, for example to prepare GalNAc-conjugated nucleic acids by solid-phase means. (For example, U.S. Pat. No. 9,422,562, WO2009073809, U.S. Pat. Nos. 8,106,022, 8,828,956, 9,133,461, and 10,131,907, each of which is incorporated by reference in its entirety).

Regardless of how produced and depending on the desired properties of the conjugate agent, it may or may not be advantageous to include a spacer or linker between the payload and the binding moiety. If it is advantageous to include a linker then linkers can be of many different types and chemical compositions.

Generally linkers are designated as “cleavable” or “non-cleavable”. Cleavable linkers are typically employed when it is desired that the payload and binding moiety to which it is conjugated be released so that either or both can better carry out their function (For example, U.S. Pat. Nos. 10,808,039 and 9,463,252, each of which is incorporated by reference in its entirety.) Non-cleavable linkers are typically employed to maintain the desired activity, performance and stability of the conjugate agent, for example enzymes linked to probes or (m)Abs to facilitate ELISA assays, to increase affinity, or bi-specificity, etc. Amongst the cleavable linkers are those that are cleaved chemically, for example by hydrolysis, change in pH, reduction or oxidation, and those that are cleaved enzymatically, for example by action of a protease, an esterase, a glucosidase, a glucuronidase, galactosidase, a phosphatase, phosphodiesterase, nuclease, lipase or any enzyme that is capable of cleaving a linker to liberate the biomolecule from the other compound.

In some embodiments, a cleavable linker is or comprises a disulfide linkage, an ester, a phosphodiester, a saccharide, or a lipid.

In some embodiments, a non-cleavable linker is chemically, enzymatically, or otherwise biochemically and physiologically stable. As such, a non-cleavable linker does not contain linkages that are chemically, biochemically, enzymatically cleavable or are otherwise physiologically unstable.

In some embodiments, whether cleavable or non-cleavable, a linker can be installed by a chemical linking reaction between the payload and the binding moiety to which it is being conjugated. The payload and binding moiety may or may not be first modified to increase or facilitate reactivity towards one another. Such modification can also increase or improve the specificity of the conjugation reaction and degree of conjugation when that is desired. The linkers may be installed in a single reaction or by stepwise reactions until the desired linker and payload have been prepared.

Non-limiting examples of chemical linking reactions to form conjugate agents include reaction of various thiols to form disulfides, reaction between thiols and alkyl halides or maleimides to form thioethers, reaction of alkynes with azides to form triazoles (“Click Reaction”), reaction between aldehydes and hydrazides or amines, or aminoxy compounds to form hydrazones, imines and oxy imines, reaction between carboxylic acids and amines, thiols or alcohols (i.e., nucleophiles) to form amides, thioesters and esters. The carboxylic acids may be activated in situ in the presence of the amines, thiols or alcohols so as to be made reactive or may be pre-activated prior to addition of the nucleophile, for example by converting to activated esters of N-hydroxysuccinimide (NHS) or sulfonated—NHS. Many reviews of chemical linking reactions exist for example Spicer et al. (2018) Chem. Rev. 2018, 118, 16, 7702-7743.

The reaction of thiols with maleimides is very widely used, see for example (Revasco et al (2018) Chem. Eur. J. 10.1002/chem.201803174) as is the Click Reaction see for example (Fantoni et al., (2021) Chem. Rev., 121, 12, 7122-7154), as is hydrazide formation (See HyNic Peptide Conjugation Protocol, Dirksen et al (2006) J. Am. Chem. Soc., 128, 49, 15602-15603, Kozlov et al,. (2004) Biopolymers73 (5): 621-30. doi: 10.1002/bip.20009). Numerous companies sell chemical compounds and kits with protocols that enable conjugate agents comprising various linkers to be prepared in a straightforward fashion.

In some embodiments, a conjugate agent disclosed herein comprises about 1-30, about 2-30, about 5-30, about 15-30, about 20-30, about 25-30, about 1-25, about 1-20, about 1-15, about 1-10 or about 1-5 repeats of a linker disclosed herein. In some embodiments, a conjugate agent disclosed herein comprises about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 repeats of a linker disclosed herein.

As defined generally above and described herein, the linker is a bivalent group that connects or links the binding moiety to the payload moiety.

In some embodiments, the linker is or comprises a bivalent straight or branched C1-40 aliphatic chain, wherein one or more methylene units of the aliphatic chain are replaced by a group selected from —CH(R¹)—, —C(R¹)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur, wherein:
R¹ is an amino acid side chain; and
R is selected from hydrogen or an optionally substituted C₁₋₆ aliphatic, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, and a 5- to 6-membered heteroaryl ring having 1-4 heteroatoms independently selected from nitrogen, oxygen, and sulfur.

In some embodiments, the linker is or comprises a bivalent straight or branched C1-35 aliphatic chain, wherein one or more methylene units of the aliphatic chain are replaced by a group selected from —CH(R¹)—, —C(R¹)₂—, —O—, —S—, —N(R)—, —C(—O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur.

In some embodiments, the linker is or comprises a bivalent straight or branched C1-30 aliphatic chain, wherein one or more methylene units of the aliphatic chain are replaced by a group selected from —CH(R¹)—, —C(R¹)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(—O)N(R)—, —N(R)C(—O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur.

In some embodiments, the linker is or comprises a bivalent straight or branched C1-25 aliphatic chain, wherein one or more methylene units of the aliphatic chain are replaced by a group selected from —CH(R¹)—, —C(R¹)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur.

In some embodiments, the linker is or comprises a bivalent straight or branched C1-20 aliphatic chain, wherein one or more methylene units of the aliphatic chain are replaced by a group selected from —CH(R¹)—, —C(R¹)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur.

In some embodiments, the linker is or comprises a bivalent straight or branched C1-15 aliphatic chain, wherein one or more methylene units of the aliphatic chain are replaced by a group selected from —CH(R¹)—, —C(R¹)₂—, —O—, —S—, —N(R)—, —C(═O)—, —C(═S)—, —C(—NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(—O)O—, —OC(—O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur.

In some embodiments, the linker is or comprises a bivalent straight or branched C1-10 aliphatic chain, wherein one or more methylene units of the aliphatic chain are replaced by a group selected from —CH(R¹)—, —C(R¹)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur.

In some embodiments, the linker is or comprises a bivalent straight or branched C1-5 aliphatic chain, wherein one or more methylene units of the aliphatic chain are replaced by a group selected from —CH(R¹)—, —C(R¹)₂—, —O—, —S—, —N(R)—, —C(═O)—, —C(═S)—, —C(═NR), —N(R)C(═O)—, —C(═O)N(R)—, —N(R)C(═S)—, —C(═S)N(R)—, —OC(═O)—, —C(═O)O—, —SC(═O)—, —C(═O)S—, —N(R)C(═O)N(R)—, —N(R)C(═O)O—, —OC(═O)N(R)—, —N(R)C(═O)S—, —SC(═O)N(R)—, —OC(═O)O—, —N(R)C(═NR)—, —N(R)C(═NR)N(R)—, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur.

In some embodiments, the linker is or comprises a structure selected from

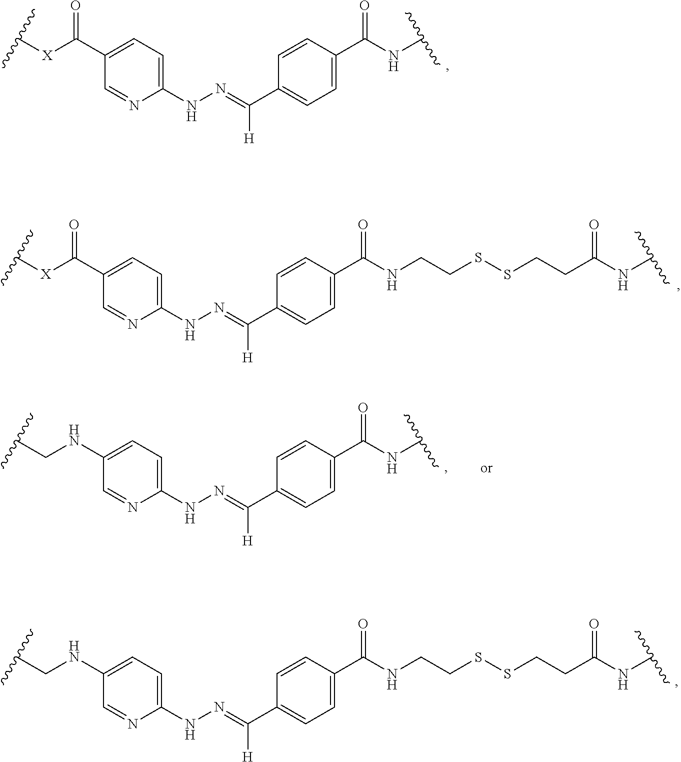

wherein X is NH or O.

In some embodiments, the cleavable linker is a cathepsin-cleavable linker. In some such embodiments, the linker is or comprises a valine-citrulline (Val-Cit) motif:

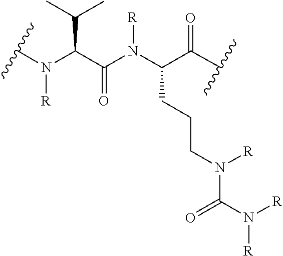

wherein R is hydrogen or C1-6 aliphatic.

In some embodiments, the valine-citrulline linker is or comprises

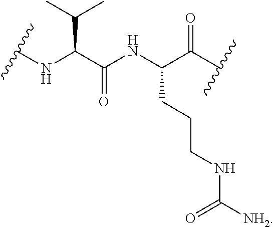 [SMILES: *N[C@H](C(=O)N[C@@H](CCCNC(N)=O)C(*)=O)C(C)C]

In some embodiments, the valine-citrulline linker is or comprises

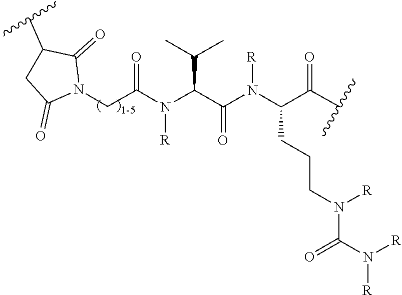

wherein R is hydrogen or C1-6 aliphatic.

In some embodiments, the linker comprises a disulfide linkage. In some embodiments, the linker comprises a poly (ethyleneglycol) moiety (e.g., —(CH₂CH₂O)_{b}—), wherein bis 1-50.

In some embodiments, the linker is or comprises a group selected from

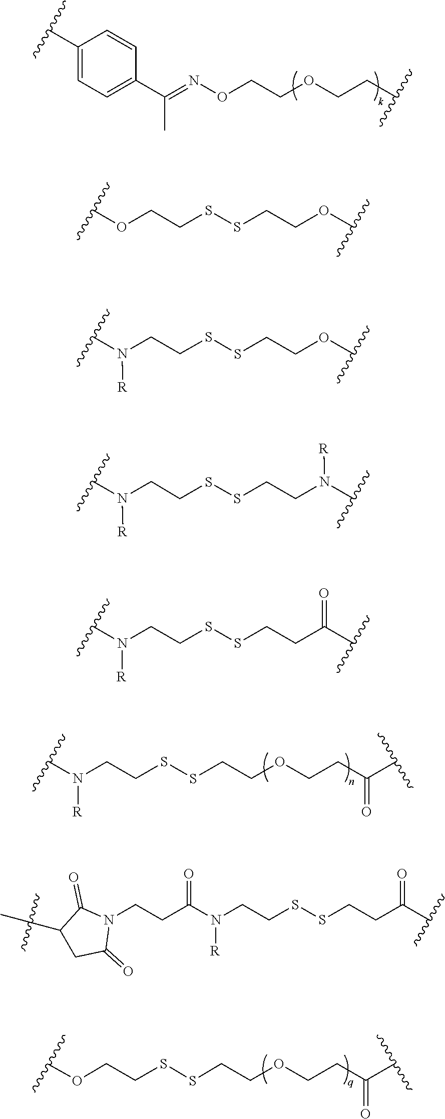

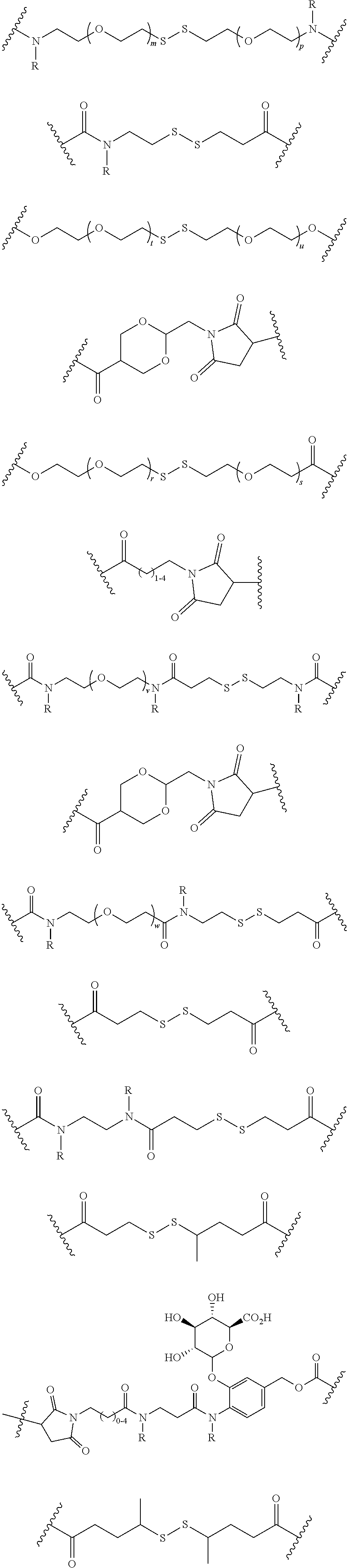

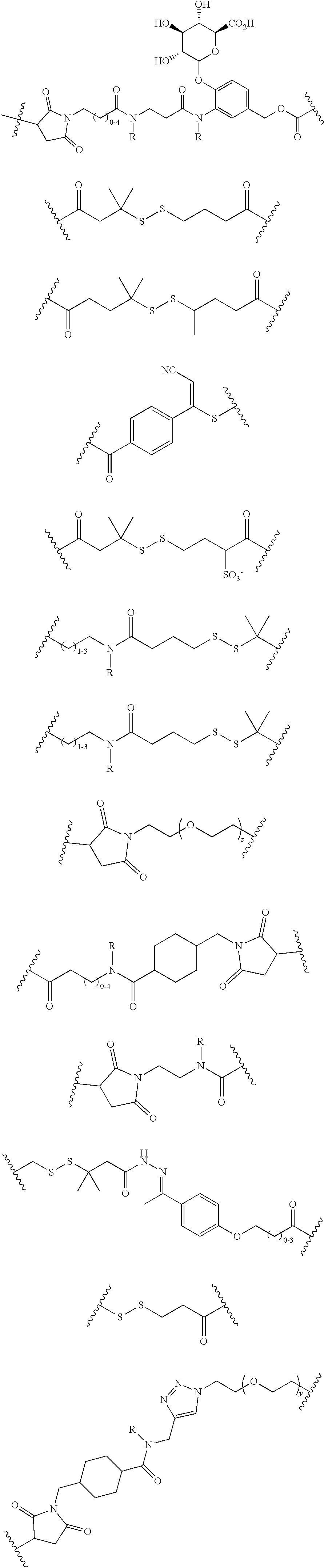

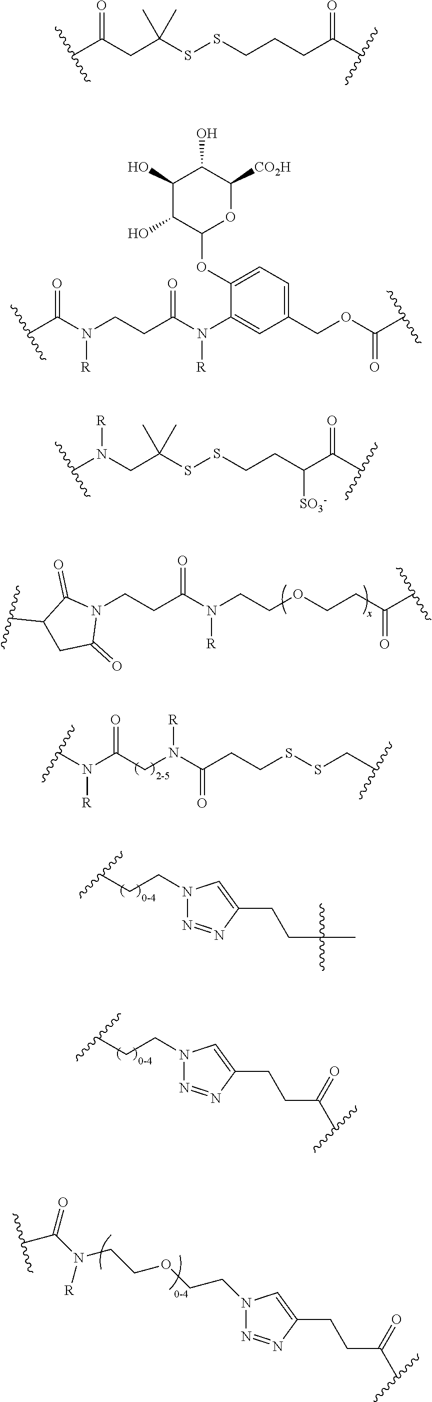

wherein each of k, m, n, p, q, r, s, t, u, v, w, x, y, and z is 1-20; and
R is hydrogen or C1-10 aliphatic.

In some embodiments, k is 3.

In some embodiments, m is 3.

In some embodiments, n is 2. In some embodiments, n is 12.

In some embodiments, p is 3.

In some embodiments, each of m and p is 3.

In some embodiments, q is 1.

In some embodiments, r is 3. In some embodiments, ris 4. In some embodiments, r is 6.

In some embodiments, s is 3. In some embodiments, s is 4. In some embodiments, s is 6.

In some embodiments, each of r and s is 3. In some embodiments, each of r and s is 4. In some embodiments, each of r and s is 6.

In some embodiments, t is 3. In some embodiments, tis 5.

In some embodiments, u is 3. In some embodiments, u is 5.

In some embodiments, each of t and u is 3. In some embodiments, each of t and u is 5.

In some embodiments, v is 3.

In some embodiments, w is 4.

In some embodiments, x is 8.

In some embodiments, y is 2.

In some embodiments, z is 1.

### Payload Moieties

In many embodiments, a payload moiety for use in the present disclosure is or comprises an entity whose presence in a relevant cell, e.g., of a tissue, achieves (e.g., correlates with) a particular effect (e.g., a particular detectable effect). In some embodiments, a relevant effect is or comprises, a particular biological and/or physiological effect. In some embodiments, a relevant effect is or comprises increase or decrease in level or activity of a particular nucleic acid (or form thereof) in the cell.

In many embodiments, an effect of a payload moiety is a change in one or more parameters of one or more target(s) of interest (an expression parameter and/or activity of the target of interest). In some embodiments, a target of interest may be a particular gene or gene product, or form (e.g., disease-associated form, splice variant form, etc) thereof. In some embodiments, a target of interest is selected from a target listed in any one of Tables 2-5, or a combination thereof.

Among other things, the present disclosure provides an insight that technologies described herein may be particularly useful and/or effective for delivery of nucleic acid agents. In some embodiments, a payload moiety is or comprises a nucleic acid. In some embodiments, a payload moiety is or comprises a single-stranded nucleic acid. In other embodiments, a payload moiety is or comprises a double-stranded nucleic acid. In some embodiments, a payload moiety is or comprises an oligonucleotide.

In some embodiments, a nucleic acid has a length within a range of about 10-50 nucleotides, about 10-49 nucleotides, about 10-48 nucleotides, about 10-47 nucleotides, about 10-46 nucleotides, about 10-45 nucleotides, about 10-44 nucleotides, about 10-43 nucleotides, about 10-42 nucleotides, about 10-41 nucleotides, about 10-40 nucleotides, about 10-39 nucleotides, about 10-38 nucleotides, about 10-37 nucleotides, about 10-36 nucleotides, about 10-35 nucleotides, about 10-34 nucleotides, about 10-33 nucleotides, about 10-32 nucleotides, about 10-31 nucleotides, about 10-30 nucleotides, about 10-29 nucleotides, about 10-28 nucleotides, about 10-27 nucleotides, about 10-26 nucleotides, about 10-25 nucleotides, about 10-24 nucleotides, about 10-23 nucleotides, about 10-22 nucleotides, about 10-21 nucleotides, about 10-20 nucleotides, about 10-19 nucleotides, about 10-18 nucleotides, about 10-17 nucleotides, about 10-16 nucleotides, about 10-15 nucleotides, about 10-14 nucleotides, about 10-13 nucleotides, about 10-12 nucleotides, about 10-11 nucleotides. In some embodiments, a nucleic acid has a length within a range of about 11-50 nucleotides, about 12-50 nucleotides, about 13-50 nucleotides, about 14-50 nucleotides, about 15-50 nucleotides, about 16-50 nucleotides, about 17-50 nucleotides, about 18-50 nucleotides, about 19-50 nucleotides, about 20-50 nucleotides, about 21-50 nucleotides, about 22-50 nucleotides, about 23-50 nucleotides, about 24-50 nucleotides, about 25-50 nucleotides, about 26-50 nucleotides, about 27-50 nucleotides, about 28-50 nucleotides, about 29-50 nucleotides, about 30-50 nucleotides, about 31-50 nucleotides, about 32-50 nucleotides, about 33-50 nucleotides, about 34-50 nucleotides, about 35-50 nucleotides, about 36-50 nucleotides, about 37-50 nucleotides, about 38-50 nucleotides, about 39-50 nucleotides, about 40-50 nucleotides, about 41-50 nucleotides, about 42-50 nucleotides, about 43-50 nucleotides, about 44-50 nucleotides, about 45-50 nucleotides, about 46-50 nucleotides, about 47-50 nucleotides, about 48-50 nucleotides, about 49-50 nucleotides.

In some embodiments, a nucleic acid is about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides in length.

In some embodiments, a nucleic acid agent, e.g., an oligonucleotide agent, for use in accordance with the present disclosure may comprise a single strand. In some embodiments, a nucleic acid may comprise more than one strand. In some embodiments, a nucleic acid may comprise one or more double-stranded portions. In some such embodiments, some or all of such portion(s) may be formed by self-hybridization of sequences on a single strand; in some embodiments some or all of such portion(s) may be formed by hybridization of separate strands. In some embodiments, a nucleic acid that includes one or more double-stranded portions may include one or more nicks or gaps and/or one or more bulges or loops.

In some embodiments, a nucleic acid agent, e.g., an oligonucleotide agent, for use in accordance with the present disclosure may include one or more structural features or characteristics relevant to its mode of action. For example, those skilled in the art are aware of extensive literature regarding structural features of, for example, oligonucleotides that trigger degradation of their targets (e.g., by recruiting RNase H(such oligonucleotides often being referred to as “antisense” agents or “ASOs”) and/or Dicer and/or other elements of the RNA-Induced Silencing Complex (RISC) (such oligonucleotides often being referred to as “siRNA” agents) and/or that modulate splicing of target transcripts (e.g., to favor production of one splice form over another) and/or that act as guide RNAs to recruit other machinery (e.g., nucleases such as CRISPR/Cas or dsRNA binding proteins, or conjugates thereof etc) to particular nucleic acid sequences, or as aptamers that bind to particular targets, etc.

In some embodiments, a nucleic acid is or comprises an interfering RNA (RNAi) agent. In some embodiments, an RNA is or comprises a short interfering RNA (siRNA) agent. In some embodiments, an RNA is or comprises a micro RNA (miRNA) agent. In some embodiments, a nucleic acid is or comprises a guide RNA (gRNA) agent.

In some embodiments, a nucleic acid is or comprises a short interfering RNA (siRNA) agent. In some embodiments, a nucleic acid comprising an siRNA agent can be linked to a targeting moiety (e.g., directly or indirectly) at a sense strand. In some embodiments, a nucleic acid comprising an siRNA agent can be linked to a targeting moiety (e.g., directly or indirectly) at an antisense strand. In some embodiments, a nucleic acid comprising an siRNA agent can be linked to a targeting moiety (e.g., directly or indirectly) at a 5′ end of an siRNA agent. In some embodiments, a nucleic acid comprising an siRNA agent can be linked to a targeting moiety (e.g., directly or indirectly) at a 3′ end of an siRNA agent.

In some embodiments, a nucleic acid is or comprises an exon skipping agent, an exon inclusion agent, or other splicing modulator.

In some embodiments, a nucleic acid is or comprises an aptamer agent.

In some embodiments, a nucleic acid agent is or comprises an antisense oligo (ASO). In some embodiments, an ASO modulates gene expression via RNase H mediated mechanisms. In some embodiments, an ASO modulates gene expression via steric hindrance.

In some embodiments, a nucleic acid agent is or comprises a phosphorodiamidate morpholino oligonucleotide (PMO).

In some embodiments, a nucleic acid agent is or comprises a peptide-nucleic acid (PNA).

In some embodiments, a nucleic acid agent is or comprises a nucleic acid analog, e.g., an RNA analog or a DNA analog, or a combination thereof.

In some embodiments, a nucleic acid can be linked to a targeting moiety (e.g., directly or indirectly) at a sense strand. In some embodiments, a nucleic acid can be linked to a targeting moiety (e.g., directly or indirectly) at an antisense strand. In some embodiments, a nucleic acid can be linked to a targeting moiety (e.g., directly or indirectly) at a 5′ end of a nucleic acid. In some embodiments, a nucleic acid can be linked to a targeting moiety (e.g., directly or indirectly) at a 3′ end of a nucleic acid.

For example, in some embodiments, a nucleic acid analog includes one or more modified (relative to canonical DNA and/or RNA)nucleotides. In some embodiments, a modified nucleotide comprises one or more of: a modified backbone, a modified nucleobase, a modified sugar (e.g., a modified ribose, or a modified deoxyribose), or a combination thereof. In some embodiments, a modified nucleotide may be or comprise one or more naturally occurring modifications; in some embodiments a modified nucleotide may be or comprise one or more non-naturally-occurring modifications.

In some embodiments, a nucleic acid analog comprises one or more linkages that is not a phosphodiester linkage (e.g., that is or comprises a phosphorothioate linkage or a phosphorodiamidate linkage).

In some embodiments, a nucleic acid analog comprises one or more morpholino subunits linked together by a phosphorus-containing linkage. In some embodiments, one or more morpholino subunits in an oligonucleotideanalog is joined by a phosphorodiamidate linkage. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337, and PCT Appn. Nos. PCT/US07/11435 (cationic linkages) and U.S. Ser. No. 08/012,804 (improved synthesis), all of which are incorporated herein by reference. Morpholino subunits linked by phosphorodiamidate linkages are disclosed in U.S. Pat. No. 11,071,749 the entire contents of which are hereby incorporated by reference. In some embodiments, a nucleic acid agent is or comprises aPMO. In some embodiments, a PMO is substantially uncharged, e.g., has a neutral charge.

In some embodiments, a nucleic acid agent has a negative charge.

In some embodiments, a nucleic acid agent is substantially uncharged, e.g., has a neutral charge.

Those skilled in the art, reading the present disclosure, will appreciate that, in some embodiments, a nucleic acid agent for use in accordance with the present disclosure may include one or more DNA residues or analogs thereof, one or more RNA residues or analogs thereof, and/or combinations thereof. Furthermore, such skilled person will appreciate that, in some embodiments, a nucleic acid agent may include one or more, or entirely, phosphodiester linkages, phosphorothioate linkages, or other suitable linkages.

In some embodiments, a nucleic acid agent comprises natural residues, e.g., DNA residues and/or RNA residues.

In some embodiments a nucleic acid agent comprises one or more analogs, e.g., DNA analogs and/or RNA analogs.

In some embodiments, a nucleic acid agent comprises DNA residues and/or RNA residues, e.g., natural residues or analogs.

In some embodiments, a nucleic acid comprises one or more chiral centers (e.g., as may be present in, for example, a phosphorothioate linkage). In some embodiments, a preparation of a nucleic acid having a chiral center is stereopure with respect to that center in that it includes only one stereoisomer of that center. In some embodiments, both stereoisomers are present. In some embodiments, the preparation represents a racemic mixture of stereoisomers at that position. In some embodiments, a preparation of a nucleic acid having more than one chiral linkage may be stereopure with respect to one or more centers and mixed (e.g., racemic) with respect to one or more others. In some embodiments, a preparation may be stereopure at all chiral centers. In some embodiments, a preparation may be racemic (e.g., at all chiral centers or overall).

In some embodiments, a nucleic acid comprises one or more modified nucleotides. In some embodiments, a modified nucleotide comprises one or more of: a modified backbone, a modified nucleobase, a modified ribose, a modified deoxyribose, or a combination thereof.

In some embodiments, a modified nucleotide is chosen from: a 2′-O-methyl modified nucleotide, a 5-methylcytidine, a 5-methyluridine, a nucleotide comprising a 5′-phosphorothioate group, a morpholino nucleotide (e.g., a PMO), a terminal nucleotide linked to a cholesteryl derivative or a dodecanoic acid bisdecylamide group, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide (e.g., PMO), a phosphoramidate, a phosphoryl guanidine (PN) based backbone, or a non-natural base comprising nucleotide, or a combination thereof.

In some embodiments, a modified nucleobase comprises a C7-modified deaza-adenine, a C7-modified deaza-guanosine, a C5-modified cytosine, a C5-modified uridine, N1-methyl-pseudouridine (ml), 1-ethyl-pseudouridine (el), 5-methoxy-uridine (mo5U), 5-methyl-cytidine (m5C), pseudouridine (w), 5-methoxymethyl uridine, 5-methylthio uridine, 1-methoxymethyl pseudouridine, 5-methyl cytidine, 5-methoxy cytidine, or a combination thereof.

In some embodiments, a modified sugar (e.g., a modified ribose, or a modified deoxyribose) comprises: a 2′fluoro modification, a 2′-O-methyl (2′OMe) modification, a locked nucleic acid (LNA), a 2′-fluoro arabinose nucleic acid (FANA), a hexitol nucleic acid (HNA), a 2′O-methoxyethyl (2′MOE) modification, or a combination thereof.

In some embodiments, a modified backbone comprises a phosphorothioate (PS) modification, a phosphoryl guanidine (PN) modification, a borano-phosphate modification, an alkyl phosphonate nucleic acid (phNA), a peptide nucleic acid (PNA), or a combination thereof.

In some embodiments, a nucleic acid comprises one or more modifications, e.g., to a 5′ end of an oligonucleotide. In some embodiments, a nucleic acid comprises a 5′ amino modification.

In some embodiments, a nucleic acid is partially modified (e.g., at least 5%) for a particular modification, e.g., throughout the length of a sequence.

In some embodiments, a nucleic acid is fully modified for a particular modification throughout the length of a sequence.

In some embodiments, at least 5% of a particular nucleotide (e.g., A, G, C, T, or U) is modified in an oligonucleotide.

In some embodiments, all (e.g., 100%) of a particular nucleotide (e.g., A, G, C, T, or U) is modified in an oligonucleotide.

In some embodiments, a nucleic acid comprises a structure comprising a first wing sequence, a gap sequence, and a second wing sequence. A nucleic acid comprising such a wing-gap-wing sequence is typically referred to as a gapmer. In some embodiments, a gap sequence is flanked by a first wing sequence and a second wing sequence. In some embodiments, a gap sequence comprises about 6-10 nucleotides. In some embodiments, a wing sequence comprises one or more nucleotides. In some embodiments, a wing sequence comprises one or more modified nucleotides, e.g., as disclosed herein. In some embodiments, a gapmer acts by recruiting RNaseH.

In some embodiments, a nucleic acid comprises an overhang. In some embodiments, an overhang is a 3′ overhang or a 5′ overhang. In some embodiments, an overhang is a 3′ overhang. In some embodiments, an overhang comprises at least 1, 2, 3, 4, 5, 6, 7, 8, 9, or 10 nucleotides. In some embodiments, a nucleic acid is double-stranded and comprises an overhang.

In some embodiments, a nucleic acid comprises at least one stem-loop structure.

An oligonucleotide disclosed herein typically comprises at least one sequence element that hybridizes with a target sequence. In some embodiments, a nucleic acid agent, e.g., an oligonucleotide, is or comprises an antisense sequence element. In some embodiments, an antisense sequence element is complementary to at least a portion of one or more of: an exon, an intron, an untranslated region, a splice junction, a promoter region, an enhancer region, or a non-coding region, e.g., in a gene transcript. In some embodiments, an antisense sequence element is complementary to a portion of a target sequence in a sense strand.

In some embodiments, a nucleic acid comprises a sequence element that is at least 80%, at least 85%, at least 90%, at least 95%, at least 96%, at least 97%, at least 98%, or at least 99% complementary to a target sequence in a sense strand. In some embodiments, a nucleic acid comprises a sequence element that is complementary (i.e. 100% complementary) to a target sequence in a sense strand.

In some embodiments, a nucleic acid comprises a sequence element that is at least 80%, at least 85%, at least 90%, at least 95%, at least 96%, at least 97%, at least 98%, or at least 99% complementary to a target sequence in an antisense strand. In some embodiments, a nucleic acid comprises a sequence element that is complementary (i.e. 100% complementary) to a target sequence in an antisense strand.

In some embodiments, a nucleic acid comprises at least one sequence element with at least 3, at least 4, at least 5, at least 6, at least 7, at least 8, at least 9, or at least 10 contiguous nucleotides having at least 80% complementarity to a portion of a target sequence. In some embodiments, a nucleic acid comprises at least one sequence element with at least 3, at least 4, at least 5, at least 6, at least 7, at least 8, at least 9, or at least 10 contiguous nucleotides having at least 85% complementarity to a portion of a target sequence. In some embodiments, a nucleic acid comprises at least one sequence element with at least 3, at least 4, at least 5, at least 6, at least 7, at least 8, at least 9, or at least 10 contiguous nucleotides having at least 90% complementarity to a portion of a target sequence. In some embodiments, a nucleic acid comprises at least one sequence element with at least 3, at least 4, at least 5, at least 6, at least 7, at least 8, at least 9, or at least 10 contiguous nucleotides having at least 95% complementarity to a portion of a target sequence. In some embodiments, a nucleic acid comprises at least one sequence element with at least 3, at least 4, at least 5, at least 6, at least 7, at least 8, at least 9, or at least 10 contiguous nucleotides having at least 96% complementarity to a portion of a target sequence. In some embodiments, a nucleic acid comprises at least one sequence element with at least 3, at least 4, at least 5, at least 6, at least 7, at least 8, at least 9, or at least 10 contiguous nucleotides having at least 97% complementarity to a portion of a target sequence. In some embodiments, a nucleic acid comprises at least one sequence element with at least 3, at least 4, at least 5, at least 6, at least 7, at least 8, at least 9, or at least 10 contiguous nucleotides having at least 98% complementarity to a portion of a target sequence. In some embodiments, a nucleic acid comprises at least one sequence element with at least 3, at least 4, at least 5, at least 6, at least 7, at least 8, at least 9, or at least 10 contiguous nucleotides having at least 99% complementarity to a portion of a target sequence. In some embodiments, a nucleic acid comprises at least one sequence element with at least 3, at least 4, at least 5, at least 6, at least 7, at least 8, at least 9, or at least 10 contiguous nucleotides having 100% complementarity to a portion of a target sequence.

In some embodiments, a nucleic acid comprises 2 or more sequence elements with at least 3, at least 4, at least 5, at least 6, at least 7, at least 8, at least 9, or at least 10 contiguous nucleotides having at least 80% complementarity to a portion of a target sequence.

In some embodiments, a nucleic acid binds to at least a portion of a target via Watson-Crick base pairing. In some embodiments, a nucleic acid binds to at least a portion of a target via Hoogsteen base pairing and/or other non-cannonical base pairing.

In some embodiments, a nucleic acid, e.g., an oligonucleotide, is characterized in that when an oligonucleotide, a composition comprising an oligonucleotide, or a conjugate agent comprising an oligonucleotide is delivered to a cell, tissue, or organism expressing a target, reduced expression and/or activity of a target is observed as compared to a cell, tissue or organism which has not been delivered an oligonucleotide, a composition comprising an oligonucleotide, or a conjugate agent comprising an oligonucleotide.

In some embodiments, a nucleic acid, e.g., an oligonucleotide, is characterized in that when an oligonucleotide, a composition comprising an oligonucleotide, or a conjugate agent comprising an oligonucleotide is delivered to a cell, tissue, or organism expressing a target, reduced expression and/or activity of a target is observed as compared to a cell, tissue or organism which does not express a target (e.g., which has no detectable expression of a target).

In some embodiments, a nucleic acid, e.g., an oligonucleotide, is characterized in that when an oligonucleotide, a composition comprising an oligonucleotide, or a conjugate agent comprising an oligonucleotide is delivered to a cell, tissue, or organism expressing a target, altered expression and/or activity of a target is observed relative to that observed with an appropriate reference agent known to have a specified impact on the target. In some embodiments, expression and/or activity of a target is altered in a manner and/or to an extent reasonably comparable to, or otherwise determined relative to, that observed with an appropriate reference agent known to have a specified impact on the target. In some embodiments, a reference agent may be a positive control reference agent. In some embodiments, a reference may be a negative control reference agent.

In some embodiments, a nucleic acid, e.g., an oligonucleotide, is characterized in that when delivered to a cell, tissue, or organism expressing a target, expression and/or activity of a target is modulated, e.g., reduced, as compared to a cell, tissue, or organism, which has not been delivered an oligonucleotide.

Without wishing to be bound by theory, it is believed that in some embodiments, a targeting moiety, e.g., a peptide as disclosed herein, can be conjugated to a payload moiety comprising a nucleic acid, e.g., an oligonucleotide.

### Payload Target

Disclosed herein, among other things, are conjugate agents comprising a payload moiety which can act on one or more targets, e.g., as disclosed herein.

In some embodiments, a target is present in a cell or tissue chosen from: immune cells (e.g., bone marrow cells, lymph node cells, thymic cells, peripheral blood mononuclear cells [e.g., myeloid and/or lymphoid cells], erythrocytes, eosinophils, neutrophils, and/or platelets); nervous system (e.g., brain tissue, cortex, cerebellum, retinal cells, spinal cord cells, nerve cells, neurons, and/or supporting cells; endothelial cells; muscle (e.g., heart muscle, smooth muscle, and/or skeletal muscle); small intestine; colon; adipocytes; kidney; liver; lung; spleen; stomach; esophagus; bladder; pancreas; thyroid; salivary gland; adrenal gland; pituitary gland; breast; skin; ovary; uterus; placenta; prostate; or testis, or a combination thereof.

In some embodiments, a target is present in a tissue or cells chosen from: renal tissue, thyroid tissue, parathyroid tissue, cells of the inner ear, and nervous system tissue.

In some embodiments, a target is present (e.g., at relatively high level(s)) on kidney cells such as proximal tubular epithelial cells and/or podocytes.

In some embodiments, a target is present in a cell associated with a kidney, e.g., a cell that is or can be found in a kidney, e.g., during development, during tissue homeostasis, or in the course of a disease or disorder. In some embodiments, a target is present in a tissue associated with a kidney, e.g., a tissue that is a part of a kidney, e.g., during development, during tissue homeostasis, or in the course of a disease or disorder.

In some embodiments, a cell, e.g., of a tissue, expressing a target also expresses a targeting moiety, e.g., as described herein.

In some embodiments, a cell, e.g., of a tissue, expressing a target also expresses a kidney-specific targeting moiety, e.g., as disclosed herein.

In some embodiments, expression and/or activity of a target can be deregulated in a disease or disorder. In some embodiments, delivery of a conjugate agent to a cell, e.g., of a tissue, expressing a target reduces the expression and/or activity of a target.

In some embodiments, delivery of a conjugate agent to an organism with aberrant expression and/or activity of a target in a cell, e.g., of a tissue, treats a disease or disorder and/or ameliorates a symptom of a disease or disorder in an organism.

In some embodiments, a target is chosen from a target provided in any one of Tables 2-5, or a combination thereof.

In some embodiments, a target is or comprises a gene product (e.g., a transcript) expressed in a particular cell (e.g., cell type) and/or tissue as described herein.

In some embodiments, a target is or comprises a non-coding RNA expressed in a particular cell (e.g., cell type) and/or tissue as described herein. In some embodiments, a target is or comprises a long non coding RNA (lncRNA), a microRNA, a Piwi-interacting RNAs (piRNA), a small nucleolar RNA (snoRNA), or a combination thereof.

In some embodiments, a target is expressed in a cell and/or tissue with an internalizing receptor on its surface. In some embodiments, a target is expressed in a cell and/or tissue with megalin on its surface. In some embodiments, a target is expressed in a cell and/or tissue with cubilin on its surface. In some embodiments, a target is expressed in kidney cell(s). In some embodiments, a target is expressed in one or more of: immune cells (e.g., bone marrow cells, lymph node cells, thymic cells, peripheral blood mononuclear cells [e.g., myeloid and/or lymphoid cells], erythrocytes, eosinophils, neutrophils, and/or platelets); nervous system cells (e.g., brain tissue, cortex, cerebellum, retinal cells, spinal cord cells, nerve cells, neurons, and/or supporting cells); endothelial cells; muscle (e.g., heart muscle, smooth muscle, and/or skeletal muscle); small intestine cells; colon cells; adipocytes; kidney cells; liver cells; lung cells; splenic cells; stomach cells; esophagus cells; bladder cells; pancreas cells; thyroid cells; salivary gland cells; adrenal gland cells; pituitary gland cells; breast cells; skin cells; ovary cells; uterus cells; placenta cells; prostate cells; or testis cells, or combinations thereof. In some embodiments, a target is expressed in renal proximal tubular epithelial cells (RPTECs), podocytes, and/or combinations thereof.

In some embodiments, a target is or comprises a gene expressed in a renal proximal tubular epithelial cell (RPTEC). In some embodiments, a target is chosen from a RPTEC gene provided in Table 2, or a combination thereof. In some embodiments, a target has one or more characteristics and/or functions provided in Table 3, or a combination thereof. In some embodiments, a target has one or more characteristics and/or functions chosen from: A-kinase anchoring proteins; Acyl-CoA dehydrogenase family; Acyl-CoA thioesterases; Aldo-keto reductases; Ankyrin repeat domain containing protein; Apolipoproteins; Basic helix-loop-helix proteins; Basic leucine zipper proteins; Beta-gamma crystallins; Blood group antigens; BPI fold containing proteins; C-type lectin domain containing proteins; Clq and TNF related; C2 domain containing protein; Cadherins; CAP superfamily; CD molecules; Chemokine ligands; Claudins; Collagens; Complement system; CTAGE family; Cytochrome P450s; Dbl family Rho GEFs; EF-hand domain containing; Erythrocyte membrane protein band 4.1; F-BAR domain containing; Fatty acid binding protein family; Fatty acid desaturases; Fibronectin type III domain containing; G protein-coupled receptors; Galectins; Gelsolin/villins; Glycoside hydrolase family 31; GOLD domain containing; GRAM domain containing; Haloacid dehalogenase like hydrolase domain containing; Heat shock proteins; Histones; Homeoboxes; I-BAR domain containing; Immunoglobulin superfamily domain containing; Interleukin receptors; Intermediate filaments; Ion channels; Kinesins; Late cornified envelope proteins; Ligand gated ion channels; Low density lipoprotein receptors; M14 carboxypeptidases; Maestro heat like repeat containing; Membrane spanning 4-domains; MetallothioneinsMethyltransferase families; Mitochondrial respiratory chain complex assembly factors; Mitochondrial respiratory chain complexes; Mucins; Myosin heavy chains; N-BAR domain containing; N-terminal EF-hand calcium binding proteins; Na+/K+ transporting ATPase interacting; NLR family; Non-coding RNAs; Oxysterol binding proteins; Paraneoplastic Ma antigens; PDZ domain containing proteins; Phospholipases; Pleckstrin homology domain containing; Protein phosphatase 1 regulatory subunits; PWWP domain containing; Ras association domain family; Ras small GTPase superfamily; Receptor accessory proteins; Receptor kinases; Receptor ligands; RNA binding motif containing proteins; Serine proteases; Serpin peptidase inhibitors; SH2 domain containing protiens; Short chain dehydrogenase/reductase superfamily; Sideroflexins; Signal transduction and activation of RNA metabolism family; Solute carriers; Sorting nexins; Sterile alpha motif domain containing proteins; STRIPAK complex; Sulfatases; Sushi domain containing proteins; Synapsins; Synaptotagmins; Tetraspanins; Tetratricopeptide repeat domain containing; Tripartite motif containing; Tubulin tyrosine ligase family; Tubulins; WD repeat domain containing; Zinc fingers; ZYG11 cell cycle regulator family, or a combination thereof.

In some embodiments, a target is or comprises a gene expressed in a podocyte. In some embodiments, a target is chosen from a podocyte gene provided in Table 4, or a combination thereof. In some embodiments, a target has one or more characteristics and/or functions provided in Table 5, or a combination thereof. In some embodiments, a target has one or more characteristics and/or functions chosen from Abhydrolase domain containing proteins; ADAM metallopeptidases with thrombospondin type 1 motif; Ankyrin repeat domain containing proteins; Apolipoproteins; Armadillo like helical domain containing; Basic leucine zipper proteins; Blood group antigens; Bone morphogenetic proteins; C-type lectin domain containing; Clq and TNF related; Carbonic anhydrases; CD molecules; Chitinases; Cilia and flagella associated; Crumbs complex; Dbl family Rho GEFs; EF-hand domain containing; F-BAR domain containing; Fibronectin type III domain containing; Forkhead boxes; Formins; G protein-coupled receptors; Gla domain containing; Glycosyltransferases; Homeoboxes; Immunoglobulin superfamily domain containing; Ion channels; Junctophilins; Kallikreins; Ligand gated ion channels; Lipocalins; Myosin light chain kinase family; Netrins; PDZ domain containing; Phospholipases; Pleckstrin homology domain containing; Potassium voltage-gated channel regulatory subunits; Protein phosphatases; Ras small GTPase superfamily; Receptor kinases; Receptor ligands; Rho GTPase activating proteins; RNA binding motif containing; Semaphorins; Serine proteases; Serpin peptidase inhibitors; Shisa family members; Solute carriers; Sterile alpha motif domain containing; Stomatin family; T cell receptors; Tetraspan junctional complex superfamily; Transcription elongation factor A like family; Troponin complex subunits; Tubulin polymerization promoting proteins; WD repeat domain containing; Wnt family; Zinc fingers, or a combination thereof.

TABLE 2
Exemplary RPTEC genes

| Gene | Gene name | Gene synonym | Uniprot | Ensembl |
|---|---|---|---|---|
| A1CF | APOBEC1 complementation factor | ACF, ACF64, ACF65, | Q9NQ94 | ENSG00000148584 |
| | | APOBEC1CF, ASP | | |
| AARD | Alanine and arginine rich domain | C8orf85, LOC441376 | Q4LEZ3 | ENSG00000205002 |
| | containing protein | | | |
| ABLIM3 | Actin binding LIM protein family member | KIAA0843 | O94929 | ENSG00000173210 |
| | 3 | | | |
| ABRACL | ABRA C-terminal like | C6orf115, Costars, | Q9P1F3 | ENSG00000146386 |
| | | HSPC280, PRO2013 | | |
| AC005726.1 | Novel protein | | | ENSG00000258472 |
| ACAD11 | Acyl-CoA dehydrogenase family member | FLJ12592 | Q709F0 | ENSG00000240303 |
| | 11 | | | |
| ACOT6 | Acyl-CoA thioesterase 6 | C14orf42 | Q3I5F7 | ENSG00000205669 |
| ADM2 | Adrenomedullin 2 | AM2, FLJ21135 | Q7Z4H4 | ENSG00000128165 |
| AFM | Afamin | ALB2, ALBA | P43652 | ENSG00000079557 |
| AFP | Alpha fetoprotein | FETA, HPAFP | P02771 | ENSG00000081051 |
| AGT | Angiotensinogen | SERPINA8 | P01019 | ENSG00000135744 |
| AIFM1 | Apoptosis inducing factor mitochondria | AIF, AUNX1, CMTX4, | O95831 | ENSG00000156709 |
| | associated 1 | DFNX5, NAMSD, PDCD8 | | |
| AKR7A3 | Aldo-keto reductase family 7 member A3 | | O95154 | ENSG00000162482 |
| ALB | Albumin | | P02768 | ENSG00000163631 |
| ALKAL2 | ALK and LTK ligand 2 | AUGA, FAM150B | Q6UX46 | ENSG00000189292 |
| AMN | Amnion associated transmembrane protein | amnionless | Q9BXJ7 | ENSG00000166126 |
| ANGPTL3 | Angiopoietin like 3 | ANGPT5 | Q9Y5C1 | ENSG00000132855 |
| ANKRD33B | Ankyrin repeat domain 33B | | A6NCL7 | ENSG00000164236 |
| ANKS4B | Ankyrin repeat and sterile alpha motif | FLJ38819, HARP | Q8N8V4 | ENSG00000175311 |
| | domain containing 4B | | | |
| APOC2 | Apolipoprotein C2 | | P02655 | ENSG00000234906 |
| APOC3 | Apolipoprotein C3 | | P02656 | ENSG00000110245 |
| APOE | Apolipoprotein E | AD2 | P02649 | ENSG00000130203 |
| APOH | Apolipoprotein H | B2G1, BG | P02749 | ENSG00000091583 |
| APOM | Apolipoprotein M | G3a, NG20 | O95445 | ENSG00000204444 |
| AQP11 | Aquaporin 11 | | Q8NBQ7 | ENSG00000178301 |
| AQP7 | Aquaporin 7 | AQP7L, AQP9, AQPap | O14520 | ENSG00000165269 |
| ARSF | Arylsulfatase F | | P54793 | ENSG00000062096 |
| ARSL | Arylsulfatase L | ARSE, CDPX, CDPX1 | P51690 | ENSG00000157399 |
| ASB15 | Ankyrin repeat and SOCS box containing | FLJ43370 | Q8WXK1 | ENSG00000146809 |
| | 15 | | | |
| ATOH7 | Atonal bHLH transcription factor 7 | bHLHa13, Math5 | Q8N100 | ENSG00000179774 |
| AVP | Arginine vasopressin | ADH, ARVP | P01185 | ENSG00000101200 |
| AZGP1 | Alpha-2-glycoprotein 1, zinc-binding | ZA2G, ZAG | P25311 | ENSG00000160862 |
| BAIAP2L2 | BAR/IMD domain containing adaptor | FLJ22582, pinkbar | Q6UXY1 | ENSG00000128298 |
| | protein 2 like 2 | | | |
| BIN1 | Bridging integrator 1 | AMPH2, AMPHL, SH3P9 | O00499 | ENSG00000136717 |
| BPHL | Biphenyl hydrolase like | Bph-rp, MCNAA, | Q86WA6 | ENSG00000137274 |
| | | VACVase | | |
| C10orf67 | Chromosome 10 open reading frame 67 | bA215C7.4, C10orf115, | Q8IYJ2 | ENSG00000179133 |
| | | LINC01552, MGC46732 | | |
| C11orf54 | Chromosome 11 open reading frame 54 | PTD012 | Q9H0W9 | ENSG00000182919 |
| C11orf86 | Chromosome 11 open reading frame 86 | FLJ22675 | A6NJI1 | ENSG00000173237 |
| C11orf97 | Chromosome 11 open reading frame 97 | LINC01171 | A0A1B0G | ENSG00000257057 |
| | | | VM6 | |
| C16orf87 | Chromosome 16 open reading frame 87 | | Q6PH81 | ENSG00000155330 |
| C1QTNF12 | C1q and TNF related 12 | ADIPOLIN, C1QDC2, | Q5T7M4 | ENSG00000184163 |
| | | CTRP12, FAM132A, | | |
| | | MGC105127 | | |
| C3orf85 | Chromosome 3 open reading frame 85 | FLJ22763 | A0A1B0G | ENSG00000241224 |
| | | | TC6 | |
| C4A | Complement C4A (Rodgers blood group) | C4, C4A2, C4A3, C4A4, | P0C0L4 | ENSG00000244731 |
| | | C4A6, C4B, C4S, CO4, | | |
| | | CPAMD2, RG | | |
| C5orf49 | Chromosome 5 open reading frame 49 | LOC134121 | A4QMS7 | ENSG00000215217 |
| CACNA1E | Calcium voltage-gated channel subunit | BII, CACH6, CACNL1A6, | Q15878 | ENSG00000198216 |
| | alpha1 E | Cav2.3 | | |
| CALML3 | Calmodulin like 3 | CLP | P27482 | ENSG00000178363 |
| CALML4 | Calmodulin like 4 | MGC4809, NY-BR-20 | Q96GE6 | ENSG00000129007 |
| CCDC150 | Coiled-coil domain containing 150 | FLJ39660 | Q8NCX0 | ENSG00000144395 |
| CCDC200 | Coiled-coil domain containing 200 | LINC00854, TMEM106A- | A0A1B0G | ENSG00000236383 |
| | | AS1 | VQ3 | |
| CDH9 | Cadherin 9 | | Q9ULB4 | ENSG00000113100 |
| CDHR2 | Cadherin related family member 2 | FLJ20124, FLJ20383, PC- | Q9BYE9 | ENSG00000074276 |
| | | LKC, PCDH24, PCLKC | | |
| CDHR3 | Cadherin related family member 3 | CDH28, FLJ23834, | Q6ZTQ4 | ENSG00000128536 |
| | | FLJ44366 | | |
| CDHR5 | Cadherin related family member 5 | FLJ20219, MU-PCDH, | Q9HBB8 | ENSG00000099834 |
| | | MUCDHL, MUPCDH | | |
| CELF3 | CUGBP Elav-like family member 3 | BRUNOL1, CAGH4, | Q5SZQ8 | ENSG00000159409 |
| | | ERDA4, MGC57297, | | |
| | | TNRC4 | | |
| CHP2 | Calcineurin like EF-hand protein 2 | | O43745 | ENSG00000166869 |
| CHRNA4 | Cholinergic receptor nicotinic alpha 4 | BFNC, EBN, EBN1 | P43681 | ENSG00000101204 |
| | subunit | | | |
| CKS2 | CDC28 protein kinase regulatory subunit 2 | | P33552 | ENSG00000123975 |
| CLDN2 | Claudin 2 | | P57739 | ENSG00000165376 |
| CLEC18A | C-type lectin domain family 18 member A | MRCL | A5D8T8 | ENSG00000157322 |
| CLEC18B | C-type lectin domain family 18 member B | MRCL2 | Q6UXF7 | ENSG00000140839 |
| CLEC18C | C-type lectin domain family 18 member C | MGC34761, MRCL3 | Q8NCF0 | ENSG00000157335 |
| CLRN3 | Clarin 3 | MGC32871, TMEM12, | Q8NCR9 | ENSG00000180745 |
| | | USH3AL1 | | |
| CLTRN | Collectrin, amino acid transport regulator | NX17, TMEM27 | Q9HBJ8 | ENSG00000147003 |
| CMBL | Carboxymethylenebutenolidase homolog | FLJ23617 | Q96DG6 | ENSG00000164237 |
| COL19A1 | Collagen type XIX alpha 1 chain | | Q14993 | ENSG00000082293 |
| COX6A2 | Cytochrome c oxidase subunit 6A2 | | Q02221 | ENSG00000156885 |
| CPN2 | Carboxypeptidase N subunit 2 | ACBP | P22792 | ENSG00000178772 |
| CPNE6 | Copine 6 | | O95741 | ENSG00000100884 |
| CRYBB3 | Crystallin beta B3 | CRYB3 | P26998 | ENSG00000100053 |
| CSDC2 | Cold shock domain containing C2 | PIPPin | Q9Y534 | ENSG00000172346 |
| CTXN3 | Cortexin 3 | | Q4LDR2 | ENSG00000205279 |
| CUBN | Cubilin | gp280, IFCR, MGA1 | O60494 | ENSG00000107611 |
| CXCL14 | C-X-C motif chemokine ligand 14 | BMAC, bolekine, BRAK, | O95715 | ENSG00000145824 |
| | | Kec, KS1, MIP-2g, NJAC, | | |
| | | SCYB14 | | |
| CYB5A | Cytochrome b5 type A | CYB5 | P00167 | ENSG00000166347 |
| CYP2B6 | Cytochrome P450 family 2 subfamily B | CPB6, CYP2B, CYPIIB6 | P20813 | ENSG00000197408 |
| | member 6 | | | |
| DAB2 | DAB adaptor protein 2 | DOC-2 | P98082 | ENSG00000153071 |
| DBI | Diazepam binding inhibitor, acyl-CoA | ACBD1, ACBP | P07108 | ENSG00000155368 |
| | binding protein | | | |
| DBX2 | Developing brain homeobox 2 | FLJ16139 | Q6ZNG2 | ENSG00000185610 |
| DCSTAMP | Dendrocyte expressed seven | DC-STAMP, FIND, | Q9H295 | ENSG00000164935 |
| | transmembrane protein | TM7SF4 | | |
| DEPDC7 | DEP domain containing 7 | | Q96QD5 | ENSG00000121690 |
| DHRS4L2 | Dehydrogenase/reductase 4 like 2 | SDR25C3 | Q6PKH6 | ENSG00000187630 |
| DLGAP2 | DLG associated protein 2 | C8orf68, DAP-2, ERICH1- | Q9P1A6 | ENSG00000198010 |
| | | AS1 | | |
| DMRTA1 | DMRT like family A1 | | Q5VZB9 | ENSG00000176399 |
| DNAJC12 | DnaJ heat shock protein family (Hsp40) | JDP1 | Q9UKB3 | ENSG00000108176 |
| | member C12 | | | |
| DNAJC22 | DnaJ heat shock protein family (Hsp40) | FLJ13236, wus | Q8N4W6 | ENSG00000178401 |
| | member C22 | | | |
| DNMT3L | DNA methyltransferase 3 like | MGC1090 | Q9UJW3 | ENSG00000142182 |
| DNPH1 | 2′-deoxynucleoside 5′-phosphate N- | C6orf108, dJ330M21.3, rcl | O43598 | ENSG00000112667 |
| | hydrolase 1 | | | |
| DOK6 | Docking protein 6 | DOK5L, HsT3226, | Q6PKX4 | ENSG00000206052 |
| | | MGC20785 | | |
| DPF3 | Double PHD fingers 3 | BAF45c, cer-d4, Cerd4, | Q92784 | ENSG00000205683 |
| | | FLJ14079 | | |
| EAF2 | ELL associated factor 2 | BM040, TRAITS, U19 | Q96CJ1 | ENSG00000145088 |
| ECHDC3 | Enoyl-CoA hydratase domain containing 3 | FLJ20909 | Q96DC8 | ENSG00000134463 |
| ELMOD1 | ELMO domain containing 1 | DKFZp547C176 | Q8N336 | ENSG00000110675 |
| EPB41L3 | Erythrocyte membrane protein band 4.1 | 4.1B, DAL1, KIAA0987 | Q9Y2J2 | ENSG00000082397 |
| | like 3 | | | |
| ERICH4 | Glutamate rich 4 | C19orf69, LOC100170765 | A6NGS2 | ENSG00000204978 |
| ERICH5 | Glutamate rich 5 | C8orf47, FLJ39553 | Q6P6B1 | ENSG00000177459 |
| ESPN | Espin | DFNB36 | B1AK53 | ENSG00000187017 |
| ETFA | Electron transfer flavoprotein subunit alpha | EMA, GA2, MADD | P13804 | ENSG00000140374 |
| ETFB | Electron transfer flavoprotein subunit beta | | P38117 | ENSG00000105379 |
| FABP1 | Fatty acid binding protein 1 | L-FABP | P07148 | ENSG00000163586 |
| FABP3 | Fatty acid binding protein 3 | FABP11, H-FABP, MDGI, | P05413 | ENSG00000121769 |
| | | O-FABP | | |
| FADS6 | Fatty acid desaturase 6 | | | ENSG00000172782 |
| FAM151A | Family with sequence similarity 151 | C1orf179, MGC27169 | Q8WW52 | ENSG00000162391 |
| | member A | | | |
| FAM205C | Family with sequence similarity 205 | FAM205CP | A6NFA0 | ENSG00000187791 |
| | member C | | | |
| FCAMR | Fc fragment of IgA and IgM receptor | CD351, FCA/MR, | Q8WWV6 | ENSG00000162897 |
| | | FKSG87 | | |
| FGF5 | Fibroblast growth factor 5 | | P12034 | ENSG00000138675 |
| FOLR1 | Folate receptor alpha | FOLR, FR√É≈Ω√Ç¬± | P15328 | ENSG00000110195 |
| FTCDNL1 | Formiminotransferase cyclodeaminase N- | FONG | E5RQL4 | ENSG00000226124 |
| | terminal like | | | |
| FTL | Ferritin light chain | MGC71996, NBIA3 | P02792 | ENSG00000087086 |
| GCHFR | GTP cyclohydrolase I feedback regulator | GFRP, HsT16933 | P30047 | ENSG00000137880 |
| GCSH | Glycine cleavage system protein H | | P23434 | ENSG00000140905 |
| GHRHR | Growth hormone releasing hormone receptor | | Q02643 | ENSG00000106128 |
| GIPC2 | GIPC PDZ domain containing family | FLJ20075, SEMCAP-2 | Q8TF65 | ENSG00000137960 |
| | member 2 | | | |
| GJB1 | Gap junction protein beta 1 | CMTX, CMTX1, CX32 | P08034 | ENSG00000169562 |
| GJB2 | Gap junction protein beta 2 | CX26, DFNA3, DFNB1, | P29033 | ENSG00000165474 |
| | | NSRD1 | | |
| GLB1L2 | Galactosidase beta 1 like 2 | | Q8IW92 | ENSG00000149328 |
| GLB1L3 | Galactosidase beta 1 like 3 | FLJ90231 | Q8NCI6 | ENSG00000166105 |
| GLIS1 | GLIS family zinc finger 1 | FLJ36155 | Q8NBF1 | ENSG00000174332 |
| GLOD5 | Glyoxalase domain containing 5 | | A6NK44 | ENSG00000171433 |
| GLRX | Glutaredoxin | GRX, GRX1 | P35754 | ENSG00000173221 |
| GLTPD2 | Glycolipid transfer protein domain containing 2 | | A6NH11 | ENSG00000182327 |
| GMNC | Geminin coiled-coil domain containing | GEMC1 | A6NCL1 | ENSG00000205835 |
| GP5 | Glycoprotein V platelet | CD42d | P40197 | ENSG00000178732 |
| GPHA2 | Glycoprotein hormone subunit alpha 2 | A2, GPA2, MGC126572, | Q96T91 | ENSG00000149735 |
| | | ZSIG51 | | |
| GPR155 | G protein-coupled receptor 155 | DEP.7, DEPDC3, | Q7Z3F1 | ENSG00000163328 |
| | | FLJ31819, PGR22 | | |
| GRAMD1B | GRAM domain containing 1B | KIAA1201, LINC01059 | Q3KR37 | ENSG00000023171 |
| GREB1 | Growth regulating estrogen receptor | KIAA0575 | Q4ZG55 | ENSG00000196208 |
| | binding 1 | | | |
| GRIA3 | Glutamate ionotropic receptor AMPA type | GluA3, GLUR3, GLURC, | P42263 | ENSG00000125675 |
| | subunit 3 | MRX94 | | |
| GRTP1 | Growth hormone regulated TBC protein 1 | FLJ22474, TBC1D6 | Q5TC63 | ENSG00000139835 |
| GYPA | Glycophorin A (MNS blood group) | CD235a, GPA, MN, MNS | P02724 | ENSG00000170180 |
| H2AC1 | H2A clustered histone 1 | bA317E16.2, H2AFR, | Q96QV6 | ENSG00000164508 |
| | | HIST1H2AA | | |
| H2BC1 | H2B clustered histone 1 | bA317E16.3, H2BFU, | Q96A08 | ENSG00000146047 |
| | | HIST1H2BA, STBP, | | |
| | | TSH2B | | |
| HAPLN4 | Hyaluronan and proteoglycan link protein | BRAL2, KIAA1926 | Q86UW8 | ENSG00000187664 |
| | 4 | | | |
| HAVCR2 | Hepatitis A virus cellular receptor 2 | CD366, FLJ14428, Tim-3, | Q8TDQ0 | ENSG00000135077 |
| | | TIM3, TIMD3 | | |
| HDHD3 | Haloacid dehalogenase like hydrolase | C9orf158, MGC12904 | Q9BSH5 | ENSG00000119431 |
| | domain containing 3 | | | |
| HHLA2 | HERV-H LTR-associating 2 | B7-H5, B7-H7, B7H7, B7y | Q9UM44 | ENSG00000114455 |
| HNF1A | HNF 1 homeobox A | HNF1, LFB1, MODY3, | | ENSG00000135100 |
| | | TCF1 | | |
| HNF4A | Hepatocyte nuclear factor 4 alpha | HNF4, MODY, MODY1, | P41235 | ENSG00000101076 |
| | | NR2A1, TCF14 | | |
| HNF4G | Hepatocyte nuclear factor 4 gamma | NR2A2 | Q14541 | ENSG00000164749 |
| HSPA4L | Heat shock protein family A (Hsp70) | APG-1, HSPH3, Osp94 | O95757 | ENSG00000164070 |
| | member 4 like | | | |
| IGF2BP1 | Insulin like growth factor 2 mRNA binding | IMP-1 | Q9NZI8 | ENSG00000159217 |
| | protein 1 | | | |
| IGSF11 | Immunoglobulin superfamily member 11 | BT-IgSF, CT119, Igsf13, | Q5DX21 | ENSG00000144847 |
| | | MGC35227, VSIG3 | | |
| IL17RB | Interleukin 17 receptor B | CRL4, EVI27, IL17BR, | Q9NRM6 | ENSG00000056736 |
| | | IL17RH1 | | |
| IL22RA1 | Interleukin 22 receptor subunit alpha 1 | CRF2-9, IL22R | Q8N6P7 | ENSG00000142677 |
| IQSEC3 | IQ motif and Sec7 domain ArfGEF 3 | KIAA1110, MGC30156 | Q9UPP2 | ENSG00000120645 |
| IRAG2 | Inositol 1,4,5-triphosphate receptor | JAW1, LRMP | Q12912 | ENSG00000118308 |
| | associated 2 | | | |
| KCNAB2 | Potassium voltage-gated channel subfamily | AKR6A5, HKvbeta2.1, | Q13303 | ENSG00000069424 |
| | A regulatory beta subunit 2 | HKvbeta2.2, KCNA2B | | |
| KCNG2 | Potassium voltage-gated channel modifier | KCNF2, Kv6.2 | Q9UJ96 | ENSG00000178342 |
| | subfamily G member 2 | | | |
| KCNH6 | Potassium voltage-gated channel subfamily | erg2, HERG2, Kv11.2 | Q9H252 | ENSG00000173826 |
| | H member 6 | | | |
| KCNJ15 | Potassium inwardly rectifying channel | IRKK, Kir1.3, Kir4.2 | Q99712 | ENSG00000157551 |
| | subfamily J member 15 | | | |
| KCNK10 | Potassium two pore domain channel | K2p10.1, PPP1R97, | P57789 | ENSG00000100433 |
| | subfamily K member 10 | TREK-2, TREK2 | | |
| KCNK5 | Potassium two pore domain channel | K2p5.1, TASK-2, TASK2 | O95279 | ENSG00000164626 |
| | subfamily K member 5 | | | |
| KHDRBS2 | KH RNA binding domain containing, | MGC26664, SLM-1, | Q5VWX1 | ENSG00000112232 |
| | signal transduction associated 2 | SLM1 | | |
| KIAA1191 | KIAA1191 | FLJ21022, p33MONOX, | Q96A73 | ENSG00000122203 |
| | | p60MONOX | | |
| KNL1 | Kinetochore scaffold 1 | AF15Q14, CASC5, CT29, | Q8NG31 | ENSG00000137812 |
| | | D40, hKNL-1, hSpc105, | | |
| | | KIAA1570, MCPH4, | | |
| | | PPP1R55, Spc7 | | |
| KRT85 | Keratin 85 | Hb-5, KRTHB5 | P78386 | ENSG00000135443 |
| LACTB2 | Lactamase beta 2 | CGI-83 | Q53H82 | ENSG00000147592 |
| LBP | Lipopolysaccharide binding protein | BPIFD2 | P18428 | ENSG00000129988 |
| LCE2D | Late cornified envelope 2D | LEP12, SPRL1A | Q5TA82 | ENSG00000187223 |
| LGALS2 | Galectin 2 | HL14 | P05162 | ENSG00000100079 |
| LGI3 | Leucine rich repeat LGI family member 3 | | Q8N145 | ENSG00000168481 |
| LGSN | Lengsin, lens protein with glutamine | GLULD1, LGS | Q5TDP6 | ENSG00000146166 |
| | synthetase domain | | | |
| LIME1 | Lck interacting transmembrane adaptor 1 | dJ583P15.4, FLJ20406, | Q9H400 | ENSG00000203896 |
| | | LIME | | |
| LIN52 | Lin-52 DREAM MuvB core complex | C14orf46 | Q52LA3 | ENSG00000205659 |
| | component | | | |
| LRP2 | LDL receptor related protein 2 | DBS, gp330 | P98164 | ENSG00000081479 |
| LRRC19 | Leucine rich repeat containing 19 | FLJ21302 | Q9H756 | ENSG00000184434 |
| LRRC4 | Leucine rich repeat containing 4 | NAG14 | Q9HBW1 | ENSG00000128594 |
| MAF | MAF bZIP transcription factor | c-MAF | O75444 | ENSG00000178573 |
| MAIP1 | Matrix AAA peptidase interacting protein | C2orf47, DKFZp666A212, | Q8WWC4 | ENSG00000162972 |
| | 1 | FLJ22555 | | |
| MAJIN | Membrane anchored junction protein | C11orf85 | Q3KP22 | ENSG00000168070 |
| MAP7D2 | MAP7 domain containing 2 | FLJ14503 | Q96T17 | ENSG00000184368 |
| MAPT | Microtubule associated protein tau | DDPAC, FLJ31424, | P10636 | ENSG00000186868 |
| | | FTDP-17, MAPTL, | | |
| | | MGC138549, MSTD, | | |
| | | MTBT1, MTBT2, PPND, | | |
| | | PPP1R103, tau | | |
| MCCD1 | Mitochondrial coiled-coil domain 1 | | P59942 | ENSG00000204511 |
| MEI4 | Meiotic double-stranded break formation protein 4 | | A8MW99 | ENSG00000269964 |
| METTL7B | Methyltransferase like 7B | ALDI, MGC17301 | Q6UX53 | ENSG00000170439 |
| MIA2 | MIA SH3 domain ER export factor 2 | cTAGE-5A, cTAGE-5B, | Q96PC5 | ENSG00000150527 |
| | | cTAGE-5C, cTAGE-5D, | | |
| | | CTAGE5, FLJ22404, | | |
| | | MEA6, MGEA, MGEA11, | | |
| | | MGEA6, TALI | | |
| MISP | Mitotic spindle positioning | C19orf21, Caprice, | Q8IVT2 | ENSG00000099812 |
| | | DKFZp686H18209, | | |
| | | MISP1 | | |
| MLXIPL | MLX interacting protein like | bHLHd14, CHREBP, | Q9NP71 | ENSG00000009950 |
| | | MIO, MONDOB, | | |
| | | WBSCR14, WS-bHLH | | |
| MRLN | Myoregulin | Linc-RAM, LINC00948, | P0DMT0 | ENSG00000227877 |
| | | M1, MLN, MUSER1 | | |
| MRO | Maestro | B29, C18orf3, FLJ30140 | Q9BYG7 | ENSG00000134042 |
| MROH2A | Maestro heat like repeat family member | HEATR7B1 | A6NES4 | ENSG00000185038 |
| | 2A | | | |
| MST1 | Macrophage stimulating 1 | D3F15S2, DNF15S2, | | ENSG00000173531 |
| | | HGFL, MSP, NF15S2 | | |
| MT1G | Metallothionein 1G | MT1, MT1K | P13640 | ENSG00000125144 |
| MT1H | Metallothionein 1H | MT1 | P80294 | ENSG00000205358 |
| MT1X | Metallothionein 1X | MT-1l, MT1 | P80297 | ENSG00000187193 |
| MTCH2 | Mitochondrial carrier 2 | SLC25A50 | Q9Y6C9 | ENSG00000109919 |
| MTFR1 | Mitochondrial fission regulator 1 | CHPPR, FAM54A2, | Q15390 | ENSG00000066855 |
| | | KIAA0009 | | |
| MTNR1A | Melatonin receptor 1A | MEL-1A-R | P48039 | ENSG00000168412 |
| MTTP | Microsomal triglyceride transfer protein | ABL, MTP | P55157 | ENSG00000138823 |
| MUC13 | Mucin 13, cell surface associated | DRCC1 | Q9H3R2 | ENSG00000173702 |
| MYO7A | Myosin VIIA | DFNA11, DFNB2, | Q13402 | ENSG00000137474 |
| | | NSRD2, USH1B | | |
| MYO7B | Myosin VIIB | | Q6PIF6 | ENSG00000169994 |
| MYOM3 | Myomesin 3 | FLJ35961 | Q5VTT5 | ENSG00000142661 |
| MYORG | Myogenesis regulating glycosidase | KIAA1161, NET37 | Q6NSJ0 | ENSG00000164976 |
| | (putative) | | | |
| NCKAP5 | NCK associated protein 5 | ERIH1, ERIH2, NAP5 | O14513 | ENSG00000176771 |
| NECAB2 | N-terminal EF-hand calcium binding | EFCBP2 | Q7Z6G3 | ENSG00000103154 |
| | protein 2 | | | |
| NGEF | Neuronal guanine nucleotide exchange | ARHGEF27 | Q8N5V2 | ENSG00000066248 |
| | factor | | | |
| NIPSNAP1 | Nipsnap homolog 1 | | Q9BPW8 | ENSG00000184117 |
| NKAIN4 | Sodium/potassium transporting ATPase | bA261N11.2, C20orf58, | Q8IVV8 | ENSG00000101198 |
| | interacting 4 | FAM77A | | |
| NLRP6 | NLR family pyrin domain containing 6 | CLR11.4, NALP6, PAN3, | P59044 | ENSG00000174885 |
| | | PYPAF5 | | |
| NOCT | Nocturnin | Ccr4c, CCR4L, CCRN4L, | Q9UK39 | ENSG00000151014 |
| | | NOC | | |
| NOX4 | NADPH oxidase 4 | KOX, KOX-1 | Q9NPH5 | ENSG00000086991 |
| NPC1L1 | NPC1 like intracellular cholesterol | SLC65A2 | Q9UHC9 | ENSG00000015520 |
| | transporter 1 | | | |
| NR1I3 | Nuclear receptor subfamily 1 group I | CAR, CAR1, MB67 | Q14994 | ENSG00000143257 |
| | member 3 | | | |
| NSG1 | Neuronal vesicle trafficking associated 1 | D4S234E, NEEP21, P21 | P42857 | ENSG00000168824 |
| NUGGC | Nuclear GTPase, germinal center | C8orf80, HMFN0672, | Q68CJ6 | ENSG00000189233 |
| | associated | SLIP-GC | | |
| NUTM2F | NUT family member 2F | DKFZp434I1117, | A1L443 | ENSG00000130950 |
| | | FAM22F | | |
| NXNL2 | Nucleoredoxin like 2 | C9orf121, RdCVF2 | Q5VZ03 | ENSG00000130045 |
| NYX | Nyctalopin | CLRP, CSNB1, CSNB1A, | Q9GZU5 | ENSG00000188937 |
| | | CSNB4 | | |
| OCIAD2 | OCIA domain containing 2 | MGC45416 | Q56VL3 | ENSG00000145247 |
| OCSTAMP | Osteoclast stimulatory transmembrane | C20orf123, dJ257E24.3 | Q9BR26 | ENSG00000149635 |
| | protein | | | |
| OIT3 | Oncoprotein induced transcript 3 | FLJ39116, LZP | Q8WWZ8 | ENSG00000138315 |
| OPRD1 | Opioid receptor delta 1 | | P41143 | ENSG00000116329 |
| OSBPL6 | Oxysterol binding protein like 6 | ORP6 | Q9BZF3 | ENSG00000079156 |
| OTOGL | Otogelin like | C12orf64, FLJ90579 | Q3ZCN5 | ENSG00000165899 |
| OXER1 | Oxoeicosanoid receptor 1 | GPCR, GPR170, TG1019 | Q8TDS5 | ENSG00000162881 |
| OXT | Oxytocin/neurophysin I prepropeptide | OT, OT-NPI, OXT-NPI | P01178 | ENSG00000101405 |
| PACC1 | Proton activated chloride channel 1 | C1orf75, FLJ10874, PAC, | Q9H813 | ENSG00000065600 |
| | | TMEM206 | | |
| PACSIN1 | Protein kinase C and casein kinase | SDPI | Q9BY11 | ENSG00000124507 |
| | substrate in neurons 1 | | | |
| PAIP2B | Poly(A) binding protein interacting protein | KIAA1155 | Q9ULR5 | ENSG00000124374 |
| | 2B | | | |
| PBLD | Phenazine biosynthesis like protein domain | FLJ14767, MAWBP, | P30039 | ENSG00000108187 |
| | containing | MAWDBP | | |
| PCDH15 | Protocadherin related 15 | CDHR15, DFNB23, | Q96QU1 | ENSG00000150275 |
| | | USH1F | | |
| PDZD3 | PDZ domain containing 3 | FLJ22756, IKEPP, PDZK2 | Q86UT5 | ENSG00000172367 |
| PDZK1 | PDZ domain containing 1 | NHERF3, PDZD1 | Q5T2W1 | ENSG00000174827 |
| PDZK1IP1 | PDZK1 interacting protein 1 | DD96, MAP17, SPAP | Q13113 | ENSG00000162366 |
| PFN3 | Profilin 3 | | P60673 | ENSG00000196570 |
| PLA2G12B | Phospholipase A2 group XIIB | PLA2G13 | Q9BX93 | ENSG00000138308 |
| PNMA6A | PNMA family member 6A | MGC15827, PNMA6C | P0CW24 | ENSG00000235961 |
| PPP1R14D | Protein phosphatase 1 regulatory inhibitor | CPI17-like, FLJ20251, | Q9NXH3 | ENSG00000166143 |
| | subunit 14D | GBPI-1, MGC119014, | | |
| | | MGC119016 | | |
| PPP1R16B | Protein phosphatase 1 regulatory subunit | ANKRD4, KIAA0823, | Q96T49 | ENSG00000101445 |
| | 16B | TIMAP | | |
| PRAP1 | Proline rich acidic protein 1 | UPA | Q96NZ9 | ENSG00000165828 |
| PRDM7 | PR/SET domain 7 | ZNF910 | Q9NQW5 | ENSG00000126856 |
| PRLR | Prolactin receptor | | P16471 | ENSG00000113494 |
| PWWP3B | PWWP domain containing 3B | FLJ33516, MUM1L1 | Q5H9M0 | ENSG00000157502 |
| PXMP2 | Peroxisomal membrane protein 2 | MPV17L3, PMP22 | Q9NR77 | ENSG00000176894 |
| R3HDML | R3H domain containing like | dJ881L22.3 | Q9H3Y0 | ENSG00000101074 |
| RAB11FIP3 | RAB11 family interacting protein 3 | eferin, KIAA0665, Rab11- | O75154 | ENSG00000090565 |
| | | FIP3 | | |
| RAB29 | RAB29, member RAS oncogene family | RAB7L, RAB7L1 | O14966 | ENSG00000117280 |
| RALYL | RALY RNA binding protein like | HNRPCL3 | Q86SE5 | ENSG00000184672 |
| RASGRF1 | Ras protein specific guanine nucleotide | CDC25, CDC25L, GNRP, | Q13972 | ENSG00000058335 |
| | releasing factor 1 | GRF1, GRF55, H-GRF55, | | |
| | | PP13187 | | |
| RASSF4 | Ras association domain family member 4 | AD037, MGC44914 | Q9H2L5 | ENSG00000107551 |
| RBP5 | Retinol binding protein 5 | CRBPIII | P82980 | ENSG00000139194 |
| REEP6 | Receptor accessory protein 6 | C19orf32, DP1L1, | Q96HR9 | ENSG00000115255 |
| | | FLJ25383, Yip2f | | |
| REPS2 | RALBP1 associated Eps domain | POB1 | Q8NFH8 | ENSG00000169891 |
| | containing 2 | | | |
| RHOBTB1 | Rho related BTB domain containing 1 | KIAA0740 | O94844 | ENSG00000072422 |
| RNF212B | Ring finger protein 212B | C14orf164 | A8MTL3 | ENSG00000215277 |
| RNF224 | Ring finger protein 224 | | P0DH78 | ENSG00000233198 |
| RTL4 | Retrotransposon Gag like 4 | FLJ46608, Mar4, Mart4, | Q6ZR62 | ENSG00000187823 |
| | | SIRH11, ZCCHC16 | | |
| RUNDC3B | RUN domain containing 3B | RPIB9, RPIP9 | Q96NL0 | ENSG00000105784 |
| SAMD5 | Sterile alpha motif domain containing 5 | dJ875H10.1 | Q5TGI4 | ENSG00000203727 |
| SCN9A | Sodium voltage-gated channel alpha | ETHA, Nav1.7, NE-NA, | Q15858 | ENSG00000169432 |
| | subunit 9 | NENA, PN1 | | |
| SDHC | Succinate dehydrogenase complex subunit | CYB560, cybL, PGL3 | Q99643 | ENSG00000143252 |
| | C | | | |
| SEMA4G | Semaphorin 4G | FLJ20590, KIAA1619 | Q9NTN9 | ENSG00000095539 |
| SERPINA6 | Serpin family A member 6 | CBG | P08185 | ENSG00000170099 |
| SERPINC1 | Serpin family C member 1 | AT3, ATIII, MGC22579 | P01008 | ENSG00000117601 |
| SERPINF2 | Serpin family F member 2 | A2AP, AAP, ALPHA-2- | P08697 | ENSG00000167711 |
| | | PI, API, PLI | | |
| SERPINI1 | Serpin family I member 1 | PI12 | Q99574 | ENSG00000163536 |
| SFXN1 | Sideroflexin 1 | FLJ12876, SLC56A1 | Q9H9B4 | ENSG00000164466 |
| SFXN2 | Sideroflexin 2 | SLC56A2 | Q96NB2 | ENSG00000156398 |
| SFXN5 | Sideroflexin 5 | BBG-TCC, SLC56A5 | Q8TD22 | ENSG00000144040 |
| SH2D6 | SH2 domain containing 6 | FLJ35993 | | ENSG00000152292 |
| SH3GL2 | SH3 domain containing GRB2 like 2, | CNSA2, EEN-B1, | Q99962 | ENSG00000107295 |
| | endophilin A1 | SH3D2A, SH3P4 | | |
| SHBG | Sex hormone binding globulin | ABP, MGC126834, | P04278 | ENSG00000129214 |
| | | MGC138391, TEBG | | |
| SLC10A2 | Solute carrier family 10 member 2 | ASBT, ISBT | Q12908 | ENSG00000125255 |
| SLC13A1 | Solute carrier family 13 member 1 | NAS1, NaSi-1 | Q9BZW2 | ENSG00000081800 |
| SLC13A2 | Solute carrier family 13 member 2 | NaDC-1 | Q13183 | ENSG00000007216 |
| SLC13A3 | Solute carrier family 13 member 3 | NADC3, SDCT2 | Q8WWT9 | ENSG00000158296 |
| SLC16A10 | Solute carrier family 16 member 10 | MCT10, TAT1 | Q8TF71 | ENSG00000112394 |
| SLC16A4 | Solute carrier family 16 member 4 | MCT4, MCT5 | O15374 | ENSG00000168679 |
| SLC16A9 | Solute carrier family 16 member 9 | C10orf36, FLJ43803, | Q7RTY1 | ENSG00000165449 |
| | | MCT9 | | |
| SLC17A1 | Solute carrier family 17 member 1 | NAPI-1, NPT1 | Q14916 | ENSG00000124568 |
| SLC17A3 | Solute carrier family 17 member 3 | NPT4 | O00476 | ENSG00000124564 |
| SLC17A4 | Solute carrier family 17 member 4 | KIAA2138 | Q9Y2C5 | ENSG00000146039 |
| SLC1A1 | Solute carrier family 1 member 1 | EAAC1, EAAT3 | P43005 | ENSG00000106688 |
| SLC22A11 | Solute carrier family 22 member 11 | OAT4 | Q9NSA0 | ENSG00000168065 |
| SLC22A12 | Solute carrier family 22 member 12 | OAT4L, RST, URAT1 | Q96S37 | ENSG00000197891 |
| SLC22A13 | Solute carrier family 22 member 13 | OAT10, OCTL1, OCTL3, | Q9Y226 | ENSG00000172940 |
| | | ORCTL3 | | |
| SLC22A18AS | Solute carrier family 22 member 18 | BWR1B, BWSCR1B, | Q8N1D0 | ENSG00000254827 |
| | antisense | ORCTL2S, p27-BWR1B, | | |
| | | SLC22A1LS | | |
| SLC22A24 | Solute carrier family 22 member 24 | MGC34821, NET46 | Q8N4F4 | ENSG00000197658 |
| SLC22A4 | Solute carrier family 22 member 4 | DFNB60, MGC34546, | Q9H015 | ENSG00000197208 |
| | | OCTN1 | | |
| SLC22A6 | Solute carrier family 22 member 6 | OAT1, PAHT, ROAT1 | Q4U2R8 | ENSG00000197901 |
| SLC22A7 | Solute carrier family 22 member 7 | NLT, OAT2 | Q9Y694 | ENSG00000137204 |
| SLC22A8 | Solute carrier family 22 member 8 | OAT3 | Q8TCC7 | ENSG00000149452 |
| SLC23A1 | Solute carrier family 23 member 1 | SLC23A2, SVCT1, YSPL3 | Q9UHI7 | ENSG00000170482 |
| SLC23A3 | Solute carrier family 23 member 3 | FLJ31168, SVCT3, Yspl1 | Q6PIS1 | ENSG00000213901 |
| SLC25A10 | Solute carrier family 25 member 10 | DIC | Q9UBX3 | ENSG00000183048 |
| SLC25A42 | Solute carrier family 25 member 42 | MGC26694 | Q86VD7 | ENSG00000181035 |
| SLC26A1 | Solute carrier family 26 member 1 | EDM4, SAT-1 | Q9H2B4 | ENSG00000145217 |
| SLC26A9 | Solute carrier family 26 member 9 | | Q7LBE3 | ENSG00000174502 |
| SLC28A1 | Solute carrier family 28 member 1 | CNT1 | O00337 | ENSG00000156222 |
| SLC28A2 | Solute carrier family 28 member 2 | CNT2, HCNT2, | O43868 | ENSG00000137860 |
| | | HsT17153, SPNT1 | | |
| SLC2A2 | Solute carrier family 2 member 2 | GLUT2 | P11168 | ENSG00000163581 |
| SLC2A5 | Solute carrier family 2 member 5 | GLUT5 | P22732 | ENSG00000142583 |
| SLC2A9 | Solute carrier family 2 member 9 | Glut9, GLUTX, URATv1 | Q9NRM0 | ENSG00000109667 |
| SLC30A2 | Solute carrier family 30 member 2 | ZNT2 | Q9BRI3 | ENSG00000158014 |
| SLC30A8 | Solute carrier family 30 member 8 | ZnT-8, ZNT8 | Q8IWU4 | ENSG00000164756 |
| SLC34A1 | Solute carrier family 34 member 1 | NAPI-3, NPT2, NPTIIa, | Q06495 | ENSG00000131183 |
| | | SLC11, SLC17A2 | | |
| SLC34A3 | Solute carrier family 34 member 3 | FLJ38680, NPTIIc | Q8N130 | ENSG00000198569 |
| SLC36A2 | Solute carrier family 36 member 2 | PAT2, TRAMD1, | Q495M3 | ENSG00000186335 |
| | | tramdorin | | |
| SLC37A4 | Solute carrier family 37 member 4 | G6PT1, G6PT2, G6PT3, GSD1b, | | ENSG00000137700 |
| | | GSD1c, GSD1d | | |
| SLC39A4 | Solute carrier family 39 member 4 | AEZ, AWMS2, ZIP4 | Q6P5W5 | ENSG00000147804 |
| SLC39A5 | Solute carrier family 39 member 5 | | Q6ZMH5 | ENSG00000139540 |
| SLC3A1 | Solute carrier family 3 member 1 | ATR1, CSNU1, D2H, | Q07837 | ENSG00000138079 |
| | | NBAT, RBAT | | |
| SLC3A2 | Solute carrier family 3 member 2 | 4F2, 4F2HC, 4T2HC, | P08195 | ENSG00000168003 |
| | | CD98, CD98HC, MDU1, | | |
| | | NACAE | | |
| SLC47A1 | Solute carrier family 47 member 1 | FLJ10847, MATE1 | Q96FL8 | ENSG00000142494 |
| SLC47A2 | Solute carrier family 47 member 2 | FLJ31196, MATE2, | Q86VL8 | ENSG00000180638 |
| | | MATE2-K | | |
| SLC4A4 | Solute carrier family 4 member 4 | hhNMC, HNBC1, NBC1, | Q9Y6R1 | ENSG00000080493 |
| | | NBC2, pNBC, SLC4A5 | | |
| SLC51A | Solute carrier family 51 subunit alpha | OSTalpha | Q86UW1 | ENSG00000163959 |
| SLC51B | Solute carrier family 51 subunit beta | OSTbeta | Q86UW2 | ENSG00000186198 |
| SLC5A10 | Solute carrier family 5 member 10 | SGLT5 | A0PJK1 | ENSG00000154025 |
| SLC5A11 | Solute carrier family 5 member 11 | KST1, SGLT6, SMIT2 | Q8WWX8 | ENSG00000158865 |
| SLC5A12 | Solute carrier family 5 member 12 | MGC52019, SMCT2 | Q1EHB4 | ENSG00000148942 |
| SLC5A2 | Solute carrier family 5 member 2 | SGLT2 | P31639 | ENSG00000140675 |
| SLC5A9 | Solute carrier family 5 member 9 | SGLT4 | Q2M3M2 | ENSG00000117834 |
| SLC6A13 | Solute carrier family 6 member 13 | GAT2 | Q9NSD5 | ENSG00000010379 |
| SLC6A19 | Solute carrier family 6 member 19 | | Q695T7 | ENSG00000174358 |
| SLC7A7 | Solute carrier family 7 member 7 | LPI, y + LAT-1 | Q9UM01 | ENSG00000155465 |
| SLC7A8 | Solute carrier family 7 member 8 | LAT2, LPI-PC1 | Q9UHI5 | ENSG00000092068 |
| SLC7A9 | Solute carrier family 7 member 9 | CSNU3 | P82251 | ENSG00000021488 |
| SLC9A3R1 | SLC9A3 regulator 1 | EBP50, NHERF, NHERF1 | O14745 | ENSG00000109062 |
| SMIM1 | Small integral membrane protein 1 (Vel | Vel | B2RUZ4 | ENSG00000235169 |
| | blood group) | | | |
| SMIM10L2A | Small integral membrane protein 10 like | LED, LINC00086, | P0DMW4 | ENSG00000178947 |
| | 2A | LINC0086, MGC39606, | | |
| | | NCRNA00086 | | |
| SMIM18 | Small integral membrane protein 18 | | P0DKX4 | ENSG00000253457 |
| SMIM24 | Small integral membrane protein 24 | C19orf77, HSPC323 | O75264 | ENSG00000095932 |
| SMIM32 | Small integral membrane protein 32 | | A0A1B0G | ENSG00000271824 |
| | | | UA5 | |
| SMLR1 | Small leucine rich protein 1 | | H3BR10 | ENSG00000256162 |
| SMPDL3A | Sphingomyelin phosphodiesterase acid like | ASM3A, ASML3a, | Q92484 | ENSG00000172594 |
| | 3A | FLJ20177, yR36GH4.1 | | |
| SNX30 | Sorting nexin family member 30 | ATG24A | Q5VWJ9 | ENSG00000148158 |
| SOWAHB | Sosondowah ankyrin repeat domain family | ANKRD56 | A6NEL2 | ENSG00000186212 |
| | member B | | | |
| SPAG5 | Sperm associated antigen 5 | DEEPEST, hMAP126, | Q96R06 | ENSG00000076382 |
| | | MAP126 | | |
| ST18 | ST18 C2H2C-type zinc finger transcription | KIAA0535, NZF-3, NZF3, | O60284 | ENSG00000147488 |
| | factor | ZC2H2C3, ZC2HC10, | | |
| | | ZNF387 | | |
| STEAP1 | STEAP family member 1 | PRSS24, STEAP | Q9UHE8 | ENSG00000164647 |
| STRA6 | Signaling receptor and transporter of | FLJ12541 | Q9BX79 | ENSG00000137868 |
| | retinol STRA6 | | | |
| STRIP2 | Striatin interacting protein 2 | FAM40B, FAR11B, | Q9ULQ0 | ENSG00000128578 |
| | | KIAA1170 | | |
| STRIT1 | Small transmembrane regulator of ion | DWORF | P0DN84 | ENSG00000240045 |
| | transport 1 | | | |
| SUSD2 | Sushi domain containing 2 | BK65A6.2, FLJ22778, | Q9UGT4 | ENSG00000099994 |
| | | W5C5 | | |
| SUSD3 | Sushi domain containing 3 | MGC26847 | Q96L08 | ENSG00000157303 |
| SVOP | SV2 related protein | DKFZp761H039 | Q8N4V2 | ENSG00000166111 |
| SYN2 | Synapsin II | SYNII, SYNIIa, SYNIIb | Q92777 | ENSG00000157152 |
| SYT6 | Synaptotagmin 6 | | Q5T7P8 | ENSG00000134207 |
| SYT9 | Synaptotagmin 9 | | Q86SS6 | ENSG00000170743 |
| TACO1 | Translational activator of cytochrome c | CCDC44 | Q9BSH4 | ENSG00000136463 |
| | oxidase I | | | |
| TCL1B | TCL1 family AKT coactivator B | TML1 | O95988 | ENSG00000213231 |
| TCN2 | Transcobalamin 2 | D22S676, D22S750, TC2 | P20062 | ENSG00000185339 |
| TEX51 | Testis expressed 51 | | A0A1B0G | ENSG00000237524 |
| | | | UA7 | |
| TEX55 | Testis expressed 55 | C3orf30, FLJ32859, | Q96M34 | ENSG00000163424 |
| | | TSCPA | | |
| TFEC | Transcription factor EC | bHLHe34, TCFEC, | O14948 | ENSG00000105967 |
| | | TFECL | | |
| TGFBR3L | Transforming growth factor beta receptor 3 like | | H3BV60 | ENSG00000260001 |
| THPO | Thrombopoietin | MGDF, MPLLG, TPO | P40225 | ENSG00000090534 |
| TM4SF5 | Transmembrane 4 L six family member 5 | | O14894 | ENSG00000142484 |
| TM6SF2 | Transmembrane 6 superfamily member 2 | Lpr4 | Q9BZW4 | ENSG00000213996 |
| TM7SF3 | Transmembrane 7 superfamily member 3 | | Q9NS93 | ENSG00000064115 |
| TMED6 | Transmembrane p24 trafficking protein 6 | MGC23911, p24g5, | Q8WW62 | ENSG00000157315 |
| | | p24gamma5 | | |
| TMEM106A | Transmembrane protein 106A | MGC20235 | Q96A25 | ENSG00000184988 |
| TMEM132E | Transmembrane protein 132E | DFNB99 | Q6IEE7 | ENSG00000181291 |
| TMEM150A | Transmembrane protein 150A | FLJ90024, TM6P1, | Q86TG1 | ENSG00000168890 |
| | | TMEM150, TTN1 | | |
| TMEM150B | Transmembrane protein 150B | DRAM3, TMEM224, | A6NC51 | ENSG00000180061 |
| | | TTN2 | | |
| TMEM174 | Transmembrane protein 174 | FLJ31268, MGC13034 | Q8WUU8 | ENSG00000164325 |
| TMEM176A | Transmembrane protein 176A | HCA112, MS4B1 | Q96HP8 | ENSG00000002933 |
| TMEM82 | Transmembrane protein 82 | | A0PJX8 | ENSG00000162460 |
| TMIGD1 | Transmembrane and immunoglobulin | TMIGD, UNQ9372 | Q6UXZ0 | ENSG00000182271 |
| | domain containing 1 | | | |
| TRIM10 | Tripartite motif containing 10 | HERF1, RFB30, RNF9 | Q9UDY6 | ENSG00000204613 |
| TRIM14 | Tripartite motif containing 14 | KIAA0129 | Q14142 | ENSG00000106785 |
| TRIM15 | Tripartite motif containing 15 | RNF93, ZNF178, ZNFB7 | Q9C019 | ENSG00000204610 |
| TRPC7 | Transient receptor potential cation channel subfamily C member 7 | | Q9HCX4 | ENSG00000069018 |
| TRPM3 | Transient receptor potential cation channel | GON-2, KIAA1616, | Q9HCF6 | ENSG00000083067 |
| | subfamily M member 3 | LTRPC3 | | |
| TSPAN18 | Tetraspanin 18 | TSPAN | Q96SJ8 | ENSG00000157570 |
| TTC29 | Tetratricopeptide repeat domain 29 | NYD-SP14 | Q8NA56 | ENSG00000137473 |
| TTC36 | Tetratricopeptide repeat domain 36 | HBP21 | A6NLP5 | ENSG00000172425 |
| TTC38 | Tetratricopeptide repeat domain 38 | FLJ20699 | Q5R3I4 | ENSG00000075234 |
| TTLL6 | Tubulin tyrosine ligase like 6 | FLJ35808 | Q8N841 | ENSG00000170703 |
| TTPA | Alpha tocopherol transfer protein | AVED | P49638 | ENSG00000137561 |
| TUBA4B | Tubulin alpha 4b | FLJ13940, TUBA4 | Q9H853 | ENSG00000243910 |
| TUBAL3 | Tubulin alpha like 3 | FLJ21665 | A6NHL2 | ENSG00000178462 |
| TXN | Thioredoxin | TRX | P10599 | ENSG00000136810 |
| TXNDC17 | Thioredoxin domain containing 17 | MGC14353, TRP14, | Q9BRA2 | ENSG00000129235 |
| | | TXNL5 | | |
| UCN3 | Urocortin 3 | SPC, UCNIII | Q969E3 | ENSG00000178473 |
| UNC5CL | Unc-5 family C-terminal like | MGC34763, ZUD | Q8IV45 | ENSG00000124602 |
| USH1C | USH1 protein network component | AIE-75, DFNB18, | Q9Y6N9 | ENSG00000006611 |
| | harmonin | harmonin, NY-CO-37, | | |
| | | NY-CO-38, PDZ-73, | | |
| | | PDZ73, PDZD7C | | |
| UTP4 | UTP4 small subunit processome | CIRH1A, CIRHIN, | Q969X6 | ENSG00000141076 |
| | component | FLJ14728, KIAA1988, | | |
| | | NAIC, TEX292 | | |
| VIL1 | Villin 1 | D2S1471, VIL | P09327 | ENSG00000127831 |
| VSTM5 | V-set and transmembrane domain | C11orf90, LOC387804 | A8MXK1 | ENSG00000214376 |
| | containing 5 | | | |
| WDR72 | WD repeat domain 72 | FLJ38736 | Q3MJ13 | ENSG00000166415 |
| ZNF804B | Zinc finger protein 804B | FLJ32110 | A4D1E1 | ENSG00000182348 |
| ZYG11A | Zyg-11 family member A, cell cycle | ZYG11 | Q6WRX3 | ENSG00000203995 |
| | regulator | | | |
| COL4A3 | Collagen type IV alpha 3 chain | | Q01955 | ENSG00000169031 |
| COL4A4 | Collagen type IV alpha 4 chain | CA44 | P53420 | ENSG00000081052 |
| COL4A5 | Collagen type IV alpha 5 chain | ASLN, ATS | P29400 | ENSG00000188153 |
| MUC1 | Mucin 1, cell surface associated | ADMCKD, ADMCKD1, | P15941 | ENSG00000185499 |
| | | CD227, MCD, MCKD, | | |
| | | MCKD1, PEM, PUM | | |
| UMOD | Uromodulin | ABC35, ABCC7, CF, | P07911 | ENSG00000169344 |
| | | CFTR/MRP, dJ760C5.1, | | |
| | | MRP7, TNR-CFTR | | |
| KIF3A | Kinesin family member 3A | FLA10, KLP-20 | Q9Y496 | ENSG00000131437 |
| CFTR | CF transmembrane conductance regulator | ABC35, ABCC7, CF, | Q20BH0 | ENSG00000001626 |
| | | CFTR/MRP, dJ760C5.1, | | |
| | | MRP7, TNR-CFTR | | |
| PKD1 | Polycystin 1, transient receptor potential | PBP, Pc-1, TRPP1 | P98161 | ENSG00000008710 |
| | channel interacting | | | |
| PKD2 | Polycystin 2, transient receptor potential | Pc-2, PC2, PKD4, TRPP2 | Q13563 | ENSG00000118762 |
| | channel interacting | | | |
| PKHD1 | PKHD1 ciliary IPT domain containing | ARPKD, FCYT, FPC, | P08F94 | ENSG00000170927 |
| | fibrocystin/polyductin | TIGM1 | | |
| SLC15A1 | Solute carrier family 15 member 1 | HPECT1, HPEPT1, PEPT1 | P46059 | ENSG00000088386 |
| SLC15A2 | Solute carrier family 15 member | PEPT2 | Q16348 | ENSG00000163406 |
| KL | Klotho | | Q9UEF7 | ENSG00000133116 |
| FGFR1 | Fibroblast growth factor receptor 1 | BFGFR, CD331, CEK, | P11362 | ENSG00000077782 |
| | | FLG, FLT2, H2, H3, H4, | | |
| | | H5, KAL2, N-SAM | | |
| APOL1 | Apolipoprotein L1 | APOL | O14791 | ENSG00000100342 |
| GATM | Glycine amidinotransferase, mitochondrial | AGAT | P50440 | ENSG00000171766 |
| CFHR1 | Complement factor H-related protein 1 | CFHL, CFHL1, CFHL1P, | Q03591 | ENSG00000244414 |
| | | CFHR1P, FHR1, HFL1, | | |
| | | HFL2 | | |
| CFHR2 | Complement factor H-related protein 2 | CFHL2, FHR2, HFL3 | P36980 | ENSG00000080910 |
| CFHR3 | Complement factor H-related protein 3 | CFHL3, FHR3 | Q02985 | ENSG00000116785 |
| CFHR4 | Complement factor H-related protein 4 | CFHL4, FHR4 | Q92496 | ENSG00000134365 |
| CFHR5 | Complement factor H-related protein 5 | CFHL5, FHR5 | Q9BXR6 | ENSG00000134389 |
| SLC9A3 | Sodium/hydrogen exchanger 3 | NHE3 | P48764 | ENSG00000066230 |

TABLE 3
Exemplary characteristics and/or functions of RPTEC genes

A-kinase anchoring proteins
MYO7A
Acyl-CoA dehydrogenase family
ACAD11
Acyl-CoA thioesterases
ACOT6
Aldo-keto reductases
AKR7A3
KCNAB2
Ankyrin repeat domain containing
ANKRD33B
ANKS4B
ASB15
ESPN
PPP1R16B
SOWAHB
Apolipoproteins
APOC2
APOC3
APOE
APOH
APOL1
APOM
Basic helix-loop-helix proteins
ATOH7
MLXIPL
TFEC
Basic leucine zipper proteins
MAF
Beta-gamma crystallins
CRYBB3
Blood group antigens
C4A
GYPA
SMIM1
BPI fold containing
LBP
C-type lectin domain containing
CLEC18A
CLEC18B
CLEC18C
PKD1
C1q and TNF related
C1QTNF12
C2 domain containing
CPNE6
Cadherins
CDH9
CDHR2
CDHR3
CDHR5
PCDH15
CAP superfamily
R3HDML
CD molecules
FCAMR
GP5
MUC1
Chemokine ligands
CXCL14
Claudins
CLDN2
Collagens
COL19A1
COL4A3
COL4A4
COL4A5
Complement system
CFHR1
CFHR2
CFHR3
CFHR4
CFHR5
CTAGE family
MIA2
Cytochrome P450s
CYP2B6
Dbl family Rho GEFs
NGEF
EF-hand domain containing
CALML3
CALML4
CHP2
RAB11FIP3
REPS2
Erythrocyte membrane protein band 4.1
EPB41L3
F-BAR domain containing
PACSIN1
Fatty acid binding protein family
FABP1
FABP3
RBP5
Fatty acid desaturases
FADS6
Fibronectin type III domain containing
MYOM3
G protein-coupled receptors
GHRHR
MTNR1A
OPRD1
OXER1
Galectins
LGALS2
Gelsolin/villins
VIL1
Glycoside hydrolase family 31
MYORG
GOLD domain containing
TMED6
GRAM domain containing
GRAMD1B
Haloacid dehalogenase like hydrolase domain containing
HDHD3
Heat shock proteins
DNAJC12
DNAJC22
HSPA4L
Histones
H2AC1
H2BC1
Homeoboxes
DBX2
HNF1A
I-BAR domain containing
BAIAP2L2
Immunoglobulin superfamily domain containing
AZGP1
HAPLN4
HAVCR2
HHLA2
IGSF11
LRRC4
SEMA4G
TMIGD1
VSTM5
Interleukin receptors
IL17RB
IL22RA1
Intermediate filaments
KRT85
Ion channels
AQP11
AQP7
CACNA1E
GJB1
GJB2
KCNG2
KCNH6
KCNJ15
KCNK10
KCNK5
PKD2
SCN9A
TRPC7
TRPM3
Kinesins
KIF3A
Late cornified envelope proteins
LCE2D
Ligand gated ion channels
CFTR
CHRNA4
GRIA3
Low density lipoprotein receptors
LRP2
M14 carboxypeptidases
CPN2
Maestro heat like repeat containing
MRO
MROH2A
Membrane spanning 4-domains
TMEM176A
Metallothioneins
MT1G
MT1H
MT1X
Methyltransferase families
DNMT3L
METTL7B
Mitochondrial respiratory chain complex assembly factors
TACO1
Mitochondrial respiratory chain complexes
COX6A2
SDHC
Mucins
MUC13
Myosin heavy chains
MYO7B
N-BAR domain containing
BIN1
SH3GL2
N-terminal EF-hand calcium binding proteins
NECAB2
Na+/K+ transporting ATPase interacting
NKAIN4
NLR family
NLRP6
Non-coding RNAs
SLC22A18AS
Oxysterol binding proteins
OSBPL6
Paraneoplastic Ma antigens
PNMA6A
PDZ domain containing
GIPC2
PDZD3
PDZK1
SLC9A3R1
USH1C
Phospholipases
PLA2G12B
Pleckstrin homology domain containing
DOK6
RASGRF1
Protein phosphatase 1 regulatory subunits
KNL1
MAPT
PPP1R14D
PWWP domain containing
PWWP3B
Ras association domain family
RASSF4
Ras small GTPase superfamily
RAB29
RHOBTB1
Receptor accessory proteins
REEP6
Receptor kinases
FGFR1
Receptor ligands
ADM2
AGT
ANGPTL3
AVP
FGF5
OXT
THPO
UCN3
RNA binding motif containing
A1CF
CELF3
IGF2BP1
RALYL
Serine proteases
STEAP1
Serpin peptidase inhibitors
SERPINA6
SERPINC1
SERPINF2
SERPINI1
SH2 domain containing
SH2D6
Short chain dehydrogenase/reductase superfamily
DHRS4L2
Sideroflexins
SFXN1
SFXN2
SFXN5
Signal transduction and activation of RNA metabolism family
KHDRBS2
Solute carriers
MTCH2
NPC1L1
SLC10A2
SLC13A1
SLC13A2
SLC13A3
SLC15A1
SLC15A2
SLC16A10
SLC16A4
SLC16A9
SLC17A1
SLC17A3
SLC17A4
SLC1A1
SLC22A11
SLC22A12
SLC22A13
SLC22A24
SLC22A4
SLC22A6
SLC22A7
SLC22A8
SLC23A1
SLC23A3
SLC25A10
SLC25A42
SLC26A1
SLC26A9
SLC28A1
SLC28A2
SLC2A2
SLC2A5
SLC2A9
SLC30A2
SLC30A8
SLC34A1
SLC34A3
SLC36A2
SLC37A4
SLC39A4
SLC39A5
SLC3A1
SLC3A2
SLC47A1
SLC47A2
SLC4A4
SLC51A
SLC51B
SLC5A10
SLC5A11
SLC5A12
SLC5A2
SLC5A9
SLC6A13
SLC6A19
SLC7A7
SLC7A8
SLC7A9
SLC9A3
Sorting nexins
SNX30
Sterile alpha motif domain containing
SAMD5
STRIPAK complex
STRIP2
Sulfatases
ARSF
ARSL
Sushi domain containing
SUSD2
SUSD3
Synapsins
SYN2
Synaptotagmins
SYT6
SYT9
Tetraspanins
TSPAN18
Tetratricopeptide repeat domain containing
TTC29
TTC36
TTC38
Tripartite motif containing
TRIM14
Tubulin tyrosine ligase family
TTLL6
Tubulins
TUBA4B
TUBAL3
WD repeat domain containing
UTP4
WDR72
Zinc fingers
ABLIM3
DPF3
GLIS1
HNF4A
HNF4G
NR113
PRDM7
RNF212B
RNF224
RTL4
ST18
TRIM10
TRIM15
ZNF804B
ZYG11 cell cycle regulator family
ZYG11A

TABLE 4
Exemplary Podocyte genes

| Gene | Gene name | Gene synonym | Uniprot | Ensembl |
|---|---|---|---|---|
| ABHD12B | Abhydrolase domain containing 12B | BEM46L3, C14orf29 | Q7Z5M8 | ENSG00000131969 |
| ABLIM2 | Actin binding LIM protein family | KIAA1808 | Q6H8Q1 | ENSG00000163995 |
| | member 2 | | | |
| ADAMTS19 | ADAM metallopeptidase with | | | ENSG00000145808 |
| | thrombospondin type 1 motif 19 | | | |
| ADORA1 | Adenosine A1 receptor | RDC7 | P30542 | ENSG00000163485 |
| AIF1 | Allograft inflammatory factor 1 | AIF-1, Em: AF129756.17, IBA1, IRT-1 | P55008 | ENSG00000204472 |
| ALS2CL | ALS2 C-terminal like | DKFZp68610110, FLJ36525, | Q60127 | ENSG00000178038 |
| | | RN49018 | | |
| ANKRD30B | Ankyrin repeat domain 30B | NY-BR-1.1 | Q9BXX2 | ENSG00000180777 |
| APLN | Apelin | apelin, XNPEP2 | Q9ULZ1 | ENSG00000171388 |
| APLP1 | Amyloid beta precursor like protein 1 | APLP | P51693 | ENSG00000105290 |
| ARHGAP28 | Rho GTPase activating protein 28 | FLJ10312, KIAA1314 | Q9P2N2 | ENSG00000088756 |
| ARHGEF3 | Rho guanine nucleotide exchange | DKFZP434F2429, GEF3, STA3, XPLN | Q9NR81 | ENSG00000163947 |
| | factor 3 | | | |
| ARMH4 | Armadillo like helical domain | C14orf37, UT2 | Q86TY3 | ENSG00000139971 |
| | containing 4 | | | |
| AXDND1 | Axonemal dynein light chain domain | C1orf125, FLJ32940 | Q5T1B0 | ENSG00000162779 |
| | containing 1 | | | |
| BMP7 | Bone morphogenetic protein 7 | OP-1 | P18075 | ENSG00000101144 |
| C12orf56 | Chromosome 12 open reading frame 56 | | Q8IXR9 | ENSG00000185306 |
| C1QL1 | Complement C1q like 1 | C1QRF, C1QTNF14, CRF, CTRP14 | O75973 | ENSG00000131094 |
| CA10 | Carbonic anhydrase 10 | CA-RPX, CARPX, HUCEP-15 | Q9NS85 | ENSG00000154975 |
| CALHM4 | Calcium homeostasis modulator | C6orf78, FAM26D, FLJ32239 | Q5JW98 | ENSG00000164451 |
| | family member 4 | | | |
| CDC14A | Cell division cycle 14A | cdc14, Cdc14A1, Cdc14A2, | Q9UNH5 | ENSG00000079335 |
| | | DFNB105, DFNB32 | | |
| CDKN1C | Cyclin dependent kinase inhibitor 1C | BWCR, BWS, KIP2, P57 | P49918 | ENSG00000129757 |
| CFAP45 | Cilia and flagella associated protein 45 | CCDC19, NESG1 | Q9UL16 | ENSG00000213085 |
| CHI3L1 | Chitinase 3 like 1 | GP39, YK-40, YKL40 | P36222 | ENSG00000133048 |
| CLIC5 | Chloride intracellular channel 5 | DFNB102 | Q9NZA1 | ENSG00000112782 |
| CR1 | Complement C3b/C4b receptor 1 | CD35, KN | P17927 | ENSG00000203710 |
| | (Knops blood group) | | | |
| CRB2 | Crumbs cell polarity complex | FLJ16786, FLJ38464 | Q5IJ48 | ENSG00000148204 |
| | component 2 | | | |
| CRHBP | Corticotropin releasing hormone | CRF-BP, CRFBP | P24387 | ENSG00000145708 |
| | binding protein | | | |
| DDN | Dendrin | KIAA0749 | O94850 | ENSG00000181418 |
| DPP6 | Dipeptidyl peptidase like 6 | DPL1, DPPX | P42658 | ENSG00000130226 |
| EHD3 | EH domain containing 3 | PAST3 | Q9NZN3 | ENSG00000013016 |
| ELOVL4 | ELOVL fatty acid elongase 4 | CT118, SCA34, STGD2, STGD3 | Q9GZR5 | ENSG00000118402 |
| EVX1 | Even-skipped homeobox 1 | | P49640 | ENSG00000106038 |
| F3 | Coagulation factor III, tissue factor | CD142, TF | P13726 | ENSG00000117525 |
| F5 | Coagulation factor V | | P12259 | ENSG00000198734 |
| FGF1 | Fibroblast growth factor 1 | AFGF, ECGF, ECGF-beta, ECGFA, | P05230 | ENSG00000113578 |
| | | ECGFB, FGF-alpha, FGFA, GLIO703, | | |
| | | HBGF1 | | |
| FMN2 | Formin 2 | | Q9NZ56 | ENSG00000155816 |
| FOXD2 | Forkhead box D2 | FKHL17, FREAC9 | O60548 | ENSG00000186564 |
| FOXE3 | Forkhead box E3 | FKHL12, FREAC8 | Q13461 | ENSG00000186790 |
| GJA3 | Gap junction protein alpha 3 | CX46, CZP3 | Q9Y6H8 | ENSG00000121743 |
| GRIK2 | Glutamate ionotropic receptor kainate | GluK2, GLUR6, MRT6 | Q13002 | ENSG00000164418 |
| | type subunit 2 | | | |
| HOXA13 | Homeobox A13 | HOX1, HOX1J | P31271 | ENSG00000106031 |
| HTRA1 | HtrA serine peptidase 1 | ARMD7, HtrA, IGFBP5-protease, | Q92743 | ENSG00000166033 |
| | | PRSS11 | | |
| ISX | Intestine specific homeobox | RAXLX | Q2M1V0 | ENSG00000175329 |
| JAKMIP2 | Janus kinase and microtubule | JAMIP2, KIAA0555 | Q96AA8 | ENSG00000176049 |
| | interacting protein 2 | | | |
| JPH1 | Junctophilin 1 | JP-1 | Q9HDC5 | ENSG00000104369 |
| KIRREL2 | Kirre like nephrin family adhesion | DKFZp564A1164, FILTRIN, | Q6UWL6 | ENSG00000126259 |
| | molecule 2 | MGC15718, NEPH3, NLG1 | | |
| KLK6 | Kallikrein related peptidase 6 | Bssp, Klk7, neurosin, PRSS18, PRSS9 | Q92876 | ENSG00000167755 |
| KLK7 | Kallikrein related peptidase 7 | PRSS6, SCCE | P49862 | ENSG00000169035 |
| LCNL1 | Lipocalin like 1 | FLJ45224 | Q6ZST4 | ENSG00000214402 |
| LMX1B | LIM homeobox transcription factor 1 | NPS1 | O60663 | ENSG00000136944 |
| | beta | | | |
| MAFB | MAF bZIP transcription factor B | KRML | Q9Y5Q3 | ENSG00000204103 |
| MANSC4 | MANSC domain containing 4 | | A6NHS7 | ENSG00000205693 |
| MAP6 | Microtubule associated protein 6 | FLJ41346, KIAA1878, MAP6-N, STOP | Q96JE9 | ENSG00000171533 |
| MINAR2 | Membrane integral NOTCH2 | KIAA1024L | P59773 | ENSG00000186367 |
| | associated receptor 2 | | | |
| MME | Membrane metalloendopeptidase | CALLA, CD10, NEP | P08473 | ENSG00000196549 |
| MYLK3 | Myosin light chain kinase 3 | caMLCK, MLCK | Q32MK0 | ENSG00000140795 |
| NDNF | Neuron derived neurotrophic factor | C4orf31, FLJ23191 | Q8TB73 | ENSG00000173376 |
| NPHS1 | NPHS1 adhesion molecule, nephrin | CNF, NPHN | O60500 | ENSG00000161270 |
| NPHS2 | NPHS2 stomatin family member, | PDCN, SRN1 | Q9NP85 | ENSG00000116218 |
| | podocin | | | |
| NPNT | Nephronectin | EGFL6L, POEM | Q6UX19 | ENSG00000168743 |
| NTNG1 | Netrin G1 | KIAA0976, Lmnt1 | Q9Y212 | ENSG00000162631 |
| NXF3 | Nuclear RNA export factor 3 | | Q9H4D5 | ENSG00000147206 |
| PARD6G | Par-6 family cell polarity regulator | PAR-6G, PAR6gamma | Q9BYG4 | ENSG00000178184 |
| | gamma | | | |
| PCARE | Photoreceptor cilium actin regulator | C2orf71, FLJ34931, RP54 | A6NGG8 | ENSG00000179270 |
| PCOLCE2 | Procollagen C-endopeptidase | PCPE2 | Q9UKZ9 | ENSG00000163710 |
| | enhancer 2 | | | |
| PDPN | Podoplanin | aggrus, Gp38, GP40, PA2.26, T1A-2 | Q86YL7 | ENSG00000162493 |
| PHYHIP | Phytanoyl-CoA 2-hydroxylase | DYRK1AP3, KIAA0273, PAHX-AP | Q92561 | ENSG00000168490 |
| | interacting protein | | | |
| PLA2R1 | Phospholipase A2 receptor 1 | CLEC13C, PLA2-R, PLA2G1R, PLA2IR | Q13018 | ENSG00000153246 |
| PLCE1 | Phospholipase C epsilon 1 | KIAA1516, NPHS3, PLCE | Q9P212 | ENSG00000138193 |
| PODXL | Podocalyxin like | Gp200, PC, PCLP | O00592 | ENSG00000128567 |
| POSTN | Periostin | OSF-2, periostin, PN | Q15063 | ENSG00000133110 |
| PPFIA4 | PTPRF interacting protein alpha 4 | | O75335 | ENSG00000143847 |
| PTGDS | Prostaglandin D2 synthase | L-PGDS, PGDS | P41222 | ENSG00000107317 |
| PTPRO | Protein tyrosine phosphatase receptor | GLEPP1, NPHS6, PTP-oc, PTP-U2, | Q16827 | ENSG00000151490 |
| | type O | PTPU2 | | |
| PTPRQ | Protein tyrosine phosphatase receptor | DFNB84 | | ENSG00000139304 |
| | type Q | | | |
| RAB6C | RAB6C, member RAS oncogene family | WTH3 | Q9H0N0 | ENSG00000222014 |
| RAET1E | Retinoic acid early transcript 1E | bA350J20.7, LETAL, ULBP4 | Q8TD07 | ENSG00000164520 |
| RBFOX1 | RNA binding fox-1 homolog 1 | A2BP1, FOX-1, HRNBP1 | Q9NWB1 | ENSG00000078328 |
| RHBDL3 | Rhomboid like 3 | RHBDL4, VRHO | P58872 | ENSG00000141314 |
| ROBO2 | Roundabout guidance receptor 2 | KIAA1568 | Q9HCK4 | ENSG00000185008 |
| RXFP1 | Relaxin family peptide receptor 1 | LGR7, RXFPR1 | Q9HBX9 | ENSG00000171509 |
| SEMA5A | Semaphorin 5A | SEMAF, semF | Q13591 | ENSG00000112902 |
| SERPINI2 | Serpin family I member 2 | MEPI, PANCPIN, PI14, TSA2004 | O75830 | ENSG00000114204 |
| SHISA2 | Shisa family member 2 | bA398O19.2, C13orf13, hShisa, | Q6UWI4 | ENSG00000180730 |
| | | PRO28631, TMEM46, WGAR9166 | | |
| SLC45A1 | Solute carrier family 45 member 1 | DNB5 | Q9Y2W3 | ENSG00000162426 |
| SMCO3 | Single-pass membrane protein with | C12orf69, LOC440087 | A2RU48 | ENSG00000179256 |
| | coiled-coil domains 3 | | | |
| SPOCK1 | SPARC (osteonectin), cwcv and kazal | SPOCK, testican-1, TIC1 | Q08629 | ENSG00000152377 |
| | like domains proteoglycan 1 | | | |
| SPOCK2 | SPARC (osteonectin), cwcv and kazal | KIAA0275, testican-2 | Q92563 | ENSG00000107742 |
| | like domains proteoglycan 2 | | | |
| SPTB | Spectrin beta, erythrocytic | | P11277 | ENSG00000070182 |
| SRGAP2C | SLIT-ROBO Rho GTPase activating | SRGAP2P1 | P0DJJ0 | ENSG00000171943 |
| | protein 2C | | | |
| SSTR3 | Somatostatin receptor 3 | | P32745 | ENSG00000278195 |
| ST6GALNAC3 | ST6 N-acetylgalactosaminide alpha- | SIAT7C | Q8NDV1 | ENSG00000184005 |
| | 2,6-sialyltransferase 3 | | | |
| TACR3 | Tachykinin receptor 3 | NK3R, NKR, TAC3R | P29371 | ENSG00000169836 |
| TCEAL6 | Transcription elongation factor A like | WEX2 | Q6IPX3 | ENSG00000204071 |
| | 6 | | | |
| TENM2 | Teneurin transmembrane protein 2 | KIAA1127, ODZ2, Ten-M2, TEN2 | Q9NT68 | ENSG00000145934 |
| TMEM150C | Transmembrane protein 150C | FLJ12993, TTN3 | B9EJG8 | ENSG00000249242 |
| TMEM200C | Transmembrane protein 200C | TTMA | A6NKL6 | ENSG00000206432 |
| TMEM266 | Transmembrane protein 266 | C15orf27, FLJ38190 | Q2M3C6 | ENSG00000169758 |
| TMEM88B | Transmembrane protein 88B | | A6NKF7 | ENSG00000205116 |
| TMIE | Transmembrane inner ear | DFNB6 | Q8NEW7 | ENSG00000181585 |
| TNNT2 | Troponin T2, cardiac type | CMD1D, CMH2, CMPD2 | P45379 | ENSG00000118194 |
| TPPP3 | Tubulin polymerization promoting | CGI-38, p20, p25gamma | Q9BW30 | ENSG00000159713 |
| | protein family member 3 | | | |
| TRAV8-2 | T cell receptor alpha variable 8-2 | | A0A0B4J237 | ENSG00000211786 |
| TRIM54 | Tripartite motif containing 54 | MURF, MURF-3, RNF30 | Q9BYV2 | ENSG00000138100 |
| TYRO3 | TYRO3 protein tyrosine kinase | Brt, Dtk, Etk-2, Rek, RSE, Sky, Tif | Q06418 | ENSG00000092445 |
| VEGFA | Vascular endothelial growth factor A | VEGF, VEGF-A, VPF | P15692 | ENSG00000112715 |
| VSIG8 | V-set and immunoglobulin domain | | P0DPA2 | ENSG00000243284 |
| | containing 8 | | | |
| WDR49 | WD repeat domain 49 | FLJ33620 | Q8IV35 | ENSG00000174776 |
| WIPF3 | WAS/WASL interacting protein family | CR16, FLJ36931 | A6NGB9 | ENSG00000122574 |
| | member 3 | | | |
| WNT8B | Wnt family member 8B | | Q93098 | ENSG00000075290 |
| WT1 | WT1 transcription factor | AWT1, GUD, NPHS4, WAGR, WIT-2 | P19544 | ENSG00000184937 |
| ZDHHC6 | Zinc finger DHHC-type | FLJ21952, ZNF376 | Q9H6R6 | ENSG00000023041 |
| | palmitoyltransferase 6 | | | |
| ZNF804A | Zinc finger protein 804A | C2orf10 | Q7Z570 | ENSG00000170396 |
| SLC15A1 | Solute carrier family 15 member 1 | HPECT1, HPEPT1, PEPT1 | P46059 | ENSG00000088386 |
| SLC15A2 | Solute carrier family 15 member | PEPT2 | Q16348 | ENSG00000163406 |
| KL | Klotho | | Q9UEF7 | ENSG00000133116 |
| FGFR1 | Fibroblast growth factor receptor 1 | BFGFR, CD331, CEK, FLG, FLT2, H2, | P11362 | ENSG00000077782 |
| | | H3, H4, H5, KAL2, N-SAM | | |
| APOL1 | Apolipoprotein L1 | APOL | O14791 | ENSG00000100342 |
| CD2AP | CD2 associated protein | | Q9Y5K6 | ENSG00000198087 |
| TRPC6 | transient receptor potential cation | | Q9Y210 | ENSG00000137672 |
| | channel subfamily C member 6 | | | |
| SMARCAL1 | SWI/SNF related, matrix associated, | HARP | Q9NZC9 | ENSG00000138375 |
| | actin dependent regulator of | | | |
| | chromatin, subfamily a like 1 | | | |
| E2F3 | E2F transcription factor 3 | KIAA0075 | O00716 | ENSG00000112242 |
| NXF5 | nuclear RNA export factor 5 | TAPL1 | Q9H1B4 | ENSG00000126952 |
| PAX2 | paired box 2 | | Q02962 | ENSG00000075891 |
| WDR73 | WD repeat domain 73 | | Q6P412 | ENSG00000177082 |
| ACTN4 | actinin alpha 4 | | O43707 | ENSG00000130402 |
| MYH9 | myosin heavy chain 9 | | P35579 | ENSG00000100345 |
| INF2 | inverted formin 2 | C14orf151, C14orf173 | Q27J81 | ENSG00000203485 |
| SYNPO | synaptopodin | KIAA1029 | Q8N3V7 | ENSG00000171992 |
| MYO1E | myosin IE | MYO1C | Q12965 | ENSG00000157483 |
| ARHGAP24 | Rho GTPase activating protein 24 | FILGAP | Q8N264 | ENSG00000138639 |
| ARHGDIA | Rho GDP dissociation inhibitor alpha | GDIA1 | P52565 | ENSG00000141522 |
| ANLN | anillin actin binding protein | | Q9NQW6 | ENSG00000011426 |
| EMP2 | epithelial membrane protein 2 | XMP | P54851 | ENSG00000213853 |
| CUBN | cubilin | IFCR | O60494 | ENSG00000107611 |
| GPC5 | glypican 5 | | P78333 | ENSG00000179399 |
| TTC21B | tetratricopeptide repeat domain 21B | KIAA1992, IFT139 | Q7Z4L5 | ENSG00000123607 |
| CLTA4 | | CD152 | P16410 | ENSG00000163599 |
| MTTL1 | mitochondrially encoded tRNA-Leu | | | ENSG00000209082 |
| | (UUA/G) 1 | | | |
| COQ2 | coenzyme Q2, polyprenyltransferase | CL640 | Q96H96 | ENSG00000173085 |
| COQ6 | coenzyme Q6, monooxygenase | | Q9Y2Z9 | ENSG00000119723 |
| ZMPSTE24 | zinc metallopeptidase STE24 | FACE1, STE24 | O75844 | ENSG00000084073 |
| PDSS2 | decaprenyl diphosphate synthase | C6orf210, DLP1 | Q86YH6 | ENSG00000164494 |
| | subunit 2 | | | |
| ADCK4 | coenzyme Q8B | ADCK4 | Q96D53 | ENSG00000123815 |
| CYP11B2 | cytochrome P450 family 11 subfamily | | P19099 | ENSG00000179142 |
| | B member 2 | | | |
| LAMB2 | laminin subunit beta 2 | LAMS | P55268 | ENSG00000172037 |
| ITGB4 | integrin subunit beta 4 | | P16144 | ENSG00000132470 |
| ITGB3 | integrin subunit beta 3 | GP3A | P05106 | ENSG00000259207 |
| LMNA | lamin A/C | LMN1 | P02545 | ENSG00000160789 |
| COL4A3 | collagen type IV alpha 3 chain | | Q01955 | ENSG00000169031 |
| COL4A4 | collagen type IV alpha 4 chain | CA44 | P53420 | ENSG00000081052 |
| COL4A5 | | ASLN, ATS | P29400 | ENSG00000188153 |
| CD151 | | TSPAN24 | P48509 | ENSG00000177697 |
| PMM2 | | | O15305 | ENSG00000140650 |
| ALG1 | | HMAT1, HMT1 | Q9BT22 | ENSG00000033011 |
| SCARB2 | | CD36L2, LIMP2, LIMPII | Q14108 | ENSG00000138760 |

TABLE 5
Exemplary characteristics and/or functions of podocytes genes

Abhydrolase domain containing
ABHD12B
ADAM metallopeptidases with thrombospondin type 1 motif
ADAMTS19
Ankyrin repeat domain containing
ANKRD30B
Apolipoproteins
APOL1
Armadillo like helical domain containing
ARMH4
Basic leucine zipper proteins
MAFB
Blood group antigens
CR1
Bone morphogenetic proteins
BMP7
C-type lectin domain containing
PLA2R1
C1q and TNF related
C1QL1
Carbonic anhydrases
CA10
CD molecules
F3
MME
Chitinases
CHI3L1
Cilia and flagella associated
CFAP45
Crumbs complex
CRB2
Dbl family Rho GEFs
ALS2CL
ARHGEF3
EF-hand domain containing
AIF1
EHD3
RHBDL3
SPOCK1
SPOCK2
F-BAR domain containing
SRGAP2C
Fibronectin type III domain containing
NPHS1
PTPRO
PTPRQ
ROBO2
Forkhead boxes
FOXD2
FOXE3
Formins
FMN2
G protein-coupled receptors
ADORA1
RXFP1
SSTR3
TACR3
Gla domain containing
POSTN
Glycosyltransferases
ST6GALNAC3
Homeoboxes
EVX1
HOXA13
ISX
Immunoglobulin superfamily domain containing
KIRREL2
VSIG8
Ion channels
CLIC5
GJA3
Junctophilins
JPH1
Kallikreins
KLK6
KLK7
Ligand gated ion channels
GRIK2
Lipocalins
PTGDS
Myosin light chain kinase family
MYLK3
Netrins
NTNG1
PDZ domain containing
PARD6G
Phospholipases
PLCE1
Pleckstrin homology domain containing
SPTB
Potassium voltage-gated channel regulatory subunits
DPP6
Protein phosphatases
CDC14A
Ras small GTPase superfamily
RAB6C
Receptor kinases
FGFR1
TYRO3
Receptor ligands
APLN
FGF1
VEGFA
Rho GTPase activating proteins
ARHGAP28
RNA binding motif containing
RBFOX1
Semaphorins
SEMA5A
Serine proteases
HTRA1
Serpin peptidase inhibitors
SERPINI2
Shisa family members
SHISA2
Solute carriers
SLC15A1
SLC15A2
SLC45A1
Sterile alpha motif domain containing
PPFIA4
Stomatin family
NPHS2
T cell receptors
TRAV8-2
Tetraspan junctional complex superfamily
CALHM4
Transcription elongation factor A like family
TCEAL6
Troponin complex subunits
TNNT2
Tubulin polymerization promoting proteins
TPPP3
WD repeat domain containing
WDR49
Wnt family
WNT8B
Zinc fingers
ABLIM2
LMX1B
TRIM54
WT1
ZDHHC6
ZNF804A

Identification and/or Characterization of Conjugate Agents

In some embodiments, conjugate agent(s) as provided and/or utilized in accordance with the present disclosure are characterized in that, for example, when they are provided to a relevant system (e.g., comprising one or more cell(s), tissue(s), organ(s), or organism(s)) they impact expression and/or activity of one or more targets or form(s) thereof.

In some embodiments, a relevant agent is characterized by its impact on RNA (e.g., mRNA) and/or protein (e.g., encoded by an mRNA) targeted by its nucleic acid payload. In some such embodiments, such impact is assessed in vivo (i.e., in an organism). Alternatively or additionally, in some such embodiments, impact is assessed in vitro (e.g., in cell lines).

In some embodiments, conjugate agent(s) as described and/or utilized in accordance with the present disclosure are characterized relative to an unconjugated nucleic acid payload. In some embodiments, when assessed under comparable conditions, significantly greater impact is observed when an appropriate in vivo or in vitro system is contacted with a conjugate agent described herein than is observed when the system is contacted with an unconjugated payload under otherwise comparable conditions.

### Pharmaceutical Compositions

The present disclosure, among other things, provides pharmaceutical compositions that comprise or otherwise deliver a conjugate agent; typically, such pharmaceutical compositions comprise an active agent (e.g., a conjugate agent or a composition comprising the same) and one or more pharmaceutically or physiologically acceptable carriers, diluents, or excipients.

In some embodiments, pharmaceutical compositions described herein may comprise buffers including neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); and preservatives. In some embodiments, a pharmaceutical composition is substantially free of contaminants, e.g., there are no detectable levels of a contaminant (e.g., an endotoxin).

In some embodiments, pharmaceutical compositions described herein may be administered in a manner appropriate to the disease, disorder, or condition to be treated or prevented. In some embodiments, quantity and/or frequency of administration may be determined by such factors as condition of a patient, and/or type and/or severity of a patient's disease, disorder, or condition, although appropriate dosages may be determined by clinical trials.

In some embodiments, a pharmaceutical composition provided by the present disclosure may be in a form such as, for example, liquid, semi-solid and solid dosage forms, such as liquid solutions (e.g., injectable and infusible solutions), dispersions or suspensions, liposomes, and suppositories. Typically, pharmaceutical compositions that comprise or deliver antibody agents are injectable or infusible solutions; in some such embodiments, such compositions can be formulated for administration intravenously, subcutaneously, intradermally, intratumorally, intranodally, intramedullary, intramuscularly, transarterially, sublingually, intranasally, topically or intraperitoneally. In some embodiments, provided pharmaceutical compositions are formulated for intravenous administration. In some embodiments, provided pharmaceutical compositions are formulated for subcutaneous administration.

Pharmaceutical compositions described herein can be formulated for administration by using infusion techniques that are commonly known in the field (See, e.g., Rosenberg et al., New Eng. J. of Med. 319:1676, 1988, which is hereby incorporated by reference in its entirety).

In some embodiments, pharmaceutical compositions described herein are administered in combination with (e.g., before, simultaneously, or following) an additional therapy for a symptom, disease or disorder, e.g., a SOC therapy for a symptom, disease or disorder. In some embodiments, pharmaceutical compositions described herein may be administered before or following surgery.

In some embodiments, a dosage of any aforementioned therapy to be administered to a subject will vary with a disease, disorder, or condition being treated and based on a specific subject. Scaling of dosages for human administration can be performed according to art-accepted practices.

### Exemplary Uses of Conjugate Agents

### Cell Type for Delivery

In some embodiments, a conjugate agent disclosed herein is delivered to a cell, e.g., of a tissue, in which a cell surface factor is present.

In some embodiments, a cell is or comprises a cell (e.g., of a tissue) chosen from: immune cells (e.g., bone marrow cells, lymph node cells, thymic cells, peripheral blood mononuclear cells [e.g., myeloid and/or lymphoid cells], erythrocytes, eosinophils, neutrophils, and/or platelets); nervous system cells (e.g., brain tissue, cortex, cerebellum, retinal cells, spinal cord cells, nerve cells, neurons, and/or supporting cells; endothelial cells; muscle (e.g., heart muscle, smooth muscle, and/or skeletal muscle); small instetine cells; colon cells; adipocytes; kidney cells; liver cells; lung cells; splenic cells; stomach cells; esophagus cells; bladder cells; pancreas cells; thyroid cells; salivary gland cells; adrenal gland cells; pituitary gland cells; breast cells; skin cells; ovary cells; uterus cells; placenta cells; prostate cells; or testis cells, or a combination thereof.

In some embodiments, a cell is or comprises a cell (e.g., of a tissue) chosen from: renal cells, thyroid cells, parathyroid cells, cells of the inner ear, or nervous system cells.

In some embodiments, a cell is or comprises a kidney cell, e.g., as described herein. In some embodiments, a cell is or comprises a proximal tubular epithelial cell, a podocyte, or both.

In some embodiments, a cell to which a conjugate disclosed herein is delivered expresses both a cell surface factor (e.g., Megalin and/or Cubilin) and a target of a payload moiety.

In some embodiments, a conjugate agent disclosed herein is administered to a subject having a disease or disorder, e.g., as disclosed herein. In some embodiments, a disease or disorder comprises a cell in which a surface cell factor (e.g., Megalin and/or Cubilin) and/or a target of a payload moiety is present.

### Indications

In some embodiments, a conjugate agent disclosed herein is used to treat and/or prevent a symptom of, a disease or disorder disclosed herein.

In some embodiments, a disease or disorder to which a conjugate disclosed herein is provided, has elevated or aberrant expression of a cell surface factor such as Megalin and/or Cubilin.

In some embodiments, Megalin expression is reported to be enriched in the following tissues and/or cells in particular: renal tissue, thyroid tissue, parathyroid tissue, cells of the inner ear, and nervous system tissue. In some embodiments, Megalin is expressed (e.g., at relatively high level(s)) on surfaces of kidney cells such as proximal tubular epithelial cells and podocytes.

In some embodiments, a disease or disorder is chosen from: a glomerular disorder, a renal tubular disorder, other renal disorders, an inborn error of metabolism, a systemic metabolic disorder, a disorder of the thyroid, a disorder of the parathyroid, a disorder of the inner ear, a neurological disorder, or a viral infection, or a combination thereof.

In some embodiments, a disease or disorder is or comprises a glomerular disorder. In some embodiments, a glomerular disorder is chosen from: Lupus nephritis, Goodpasture syndrome, IgA nephropathy, Alport syndrome, glomerulosclerosis, diabetic nephropathy, focal segmental glomerulosclerosis, membranous nephropathy, minimal change disease, ApoL1 nephropathy, post-infection glomerulonephritis, membranoproliferative glomerulonephritis, mesangioproliferative glomerulonephritis, nephrotic syndrome, nephritic syndrome, Anti-LRP2 nephropathy, C3 glomerulopathy, or a combination thereof.

In some embodiments, a disease or disorder is or comprises a renal tubular disorder. In some embodiments, a renal tubular disorder is chosen from: Fanconi syndrome, cystinuria, Lowe syndrome, Dent syndrome, Light Chain Proximal Tubulopathy, Gitelman syndrome, renal tubular acidosis, nephrogenic diabetes insipidus, Bartter syndrome, Liddle syndrome, hereditary aminoaciduria, hereditary salt wasting disorders, hereditary phosphate wasting disorders, porphyria associated renal disease, nephropathic cystinosis, autosomal dominant tubulointerstitial kidney disease, or a combination thereof.

In some embodiments, a disease or disorder is or comprises other renal disorders. In some embodiments, other renal disorders are chosen from: ADPKD, ARPKD, Nephronophthisis, Chronic Kidney Disease, nephrolithiasis, acute kidney injury, Alagille syndrome, cardiorenal syndrome, renal cell carcinoma, renal osteodystrophy, or a combination thereof.

In some embodiments, a disease or disorder is or comprises an inborn error of metabolism. In some embodiments, an inborn error of metabolism is chosen from: phenylketonuria, urea cycle disorder, maple syrup urine disease, galactosemia, hereditary tyrosinemia, glutamic academia, isovaleric acidemia, very long/long/medium/short chain acyl-CoA dehydrogenase deficiency, methylmalonic academia, primary hyperoxaluria, propionic academia, porphyria, Wilson disease, Pyruvate dehydrogenase deficiency, homocystinuria, hereditary fructose intolerance, nonketotic hyperglycinemia, or a combination thereof.

In some embodiments, a disease or disorder is or comprises a systemic metabolic disorder. In some embodiments, a systemic metabolic disorder is chosen from: diabetes, obesity, hypertension, gout, polyneuropathy, hypoglycemia, vitamin B deficiencies, liver cirrhosis, coronary heart disease, stroke, lipodystrophy, or a combination thereof.

In some embodiments, a disease or disorder is or comprises a disorder of the thyroid. In some embodiments, a disorder of the thyroid is chosen from: Hashimoto disease, Graves' disease, hypothyroidism, hyperthyroidism, goiter, thyroid nodules, thyroiditis, thyroid cancer, thyrotropinoma, thyroid hormone resistance, MCT8 deficiency, Riedel's thyroiditis, Pendred syndrome, sarcoidosis, McCune-Albright syndrome, familial dysalbuminemic hyperthyroxinemia, thyroxin binding globulin (TBG) deficiency, or a combination thereof.

In some embodiments, a disease or disorder is or comprises a disorder of the parathyroid. In some embodiments, a disorder of the parathyroid is chosen from: hyperparathyroidism/hypercalcemia, hypoparathyroidism/hypocalcemia, nephrolithiasis (kidney stone), pancreatitis, granulomatous disease, Addison's disease, pernicious anemia (many of these belong to hyperparathyroidism and hypoparathyroidism).

In some embodiments, a disease or disorder is or comprises a disorder of the inner ear. In some embodiments, a disorder of the inner ear is chosen from: inherited sensorineural hearing loss, vestibular neuritis, Meniere's syndrome, benign paroxysmal positional vertigo, tinnitus, age related hearing loss, bilateral vestibular loss, perilymphatic fistula (PLF), superior semicircular canal dehiscence syndrome (SCD), drug-induced ototoxicity, herpes zoster oticus, purulent labyrinthitis, vestibular schwannoma.

In some embodiments, a disease or disorder is or comprises a neurological disorder, e.g., a neurodegenerative disease. In some embodiments, a neurological disorder is chosen from: Alzheimer's disease, Parkinson's disease, Huntington's disease, A.L.S., multiple sclerosis, neuro-AIDS, brain cancer, stroke, brain injury, spinal cord injury, autism, lysosomal storage disorders, fragile X syndrome, inherited mental retardation, inherited ataxias, blindness, paralysis, stroke, traumatic brain injury and spinal cord injury, and lysosomal storage diseases such as MPS I, MPS II, MPS III A, MPS III B, Metachromatic Leukodystrophy, Gaucher, Krabbe, Pompe, CLN2, Niemann-Pick and Tay-Sachs disease, or a combination thereof.

In some embodiments, a disease or disorder is or comprises a viral infection. In some embodiments, a viral infection comprises a polyoma virus (e.g., BK virus)-mediated nephropathy.

### Dosing Regimens

Those skilled in the art will be able to determine, according to known methods, the appropriate amount, dose or dosage of a conjugate agent, to administer to a patient, taking into account factors such as age, weight, general health, the route of administration, the nature of the symptom, disease or disorder requiring treatment, and the presence of other medications. For example, various dosing regimens for antibodies are disclosed in Hendrikx J et al. (2017) Oncologist 22 (10): 1212-1221, PMID: 28754722, the entire contents of which is hereby incorporated by reference.

In some embodiments, a conjugate agent is administered at a fixed dose, i.e. independent of body weight. In some embodiments, a fixed dose reduces interpatient variability, e.g., efficacy and/or PK/PD parameters.

In some embodiments, a conjugate agent is administered based on body weight, e.g., in a mg/kg dosing.

In some embodiments, a conjugate agent is administered at an initial dose. In some embodiments, an initial dose may be followed by one or more subsequent doses. In some embodiments, one or more subsequent dose may be administered daily, weekly, or monthly, or at other intervals in between. In some embodiments, a dosing regimen disclosed herein may be repeated for one or more times.

### Combination Therapies

In some embodiments, a conjugate agent disclosed herein, or a composition comprising the same is administered in combination with an additional agent, e.g., additional therapy. In some embodiments, an additional therapy comprises a therapy for a disease or disorder, e.g., a standard of care (SOC) therapy, for a symptom, disease or disorder. In some embodiments, a conjugate agent is administered before, concurrently with or after administration of an additional therapy, e.g., a SOC therapy.

### Exemplary Embodiments

Embodiment 1. A conjugate agent comprising:
(i) a targeting moiety; directly or indirectly conjugated with
(ii) a payload moiety.

Embodiment 2. The conjugate agent of embodiment 1, wherein the targeting moiety and payload moiety are indirectly conjugated by way of a linker.

Embodiment 3. The conjugate agent of embodiment 1 or 2, wherein the targeting moiety specifically binds a cell surface factor.

The conjugate agent of embodiment 5, wherein the cell Embodiment 4. surface factor is or comprises a kidney cell surface factor.

Embodiment 5. The conjugate agent of embodiment 6, wherein the kidney cell surface factor is internalized when bound by the targeting moiety.

Embodiment 6. The conjugate agent of embodiment 5 or 6, wherein the kidney cell surface factor is a receptor.

Embodiment 7. The conjugate agent of embodiment 8, wherein the receptor is Megalin or Cubilin.

Embodiment 8. The conjugate agent of any one of the preceding embodiments, wherein the targeting moiety is chosen from: a polypeptide, an aminoglycoside, an endogenous ligand (e.g., a ligand disclosed in Table 1), a xenobiotic, an antibody or a fragment thereof, an aptamer, a small molecule, or a combination thereof.

Embodiment 9. The conjugate agent of embodiment 8, wherein the targeting moiety is or comprises an endogenous ligand, e.g., a ligand disclosed in Table 1.

Embodiment 10. The conjugate agent of any one of the preceding embodiments, wherein the targeting moiety is or comprises a vitamin.

Embodiment 11. The conjugate agent of embodiment 10, wherein the vitamin is or comprises a vitamin provided in Table 1.

Embodiment 12. The conjugate agent of embodiment 10 or 11, wherein the vitamin is or comprises vitamin B12.

Embodiment 13. The conjugate agent of embodiment 8, wherein the targeting moiety is or comprises a polypeptide.

Embodiment 14. The conjugate agent of embodiment 13, wherein the polypeptide is chosen from: a peptide having a KKEEE motif; a fragment of receptor associated protein (RAP), a peptide derived from a radiopharmaceutical conjugates such as ocreotide, ocreotate, exendin, minigastrin, and/or neurotensin; or a combination thereof.

Embodiment 15. The conjugate agent of embodiment 14, wherein the polypeptide is or comprises a KKEEE motif.

Embodiment 16. The conjugate agent of embodiment 15, wherein the polypeptide comprises the sequence of SEQ ID NO: 1.

Embodiment 17. The conjugate agent of embodiment 14, wherein the polypeptide comprises a RAP fragment, or a variant thereof.

Embodiment 18. The conjugate agent of embodiment 17, wherein the RAP fragment comprises a polypeptide comprising residues 219-323 of RAP.

Embodiment 19. The conjugate agent of embodiment 13, wherein the polypeptide is or comprises a peptide derived from a radiopharmaceutical conjugates such as ocreotide, ocreotate, exendin, minigastrin, and/or neurotensin.

Embodiment 20. The conjugate agent of any one of embodiments 13-19, wherein the polypeptide is or comprises a knotted peptide.

Embodiment 21. The conjugate agent of embodiment 8, wherein the targeting moiety is or comprises an aminoglycoside.

Embodiment 22. The conjugate agent of embodiment 21, wherein the aminoglycoside is chosen from one or more, or all of: streptomycin, neomycin, kanamycin, paromomycin, gentamicin, G-418 (geneticin) ELX-202, tobramycin, amikacin, netilmicin, spectinomycin, sisomicin, dibekacin, isepamicin, framycetin, paromomycin, apramycin, fradiomycin, arbekacin, plazomicin, or a derivative, or a fragment, or a variant thereof.

Embodiment 23. The conjugate agent of embodiment 8, wherein the targeting moiety is or comprises a xenobiotic.

Embodiment 24. The conjugate agent of embodiment 23, wherein the xenobiotic is or comprises polymixin, aprotinin, trichosanthin, or a combination thereof.

Embodiment 25. The conjugate agent of embodiment 8, wherein the targeting moiety is or comprises an antibody of a fragment thereof.

Embodiment 26. The conjugate agent of embodiment 25, wherein the antibody or fragment thereof selectively binds Megalin, Cubilin, or both.

Embodiment 27. The agent of embodiment 25 or 26, wherein the antibody or fragment thereof specifically binds Megalin.

Embodiment 28. The agent of embodiment 25 or 26, wherein the antibody or fragment thereof specifically binds Cubilin.

Embodiment 29. The conjugate agent of any one of embodiments 25-28, wherein the antibody of fragment thereof is a bispecific antibody or a multi-specific antibody.

Embodiment 30. The conjugate agent of any one of embodiments 25-29, wherein the antibody comprises one or more modifications of an Fc domain, e.g., an Fc variant.

Embodiment 31. The conjugate agent of any one of embodiments 1-30, wherein the targeting moiety binds the receptor at one or more extracellular domains on the receptor.

Embodiment 32. The conjugate agent of embodiment 31, wherein the targeting moiety binds the receptor at or near one or more complement type repeat domains.

Embodiment 33. The conjugate agent of any one of the preceding embodiments, wherein the payload moiety acts on a target chosen from a target provided in any one of Tables 2-5, or a combination thereof.

Embodiment 34. The conjugate agent of any one of the preceding embodiments, wherein the payload moiety is or comprises a nucleic acid.

Embodiment 35. The conjugate agent of embodiment 34, wherein the nucleic acid is or comprises an antisense sequence element.

Embodiment 36. The conjugate agent of embodiment 35, wherein the antisense sequence element is complementary to at least a portion of one or more of: an exon, an intron, an untranslated region, a splice junction, a promoter region, an enhancer region, or a non-coding region in a target sequence.

Embodiment 37. The conjugate agent of any one of the preceding embodiments, wherein the nucleic acid comprises a sequence element that is at least 80% complementary to a target sequence in a sense strand.

Embodiment 38. The conjugate agent of any one of embodiments 1-36, wherein the nucleic acid comprises a sequence element that is at least 80% complementary to a target sequence in an antisense strand.

Embodiment 39. The conjugate agent of any one of the preceding embodiments, wherein the nucleic acid comprises at least one sequence element with at least 3 contiguous nucleotides having at least 80% complementarity to a portion of a target sequence.

Embodiment 40. The conjugate agent of any one of embodiments 34-39, wherein the nucleic acid is single stranded.

Embodiment 41. The conjugate agent of any one of embodiments 34-39, wherein the nucleic acid is double stranded.

Embodiment 42. The conjugate agent of any one of embodiments 34-41, wherein the nucleic acid is or comprises RNA.

Embodiment 43. The conjugate agent of embodiment 42, wherein the RNA is or comprises an interfering RNA (RNAi).

Embodiment 44. The conjugate agent of embodiment 43, wherein the RNAi is or comprises a short interfering RNA (siRNA) or a micro RNA (miRNA).

Embodiment 45. The conjugate agent of embodiment 42, wherein the RNA is or comprises a guide RNA (gRNA).

Embodiment 46. The conjugate agent of any one of embodiments 42-45, wherein the RNA comprises about 15-25 nucleotides.

Embodiment 47. The conjugate agent of any one of embodiments 42-46, wherein the RNA comprises one or more modified nucleotides.

Embodiment 48. The conjugate agent of any one of embodiments 34-41, wherein the nucleic acid is or comprises DNA.

Embodiment 49. The conjugate agent of embodiment 48, wherein the DNA is or comprises a DNA analog.

Embodiment 50. The conjugate agent of embodiment 49, wherein the DNA analog comprises one or more morpholino subunits linked together by phosphorus-containing linkage.

Embodiment 51. The conjugate agent of embodiment 50, wherein the DNA analog is or comprises a phosphorodiamidate morpholino nucleic acid (PMO).

Embodiment 52. The conjugate agent of embodiment 51, wherein the PMO comprises about 12-40 nucleotides.

The conjugate agent of any one of embodiments 34-52, Embodiment 53, wherein the nucleic acid is or comprises an antisense oligo (ASO).

Embodiment 54. The conjugate agent of any one of embodiments 34-41 or 48-52, wherein the nucleic acid is or comprises a peptide nucleic acid (PNA).

Embodiment 55. The conjugate agent any one of embodiments 34-54, wherein the nucleic acid comprises a modification comprising: a modified backbone, a modified nucleobase, a modified ribose, a modified deoxyribose, or a combination thereof.

Embodiment 56. The conjugate agent of embodiment 55, wherein the modification is chosen from: a 2′-O-methyl modified nucleotide, a 5-methylcytidine, a 5-methyluridine, a nucleotide comprising a 5′-phosphorothioate group, a morpholino nucleotide (e.g., a PMO), a terminal nucleotide linked to a cholesteryl derivative or a dodecanoic acid bisdecylamide group, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide (e.g., PMO), a phosphoramidate, a phosphoryl guanidine-based backbone, or a non-natural base comprising nucleotide, or a combination thereof.

Embodiment 57. The conjugate agent of embodiment 55 or 56, wherein the modification is chosen from: a C7-modified deaza-adenine, a C7-modified deaza-guanosine, a C5-modified cytosine, a C5-modified uridine, N1-methyl-pseudouridine (mlψ), 1-ethyl-pseudouridine (elψ), 5-methoxy-uridine (mo5U), 5-methyl-cytidine (m5C), pseudouridine (ψ), 5-methoxymethyl uridine, 5-methylthio uridine, 1-methoxymethyl pseudouridine, 5-methyl cytidine, 5-methoxy cytidine, or a combination thereof.

Embodiment 58. The conjugate agent of any one of embodiments 55-57, wherein the modification is chosen from: a 2′fluoro modification, a 2′-O-methyl (2′OMe) modification, a locked nucleic acid (LNA), a 2′-fluoro arabinose nucleic acid (FANA), a hexitol nucleic acid (HNA), a 2′O-methoxyethyl (2′MOE) modification, or a combination thereof.

Embodiment 59. The conjugate agent of any one of embodiments 55-58, wherein the modification is chosen from: a phosphorothioate (PS) modification, a phosphoryl guanidine (PN) modification, a borano-phosphate modification, an alkyl phosphonate nucleic acid (phNA), a peptide nucleic acid (PNA), or a combination thereof.

Embodiment 60. The conjugate agent of any one of embodiments 34-59, wherein the nucleic acid comprises one or more modification to a 5′ end of the nucleic acid.

Embodiment 61. The conjugate agent of embodiment 60, wherein the nucleic acid comprise a 5′ amino modification.

Embodiment 62. The conjugate agent of any one of embodiments 34-61, wherein the nucleic acid is characterized in that when delivered to a cell expressing the target, reduced expression and/or activity of the target is observed as compared to a cell which has not been delivered the nucleic acid or a cell which does not express the target.

Embodiment 63. The conjugate agent of any one of the preceding embodiments, wherein the payload moiety is conjugated to the targeting moiety at a 5′ end of the payload moiety.

Embodiment 64. The conjugate agent of any one of embodiments 1-62, wherein the payload moiety is conjugated to the targeting moiety at a 3′ end of the payload moiety.

Embodiment 65. The conjugate agent of any one of embodiments 2-64, wherein the payload moiety is conjugated to the targeting moiety by way of a linker, wherein: (i) the linker is a cleavable linker; (ii) the linker becomes cleaved when exposed to a cell-internal environment; or both (i) and (ii).

Embodiment 66: The conjugate agent of embodiment 65, wherein the linker comprises about 1-30 repeats.

Embodiment 67. The conjugate agent of any one of the preceding embodiments, wherein the targeting moiety and the payload moiety are conjugated by a linker comprising the structure:

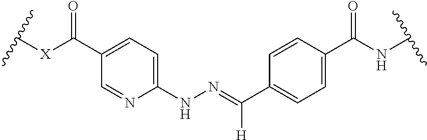

wherein X is NH or O.

Embodiment 68. The conjugate agent of any one of embodiments 1-64, wherein the targeting moiety and the payload moiety are conjugated by a linker comprising the structure:

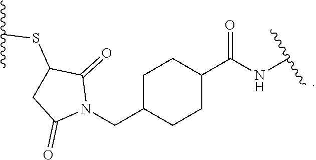 [SMILES: *NC(=O)C1CCC(CN2C(=O)CC(S*)C2=O)CC1]

Embodiment 69. A conjugate agent comprising:
(i) a targeting moiety; directly or indirectly conjugated with
(ii) a payload moiety comprising a nucleic acid that targets a target which is present: in a cell in which a cell surface factor is present.

Embodiment 70. The conjugate agent of embodiment 69, wherein the targeting moiety comprises a kidney-specific targeting moiety.

Embodiment 71. The conjugate agent of embodiment 70, wherein the cell is chosen from: immune cells; nervous system cells; muscle cells; small intestine cells; colon cells; adipocytes; kidney cells; liver cells; lung cells; splenic cells; stomach cells; esophagus cells; bladder cells; pancreas cells; thyroid cells; salivary gland cells; adrenal gland cells; pituitary gland cells; breast cells; skin cells; ovary cells; uterus cells; placenta cells; prostate cells; or testis cells, or a combination thereof.

Embodiment 72. The conjugate agent of embodiment 70, wherein the cell is chosen from: renal cells, thyroid cells, parathyroid cells, cells of the inner ear, nervous system cells, or a combination thereof.

Embodiment 73. The conjugate agent of embodiment 70, wherein the cell is chosen from a proximal tubular epithelial cell and/or a podocyte.

Embodiment 74. The conjugate of any one of embodiments 69-73, wherein the cell surface factor is or comprises a kidney cell surface factor.

Embodiment 75. The conjugate agent of embodiment 74, wherein the kidney cell surface factor is or comprises Megalin, Cubilin, or both.

Embodiment 76. A conjugate agent comprising:
(i) a targeting moiety specific for an internalizing cell surface factor; and
(ii) a payload moiety comprising a nucleic acid agent,
wherein the targeting moiety and nucleic acid agent are conjugated to one another by way of a cleavable linker so that the conjugate agent is in a first, associated state, when extracellular to a kidney cell and a second, disassociated state, when internal to a cell in which a cell surface factor is present.

Embodiment 77. The conjugate agent of any one of the preceding embodiments, characterized in that when delivered to a cell, tissue or organism, the payload moiety is delivered to, and/or expressed in, at least 5% more target cells compared to an otherwise similar cell, tissue or organism delivered an unconjugated payload moiety.

Embodiment 78. The conjugate agent of any one of the preceding embodiments, characterized in that when delivered to a tissue or organism, the payload moiety is delivered to, and/or expressed in, at least 5% more target cells compared to non-target cells.

Embodiment 79. The conjugate agent of embodiment 77 or 78, wherein the target cell is or comprises a cell chosen from: immune cells; nervous system cells; muscle cells; small intestine cells; colon cells; adipocytes; kidney cells; liver cells; lung cells; splenic cells; stomach cells; esophagus cells; bladder cells; pancreas cells; thyroid cells; salivary gland cells; adrenal gland cells; pituitary gland cells; breast cells; skin cells; ovary cells; uterus cells; placenta cells; prostate cells; or testis cells, or a combination thereof.

Embodiment 80. The conjugate agent of embodiment 77 or 78, wherein the target cell is chosen from: renal cells, thyroid cells, parathyroid cells, cells of the inner ear, nervous system cells, or a combination thereof.

Embodiment 81. The conjugate agent of embodiment 77 or 78, wherein the target cell is or comprises a kidney cell chosen from: a proximal tubular epithelial cell, a podoctye, or both.

Embodiment 82. The conjugate agent of any one of embodiments 77-81, wherein the target cell is or comprises a cell that expresses (e.g., detectably expresses) a cell surface factor.

Embodiment 83. The conjugate agent of embodiment 82, wherein the cell surface factor is or comprises a kidney cell surface factor.

Embodiment 84. The conjugate agent of embodiment 83, wherein the kidney cell surface factor is Megalin, or a variant, or a fragment thereof.

Embodiment 85. The conjugate agent of embodiment 83, wherein the kidney cell surface factor is Cubilin, or a variant, or a fragment thereof.

Embodiment 86. The conjugate agent of any one of embodiments 77-85, wherein the target cell expresses one or more targets chosen from: a target provided in any one of Tables 2-5.

Embodiment 87. The conjugate agent of any one of embodiment 78-86, wherein the non-target cell is or comprises a cell that does not express (e.g., has no detectable expression of) a cell surface factor.

Embodiment 88. The conjugate agent of embodiment 87, wherein the non-target cell is or comprises a cell that does not express (e.g., has no detectable expression of) a kidney cell surface factor (e.g., Megalin and/or Cubilin).

Embodiment 89. The conjugate agent of any one of the preceding embodiments, characterized in that when delivered to a cell, tissue or organism, expression and/or activity of the target of the payload moiety is modulated, e.g., reduced, by at least 5% compared to an otherwise similar cell, tissue or organism delivered an unconjugated payload moiety.

Embodiment 90. A conjugate comprising the structure of Formula I:

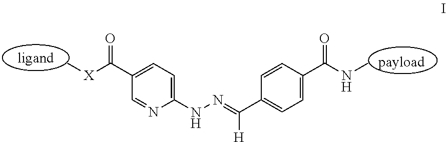

wherein
X is NH or O;
the ligand is a targeting moiety; and
the payload is a payload moiety.

Embodiment 91. A conjugate comprising the structure of Formula II:

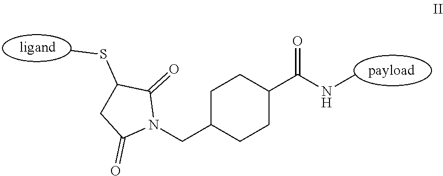

wherein the ligand is a targeting moiety; and
the payload is a payload moiety.

Embodiment 92. The conjugate of any one of the preceding embodiments, wherein the targeting moiety is Gentamicin or a derivative or variant thereof.

Embodiment 93. The conjugate of embodiment 92, wherein the linker is attached to ring 2 of the targeting moiety as provided in Formula III.

Embodiment 94. A conjugate comprising the structure of Formula III:

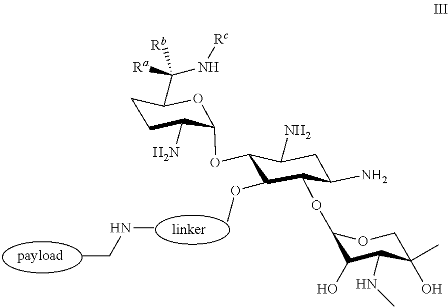

wherein each of R^{a}, R^{b}, and R^{c} is selected from H and CH₃;
the linker is a bivalent linker; and
the payload is a payload moiety.

Embodiment 95. The conjugate of any one of embodiments 69 to 90, wherein the conjugate comprises a payload moiety provided in any one of embodiments 33-64.

Embodiment 96. The conjugate of any one of embodiments 69 to 90, wherein the conjugate comprises a linker provided in any one of embodiments 65-68.

Embodiment 97. A pharmaceutical composition that comprises or delivers the conjugate agent of any one of the preceding embodiments.

Embodiment 98. The pharmaceutical composition of embodiment 97, formulated for intravenous, subcutaneous, intramuscular, parenteral, or oral delivery.

Embodiment 99. The pharmaceutical composition of embodiment 97 or 98, comprising one or more pharmaceutically or physiologically acceptable carriers, diluents, or excipients.

Embodiment 100. The pharmaceutical composition of any one of embodiments 97-99, wherein the composition comprises less than 5% of an impurity.

Embodiment 101. The pharmaceutical composition of embodiment 100, wherein the impurity comprises one or more of: an endotoxin, a cellular component, or an aggregate.

Embodiment 102. A cell with a conjugate agent of any one of embodiments 1-96 bound thereto.

Embodiment 103. The cell of embodiment 102, wherein the cell is in a tissue, an organ, or an organism.

Embodiment 104. The cell of embodiment 102 or 103, wherein the conjugate agent is internalized upon binding to a cell surface factor.

Embodiment 105. The cell of embodiment 104, wherein internalization of the conjugate agent delivers the payload moiety into an internal compartment of, or a vesicle in a cell.

Embodiment 106. A payload moiety comprising a nucleic acid recognizing a target, linked to a cleaved first portion of a linker.

Embodiment 107. The payload moiety of embodiment 106, wherein the payload moiety is in a cell in which a cell surface factor is present.

Embodiment 108. The payload moiety of embodiment 107, wherein the cell further comprises a targeting moiety linked to a cleaved second portion of the linker.

Embodiment 109. A method of delivering a conjugate agent to a subject, the method comprising a step of:

administering to the subject, the conjugate agent comprising a targeting moiety directly or indirectly linked with a payload moiety of any one of embodiments 1-96, or the pharmaceutical composition of any one of embodiments 97-101.

Embodiment 110. The method of embodiment 109, wherein the payload reduces expression and/or activity of a target provided in any one of Tables 2-5, or a combination thereof.

Embodiment 111. The method of embodiment 109 or 110, wherein the conjugate agent is delivered to a cell expressing a cell surface factor.

Embodiment 112. The method of embodiment 111, wherein the cell surface factor is a kidney cell surface factor.

Embodiment 113. The method of embodiment 112, wherein the kidney cell surface factor is chosen from megalin and/or cubilin.

Embodiment 114. The method of any one of embodiments 109-113, wherein the conjugate agent is delivered to a tissue, organ, or fluid compartment.

Embodiment 115. A method of treating a disease or disorder, the method comprising a step of:
administering to a subject suffering from or susceptible to the disease or disorder, the conjugate agent comprising a targeting moiety directly or indirectly linked with a payload moiety of any one of embodiments 1-96, or the pharmaceutical composition of any one of embodiments 97-101.

Embodiment 116. The method of embodiment 115, wherein the disease is a disease associated with expression of a cell surface receptor.

Embodiment 117. The method of embodiment 115 or 116, wherein the disease is a disease comprising a cell in which both a cell surface receptor and a target recognized by the payload moiety are present.

Embodiment 118. The method of any one of embodiments 115-117, wherein the disease or disorder is chosen from: a glomerular disorder, a renal tubular disorder, other renal disorders, an inborn error of metabolism, a systemic metabolic disorder, a disorder of the thyroid, a disorder of the parathyroid, a disorder of the inner ear, a neurological disorder, a viral infection, or a combination thereof.

Embodiment 119. In a method of treating a disease with a nucleic acid, the improvement comprising a step of:
administering the nucleic acid as a conjugate with a targeting moiety.

Embodiment 120. The method of embodiment 119, wherein the disease is a disease associated with expression of a cell surface receptor.

Embodiment 121. The method of embodiment 119 or 120, wherein the disease is a disease comprising a cell in which both: a cell surface receptor and a target recognized by the payload moiety are present.

Embodiment 122. A method of improving delivery of an agent to a cell, the method comprising contacting a system or subject comprising at least one cell with a conjugate agent of any one of embodiments 1-96 or a pharmaceutical composition of any one of embodiments 97-101.

Embodiment 123. A method of improving delivery of an agent to a cell, the method comprising contacting a system or subject comprising at least one cell with a conjugate agent of any one of embodiments 1-96 or a pharmaceutical composition of any one of embodiments 97-101, wherein the cell is a cell that expresses a cell surface receptor.

Embodiment 124. The method of any one of embodiments 111-118 or 122-123, wherein the cell is chosen from: immune cells; nervous system cells; muscle cells; small intestine cells; colon cells; adipocytes; kidney cells; liver cells; lung cells; splenic cells; stomach cells; esophagus cells; bladder cells; pancreas cells; thyroid cells; salivary gland cells; adrenal gland cells; pituitary gland cells; breast cells; skin cells; ovary cells; uterus cells; placenta cells; prostate cells; or testis cells, or a combination thereof.

Embodiment 125. The method of any one of embodiments 111-118 or 123-124, wherein the cell is chosen from: renal cells, thyroid cells, parathyroid cells, cells of the inner ear or nervous system cells, or a combination thereof.

Embodiment 126. The method of any one of embodiments 111-118 or 123-124, wherein the cell is chosen from a proximal tubular epithelial cell and/or a podocyte.

Embodiment 127. The method of any one of embodiments 122-126, wherein contacting comprises administering the conjugate agent to:
the cell; a tissue comprising the cell; or an organism comprising the cell.

Embodiment 128. The method of any one of embodiments 109-127, wherein administering the conjugate agent to the cell, tissue or organism, delivers the payload moiety to at least 5% more target cells compared to an otherwise similar cell, tissue or organism delivered an unconjugated payload moiety.

Embodiment 129. The method of any one of embodiments 109-127, wherein administering the conjugate agent to the cell, tissue or organism, delivers the payload moiety to at least 5% more target cells compared to non-target cells.

Embodiment 130. The method of embodiment 128 or 129, wherein the target cell is or comprises a kidney cell.

Embodiment 131. The method of any one of embodiments 128-130, wherein the target cell is or comprises a cell that has expression of a cell surface factor.

Embodiment 132. The method of any one of embodiments 128-131, wherein the non-target cell is or comprises a cell that has no expression of (e.g., no detectable expression of) a cell surface factor.

Embodiment 133. The method of any one of embodiments 109-132, wherein administering the conjugate agent to the cell, tissue or organism, reduces expression and/or activity of the target of the payload moiety by at least 5% compared to an otherwise similar cell, tissue or organism delivered an unconjugated payload moiety.

Embodiment 134. The method of any one of embodiments 109-133, wherein the conjugate agent is delivered intravenously, subcutaneously, intramuscularly, parenterally or orally.

Embodiment 135. The method of any one of embodiments 109-134, wherein the conjugate agent is delivered in one or more doses.

Embodiment 136. The method of any one of embodiments 109-135, wherein the conjugate agent is delivered in combination with one or more additional conjugate agents.

Embodiment 137. The method of embodiment 136, wherein the one or more additional conjugate agents comprises a different payload moiety, a different linker, a different targeting moiety, or a combination thereof.

Embodiment 138. The method of any one of embodiments 109-137, wherein the conjugate agent is delivered in combination with one or more additional therapeutic modalities.

Embodiment 139. Use of a conjugate agent according to any one of embodiments 1-96, or a pharmaceutical composition of any one of embodiments 97-101, in the preparation of a medicament for delivering a conjugate agent to a subject.

Embodiment 140. A composition comprising the conjugate agent of any one of embodiments 1-96, or the pharmaceutical composition of any one of embodiments 97-101, for use in delivering the conjugate agent to a subject.

Embodiment 141. The use of embodiment 139, or the composition for use of embodiment 140, wherein the conjugate agent or the pharmaceutical composition is administered to the subject.

Embodiment 143. The use of embodiment 139 or the composition for use of embodiment 140, wherein the payload reduces expression and/or activity of a target provided in any one of Tables 2-5, or a combination thereof.

Embodiment 144. The use of embodiment 139, or the composition for use of embodiment 140, wherein the conjugate agent is delivered to a cell expressing a cell surface factor.

Embodiment 145. The use of embodiment 144, or the composition for use of embodiment 144, wherein the cell surface factor is a kidney cell surface factor.

Embodiment 146. The use of embodiment 145, or the composition for use of embodiment 145, wherein the kidney cell surface factor is chosen from megalin and/or cubilin.

Embodiment 147. The use of embodiment 139, or the composition for use of embodiment 140, wherein the conjugate agent is delivered to a tissue, organ, or fluid compartment.

Embodiment 148. Use of a conjugate agent according to any one of embodiments 1-96, or a pharmaceutical composition of any one of embodiments 97-101, in the preparation of a medicament for treating a disease or disorder in a subject suffering from or susceptible to the disease or disorder.

Embodiment 149 A composition comprising the conjugate agent of any one of embodiments 1-96, or the pharmaceutical composition of any one of embodiments 97-101, for use in treating a disease or disorder in a subject suffering from or susceptible to the disease or disorder.

Embodiment 150: The use of embodiment 148, or the composition for use of embodiment 149, wherein the conjugate agent or the pharmaceutical composition is administered to the subject.

Embodiment 151. The use of embodiment 148, or the composition for use of embodiment 149, wherein the disease is a disease associated with expression of a cell surface receptor.

Embodiment 152. The use of embodiment 148, or the composition for use of embodiment 149, wherein the disease is a disease comprising a cell in which both a cell surface receptor and a target recognized by the payload moiety are present.

Embodiment 153. The use of embodiment 148, or the composition for use of embodiment 149, wherein the disease or disorder is chosen from: a glomerular disorder, a renal tubular disorder, other renal disorders, an inborn error of metabolism, a systemic metabolic disorder, a disorder of the thyroid, a disorder of the parathyroid, a disorder of the inner ear, a neurological disorder, a viral infection, or a combination thereof.

Embodiment 154. Use of a conjugate agent according to any one of embodiments 1-96, or a pharmaceutical composition of any one of embodiments 97-101, in the preparation of a medicament for improving delivery of an agent to a cell.

Embodiment 155. A composition comprising the conjugate agent of any one of embodiments 1-96, or the pharmaceutical composition of any one of embodiments 97-101, for use in improving delivery of an agent to a cell.

Embodiment 156. Use of a conjugate agent according to any one of embodiments 1-96, or a pharmaceutical composition of any one of embodiments 97-101, in the preparation of a medicament for improving delivery of an agent to a cell, wherein the cell is a cell that expresses a cell surface receptor.

Embodiment 157: A composition comprising the conjugate agent of any one of embodiments 1-96, or the pharmaceutical composition of any one of embodiments 97-101, for use in improving delivery of an agent to a cell, wherein the cell is a cell that expresses a cell surface receptor.

Embodiment 158: The use of embodiment 154 or 156, or the composition of use 155 or 157, comprising contacting a system or subject comprising at least one cell with the conjugate agent or the pharmaceutical composition.

Embodiment 159. The use of any one of embodiments 144, 152, 154, or 157, or the composition for use of any one of embodiment 144, 152, 155, or 158, wherein the cell is chosen from: immune cells; nervous system cells; muscle cells; small intestine cells; colon cells; adipocytes; kidney cells; liver cells; lung cells; splenic cells; stomach cells; esophagus cells; bladder cells; pancreas cells; thyroid cells; salivary gland cells; adrenal gland cells; pituitary gland cells; breast cells; skin cells; ovary cells; uterus cells; placenta cells; prostate cells; or testis cells, or a combination thereof.

Embodiment 160. The use of any one of embodiments 144, 152, 154, or 156, or the composition for use of any one of embodiment 144, 152, 155, or 157, wherein the cell is chosen from: renal cells, thyroid cells, parathyroid cells, cells of the inner ear or nervous system cells, or a combination thereof.

Embodiment 161. The use of any one of embodiments 144, 152, 154, or 156, or the composition for use of any one of embodiment 144, 152, 155, or 157, wherein the cell is chosen from a proximal tubular epithelial cell and/or a podocyte.

Embodiment 162. The use of embodiment 158, or the composition for use of embodiment 158, wherein contacting comprises administering the conjugate agent to the cell; a tissue comprising the cell; or an organism comprising the cell.

Embodiment 163. The use of any one of embodiments 141, 150, or 158, or the composition for use of any one of embodiments 141, 150, or 158, wherein administering the conjugate agent to the cell, tissue or organism, delivers the payload moiety to at least 5% more target cells compared to an otherwise similar cell, tissue or organism delivered an unconjugated payload moiety.

Embodiment 164. The use of any one of embodiments 141, 150, or 158, or the composition for use of any one of embodiments 141, 150, or 158, wherein administering the conjugate agent to the cell, tissue or organism, delivers the payload moiety to at least 5% more target cells compared to non-target cells.

Embodiment 165. The use of embodiment 163 or 164, or the composition for use of embodiment 163 or 164, wherein the target cell is or comprises a kidney cell.

Embodiment 166. The use of any one of embodiments 163-165, or the composition for use of any one of embodiments 163-165, wherein the target cell is or comprises a cell that has expression of a cell surface factor.

Embodiment 167. The use of any one of embodiments 163-166, or the composition for use of any one of embodiments 163-166, wherein the non-target cell is or comprises a cell that has no expression of (e.g., no detectable expression of) a cell surface factor.

Embodiment 168. The use of any one of embodiments 139, 148, 154 or 157, or the composition for use any one of embodiments 140, 149, 155 or 158, wherein administering the conjugate agent to the cell, tissue or organism, reduces expression and/or activity of the target of the payload moiety by at least 5% compared to an otherwise similar cell, tissue or organism delivered an unconjugated payload moiety.

Embodiment 169. The use of any one of embodiments 139, 148, 154 or 157, or the composition for use any one of embodiments 140, 149, 155 or 158, wherein the conjugate agent is delivered intravenously, subcutaneously, intramuscularly, parenterally or orally.

Embodiment 170. The use of any one of embodiments 139, 148, 154 or 157, or the composition for use any one of embodiments 140, 149, 155 or 158, wherein the conjugate agent is delivered in one or more doses.

Embodiment 171. The use of any one of embodiments 139, 148, 154 or 157, or the composition for use any one of embodiments 140, 149, 155 or 158, wherein the conjugate agent is delivered in combination with one or more additional conjugate agents.

Embodiment 172. The use of embodiment 171, or the composition for use of embodiment 171, wherein the one or more additional conjugate agents comprises a different payload moiety, a different linker, a different targeting moiety, or a combination thereof.

Embodiment 173. The use of any one of embodiments 139, 148, 154 or 157, or the composition for use any one of embodiments 140, 149, 155 or 158, wherein the conjugate agent is delivered in combination with one or more additional therapeutic modalities.

Embodiment 174: The conjugate agent of any one of embodiments 1-96, or the pharmaceutical composition of any one of embodiments 97-101, characterized in that when the targeting moiety comprises an aminoglycoside, the conjugated aminoglycoside has similar antimicrobial activity to an otherwise similar but unconjugated aminoglycoside.

Embodiment 175: The conjugate agent of any one of embodiments 1-96, or the pharmaceutical composition of any one of embodiments 97-101, characterized in that when the targeting moiety comprises an aminoglycoside, the conjugated aminoglycoside has lesser antimicrobial activity to an otherwise similar but unconjugated aminoglycoside.

Embodiment 176. The conjugate agent of embodiment 174 or 175, wherein antimicrobial activity is determined by evaluating a minimum inhibitory concentration (MIC) of the aminoglycoside.

Embodiment 177. The conjugate agent of embodiment 175 or 176, wherein the conjugated aminoglycoside has at least 1.5-fold, at least 2-fold, at least 5-fold, at least 10-fold, at least 20-fold or more lower MIC compared to an otherwise similar unconjugated aminoglycoside.

Embodiment 178. The conjugate agent of embodiment 176, wherein MIC is measured with gram negative bacteria.

Embodiment 179. The conjugate agent of embodiment 176, wherein MIC is measured with gram positive bacteria.

### INCORPORATION BY REFERENCE

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following example. An example is provided for illustrative purposes only. It is not to be construed as limiting the scope or content of the disclosure in any way.

### Examples

The following examples are provided so as to describe to the skilled artisan how to make and use methods and compositions described herein, and are not intended to limit the scope of the present disclosure.

### Example 1: Preparation of exemplary conjugate agents

The present Example describes preparation of certain exemplary conjugate agents useful in accordance with the present disclosure.

### Example 1.1. Tritylation of Gentamicin C1a and Gentamicin Isomer Mixture

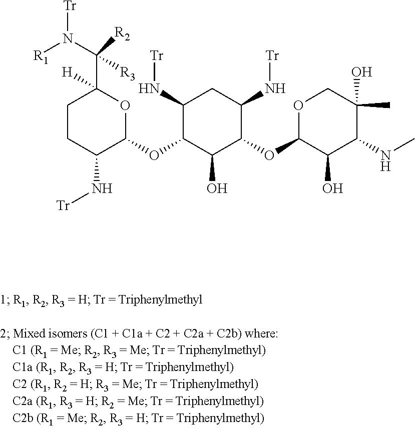

Commercially available Gentamicin C1a sulfate is dissolved in a water/methanol mixture and the resulting solution treated with a basic ion exchange resin to convert the gentamicin sulfate to gentamicin free-base form. The resin is removed by filtration and the solution is evaporated to dryness and the resulting residue is co-evaporated with dimethyl formamide (DMF) to remove residual methanol and water. To the remaining material is added chloroform, triphenylmethyl chloride (trityl chloride, 8 equ.) and triethylamine (30 equ.). The mixture is stirred for 3 days at room temperature. The reaction is concentrated to dryness and purified by silica gel or alumina chromatography to provide per-tritylated gentamicin C1a (1). Starting from gentamicin sulfate isomer mixture, per-tritylated gentamicin isomer mixture (2) is similarly prepared. When starting from the available free-base of gentamicin C1a or free-base of the isomer mixture then treatment with basic ion-exchange resin is unnecessary and the compounds can be reacted directly with trityl chloride as described above.

### Example 1.2. Alkylation of Per-Tritylated Gentamicin C1a and Per-Tritylated Gentamicin Isomer Mixture

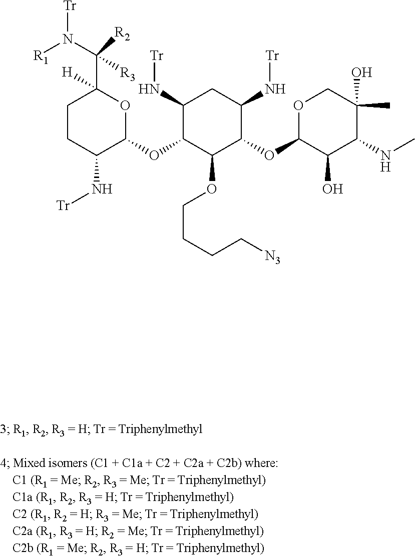

Per-tritylated gentamicin C1a (1) from Example 1.1 is co-evaporated with DMF and then dissolved in DMF under inert atmosphere. Sodium hydride (6 equ.) is then added followed by 1-bromo-4-azido-n-butane (3 equ.). The reaction is stirred for 12 hours at room temperature. Ethanol is carefully added to quench excess sodium hydride. The mixture is poured into water to form a precipitate which is isolated and purified by silica gel or alumina chromatography to give per-tritylated gentamicin C1a alkyl azide (3). Per-tritylated gentamicin isomer mixture is similarly alkylated to prepare per-tritylated gentamicin isomer mixture alkyl azide (4).

### Example 1.3. Boc-Protection of Gentamicin C1a and Gentamicin Isomer Mixture

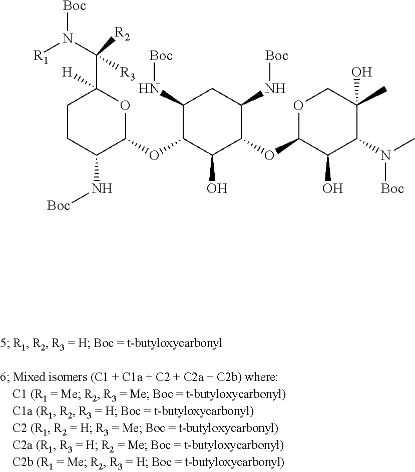

Boc protection of GM-C1a was synthesized according to the reported procedure (J. Control. Release, 324, 366-378).

The Boc protected gentamicin mixed isomers are synthesized according to the same reported procedure as for gentamicin C1a.

### Example 1.4. Succinylation of Per-Boc Gentamicin C1a

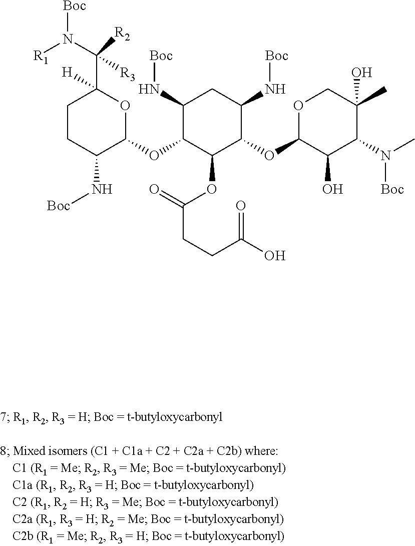

Compound 5 from Example 1.3 is dissolved in dichloromethane and pyridine. To the resulting solution is added 3 equivalents of succinic anhydride and a catalytic amount of 4-dimethylaminopyridine. The reaction is stirred for 12 hours at room temperature then concentrated to dryness. Purification by silica gel or alumina chromatography provides succinylated per-boc gentamicin C1a (7). Succinylated per-boc gentamicin isomer mixture (8) is prepared from per-boc gentamicin isomer mixture (6) in a similar fashion.

### Example 1.5. Amidation of succinylated per-boc gentamicin C1a

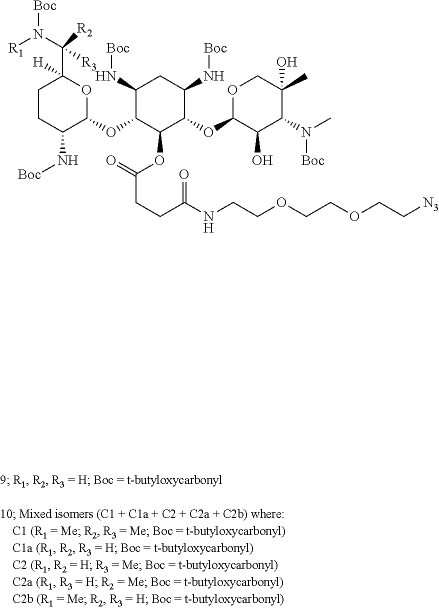

Compound 7 from Example 1.4 is dissolved in dichloromethane and are added 1-[bis(dimethylamino)methylene]-1H-1,2,3-triazolo[4,5-b]pyridinium 3-oxide hexafluorophosphate (HATU) (1 equ.) and trimethylamine (2 equ.). The reaction is stirred for 5 minutes and then is added 2-[2-(2-Azidoethoxy) ethoxy]ethanamine (1.1 equ.). The reaction is stirred at room temperature for 12 hours then concentrated to dryness and the single isomer gentamicin amide product (9) is purified and isolated by silica gel or alumina chromatography. The mixed isomer gentamicin amide product (10) is prepared in a similar fashion from (8).

### Example 1.6. Reduction of Amidated Succinylated Per-Boc Gentamicin C1a

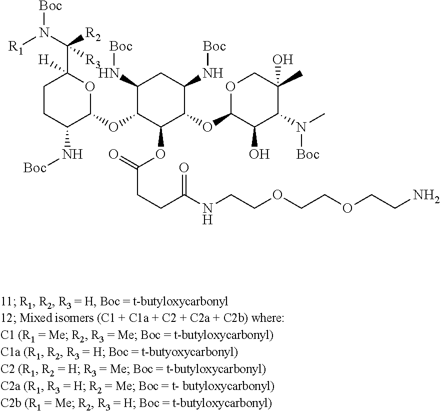

Compound 9 and 10% Pd/Carbon catalyst (unreduced form) are placed under inert atmosphere and a mixture of methanol and chloroform are added. The suspension is rapidly stirred and triethylsilane is added dropwise by syringe leading to rapid gas liberation. The mixture is then filtered through Celite and the Celite washed with methanol. The filtrate and washings are concentrated to dryness to provide the amine compound (11) as its hydrochloride salt. The mixed isomer gentamicin amine product (12) is prepared in similar fashion from (9).

### Example 1.7. Detritylation of Per-Trityl Gentamicin C1a Alkyl Azide

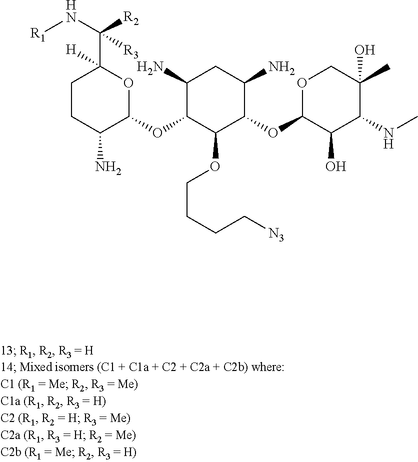

Compound 3 from Example 1.2 is dissolved in dichloromethane and the mixture is cooled in an ice bath with stirring. Trifluoroacetic acid is added and the mixture stirred until analysis by thin-layer chromatography or reversed-phase HPLC shows complete removal of trityl groups. The mixture is concentrated to dryness and the residue portioned between water and a water immiscible organic solvent such as toluene. The water layer is separated and the product purified by reversed-phase HPLC to give detritylated gentamicin C1a (13) having a pendant alkylazide moiety. The mixed isomer gentamicin amine-product (14) is prepared in similar fashion from compound 4.

### Example 1.8. Boc-Removal of Per-Boc Gentamicin C1a Alkyl Azide

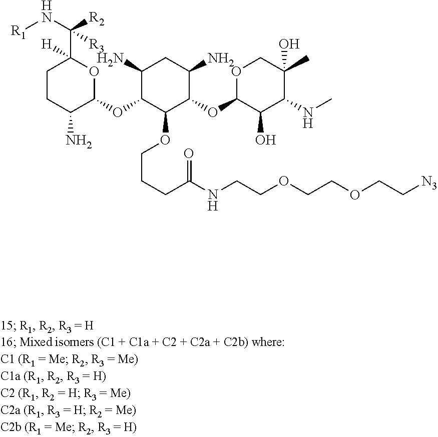

Compound 9 from Example 1.5 is dissolved in dichloromethane and the mixture is cooled in an ice bath with stirring. Trifluoroacetic acid is added and the mixture stirred until analysis by thin-layer chromatography or reversed-phase HPLC shows complete removal of Boc-groups. The mixture is concentrated to dryness and the residue portioned between water and a water immiscible organic solvent such as toluene. The water layer is separated and the product purified by reversed-phase HPLC to give de-tritylated gentamicin C1a (15) having a pendant alkyl azide moiety. The mixed isomer gentamicin amine product (16) is prepared in similar fashion from (10).

### Synthesis of Azido-Gentamicin “C”

### Example 1.9.TBS protection of per-Boc gentamicin C1a

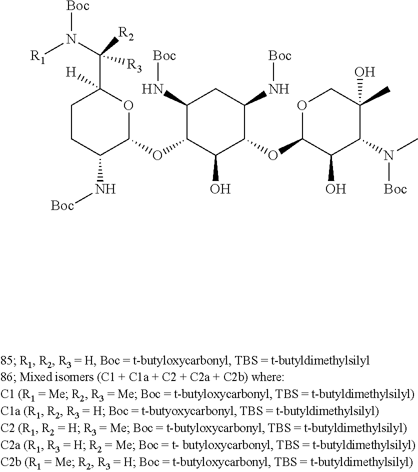

To a solution of per-Bocgentamicin C1a (prepared according to the procedure reported in J. Control. Release, 324, 366-378) (4.00 g, 4.21 mmol, 1 eq) in DMF (10 mL) from example [1.3] were added N-methyl imidazole (2.07 g, 25.26 mmol, 6 eq) and TBSCl (3.18 g, 21.05 mmol, 5 eq). The resulting solution was stirred at 20° C. for 24 h. The reaction mixture was diluted with water (20 mL) and EtOAc (20 mL), and the organic layer was washed with water (10 mL×2), brine (10 mL×2), dried over sodium sulfate, and filtered. The filtrate was concentrated to afford a yellow oil, which was purified by silica gel chromatography (DCM: MeOH, 50:1 to 15:1) to afford 85 (4.11 g, 3.86 mmol, 91% yield, 99% purity) as a white solid. ESI-MS: m/z calcd. for C₅₀H₉₂N₅O₁₇Si⁻ ([M−H]⁻), 1062.6; found 1062.4 [M−H]⁻.

The TBS protected gentamicin mixed isomers (86) are synthesized according to the same reported procedure as for gentamicin C1a.

### Example 1.10 Alkylation of Per-Boc Gentamicin C1a

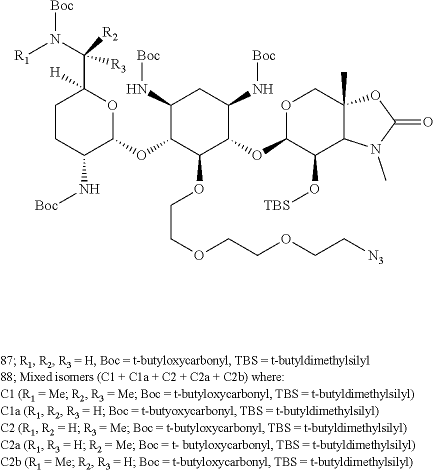

To a solution of 85 (10 g, 9.40 mmol, 1 eq) from Example 1.9 and 1-azido-2-[2-(2-bromoethoxy) ethoxy]ethane (4.47 g, 18.79 mmol, 2 eq) in THF (100 mL) was added tert-BuOK (1 M, 18.79 mL, 2 eq). The resulting solution was stirred at 20° C. for 2 h. The reaction mixture was quenched with NH4Cl (sat. aq., 100 mL), and diluted with EtOAc (50 mL). The organic layer was washed with water (100 mL×2), brine (100 mL×2), dried over sodium sulfate, and filtered. The filtrate was concentrated to afford a yellow oil, which was purified by silica gel chromatography (DCM: MeOH, 100:1 to 20:1) to afford 87 (10.3 g, 8.85 mmol, 94% yield, 98% purity) as a white solid. ESI-MS: m/z calcd. for C52H₉₃N₈O₁₈Si⁻([M−H]⁻), 1145.6; found 1145.6 [M−H]⁻.

The alkylated gentamicin mixed isomers (88) are synthesized according to the same reported procedure as for gentamicin C1a.

### Example 1.11 TBS Deprotection of Per-Boc Gentamicin C1a

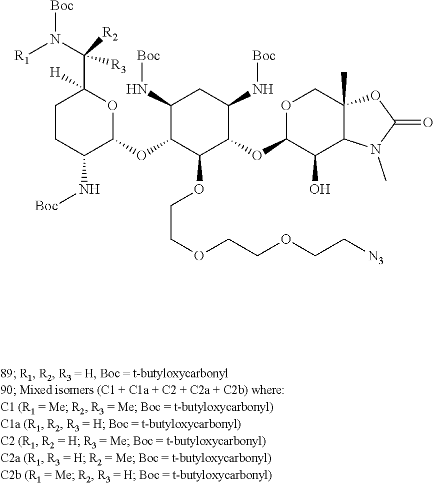

To a solution of 87 (10.3 g, 8.98 mmol, 1 eq) from Example 1.10 in THF (100 mL) was added TBAF (1 M, 11.67 mL, 1.3 eq). The resulting solution was stirred at 20° C. for 24 h. The reaction mixture was diluted with EtOAc (50 mL), and the organic layer was washed with water (50 mL×2), brine (50 mL×2), dried over sodium sulfate, and filtered. The filtrate was concentrated to afford a yellow oil, which was purified by silica gel chromatography (DCM: MeOH, 100:1 to 30:1) to afford 89 (8.5 g, 7.80 mmol, 87% yield, 95% purity) as a white solid. ESI-MS: m/z calcd. for C46H₇₉N₈O₁₈⁻ ([M−H]⁻), 1031.6; found 1031.5 [M−H]⁻.

The TBS deprotected gentamicin mixed isomers (90) are synthesized according to the same reported procedure as for gentamicin C1a.

### Example 1.12 Boc-Removal of Per-Boc Gentamicin C1a Oxazolidinone PEG2 Azide

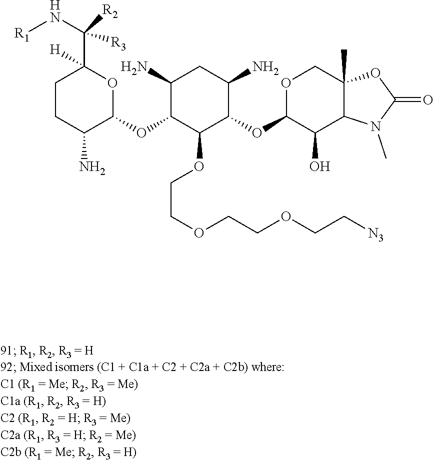

A solution of 89 (4.3 g, 4.17 mmol, 1 eq) in HCl (4 M in dioxane (43 mL) was stirred at 20° C. for 1 h. The reaction mixture was concentrated to afford a yellow solid, which was reconstituted in water (40 mL) and treated with Amberlite IRA400 HO form to adjust pH to 9-10. The resin was filtered and the filtrate was lyophilized to afford a yellow solid as a crude product, which was further purified by prep-HPLC(column: Boston Prime C18 150*30 mm*5 um; mobile phase A: water; mobile phase B ACN/THF (2/1); gradient: 0%-55% B over 9 min) to afford azido-gentamicinC(91) (1.26 g, 1.99 mmol, 48% yield, 96% purity) as a yellow solid. ESI-MS: m/z calcd. for C26H₄₉N₈O₁₀⁺ ([M+H]⁺), 633.4; found 633.4 [M+H]⁺. ¹H NMR (400 MHZ, D20) 8 ppm 5.62 (d, J=3.0 Hz, 1H), 4.96 (d, J=3.5 Hz, 1H), 4.37 (t, J=3.8 Hz, 1H), 4.20-4.09 (m, 2H), 4.01 (d, J=12.8 Hz, 1H), 3.87-3.81 (m, 1H), 3.81-3.73 (m, 4H), 3.70-3.64 (m, 8H), 3.51-3.43 (m, 3H), 3.39-3.30 (m, 1H), 3.28-3.20 (m, 1H), 3.17 (dd, J=3.0, 13.6 Hz, 1H), 3.02 (dd, J=8.3, 13.5 Hz, 1H), 2.84 (s, 3H), 2.33-2.22 (m, 1H), 2.03-1.92 (m, 2H), 1.85 (s, 10H), 1.72-1.59 (m, 1H), 1.59-1.50 (m, 1H), 1.36 (s, 3H).

The Boc deprotected gentamicin mixed isomers (92) are synthesized according to the same reported procedure as for gentamicin C1a.

### Synthesis of Azido-Gentamicin “A”

### Example 1.13 Oxazolidinone-Removal of Per-Boc Gentamicin C1a PEG2 Azide

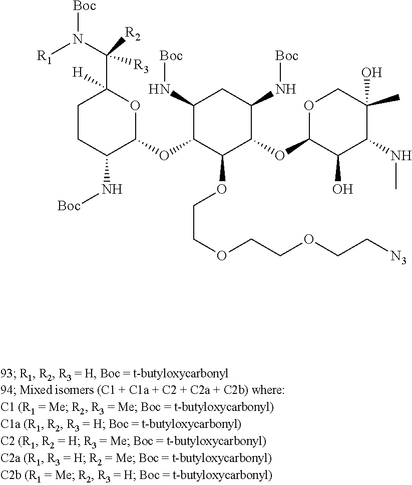

To a solution of 89 (9 g, 8.71 mmol, 1 eq) in 10% KOH aq. (45 mL) and EtOH (45 mL). The resulting solution was stirred at 20° C. for 24 h. TLC(DCM/MeOH, 10/1) showed a major spot with slightly lower polarity. The reaction mixture was diluted with water (100 mL) and EtOAc (100 mL), and the organic layer was washed with water (200 mL×2), brine (200 mL×2), dried over sodium sulfate, and filtered. The filtrate was concentrated to afford a yellow oil, which was purified by silica gel chromatography (DCM: MeOH, 100:1 to 30:1) to afford 93 (5.3 g, 5.04 mmol, 57.85% yield, 95.77% purity) as a white solid. ESI-MS: m/z calcd. for C45H₈₁N₈O₁₇⁻([M−H]⁻), 1005.6; found 1005.5 [M−H]⁻.

The oxazolidinone removed gentamicin mixed isomers (94) are synthesized according to the same reported procedure as for gentamicin C1a.

### Example 1.14 Boc-Removal of Per-Boc Gentamicin C1a PEG2 Azide

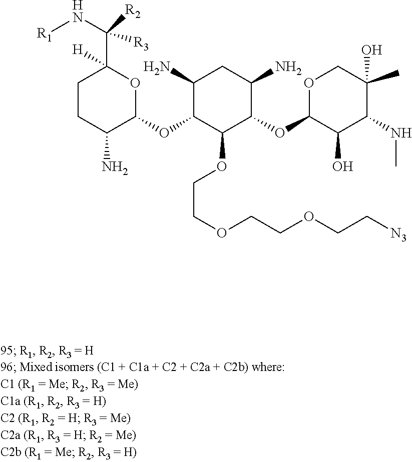

A solution of 93 (5.3 g, 5.26 mmol, 1 eq) in 4 M HCl/dioxane (53 mL) was stirred at 20° C. for 1 h. LCMS showed the starting material was consumed completely. The reaction mixture was concentrated to afford a yellow solid, which was reconstituted in water (40 mL) and treated with Amberlite IRA400 HO form to adjust pH to 9-10. The resin was filtered and the filtrate was lyophilized to afford a yellow solid as a crude product, which was further purified by prep-HPLC(column: Boston Prime C18 150*30 mm*5 um; mobile phase A: water; mobile phase B: ACN/THF (2/1); gradient: 0%-30% B over 9 min) to afford azido-gentamicin A (95) (1.5 g, 2.48 mmol, 46.9% yield, 99.48% purity) as a white solid. ESI-MS: m/z calcd. for C₂₆H₅₁N₈O₉⁺ ([M+H]⁺), 607.4; found 607.3 [M+H]⁺.

The Boc deprotected gentamicin mixed isomers (96) are synthesized according to the same reported procedure as for gentamicin C1a.

### Synthesis of Azido-Aminoglycoside “B”

Azido-gentamicin “B” was synthesized following the procedures for azido-aminoglycosides “C” and “A” utilizing 1-azido-4-bromobutane in place of 1-azido-2-[2-(2-bromoethoxy) ethoxy]ethane.

TABLE 7
Azido-aminoglycosides

| Azido- | | |
|---|---|---|
| Aminoglycoside | | |
| name | structure | aminoglycoside ID |
| Gentamicin 5-O-ether | 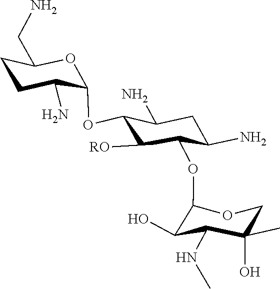 | A, B |
| | 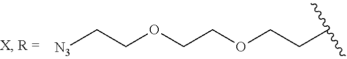 [SMILES: *CCOCCOCCN=[N+]=[N-]] | |
| | 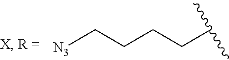 [SMILES: *CCCCN=[N+]=[N-]] | |
| Gentamicin 5-O-ether 3″,4″-oxazolidinone | 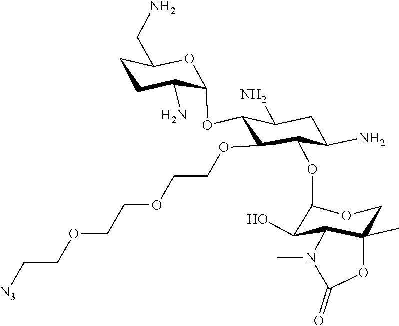 | C |

### Example 1.15. General Procedure for Synthesis of 5′-Alkynyl-PMO's

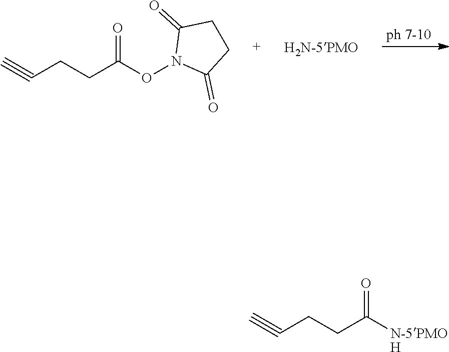

Certain 5′-alkynyl-PMOs are available and purchased from Gene Tools. Alternatively, phosphomorpholino oligos (PMOs) bearing a 5′-terminal primary amine group can be purchased from Gene Tools (Philomath, OR) and modified according to the following procedure. The 5′-amine group of each PMO is reacted with the NHS or nitrophenyl ester of 1,2-alkynylpentanoic acid by combining the PMO with the ester in an aqueous solution which contains a water miscible co-solvent such as acetonitrile, DMF or DMSO, and a buffer such as sodium bicarbonate or organic base such as N-methylmorpholine so as to maintain a pH of approximately 7-10.

It will be appreciated that other types of nucleic acids, e.g., siRNAs, can be functionalized in a similar manner.

TABLE 8
5′-pentynamide PMOs

| 5′-Amine | PMO Sequence | 5′-pentyn- | Compound |
|---|---|---|---|
| PMO | 5′-3′ | amide PMO | No. |
| H2N-muHPRT | TCGAGGTCTTACT | pentynamide- | 76 |
| | AACCTGTCCATA | muHPRT | |
| H2N-huHPRT | AAATGTCATACAT | pentynamide- | 77 |
| | ACCTTGCGACCT | huHPRT | |
| H2N- | CCCTTCACCTCCA | pentynamide- | 78 |
| muExemplary | TACTCACTGCAC | muExemplary | |
| Target 1 | | Target 1 | |

Following the reaction the PMO compounds 76-78 bearing a 5′-pentynamide group are purified and isolated by reversed-phase HPLC or by cation exchange chromatography. Identity of each compound is confirmed by mass spectrometry and UV absorbance is used to determine the amount of obtained material.

### Example 1.16. Preparation of Gentamicin C1a and Gentamicin Mixed Isomer PMO Conjugates

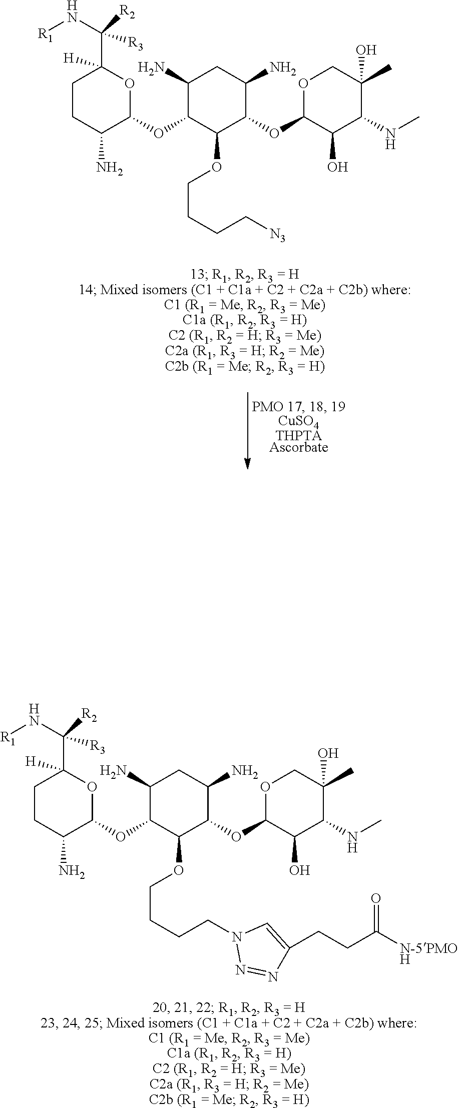

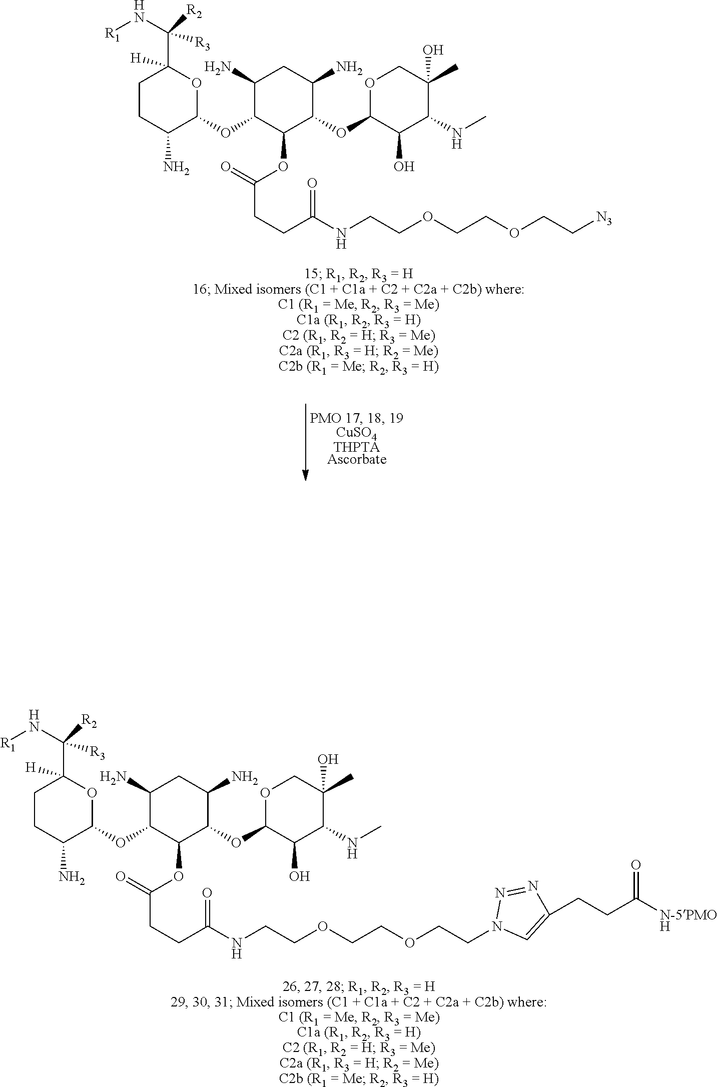

General Click Chemistry Protocol: Amino glycoside and PMO conjugates were custom synthesized based on similar literature protocol from Fahmi H. et. al. J. Am. Chem. Soc. 2005, 127, 210-216, (https://doi.org/10.1021/ja0471525). Briefly, Azido-amino glycoside (3 eqv.) and 5′-Alkyne-PMO (1 eqv.) compounds 17, 18, or 19 (purchased from Gene Tools, Philomath, OR) were first dissolved in sodium phosphate buffer, followed by CuSO₄ (2 eqv.) and ascorbic acid (5 eqv.) solutions in water. After mixing for 2-3 hours, the product was purified and isolated by a buffer exchange using 20 mM ammonium acetate or using HPLC as described by Belanger A. M. et. al. JCI Insight. 2108: 3 (14): e121762 (https://doi.org/10.1172/jci.insight.121762.). Products were lyophilized and characterized by ESI-MS.

TABLE 9
5′-alkyne PMOs

| 5′-alkyne | | Compound |
|---|---|---|
| PMO | PMO Sequence 5′-3′ | No. |
| alkyne- | TCGAGGTCTTACTAACCT | 17 |
| muHPRT | GTCCATA | |
| alkyne- | AAATGTCATACATACCTT | 18 |
| huHPRT | GCGACCT | |
| alkyne- | CCCTTCACCTCCATACTC | 19 |
| muExemplary | ACTGCAC | |
| Target 1 | | |

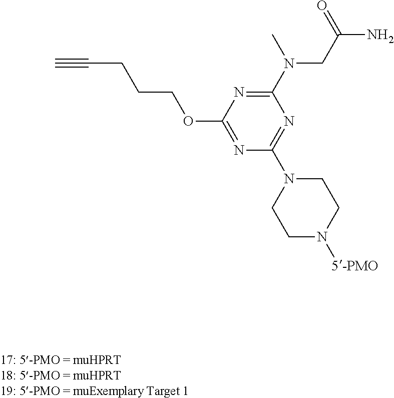

In this way azide-compound B is conjugated to each 5′-alkyne-PMO compound 18 and 19.

TABLE 10
Azide compounds

| | Azide | | PMO | Conjugate | MW | MS | |
|---|---|---|---|---|---|---|---|
| Azide | Description | PMO | Description | Description | (calc) | analysis | purity |
| B | Gentamicin | 18 | Alkyne- | gentamine | 9178.73 | 1312.00 | >99% |
| | 5-O- | | huHPRT | 5-O-ether | | (M + 7H)⁷⁺ | |
| | butylazide | | | PEG2-azide | | | |
| | | | | (AG005) | | | |
| | | | | Click - | | | |
| | | | | muExemplary | | | |
| | | | | Target 1 | | | |
| | | | | siRNA | | | |
| | | 19 | Alkyne- | gentamicin | 9011.59 | 1002.18 | 99% |
| | | | muExemplary | 6′-amide | | (M + 9H)⁹⁺ | |
| | | | Target 1 | amidopentyl- | | | |
| | | | | azide | | | |
| | | | | (AG006) | | | |
| | | | | Click - | | | |
| | | | | muExemplary | | | |
| | | | | Target 1 | | | |
| | | | | siRNA | | | |

### Example 1.17. General Procedure for Synthesis of 5′-Formylbenzoyl-PMOs

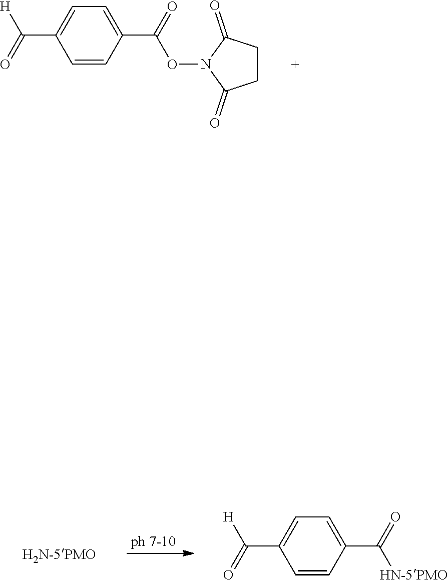

General HyNic Protocol: Peptide PMO conjugates were custom synthesized at CellMosaic Inc. (Woburn, MA) based on similar literature protocol from Belanger A. M. et. al. JCI Insight. 2108: 3 (14): e121762 (https://doi.org/10.1172/jci.insight.121762.). Briefly, 5′-Amino PMOs were functionalized with formyl group using a slightly modified protocol as described by Belanger A. M. Succinimidyl p-formylbenzoate was used instead of sulfo —N-succinimidyl-4-formylbenzoate. 5′-formyl PMOs were purified by HPLC.

TABLE 11
5′-Formyl PMOs

| 5′-Amine | | 5′-Formyl- | Compound |
|---|---|---|---|
| PMO | PMO Sequence 5′-3′ | PMO | No. |
| H₂N-muHPRT | TCGAGGTCTTACTAACCT | Formyl- | 32 |
| | GTCCATA | muHPRT | |
| H₂N-huHPRT | AAATGTCATACATACCTT | Formyl- | 33 |
| | GCGACCT | huHPRT | |
| H₂N- | CCCTTCACCTCCATACTC | Formyl- | 34 |
| muExemplary | ACTGCAC | muExemplary | |
| Target 1 | | Target 1 | |

UV absorbance was used to determine the amount of obtained material.

### Example 1.18. General Procedure for Preparation of Hydrazide-Peptide-PMO Conjugates

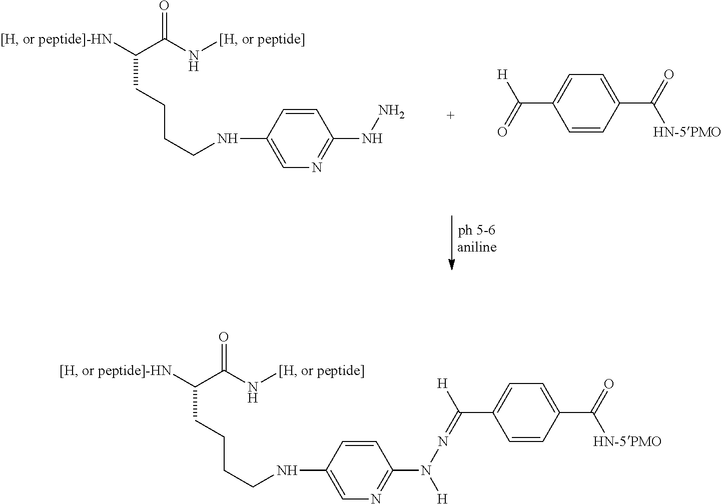

Peptides bearing a hydrazinonicotinic amide (HyNic) coupled to the side chain of a lysine residue were purchased from Vivitide, Gardner MA.

TABLE 12
HyNic peptides

| HyNic- | N. | | C- |
|---|---|---|---|
| Peptide | terminus | Sequence | terminus |
| KKEEE | H₂N— | K(Hynic)KKEEEKKEEEKKEEE | —OH |
| G3C12 | H₂N— | K(Hynic)ANTPCGPYTHDCPVKR | —OH |
| PeptideJ | Ac—NH— | rXrrXrrXrrXrXBK(HyNic) | —NH₂ |

“r” indicates D-arginine;
“X” indicates 6-amino-hexanoic acid;
“B” indicates β-alanine; and
“K(HyNic)” indicates L-lysine-e-HyNic

5′-formyl-PMOs were conjugated to peptides by reaction with 6-hydrazino-nicotinamide (HyNic)-peptides under mildly acidic conditions for 3-4 h and then purified by HPLC as described by Belanger A. M. Products were lyophilized and characterized by ESI-MS.

TABLE 13
Formyl PMOs

| | Formyl- | | | | | | |
|---|---|---|---|---|---|---|---|
| Formyl- | PMO | Hynic- | | Conjugate | MW | MS | |
| PMO | Description | Peptide | Conjugate | Description | (calc) | analysis | purity |
| 32 | | KKEEE | 35 | KKEEE-Hy- | 11028.46 | 11028.1 | >99% |
| | | | | muHPRT | | (M + H) | |
| | Formyl- | G3C12 | 36 | G3C12-Hy- | 10836.57 | 10835.7 | >99% |
| | muHPRT | | | muHPRT | | (M + H) | |
| | | PeptideJ | 37 | PeptideJ-Hy- | | | |
| | | | | muHPRT | | | |
| 33 | Formyl- | KKEE | 38 | KKEE-Hy- | 11021.48 | 11021.3 | >99% |
| | huHPRT | | | huHPRT | | (M + H) | |
| | | G3C12 | 39 | G3C12-Hy- | | | |
| | | | | huHPRT | | | |
| | | PeptideJ | 40 | PeptideJ-Hy- | | | |
| | | | | huHPRT | | | |
| 34 | Formyl- | KKEE | 41 | KKEE-Hy- | 10854.44 | 10854.3 | >99% |
| | muExemplary | | | muExemplary | | (M + H) | |
| | Target 1 | | | Target 1 | | | |
| | | G3C12 | 42 | G3C12-Hy- | 10662.45 | 10662.7 | |
| | | | | muExemplary | | (M + H) | |
| | | | | Target 1 | | | |
| | | PeptideJ | 43 | PeptideJ-Hy- | | | |
| | | | | muExemplary | | | |
| | | | | Target 1 | | | |

### Example 1.19. General Procedure for Preparation of Maleimide-PMOs (MM-PMOs)

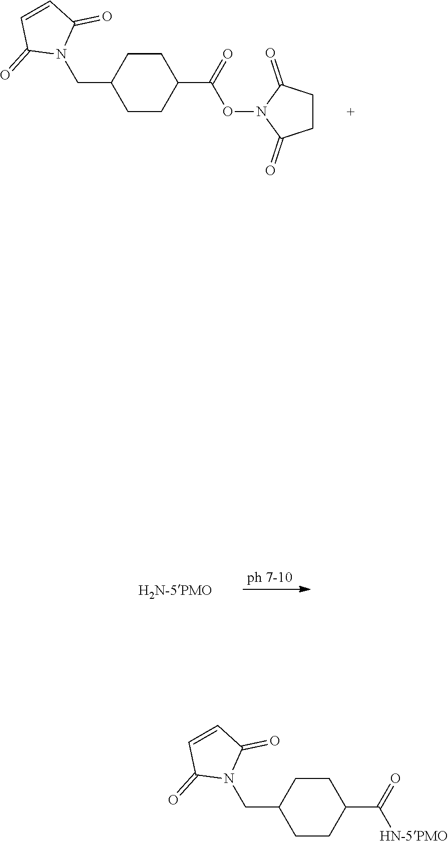

Phosphomorpholino oligos (PMOs) bearing a 5′-terminal primary amine group were purchased from Gene Tools (Philomath, OR). The 5′-amine group of each PMO is reacted with succinimidyl 4-(N-maleimidomethyl)cyclohexane-1-carboxylate SMCC) or its analogue sulfosuccinimidyl 4-(N-maleimidomethyl)cyclohexane-1-carboxylate (Sulfo-SMCC) by combining the PMO with the SMCC or sulfo-SMCC in an aqueous solution which contains a water miscible co-solvent such as acetonitrile, DMF or DMSO, and a buffer such as sodium bicarbonate or organic base such as N-methylmorpholine so as to maintain a pH of approximately 7-10.

TABLE 14
5′-MM PMOs

| 5′-Amine | | | Compound |
|---|---|---|---|
| PMO | PMO Sequence 5′-3′ | 5′-MM-PMO | No. |
| H₂N-muHPRT | TCGAGGTCTTACTAACCT | MM-muHPRT | 44 |
| | GTCCATA | | |
| H₂N-huHPRT | AAATGTCATACATACCTT | MM-huHPRT | 45 |
| | GCGACCT | | |
| H₂N- | CCCTTCACCTCCATACTC | MM- | 46 |
| muExemplary | ACTGCAC | muExemplary | |
| Target 1 | | Target 1 | |

Following the reaction compounds 44-46 bearing a 5′-maleimide group (MM) are purified and isolated by desalting, or reversed-phase HPLC or by cation exchange chromatography. Identity of each compound is confirmed by mass spectrometry and UV absorbance is used to determine the amount of obtained material.

### Example 1.20. General Procedure for Preparation of Maleimide-PMO-Peptide Conjugates

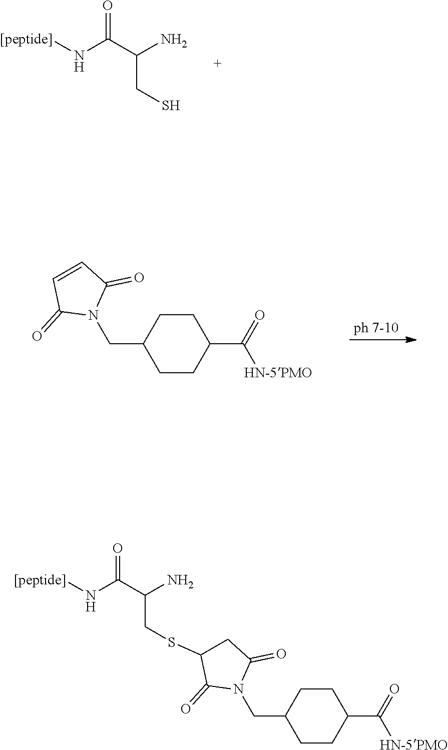

Peptides bearing an N-terminal cysteine residue having a free thiol side chain were purchased from Vivitide, Gardner MA.

TABLE 15
Cys-Peptide conjugates

| Cys- | N- | | C- |
|---|---|---|---|
| Peptide | terminus | Sequence | terminus |
| CKKEE | H₂N— | CKKEEEKKEEEKKEEE | —OH |
| CG3C12 | H₂N— | CANTPCGPYTHDCPVKR | —OH |

Each peptide in aqueous solution was combined with each of 5′-MM-PMOs 44-46 and buffer such as sodium bicarbonate or an organic base such as N-methylmorpholine so as to maintain a pH of approximately 7-9. After conjugation, conjugates were purified if desired, or buffer exchanged into storage buffer. Size exclusion chromatography, ion exchange chromatography, hydrophobic interaction chromatography, and other methods such as RP-HPLC are used to purify conjugates prepared by the maleimide/thiol conjugation procedure.

TABLE 16
MM PMOs

| MM- | MM-PMO | Cys- | | Conjugate | MW | MS | |
|---|---|---|---|---|---|---|---|
| PMO | Description | Peptide | Conjugate | Description | (calc) | analysis | Purity |
| 44 | MM- | CKKEE | 47 | CKKEE-MM- | 10972.49 | 10973.2 | 86% |
| | muHPRT | | | muHPRT | | (M + H) | |
| | | CG3C12 | 48 | CG3C12-MM- | 10782.39 | 10781.0 | 74% |
| | | | | muHPRT | | (M + H) | |
| 45 | MM- | CKKEE | 49 | CKKEE-MM- | 10965.51 | 10966.2 | 86% |
| | huHPRT | | | huHPRT | | (M + H) | |
| | | CG3C12 | 50 | CG3C12-MM- | 10775.41 | 10774.0 | 73% |
| | | | | huHPRT | | (M + H) | |
| 46 | MM- | CKKEE | 51 | CKKEE-MM- | 10798.37 | 10799.2 | 91% |
| | muExemplary | | | muExemplary | | (M + H) | |
| | Target 1 | | | Target 1 | | | |
| | | CG3C12 | 52 | CG3C12-MM- | | | |
| | | | | muExemplary | | | |
| | | | | Target 1 | | | |

### Example 1.21. General Procedure for Preparation of Activated Disulfide-PMOs (SS-PMOs)

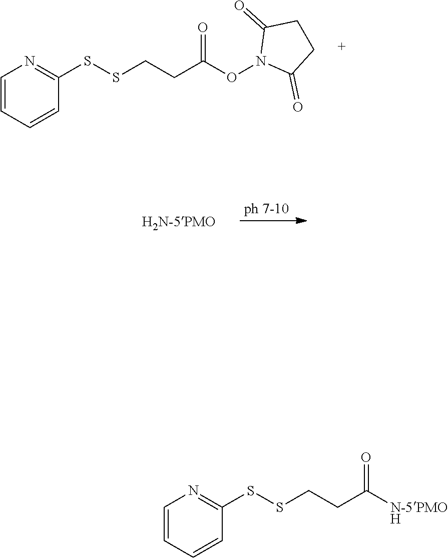

Phosphomorpholino oligos (PMOs) bearing a 5′-terminal primary amine group were purchased from Gene Tools (Philomath, OR). The 5′-amine group of each PMO is reacted with succinimidyl 3-(2-pyridyldithio) propionate (SPDP) or its analogue sulfo-succinimidyl 3-(2-pyridyldithio) propionate (Sulfo-SPDP) by combining the PMO with the SPDP or sulfo-SPDP in an aqueous solution which contains a water miscible co-solvent such as acetonitrile, DMF or DMSO, and a buffer such as sodium bicarbonate or organic base such as N-methylmorpholine so as to maintain a pH of approximately 7-10.

TABLE 17
5′-SS PMOs.

| 5′-Amine | | | Compound |
|---|---|---|---|
| PMO | PMO Sequence 5′-3′ | 5′-SS-PMO | No. |
| H₂N-muHPRT | TCGAGGTCTTACTAACCT | SS-muHPRT | 53 |
| | GTCCATA | | |
| H₂N-huHPRT | AAATGTCATACATACCTT | SS-huHPRT | 54 |
| | GCGACCT | | |
| H₂N- | CCCTTCACCTCCATACTC | SS- | 55 |
| muExemplary | ACTGCAC | muExemplary | |
| Target 1 | | Target 1 | |

Following the reaction compounds 53-55 bearing a 5′-pyridyldithio disulfide (SS) are purified and isolated by desalting, or reversed-phase HPLC or by cation exchange chromatography. Identity of each compound is confirmed by mass spectrometry and UV absorbance is used to determine the amount of obtained material.

### Example 1.22. General Procedure for Preparation of Peptide-Disulfide-PMO Conjugates

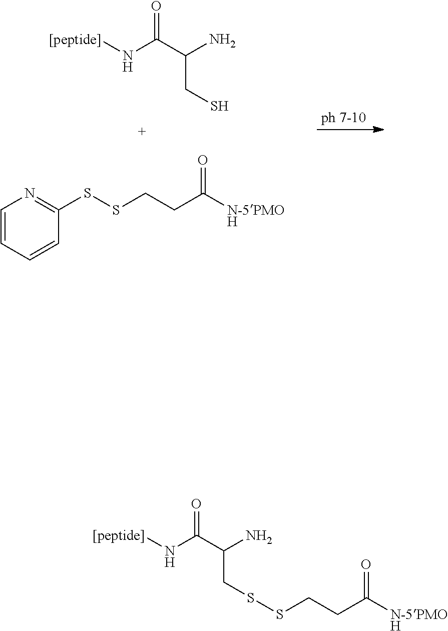

Peptides bearing an N-terminal cysteine residue having a free thiol side chain were purchased from Vivitide, Gardner MA.

TABLE 18
Cys-peptide conjugates

| Cys- | N. | | C- |
|---|---|---|---|
| Peptide | terminus | Sequence | terminus |
| CKKEE | H₂N— | CKKEEEKKEEEKKEEE | —OH |
| CG3C12 | H₂N— | CANTPCGPYTHDCPVKR | —OH |

Each cys-peptide in aqueous solution is combined with each of 5′-SS-PMOs 53-55 and a buffer such as sodium bicarbonate or and organic base such as N-methylmorpholine so as to maintain a pH of approximately 7-9. After conjugation, conjugates are purified if desired, or buffer exchanged into storage buffer. Size exclusion chromatography, ion exchange chromatography, hydrophobic interaction chromatography, and other methods such as RP-HPLC are used to purify conjugates prepared by the maleimide/thiol conjugation procedure.

TABLE 19
SS-PMO conjugates

| | SS-PMO | Cys- | | |
|---|---|---|---|---|
| SS-PMO | Description | Peptide | Conjugate | Conjugate Description |
| 53 | MM-muHPRT | CKKEE | 56 | CKKEE-SS-muHPRT |
| | | CG3C12 | 57 | CG3C12-SS-muHPRT |
| 54 | SS-huHPRT | CKKEE | 58 | CKKEE-SS-huHPRT |
| | | CG3C12 | 59 | CG3C12-SS-huHPRT |
| 55 | SS- | CKKEE | 60 | CKKEE-SS- |
| | muExemplary | | | muExemplary Target 1 |
| | Target 1 | CG3C12 | 61 | CG3C12-SS- |
| | | | | muExemplary Target I |

### Example 1.23. Preparation of siRNA Gentamicin Conjugates

A 5′-amino-modified duplex siRNA targeted to the HPRT gene mRNA (siHPRT) was purchased from IDT, Coralville IA. The passenger strand was conjugated to the gentamicin targeting moiety according to the methods described above and annealed to the guide strand. In this example, a 5′-aminehexamethylene linker was attached to the S′ end of the passenger strand. The guide and passenger strands have the compositions and sequences shown in the table below:

TABLE 20
siRNA sequences

| SiRNA | | Strand | |
|---|---|---|---|
| No | Target Gene | Type | Sequence (IDT Nomenclature) |
| S01 | muExemplary | Passenger | mU*mU*mUmUmCmA/i2FC/mA/i2FC//i2FC// |
| | Target 1 | | i2FC/mAmAmUmAmUmCmAmC*mA*mA |
| | (mouse/rat | Guide | mU*/i2FU/*mGmUmG/i2FA/mUmAmUmUmGmGm |
| | cross-reactive) | | G/i2FU/mG/i2FU/mGmAmAmAmA*mG*mG |
| S02 | HPRT | Passenger | mU*mC*mCmUmAmU/i2FG/mA/i2FC//i2FU// |
| | (mouse/human | | i2FG/mUmAmGmAmUmUmUmU*mA*mA |
| | cross-reactive) | Guide | mU*/i2FU/*mAmAmA/i2FA/mUmCmUmAmCmA |
| | | | mG/i2FU/mC/i2FA/mUmAmGmGmA*mA*mU |

Abbreviations:
mU is 2′-methoxy uridine;
mC is 2′-methoxy cystidine;
mA is 2′-methoxy adenosine;
mG is 2′-methoxy guanosine,
/i2FG/ is 2′-fluoro guanosine,
/i2FC/ is 2′-fluoro cytosine;
/i2FU/ is 2′-fluoro uridine;
/i2FA/ is 2′-fluoro adenine;
“*” is a phosphorothioate internucleotide linkage

Using the methods above, gentamicin-siRNA conjugates shown in Table 20 were prepared with copper “Click” chemistry.

To prepare 5′-alkynyl modified passenger strand siRNA, a 5′-hexyl-amine-modified passenger strand siRNA was treated with 4-pentynoic acid N-hydroxysuccinimide ester (4.0 eq.) and N,N-Diisopropylethylamine (10.0 eq.) in acetonitrile (0.5 mL) was added a solution of oligo-amine (1 umol) in water (0.5 mL) at room temperature. The resulting solution was shaken at room temperature for 3 h. Upon completion, the resulting solution was lyophilized to give a crude product as off-white solid, which was further purified by prep-HPLC.

### General Procedure for CuAAC Conjugation

The 5′-alkyne modified oligo (purified product) was used for copper-catalyzed alkyne azide cyclization (CuAAC) with azide using conditions outlined in Table 21.

TABLE 21
CuAAC reaction conditions (reactant equivalent)

| Reagents | Equivalent | |
|---|---|---|
| CuSO₄ | 1.5 | eq. |
| THPTA | 3.0 | eq. |
| Sodium Ascorbate | 18.0 | eq. |
| GM C1a (oxazolidone analog) | 3.0 | eq. |
| siliaMetS ® TAAcONa resin | 15.0 | eq. |

### Cu-THPTA Solution Preparation

A solution of CuSO₄ (aq., 25 mM) and 50 mM tris (3-hydroxypropyltrizaolylmethyl)amine (THPTA) aq. were mixed (1:1, v/v, 1:2 molar ratio) and allowed to stand at room temperature for 1 h, which was used for CuAAC conjugation.

CuAAC conjugation

To a solution of 1 μmol 5′-alkyne modified oligo in 0.62 mL milli Q water and 0.1 mL tert-BuOH(10% of final volume) in a 4 mL centrifugal tube were added a solution of GM C1a (oxazolidone analog) in water (75 mM, 0.04 mL), pre-formed Cu-THPTA solution (0.12 mL), followed by sodium ascorbate aqueous solution (150 mM, 0.12 mL). The resulting mixture was shaken at room temperature for 3 h. siliaMetS® TAAcONa resin (510 μmol/g, 30 mg) was added, and the resulting mixture was further shaken at room temperature for 1 h. The mixture was filtrated through a 0.45 μm filter, and the filtrate was lyophilized to afford crude product as a yellow solid, which was reconstituted in water for prep-HPLC purification.

### 1. Annealing to Form siRNA Duplex

Duplex siRNA was obtained by mixing equivalent amount of sense strand and antisense strand in aqueous solution. The formation of siRNA duplex was confirmed by size-exclusion HPLC(SEC-HPLC), using the conditions outlined in Table 22.

TABLE 22
Sec-HPLC conditions

| | |
|---|---|
| Column | Agilent Advance BIOS SEC 4.6*300 mm |
| Mobile phase | 200 mg Tween 80 and 40 mL 10x PBS buffer |
| | (pH 7.4) diluted to 200 mL with Milli Q water |
| Flow rate | 0.2 mL/min |
| Column | 25° C. |
| temperature | |
| Gradient | Isocratic, 25 min |

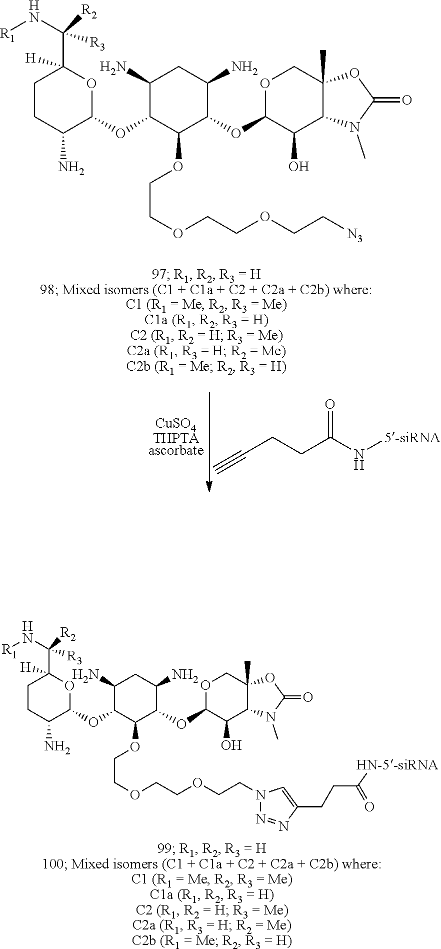

TABLE 23
siRNA conjugates.

| | siRNA | | | |
|---|---|---|---|---|
| siRNA | description | AG | Conjugate | Conjugate Description |
| S01 | muExemplary | C | 81 | Gentamicin-5-O-ether (3″,4″- |
| | Target 1 | | | oxazolidinone) PEG2-azide (AG004) |
| | siRNA | | | Click - muExemplary Target 1 siRNA |
| | | A | 82 | gentamicin 5-O-ether PEG2-azide |
| | | | | (AG003) Click - muExemplary Target |
| | | | | 1 siRNA |
| S02 | HPRT | C | 83 | Gentamicin-5-O-ether (3″,4″- |
| | siRNA | | | oxazolidinone) PEG2-azide (AG004) |
| | | | | Click - HPRT siRNA |
| | | A | 84 | gentamicin 5-O-ether PEG2-azide |
| | | | | (AG003) Click - HPRT siRNA |

TABLE 24
Analytical data for siRNA-gentamicin conjugates:

| | MW SS | MW AS | MS | MS | Purity |
|---|---|---|---|---|---|
| Conjugate | (calc) | (calc) | analysis SS | analysis AS | (duplex) |
| 81 | 7745.9 | 7808.2 | 7747.8 | 7808.8 | 94% |
| | | | (M + H) | (M + H) | |
| 82 | 7720.6 | 7808.2 | 7721.5 | 7808.7 | 94% |
| | | | (M + H) | (M + H) | |
| 83 | 7822.5 | 7663.1 | 7822.2 | 7663.1 | 94% |
| | | | (M + H) | (M + H) | |
| 84 | 7796.5 | 7663.1 | 7796.4 | 7663.1 | 96% |
| | | | (M + H) | (M + H) | |

### Example 1.24. Preparation of siRNA Hydrazide Peptide Conjugates

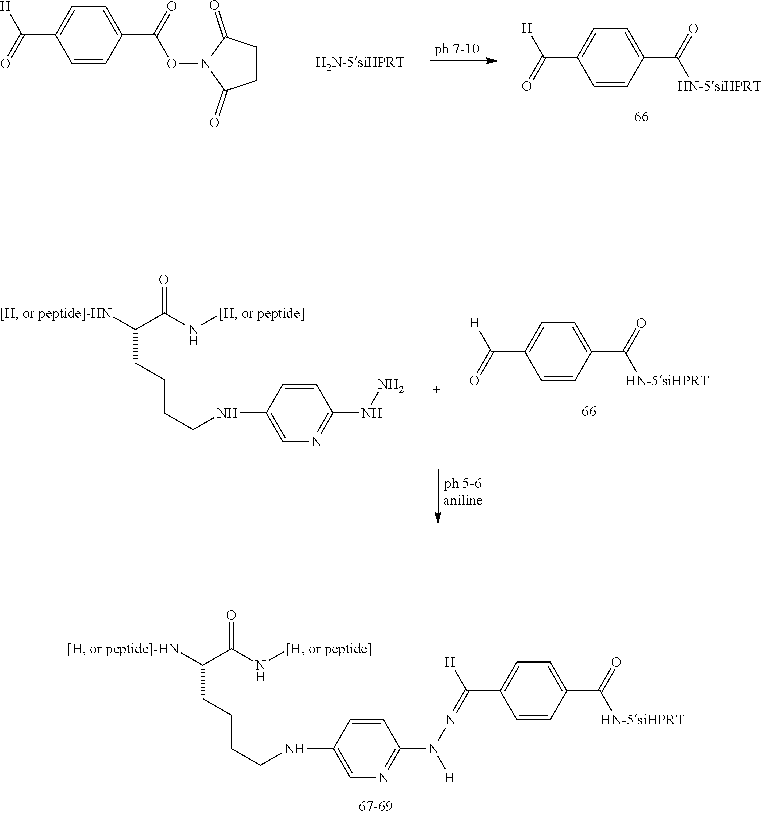

Using the methods in Examples 1.11 and 1.12 and the peptides from Example 1.12 were prepared formyl-siHPRT 66 and formyl-si muExemplary Target 1 and then peptide-siRNA conjugates 67-69 by hydrazide chemistry and QC data for hydrazide peptide-siRNA conjugates:

TABLE 25
Formly siRNAs

| | | | | MW | MW | MS | MS | |
|---|---|---|---|---|---|---|---|---|
| Formyl- | Hynic- | | Conjugate | SS | AS | analysis | analysis | Purity |
| siRNA | Peptide | Conjugate | Description | (calc) | (calc) | SS | AS | (duplex) |
| S01 | KKEE | 67 | KKEE-Hy-si | 9360.9 | 7808.2 | 9361.6 | 7809.2 | >99% |
| muExemplary | | | muExemplary | | | (M + H) | (M + H) | |
| Target 1 | | | Target 1 | | | | | |
| S002 | KKEE | 68 | KKEE-Hy- | 9436.2 | 7663.2 | 9436.9 | 7663.9 | >99% |
| HPRT | | | siHPRT | | | (M + H) | (M + H) | |
| S002 | Peptide | 69 | PeptideJ-Hy- | | | | | |
| HPRT | J | | siHPRT | | | | | |

### Example 1.25. Preparation of siRNA Maleimide Peptide Conjugates

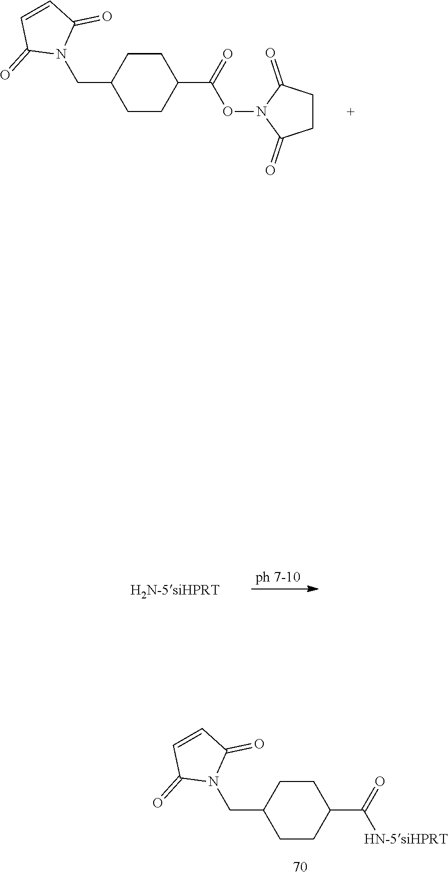

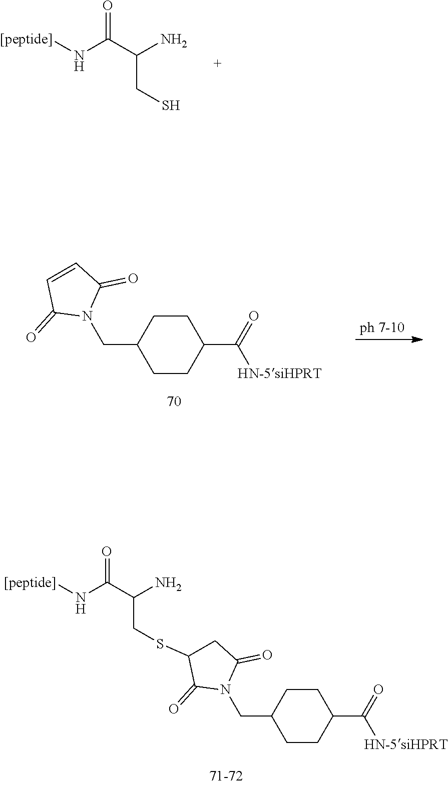

Peptide-siRNA conjugates 71-72 were prepared by SMCC maleimide chemistry using the methods in the Example 1.22:

TABLE 26
maleimide-siHPRT conjugates

| Maleimide- | Cys- | | Conjugate |
|---|---|---|---|
| siHPRT | peptide | Conjugate | Description |
| siHPRT (S02) | CKKEE | 71 | CKKEE-MM-siHPRT |
| | CG3C12 | 72 | CG3C12-MM-siHPRT |

TABLE 27
QC data for hydrazide peptide-siRNA conjugates:

| | MW SS | MW AS | MS | MS | Purity |
|---|---|---|---|---|---|
| Conjugate | (calc) | (calc) | analysis SS | analysis AS | (duplex) |
| 71 | 9380.2 | 7663.2 | 9381.2 | 7662.1 | 95% |
| | | | (M − H) | (M − H) | |

### Example 1.26. Preparation of siRNA Disulfide Peptide Conjugates

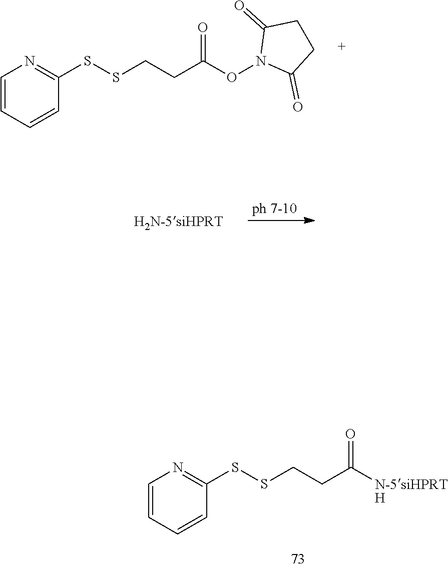

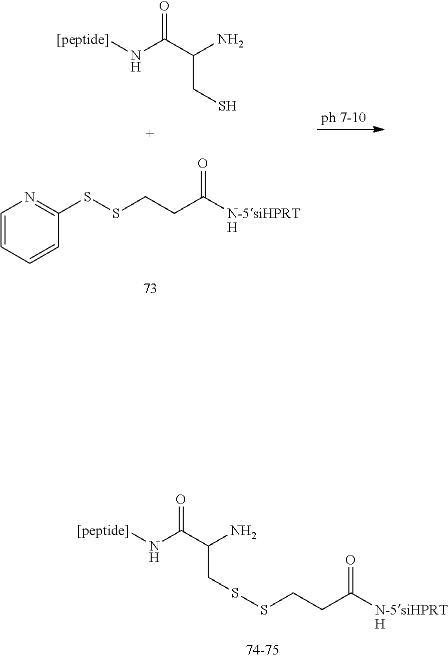

Peptide-siRNA conjugates 74-75 were prepared by SPDP disulfide chemistry using the methods in the Example 1.22:

TABLE 28
Peptide-siRNA conjugates

| ss | Cys-peptide | Conjugate | Conjugate Description |
|---|---|---|---|
| 73 | CKKEE | 74 | CKKEE-SS-siHPRT |
| | CG3C12 | 75 | CG3C12-SS-siHPRT |

### Example 2. Exemplary In Vitro Characterization

The present Example describes exemplary assessments, some or all of which can be used to characterize conjugate agent(s) provided and/or utilized in accordance with the present disclosure, including for example to demonstrate increased level of knockdown of target genes relative to unconjugated control nucleic acid agents, and/or to validate that such improved potency of target gene modulation is Megalin dependent. Both vitro and in vivo approaches are described. In some embodiments, a conjugate agent is characterized by performance in one or more of the assays exemplified herein, and in many embodiments by more than one. In some embodiments, a conjugate agent is characterized by performance in at least one in vivo assay. In some embodiments, a conjugate agent is characterized by improved potency relative to an otherwise comparable unconjugated nucleic acid agent in the same assay (e.g., the same in vitro and/or in vivo assay(s)).

In various embodiments, conjugate molecules are expected to show greater reduction (e.g., on a molar basis) in mRNA and/or protein level(s) of a gene targeted by its nucleic acid payload than is observed when the same nucleic acid is administered in an unconjugated format.

### Materials and Methods

Cell line selection and megalin gene expression by RT-PCR—To demonstrate megalin-mediated uptake of GTTR (Texas Red gentamincin conjugate) and megalin binder conjugate nucleic acids, cell lines are selected based on their tissue of origin and status of megalin expression and additional markers. Cells are seeded in tissue culture treated plates and allowed to grow to ˜80% confluency at the time of collection. Once harvested, cells are washed with PBS and harvested for RT-qPCR. RNA is extracted using the RNA RNeasy Mini Kit from Qiagen. cDNA is typically generated from 500 ng to 1 μg of RNA, using Reverse transcription cDNA synthesis kit from Superscript. RNA expression is evaluated by qPCR using Taqman probes and Taqman Universal PCR Master Mix with Quantstudio 6 Pro System. Initially, HK2, HEK-293, and RPTEC, cells are analyzed for level of megalin, cubulin, LRPAP1, SLC6A19, SLC13A3, and/or CD24, etc., with n=3 technical replicates. HMBS is designated as the house keeping gene for normalization and the RPTEC data are used to calculate relative gene expression for other cell lines.

Megalin protein expression by immunoblotting —Cells are seeded in tissue culture treated plates and allowed to grow to ˜80% confluency at the time of collection. Once harvested, cells are washed with PBS and lysed using RIPA buffer with protease and phosphatase inhibitors. The total protein concentration was estimated by Bicinchoninic acid (BCA) protein assay. Samples are prepared for SDS-PAGE by boiling the lysates with SDS and reducing buffers. SDS-PAGE gradient gels are typically loaded with 15-20 ug protein. Gels are transferred to membrane and the membrane is probed for megalin and beta-actin as a loading control.

GTTR internalization assay—To confirm megalin-mediated GTTR internalization, 10,000 cells are seeded in PDL-coated 384-well cell carrier ultra plates and allowed to attach overnight. After 24 hours, the cells are treated with increasing concentrations (0.125, 0.25, 0.5, 1 ug) of GTTR (GTTR stock concentration=10 μg/ml in PBS). After GTTR addition, cells are incubated up to 24 hours (1, 4, 24 hours) at 37° C. or at 4° C. as a control condition. Plates are washed twice with PBS and imaged by Opera High Content Screening System (PerkinElmer) using Brightfield or 448/560 nm filter sets. Resulting images are analyzed for internalized GTTR signal Harmony High-Content Image Analysis software (PerkinElmer).

To validate megalin-dependent GTTR internalization, cells are seeded in 6-well plates and allowed to attach overnight. After 24 hours, the cells are incubated with megalin-targeting siRNA in 4 doses, non-targeting control siRNA (NTC) and Cyclophilin B control siRNA (Mock) in serum-free media. On the following day, serum-free media is replaced with complete media. After 48 to 96 hours, gene and protein expression of megalin is evaluated by the previously described methods and a desired siRNA concentration is determined for optimal knockdown level of megalin. For the GTTR internalization assay in the presence and absence of megalin, 10,000 cells are seeded in PDL-coated 96-well cell carrier ultra plates and allowed to attach overnight. After 24 hours, the cells are treated with megalin-targeting, NTC and Mock siRNAs in serum free media. On the following day, serum-free media is replaced with complete media. After 48 to 96 hours, the cells are incubated with GTTR and imaged as described above.

Megalin binder conjugate nucleic acid uptake assay—For targeting human HPRT, ASO, PMOs and/or siRNAs targeting the HPRT gene are evaluated as nucleic acid payloads linked to a megalin binding moiety. The reduction in HPRT mRNA level is measured by qPCR and/or one or more other techniques such as RNA sequencing and Northern blot. Reduction in protein levels is assessed by immunoblotting and/or is determined by other quantitative/qualitative techniques such as mass spectrometry, ELISA, immunofluorescence and IHC. In some embodiments, a conjugate agent as described herein shows at least 3-fold greater potency (e.g., reduction in target transcript and/or encoded protein level) as compared with an unconjugated nucleic acid agent (e.g., siRNA). In some embodiments, a conjugate agent shows at least 10-fold greater potency as compared with an unconjugated nucleic acid agent.

In order to demonstrate megalin dependence of an observed increase in potency that characterizes conjugate agents provided and/or utilized herein, assessments can be performed or repeated under conditions where megalin itself is knocked down by RNAi or where the cell lines are engineered via CRISPR or another gene editing technology so that they no longer can express a functional megalin protein. In the latter case it is possible to rescue target modulation by re-administration of a megalin encoding plasmid, thereby returning megalin expression to the engineered cells. Alternatively or additionally, megalin-dependence can be assessed by performing, for example, one or more of competition assays in which an excess of a known megalin binder (including the binding moiety of the conjugate itself, without the nucleic acid portion of the molecule) is administered at the same time as the conjugate, or when megalin shedding is induced by maleate administration or internalization of the protein induced by a known binder, endogenous substrate, or binding antibody, or other molecule, or by reducing the ambient temperature of the experiment such that active endocytosis does not occur.

Alternatively or additionally, an increase in potency of knockdown (or other forms of target engagement such as exon skipping) can be assessed in other cell types besides HEK293, and with other nucleic acid payloads beyond HPRT. Any cell line that express megalin either natively or that has been engineered to do so may be useful, and these may be human cells or other species which express human or other species forms of megalin. In some embodiments, it is anticipated that expression of one or more members of the Megalin internalization and endocytosis pathway such as RAP would be required. It is particularly desirable for assessments to be performed in a human cell system, and preferably as close as physiologically possible to the proximal tubular epithelial cell that is one of the in vivo targets; in certain embodiments, assessments are performed in the hTERT immortalized human RPTEC cell line. In this case it is possible not only to assess the increased potency of knockdown of HPRT but also other genes expressed by human RPTECs including solute carriers such as SLC6A19.

Alternative or additional assessments that may be performed, particularly to troubleshoot or confirm results of assessments disclosed herein, include, for example assessments of:

(i) Megalin-mediated uptake into cells: The capacity of the test cells to take up compounds in a Megalin-dependent manner can be assessed by tracking the uptake of fluorescently labeled analogs of binder molecules which were previously demonstrated to be taken up via Megalin (e.g., FITC-KKEEE, Texas red gentamicin) (Schmitz et al, J Biol Chem 2002 (megalin); Wischnjow et al, Bioconjugate Chem. 2016). More specifically, it is determined by whether the fluorescent signal is present within cells that the uptake is megalin dependent;
(ii) Instability or aggregation of conjugates: This can be assessed by size exclusion chromatography, nuclear magnetic resonance spectroscopy, circular dichroism, or alternative techniques;
(iii) Megalin binding capability of conjugates: To ascertain whether the conjugation process eliminated Megalin binding activity of the conjugate, any number of biophysical techniques such as surface plasmon resonance (SPR), nuclear magnetic resonance (NMR) or others can be employed, for example, using surface immobilized megalin or megalin fragment.;
(iv) Endocytosis after binding: In some embodiments, even if the conjugate binds to megalin, there may be no or an inadequate level of endocytosis after binding between megalin and the conjugate—this can be assessed by fluorescently labeling megalin and monitoring its trafficking from the plasma membrane to intracellular compartments, or by evaluating the presence of conjugate intracellularly by centrifugation and ELISA, qPCR, or mass spectrometry evaluation detection of conjugate in the cell pellet after supernatant is removed;
(v) Trafficking of conjugates: In some embodiments, Megalin may be trafficked in pathways that are incompatible with cytosolic release of the conjugate, such as transcytosis pathways—this can be evaluated by co-staining of a fluorescently labeled megalin or conjugate with endosomal and/or lysosomal markers such as LysoTracker;
(vi) Affinity of conjugate to Megalin: In some embodiments, the affinity of the conjugate may be either too high or too low, such that the conjugate is either not released or improperly co-trafficked with megalin within endosomes. The affinity of the conjugates can be ascertained using biophysical techniques, for example, the same techniques employed in Step 3 above. Alternatively or in addition, a series of related conjugates with varying affinities for megalin can be tested to determine the effect of altered affinity in uptake;
(vii) Degree of endosomal/lysosomal escape of the conjugates: In some embodiments, the conjugate molecule may not escape from the endosomal or lysosomal compartments to engage the target gene sequence within the cytosol or the nucleus—this can be assessed by the use of endosomal release agents such as Chloroquine and Bafilomycin A1.
(viii) Payload release: In some embodiments, it is possible that the nucleic acid payload must be cleaved from the binding moiety in order to engage its gene target with the cell—this can be assessed by the comparison of cleavable and non-cleavable linkers connecting the binding moiety with the nucleic acid payload. Alternatively or additionally, conjugates may be introduced directly into cells, for example by transfection reagents to determine whether the conjugates, particularly containing non-cleavable linkers, are active in modulating gene expression and/or mRNA levels of the target gene. This may tell us more about endosomal escape than cleavage, but if we are using pH or endosomal protease sensitive linkers then perhaps it captures both.

### Results

In some embodiments, a conjugate agent provided and/or utilized in accordance with the present disclosure is demonstrated to:
(i) show specific binding to megalin, for example, when assessed in cell culture or in a cell-free system
(ii) be endocytosed, for example, in a megalin-dependent manner, by megalin-expressing cells;
(iii) release its payload and/or otherwise be appropriately trafficked once internalized into cells (e.g., in cell culture or in vivo); and/or
(iv) effect inhibition of its target gene (e.g., at the mRNA or protein level).

### Example 3: Exemplary In Vivo Characterization

### INTRODUCTION

The present Example describes exemplary assessments that can be used to characterize conjugate agents(s) provided and/or utilized in accordance with the present disclosure, including for example to demonstrate selective uptake and target engagement of genes expressed by cells that express megalin, in particular cell populations in the kidney such as proximal tubule epithelial cells. Among the key questions addressed with experiments described in this Example are: (i) in what cell types can megalin-binding conjugate nucleic acids be detected, and how does this compare quantitatively to the megalin binder and the nucleic acid payload when delivered separately; (ii) whether target engagement can be observed (in particular knockdown, but potentially also exon skipping or other mechanisms) of the gene targeted by the nucleic acid payload in these cells and/or tissues; and (iii) whether a physiologic or phenotypic change can be observed downstream of that gene expression such as change in the level or function of the protein.

PMOs and ASOs when dosed in their unconjugated from show distribution mostly to the liver, kidney, and spleen, while siRNAs show low levels of tissue uptake but in similar tissues. Unconjugated ASOs and PMOs must be administered at relatively high doses often >10 mg/kg in order to show levels in tissues such as the kidney that produce effective target engagement such as gene knockdown. siRNAs however are often unable to be administered at sufficiently high doses to yield cellular uptake and gene knockdown when dosed in an unconjugated form.

Fluorescently-labeled megalin binder uptake assay—To identify organs, tissues, and cell types in which megalin-binder conjugated nucleic acids show increased uptake, the distribution of fluorescently labeled megalin binders without a nucleic acid payload, such as Texas Red gentamicin or FITC-KKEEE3K, is first evaluated. The presence or absence of these fluorophores is evaluated in tissues throughout the nephron including the podocyte/glomerulus, proximal convoluted tubule, proximal straight tubule, loop of Henle, distal convoluted tubule, and collecting duct. The highest fluorescent signal is anticipated in the proximal convoluted tubule, followed by the proximal straight tubule, and fluorescent signal in the remaining tissues may or may not be observed. Fluorescent signal is also expected to observed in podocytes in the event of glomerular injury and/or proteinuria. This is assessed by collecting tissues post necropsy and evaluating slice histology samples by high content microscopy.

Unconjugated ASO/PMO/siRNA tissue distribution-Unconjugated ASOs, PMOs, or siRNAs will be administered to evaluate their tissue distribution without a megalin binding moiety and quantifying the level of material that can be detected in samples taken from tissues including the liver, kidney and others. The levels of these agents are assessed by bioanalytical methods including different chromatographic and electromigration techniques, such as high-performance liquid chromatography (HPLC) coupled with ultraviolet detection (UV) and/or mass spectrometry (MS), capillary gel electrophoresis with UV detection. Moreover, ligand-binding assays (hybridization-based enzyme-linked immunosorbent assay, ELISA) and qPCR are also used. In general, an ELISA is an appropriate approach for the quantification of ASO/PMO/siRNA in different biological samples (including plasma, urine, and tissue homogenates). It is particularly useful in pharmacokinetics studies, especially for the post-distribution phase plasma concentrations (>24 hour after administration) (Andersson S et al. (2018) Drug Discovery Today 23 (10): 1733-1745, PMID: 29852223; Yu R et al. (2013). Expert Opin. Drug Metab. Toxicol 9 (2): 169-182, PMID: 23231725) This method is characterized by relatively high sensitivity, thereby enabling monitoring of very low concentrations of nucleic acids in the elimination phase, as well as providing information about ultimate tissue exposure of the administered drug. ELISAs may be also used for the determination of full-length ASO/PMO/siRNA in urine over 24 hours after administration. Application of ultrasensitive noncompetitive hybridization-ligation heterogeneous enzyme-linked immunosorbent assay, allows for the determination of the different phosphorothioate/2′—O-methyl phosphorothioate ASO and PMO plasma half-lives with minimal cross-reactivity for end truncated metabolites. Chromatographic methods coupled with different detectors, used for ASO/PMO/siRNA bioanalysis are well established by previously published methods (Kaczmarkiewicz A et al. (2019) Crit Rev Anal Chem 49 (3): 256-270, PMID: 30612436; Goyon A et al. (2020) J Pharm Biomed Anal 182:113105, PMID: 32004766; McGinnis et al. (2013) Rapid Commun. Mass Spectrom 27 (23): 2655-2664, PMID: 24591027). HPLC coupled with triple quadrupole MS (MS/MS) or quadrupole time-of-flight (Q-TOF-MS) is commonly used for the separation, identification, and quantification of the full-length nucleic acids and their metabolites. This technique is more suitable for monitoring of plasma distribution phase (<24 hours) and determination of ASO/PMO/siRNA in tissues and urine since their concentrations in such samples are significantly greater compared to plasma concentration in the elimination phase. Although different modes of LC including hydrophilic interaction liquid chromatography (HILIC) and ion-exchange chromatography (IEC), are applied for oligonucleotide analysis, MS coupled with ion pair chromatography (IPC-MS) is the commonly used technique for oligonucleotide bioanalysis, since it provides an appropriate compromise between method sensitivity and separation capacity (Kaczmarkiewicz A et al. (2019) Crit Rev Anal Chem 49 (3): 256-270, PMID: 30612436; Norri D et al. (2019) Bioanalysis 11 (21): 1909-1912, PMID: 31648523; Deng P et al. (2010) J Pharm Biomed Anal 52 (4): 571-579, PMID: 20153130). IEC with UV or fluorescent detector and HILIC coupled with MS are other techniques used for ASO/PMO/siRNA analysis (Kaczmarkiewicz A et al. (2019) Crit Rev Anal Chem 49 (3): 256-270, PMID: 30612436). Besides the aforementioned methods, qPCR-based approach is available for reliable detection and quantification of ASO/PMO as described in Shin M et al. (2022) Nucleic Acid Therapeutics 32 (1): 66-73, PMID: 34928745. In this method, the ASO acts as a splint to direct the ligation of complementary probes and quantitative real-time PCR is used to monitor ligation products. For example, low levels of 2—O-methoxyethyl (2′-O-MOE) gapmer ASO in serum, liver, kidney, lung, heart, muscle, and brain tissues can be detected over a 6-log linear range for detection using this method.

The level of target engagement such as gene knockdown will be assessed from these same samples by techniques such as qPCR, RNAseq, Northern blot, or others as described in the in vitro characterization of Example 1.

Megalin binder conjugated nucleic acid payload tissue distribution —Conjugate molecules are dosed at equimolar quantities relative to the unconjugated fluorescently labeled binder or unconjugated PMO/ASO/siRNA. Tissue levels of the conjugate are assessed in liver, kidney and other tissues by the methods described above. In particular, the ELISA approach is useful for the determination of binder-conjugated ASO/PMO plasma half-lives, as well as their determination in plasma and tissue lysates (kidney, liver, muscle, brain) (Burki U et al. (2015) Nucleic Acid Ther 25 (5): 275-284, PMID: 26176274). In some embodiments, it is anticipated that the level of conjugate and/or nucleic acid payload detected will be greater in animals dosed with the conjugate molecule than in the animals dosed with the nucleic acid in unconjugated form. In some embodiments, it is expected that this will be restricted to the cells and tissues for which fluorescent signal was observed in the animals dosed with the fluorescently labeled binder alone.

Alternative or additional assessments that may be performed, particularly to troubleshoot or confirm results of assessments described herein, include, for example assessments of:
(i) Conjugate aggregation or instability in biologic matrices: To evaluate by determining serum and/or urinary levels of the intact conjugate and any metabolic products of the conjugate—this would likely be done by mass spectrometry but could also be done by SDS-gel electrophotoresis, ELISA, or other techniques. Assays determining conjugate aggregation can also be performed in quality control assessment of the conjugate (or a composition comprising the same).
(ii)Neutralization of conjugate molecules: In some embodiments, the in vivo matrices encountered by the conjugate molecule may otherwise neutralize its activity, such as by producing neutralizing antibodies, by high levels of binding to circulating proteins, or by high levels of binding to other tissue macromolecules. This might be investigated by assays for anti-drug antibodies, in vitro assays to assess plasma protein binding and the fraction of free drug, or similarly to assess the levels of free drug after incubation with tissue slices or lysates—in these cases applying techniques such as mass spectrometry, qPCR, ELISA, or others to the supernatant of these centrifuged samples.
(iii) Receptor specificity: In some embodiments, the conjugate molecule may bind to other receptors besides megalin or be taken up by cells nonspecifically in a non-receptor dependent manner—this would be assessed primarily in in vitro experiments, though additional cell types may be required for in vitro analysis if this is identified in vivo. Additionally, the dose and/or regimen may need to be optimized in order to match the kinetics of the conjugate molecule mechanism of action-timepoints for evaluating drug levels and/or gene expression changes likely need to fit the kinetics of excretion into the ultrafiltrate, the internalization of the conjugate into megalin expressing cells, and the engagement of the target mRNA by RnaseH, RISC, or other mechanisms of oligonucleotide mediated knockdown.
(iv)Nephrotoxicity assessment—In some embodiments, nephrotoxicity is a known effect of both megalin binders such as aminoglycosides as well as nucleic acid payloads such as PMOs and ASOs. In some embodiments, achieving a therapeutic index will be predicated on achieving high levels of productive uptake and target engagement to avoid the toxic effects of accumulating binder or payload. Determining a dose relationship with nephrotoxicity as compared to target engagement is evaluated by histological analysis of tubular degeneration or by renal injury biomarkers including kidney injury molecule-1 (KIM-1), neutrophil gelatinase-associated lipocalin (NGAL), interleukin-18 (IL-18), liver-type fatty acid-binding protein (L-FABP), N-acetyl-β-D-glucosaminidase (NAG), tissue inhibitor of metalloproteinase-2 (TIMP-2) and insulin-like growth factor-binding protein 7 (IGFBP7) (Duijl T et al. (2019) Clin Biochem Rev 40 (2): 79-97, PMID: 31205376). Ototoxicity and vestibular toxicity are other known effects of megalin binders such as aminoglycosides, demonstrating lack of which by histology will be important for conjugates (Fu X et al. (2021) Front Cell Neurosci 15:692762, PMID: 34211374).

### Example 4: Expression of Megalin in HEK293 Cells

This Example describes higher levels of endogenous Megalin in HEK293 cells as compared to HK2 cells and Primary Renal Proximal Tubule Epithelial Cells (RPTEC).

To evaluate the appropriate model(s) for in vitro studies, HEK293, HK2 and Primary Renal Proximal Tubule Epithelial Cells (RPTEC) were profiled for expression of Megalin, Cubilin, LRPAP1, SLC6A19, SLC13A3, and CD24 genes. RT-qPCR was performed as described in the Materials and Methods section of Example 1 herein, using TaqMan probes and gene expression was measured using Quantstudio 6 Pro System. RPTEC cells were used as a baseline measure of Megalin and Cubilin. HEK293 cells demonstrated higher levels of Megalin than HK2 cells relative to RPTEC cells. HK2 cells expressed higher levels of the epithelial marker CD24 as expected. Both HEK293 and HK2 cells had negligible levels of Cubilin, SLC6A19, and SLC13A3 (FIG. 1A).

To further validate the in vitro models, protein levels of Megalin were assessed by immunoblotting using a rabbit polyclonal anti-Megalin antibody. Consistent with the RT-qPCR, HEK293 expressed Megalin protein whereas HK2 and RPTEC cells did not display Megalin protein by immunoblotting (FIG. 1B). Together, these results demonstrate that HEK293 is a suitable model for studying Megalin binding assays in vitro amongst the models tested.

### Example 5: Megalin-Dependent Internalization of Gentamicin

This Example describes internalization of Texas red conjugated Gentamicin (GTTR) in HEK293 cells. The internalization of GTTR is temperature, time and concentration-dependent, as well as dependent on expression of Megalin.

To demonstrate if Megalin is responsible for cellular uptake of conjugated payloads, an internalization assay using Texas red conjugated Gentamicin (GTTR) was developed in HEK293 cells. HEK293 cells were incubated with GTTR at varying concentrations and Texas red fluorescence was measured at varying time points at 37° C. or 4° C. (control) by Opera Phoenix High content screening system.

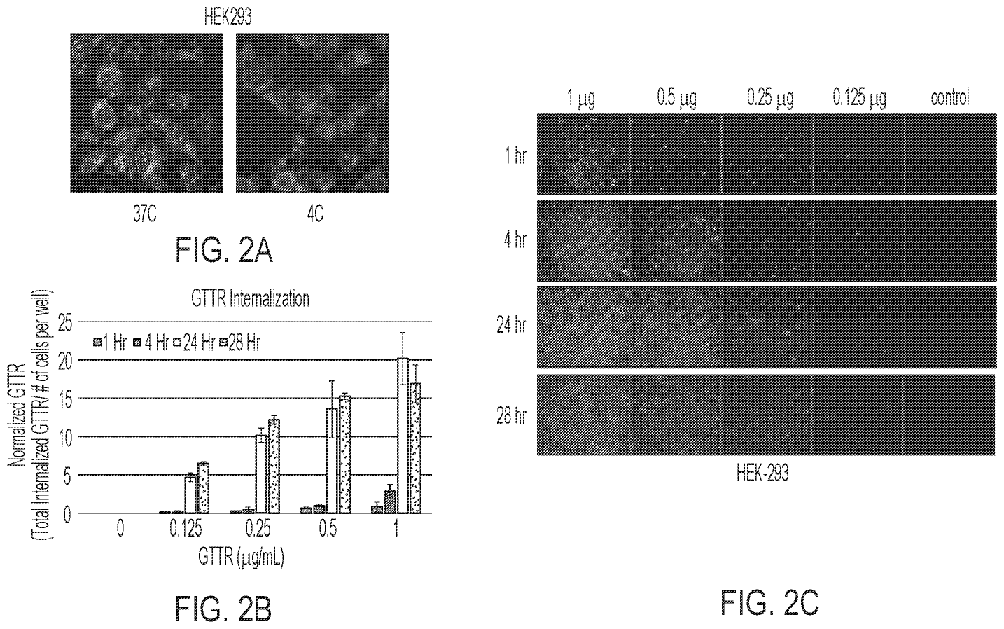

The results show that intracellular uptake of GTTR was temperature dependent. Cells incubated at 37° C. showed greater Texas red fluorescence than those at 4° C. (FIG. 2A). To determine if GTTR uptake is affected by concentration and incubation time of GTTR, cells were incubated with 0, 0.125, 0.25, 0.5, 1 μg/ml of GTTR per well and fluorescence was measured at 1, 4, 24 and 28 hours following GTTR addition. GTTR internalization was found to be time and concentration dependent (FIG. 2B). The images were analyzed, and fluorescence was quantified using Columbus software (FIG. 2C).

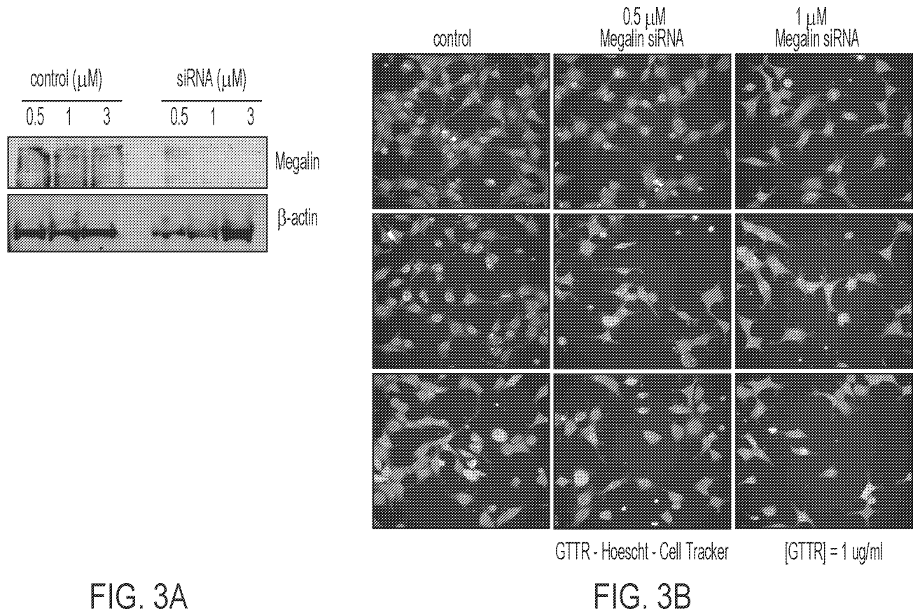

To assess if GTTR uptake is mediated by Megalin, HEK293 cells were treated with siRNA against Megalin to knockdown Megalin protein and measure GTTR uptake. As a first step, cells were treated with three different concentrations of Megalin siRNA to determine the optimal siRNA concentration. All three concentrations tested lead to loss of Megalin protein, thereby confirming the knockdown (FIG. 3A). Next, Megalin dependent uptake of GTTR was measured by the Opera Phoenix system using the method described previously with two different concentrations of Megalin siRNA and a non-targeting scrambled siRNA as a control. As seen in FIG. 3B, loss of Megalin appears to correlate with a decrease in GTTR uptake, suggesting that GTTR internalization is Megalin-dependent.

Taken together, these data suggest that the intracellular uptake of gentamicin conjugates such as GTTR is dependent on the expression of Megalin. This data supports the development of aminoglycosides as a megalin targeting moiety to deliver conjugates to Megalin-expressing cells, and can be extrapolated to the use of other Megalin-targeting moieites to deliver conjugates to Megalin-expressing cells.

### Example 6: CRISPR/Cas9 Knockout (KO) of Megalin

Generation and Confirmation of Megalin KO cell lines —CRISPR/Cas9 genome editing system was used to generate stable Megalin KO cell lines in HEK293 cells. Three single guide RNAs (sgRNAs) with high specificity score and spanning different exons of Megalin transcript region (Exon1, Exon18, and Exon 74) were designed using the Crispor website (http://crispor.tefor.net/). 3×3 primer pairs were selected for each sgRNA and validated by PCR to arrive at the optimal primer pair for each sgRNA. HEK293 cells were electroporated with Cas9/sgRNA mixture using Neon® Transfection System (Thermo Fisher) at manufacturer recommended parameters. Following electroporation, the cells were harvested on day 2 and day 4 and validated by PCR, Sanger sequencing and FACS for expression of Megalin to determine the KO efficiency of the sgRNAs. 6 single-cell clones were generated from polyclonal cell populations by limiting dilution. The edited cell lines 11, 45, 48, 49, 50 and 52 were then confirmed for deletion of Megalin by sanger Sequencing (not shown).

TABLE 29
Guide RNA sequences

| | Position | Strand | gRNA | PAM |
|---|---|---|---|---|
| sgRNA1 | EXON1 | fw | TCTCGTCGCCTGCCTAGCGC | CGG |
| sgRNA2 | EXON74 | fw | ATCCTCTTGATCGTCGTAAT | TGG |
| sgRNA3 | EXON18 | fw | CCAATGGCTTGGCCATCGAT | TGG |

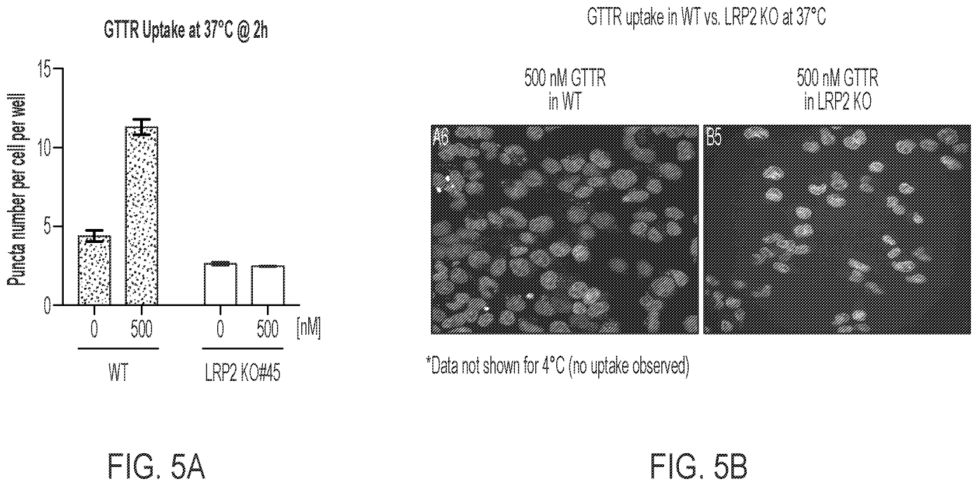

Reduced GTTR uptake—To confirm Megalin-mediated GTTR internalization, 12,000 cells of HEK293 parental line and Megalin KO clone #45 were seeded in PDL-coated 96-well plates and allowed to attach overnight. After 48 hours, the cells were treated with 100 nM and 500 nM of GTTR and TR. After the addition of GTTR and TR, cells were incubated for 2 and 4 hours at 37° C. and at 4° C. as a control condition. 30 minutes before the indicated timepoint, Hoechst and cell Tracker Green dye mix was added to the cells to serve as nuclear and cytoplasmic markers, respectively. Plates were washed three times with Fluorobrite media and imaged by Operetta High Content Screening System (PerkinElmer) using Brightfield or 448/560 nm filter sets. Resulting images were analyzed for internalized GTTR signal (Puncta per cell) using Harmony High-Content Image Analysis software (PerkinElmer) (FIGS. 5A-5B).

TABLE 30
Raw Cq values of Genes of Interest (GOI) in different cell lines

| | Supplier/ | | muExemplary | | | | |
|---|---|---|---|---|---|---|---|
| Cell Line | Cat # | HPRT | Target 1 | HMBS | LRP2 | Cubilin | Dab2 |
| HEK293 | ATCC/ | 23-24 | >35 | 22-23 | 23-24 | 26-27 | |
| | CRL-1573 | | | | | | |
| Renca | ATCC/ | 24-25 | 32-33 | 25-26 | 26-27 | 31-32 | >35 |
| | CRL-2947 | | | | | | |
| OK | ATCC/ | 24-25 | 23-24 | 27-28 | 25-26 | TBD | TBD |
| | CRL-1840 | | | | | | |
| mRPTEC | Sciencell | 25-26 | >35 | 27-28 | 29-30 | 32-33 | 29 |
| primary | | | | | | | |
| mRPTEC | Cell | 24-25 | >35 | 26-27 | 24-25 | 31-32 | 31-32 |
| primary | Biologics | | | | | | |
| RPTEC/TERT1 | ATCC/ | 26-28 | >35 | 23-24 | >35 | 25-26 | |
| | CRL-4031 | | | | | | |
| HK2 | ATCC/ | | >35 | 19-21 | 33-35 | 29-30 | |
| | CRL-2190 | | | | | | |

### Example 7: Characterization of Oligonucleotides

The present Example describes the characterization of oligonucleotides including PMOs and SiRNAs targeting HPRT gene in HEK293 cells. Levels of knockdown of target gene caused by conjugate agents relative to unconjugated control nucleic acid agents demonstrated the potency of oligonucleotides.

### Example 7.1. HPRT PMO

To evaluate HPRT PMO activity, HEK293 cells were seeded in PDL-coated 96-well plates at a seeding density of 7,500 cells per well. Cells were cultured in a humidifier CO2 incubator and allowed to attach overnight. After 24 hours, the original plating media was removed, and the cells were treated with HPRT-targeting PMOs of different concentrations. 6 μM Endo-Porter (GeneTools), a reagent for delivering Morpholino oligos into the cytosol of cultured cells, was added in addition to PMO as a positive control. Once treated, the plates were placed in the incubator at 37° C. and 5% CO2 for 72 hours. To harvest, the cells were washed with PBS and lysed using the Cells-to-CT™ 1-Step TaqMan™ Kit (ThermoFisher Scientific). 50 ul of lysis buffer/DNase I reagent mixture was added into each well, mixed thoroughly and incubated for 5-10 minutes at room temperature. 5 μL of Stop Solution was added to each well and incubated for 5 minutes. The cell lysates were used for RT-qPCR analysis to evaluate mRNA expressions. RT-qPCR was performed on a Quant Studio 7 Pro Real-Time PCR System using a Cells-to-CT™ 1-Step TaqMan™ kit and Taqman probes (ThermoFisher Scientific). Lysates from cells treated with test PMOs against HPRT and mock were probed with HuHPRT-FAM and HuHMBS-VIC. HMBS was designated as the house keeping gene for normalization. The fold change relative to mock wells (no treatment) in HPRT mRNA expression was calculated as 2\hat{ }^{(-ΔΔCT)}. Relative expression was compiled and analyzed in GraphPad Prism.

Using the method described above, eight human HPRT PMOs were screened in HEK293 cells. The descriptions of the unconjugated and conjugated PMOs are listed in Table 31.

TABLE 31
HPRT PMO conjugates

| Compound | Oligo Name | Description |
|---|---|---|
| 101 | HuHPRT PMO | unconjugated huHPRT PMO |
| 39 | HuHPRT PMO | huHPRT PMO_K-G3-C12_hynic peptide |
| 79 | HuHPRT PMO | huHPRT-PMO Gentamicin ring 2 ether |
| 38 | HuHPRT PMO | huHPRT PMO_HyN/K(KKEEE)3 |

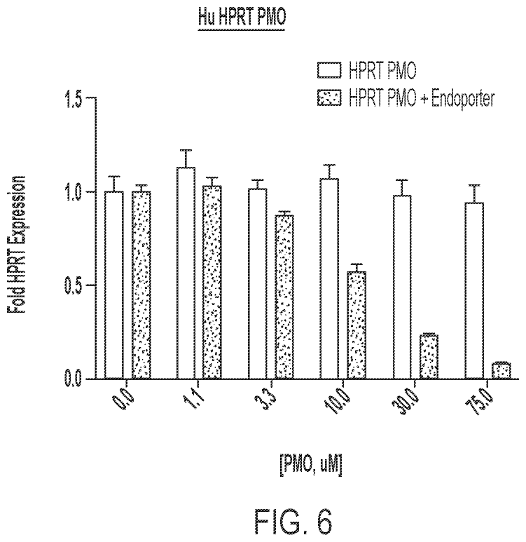

Unconjugated HPRT PMO (Compound 101) was used as a control agent to demonstrate the PMO knockdown efficiency in HEK293 cells. Dose dependent HPRT mRNA knockdown was observed only when transfected in the presence of 6-8 uM Endoporter (Gene Tools, Philomath OR), an endosomal release agent. PMO+ Endoporter was considered as a positive control for PMO screening (FIG. 6).

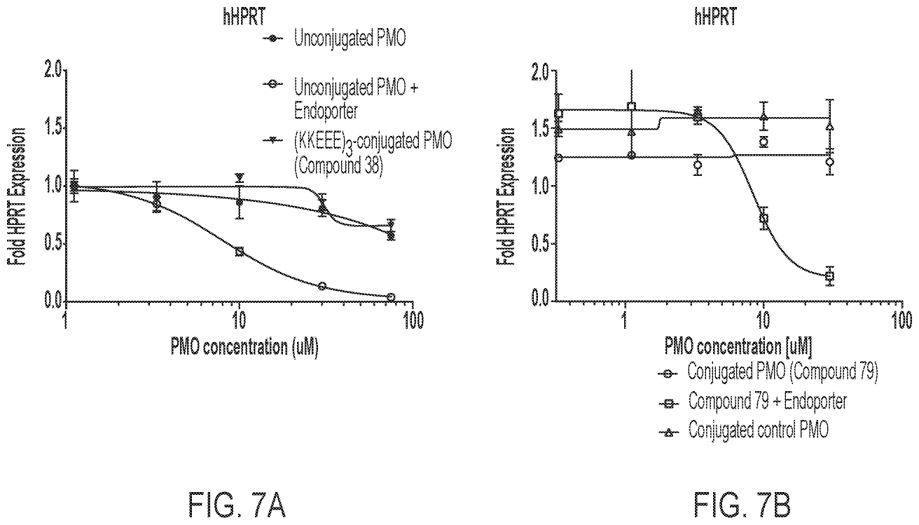

KKEEE3-PMO conjugate (Compound 38) and Gentamicin-PMO conjugate (Compound 79) were tested in HEK293 cells at doses of 0, 1, 3, 10, 30 and 100 uM with a treatment duration of 72 hours. Both the conjugates demonstrated no significant HPRT knockdown compared to unconjugated PMO (Compound 101) and non-targeting PMO conjugated to Gentamicin, a negative control agent (FIGS. 7A-7B). More PMO conjugates are designed and synthesized to improve the knockdown potency.

### Example 7. 2. HPRT siRNA

To evaluate HPRT SiRNA conjugate knockdown activity, HEK293 cells were seeded onto PDL-coated 96-well plates at a seeding density of 15,000 cells per well. SiRNAs were added into cells without, or with transfection reagent. Reverse transfection was performed using Lipofectamine RNAiMAX transfection reagent (0.2 ul/well, ThermoFisher Scientific). Cells were cultured in a humidifier CO2 incubator for 24 hours, followed by addition of 25 uM Chloroquine (CQ). After 48 hours of SiRNA treatment, the cells were lysed using the Cells-to-CT™ 1-Step TaqMan™ Kit following the manufacturer's instructions. RT-qPCR was performed on a Quant Studio 7 Pro Real-Time PCR System using a Cells-to-CT™ 1-Step TaqMan™ kit and Taqman probes. Lysates from cells treated with unconjugated and conjugated SiRNA against HPRT and mock were probed with HuHPRT-FAM and HuHMBS-VIC. HMBS was designated as the house keeping gene for normalization and the fold change relative to mock wells (no treatment) in HPRT expression was calculated as 2 \hat{ }^{(-ΔΔCt)}. Relative expression is compiled and analyzed in GraphPad Prism.

Thirteen unconjugated and conjugated human/mouse HPRT SiRNAs are screened. The descriptions of the unconjugated and conjugated PMOs are listed in Table 32.

TABLE 32
HPRT PMOs

| Compound | Oligo Name | Description |
|---|---|---|
| 102 | Hu/muHPRT SiRNA | duplex unconjugated HPRT siRNA |
| 71 | Hu/muHPRT SiRNA | HPRT siRNA_Cys(KKEEE)3 |
| | | peptide SMCC linker |
| 83 | Hu/muHPRT SiRNA | HPRT si Gentamicin ring 2 ether |
| | | (ring 3 oxazolidinone) |
| 84 | Hu/muHPRT SiRNA | HPRT si Gentamicin ring 2 ether |
| 103 | Hu/muHPRT SiRNA | HPRT 5′-amine modified siRNA |
| | | duplex (unconjugated) |

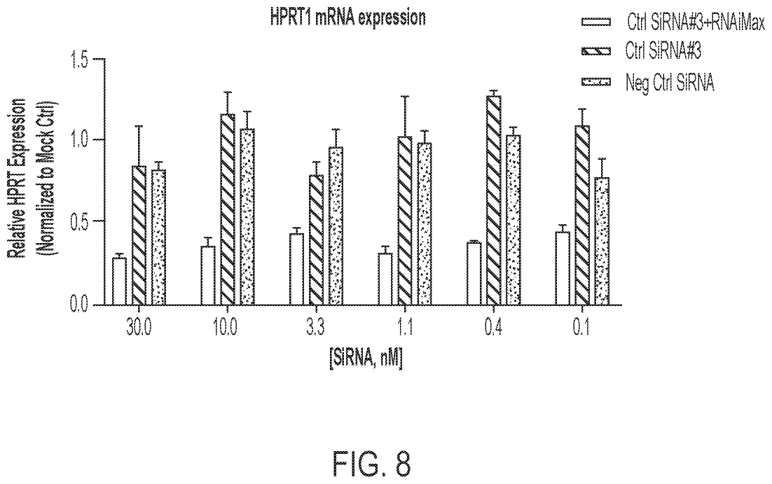

Pre-designed positive control SiRNA induced target gene knockdown in HEK293 cells only when transfected with RNAiMax, and non-targeting negative control siRNA showed no effect on HPRT gene expression (FIG. 8).

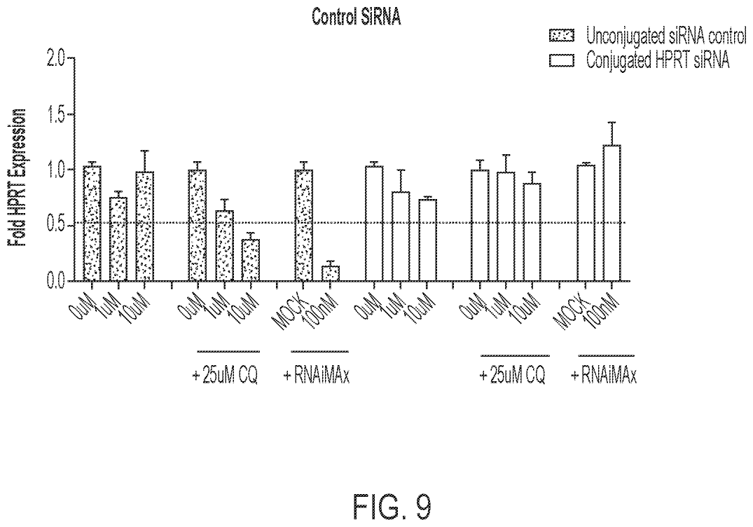

To characterize HPRT siRNA conjugates, an unconjugated HPRT siRNA and a non-targeting siRNA conjugate were used as negative controls, and RNAiMax transfected control siRNA as a positive control. (FIG. 9). No knockdown was achieved without RNAiMax transfection, which suggested that in some embodiments, the siRNAs can be trapped inside the endosome and fail to be released into the cytosol to reduce target gene mRNA expression. The endosome-disruptive Chloroquine (CQ) was then utilized to induce endosomal escape.

### Example 7.3 Endo-Porter

Endo-Porter (Gene Tools) is a novel peptide reagent for delivering PMOs, peptides or proteins into the cytosol of cultured cells. The mechanism of Endo-Porter delivery relies on PMO and Endo-Porter being taken up from the media into the same endosome. Endo-Porter is an amphiphilic peptide with a sharp transition pH, with a hydrophobic face which associates with cell membranes once added to culture medium. PMOs in the medium are then co-endocytosed with Endo-Porter. Natural acidification of the endosome protonates Endo-Porter that, in its ionic form, permeabilizes the endosome and releases the endosome contents into the cytosol, resulting in targeted gene knockdown. Endo-Porter was provided as a 1.0 mM sterile stock solution and was diluted into a final Endo-Porter concentration of 6 uM prior to use.

### Example 8: Kidney Tissue Uptake and Activity of a Gentamicin Conjugated Modified siRNA on Target Gene RNA In Vivo at a Single Dose

siRNA Conjugates Targeting an exemplary mouse target (muExemplary Target 1): A series of experiments were carried out to determine whether conjugation of double-stranded RNA (dsRNA) molecules to gentamicin results in increased kidney accumulation. In this experiment, a siRNA molecule comprising the guide and passenger strands directed to muExemplary Target 1 as described in Example 1.23, either conjugated to gentamicin (compound 81), or unconjugated S01 (containing 5′-hexylamine modification on the passenger strand) was tested in C57BL6 male mice following a single IV bolus administration at 10 mg/kg. Plasma and kidney tissues were taken at 15 minutes, 30 minutes, 1 hour, 2 hours, 4 hours, 8 hours, 16 hours, 24 hours, 48 hours, 72 hours post dose for the evaluation of oligonucleotide concentration in plasma and kidney and target gene knockdown effect in kidney tissues. Kidney tissues from a group of vehicle-treated mice sacrificed at 15 min post dose served as control group.

### Pharmacokinetic Analysis of Oligonucleotide in Kidney Tissues

Analysis oligonucleotide in kidney tissues was carried by ion-pair reverse phase UPLC coupled with high resolution mass spectrometer (HRMS) following overnight proteinase K digestion and sample preparation by liquid-liquid extraction. The LC mobile phases contain HFIP, DIPEA as ion-pair reagents. The HRMS was run at negative mode with full scan over a range of 730-1000 m/z and resolution of 70,000 for quantification. Target m/z of 974.8899 for antisense strand (AS), with a limit of quantitation of 50 nM.

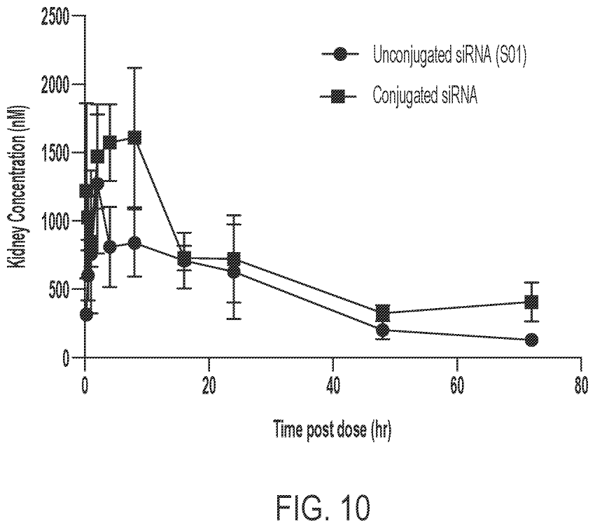

As shown in FIG. 10 and Table 33, treatment of the gentamicin-conjugated siRNA resulted in increased uptake in kidney tissues in comparison to unconjugated siRNA control.

TABLE 33
Average kidney concentration (nM)

| Time | Unconjugated | | Fold over |
|---|---|---|---|
| post dose | siRNA S01 | Conjugated | unconjugated |
| (hr) | (average) | siRNA | control |
| 0.25 | 315.000 | 1220.5 | 3.8746 |
| 0.5 | 600.667 | 1023.75 | 1.7044 |
| 1 | 757.667 | 846 | 1.1166 |
| 2 | 1270.000 | 1472.75 | 1.1596 |
| 4 | 809.333 | 1572.75 | 1.9433 |
| 8 | 839.000 | 1608.75 | 1.9175 |
| 16 | 709.000 | 728.25 | 1.0272 |
| 24 | 627.667 | 722.25 | 1.1507 |
| 48 | 200.333 | 326.5 | 1.6298 |
| 72 | 130.667 | 407 | 3.1148 |

Although somewhat variable depending on the timepoint, the gentamicin-conjugated siRNA was found to have between 1.02-3.87 fold higher accumulation in kidney tissues. There was no significant difference in plasma levels of the conjugated siRNA and unconjugated control (data not shown).

### Target Gene RNA Analysis

RNA was extracted from kidney tissues for real-time PCR analysis of measurement of RNA expression of target gene using primer probe set Mm01351206_g1 (ThermoFischer Scientific). Results are presented as percent change of RNA, relative to vehicle control, normalized to the average of mouse Actb, Gapdh and Ppia as controls.

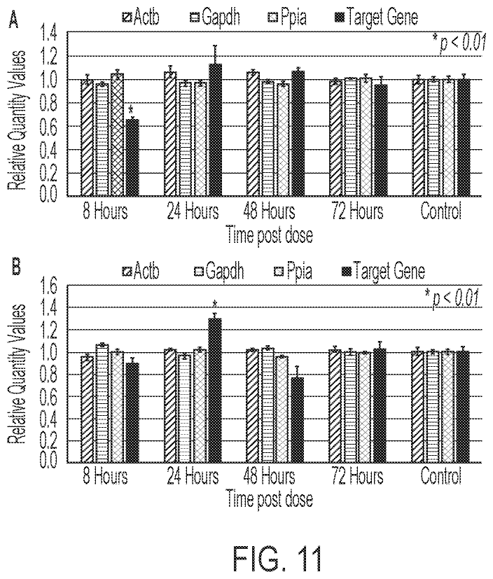

As shown in FIGS. 11A-11B, treatment of gentamicin conjugated oligonucleotide (compound 81) resulted in target gene knockdown in kidney tissues in comparison to unconjugated siRNA (alkyne modified S01).

siRNA Conjugates Targeting HPRT

Additional experiments were conducted using a second siRNA molecule. The S02 siRNA, which targets the murine HPRT gene, was either conjugated with gentamicin (compound 83) as described in Example 1.23, or unconjugated S02 (containing 5′-hexylamine modification on the passenger strand). C57BL6 mice were injected with a single IV bolus administration at 10 mg/kg as described in the previous section. Plasma and kidney tissues were taken at 15 minutes, 30 minutes, 4 hours, 8 hours, and 24 hours post dose for the evaluation of oligonucleotide concentration in plasma and kidney and target gene knockdown effect in kidney tissues. Kidney tissues from a group of vehicle-treated mice sacrificed at 15 min post dose served as control group.

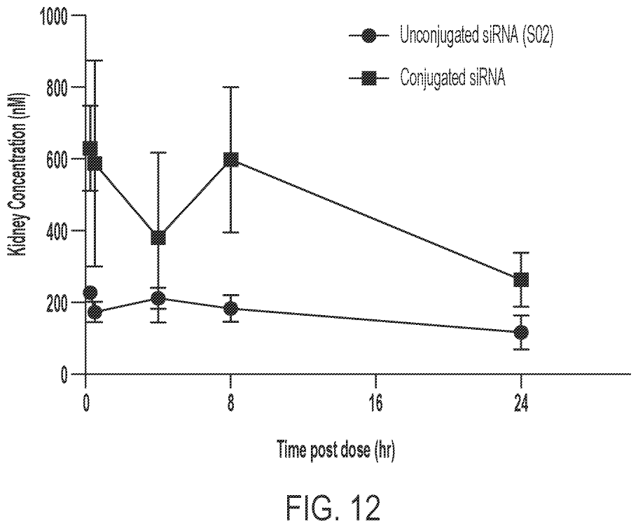

As shown in FIG. 12 and Table 34, treatment of the gentamicin-conjugated HPRT siRNA resulted in increased uptake in kidney tissues in comparison to unconjugated siRNA control.

TABLE 34
Average kidney concentration (nM)

| Time | | | |
|---|---|---|---|
| post | Unconjugated | | |
| dose | siRNA S02 | Conjugated | Fold over |
| (hr) | (average) | siRNA | unconjugated |
| 0.25 | 226.67 | 629.67 | 2.78 |
| 0.5 | 173.33 | 587.00 | 3.39 |
| 4 | 210.67 | 380.00 | 1.80 |
| 8 | 183.00 | 597.33 | 3.26 |
| 24 | 116.33 | 263.33 | 2.26 |

Although somewhat variable depending on the timepoint, the gentamicin-conjugated siRNA was found to have between ˜1.80-3.38 fold higher accumulation in kidney tissues. As in the previous experiment, there was no significant difference in plasma levels of the conjugated siRNA and unconjugated control (data not shown).

What is claimed is:

1. A conjugate agent comprising:
(i) a targeting moiety which specifically binds to a kidney cell surface factor chosen from megalin or cubilin, and is directly or indirectly conjugated with,
(ii) a payload moiety comprising a nucleic acid.

2. The conjugate agent of claim 1, wherein the targeting moiety and payload moiety are indirectly conjugated by way of a linker.

3. The conjugate agent of claim 1 or 2, wherein the kidney cell surface factor is internalized when bound by the targeting moiety.

4. The conjugate agent of any one the preceding claims, wherein the targeting moiety binds the kidney cell surface factor at one or more extracellular domains on the kidney cell surface factor.

5. The conjugate agent of any one the preceding claims, wherein the targeting moiety binds at or near one or more complement type repeat domains of the kidney cell surface factor.

6. The conjugate agent of any one the preceding claims, wherein the targeting moiety is chosen from: a polypeptide, an aminoglycoside, an endogenous ligand, a xenobiotic, an antibody or a fragment thereof, an aptamer, a small molecule, a vitamin, or combinations thereof.

7. The conjugate agent of claim 6, wherein the targeting moiety is or comprises a polypeptide.

8. The conjugate agent of claim 6, wherein the targeting moiety is or comprises an aminoglycoside.

9. The conjugate agent of claim 8, wherein the aminoglycoside is chosen from one or more, or all of: streptomycin, neomycin, kanamycin, paromomycin, gentamicin, G-418 (geneticin) ELX-202, tobramycin, amikacin, netilmicin, spectinomycin, sisomicin, dibekacin, isepamicin, framycetin, paromomycin, apramycin, fradiomycin, arbekacin, plazomicin, or a derivative, or a fragment, or a variant thereof.

10. The conjugate agent of claim 9, wherein the aminoglycoside is or comprises gentamicin or a derivative, or a fragment, or a variant thereof.

11. The conjugate agent of claim 10, wherein the linker is attached to ring 2 of gentamicin, or a derivative, fragment, or variant thereof.

12. The conjugate agent of any one of claims 2-11, wherein the linker is a cleavable linker.

13. The conjugate agent of any one of the preceding claims, wherein the targeting moiety and the payload moiety are conjugated by a linker comprising the structure:

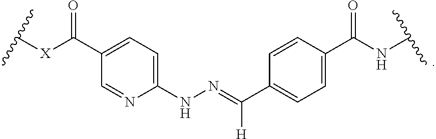

14. The conjugate agent of any one of claim 1-12, wherein the targeting moiety and the payload moiety are conjugated by a linker comprising the structure:

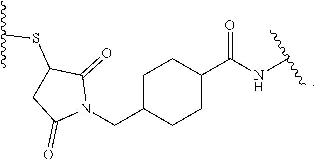 [SMILES: *NC(=O)C1CCC(CN2C(=O)CC(S*)C2=O)CC1]

15. The conjugate agent of any one of claims 2-14, wherein the conjugate agent comprises about 1-30 repeats of the linker.

16. A conjugate agent comprising the structure of Formula III: 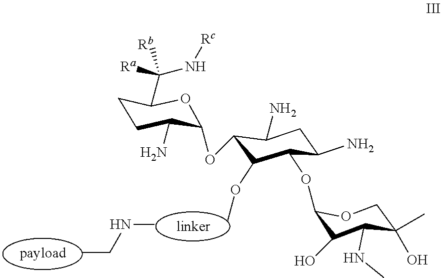
wherein each of r^{a}, r^{b}, and R^{c} is selected from h and CH₃;
the linker is a bivalent linker; and
the payload is a payload moiety.

17. The conjugate agent of claim 16, wherein the conjugate agent comprises about 1-30 repeats of the linker.

18. The conjugate agent of claim 16 or 17, wherein the payload moiety is a nucleic acid.

19. The conjugate agent of any one of claims 16-18, wherein the targeting moiety of Formula III specifically binds to a kidney cell surface factor.

20. The conjugate agent of claim 19, wherein the kidney cell surface factor is chosen from megalin or cubilin.

21. The conjugate agent of any one of the preceding claims, wherein the payload moiety acts on a target chosen from a target provided in any one of Tables 2-5, or a combination thereof.

22. The conjugate agent of any one of the preceding claims, wherein the nucleic acid is or comprises an antisense sequence element, optionally wherein, the antisense sequence element is complementary to at least a portion of one or more of: an exon, an intron, an untranslated region, a splice junction, a promoter region, an enhancer region, or a non-coding region in a target sequence.

23. The conjugate agent of any one of the preceding claims, wherein the nucleic acid comprises a sequence element that is at least 80% complementary to a target sequence in a sense strand.

24. The conjugate agent of any one of the preceding claims, wherein the nucleic acid comprises a sequence element that is at least 80% complementary to a target sequence in an antisense strand.

25. The conjugate agent of any one of the preceding claims, wherein the nucleic acid comprises at least one sequence element with at least 3 contiguous nucleotides having at least 80% complementarity to a portion of a target sequence.

26. The conjugate agent of any one of the preceding claims, wherein the nucleic acid is single stranded.

27. The conjugate agent of any one of the preceding claims, wherein the nucleic acid is double stranded.

28. The conjugate agent of any one of the preceding claims, wherein the nucleic acid is or comprises RNA.

29. The conjugate agent of claim 28, wherein the RNA is or comprises an interfering RNA (RNAi).

30. The conjugated agent of claim 29, wherein the RNAi is or comprises a short interfering RNA (siRNA).

31. The conjugate agent of any one of claims 28-30, wherein the RNA comprises about 15-25 nucleotides.

32. The conjugate agent of any one of claims 28-31, wherein the RNA comprises one or more modified nucleotides.

33. The conjugate agent of any one of claims 1-27, wherein the nucleic acid is or comprises DNA.

34. The conjugate agent of claim 33, wherein the DNA is or comprises a DNA analog, optionally wherein the DNA analog comprises one or more morpholino subunits linked together by phosphorus-containing linkage.

35. The conjugate agent of claim 34, wherein the DNA analog is or comprises a phosphorodiamidate morpholino nucleic acid (PMO).

36. The conjugate agent of claim 35, wherein the PMO comprises about 12-40 nucleotides.

37. The conjugate agent of any one of the preceding claims, wherein the nucleic acid is or comprises an antisense oligo (ASO).

38. The conjugate agent of any one of 1-27 or 33-36, wherein the nucleic acid is or comprises a peptide nucleic acid (PNA).

39. The conjugate agent of any one of the preceding claims, wherein the nucleic acid comprises a modification comprising: a modified backbone, a modified nucleobase, a modified ribose, a modified deoxyribose, or a combination thereof.

40. The conjugate agent of any one of the preceding claims, wherein the nucleic acid comprises one or more modification to a 5′ end of the nucleic acid.

41. The conjugate agent of any one of the preceding claims, wherein the payload moiety is conjugated to the targeting moiety at a 5′ end of the payload moiety, or at a 3′ end of the payload moiety.

42. The conjugate agent of any one of the preceding claims, wherein the nucleic acid is characterized in that when delivered to a cell expressing the target, reduced expression and/or activity of the target is observed as compared to a cell which has not been delivered the nucleic acid or a cell which does not express the target.

43. The conjugate agent of any one of the preceding claims, characterized in that when delivered to a cell, tissue or organism, the payload moiety is delivered to, and/or expressed in, at least 5% more target cells compared to:
(a) an otherwise similar cell, tissue or organism delivered an unconjugated payload moiety;
(b)non-target cells; or
(c) both (a) and (b).

44. The conjugate agent of claim 42 or 43, wherein the target cell is chosen from: renal cells, thyroid cells, parathyroid cells, cells of the inner ear, nervous system cells, or a combination thereof, optionally wherein the target cell is or comprises a cell that expresses a kidney cell surface factor.

45. A conjugate comprising the structure of Formula I: 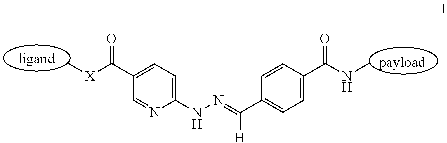
wherein
X is NH or O;
the ligand is a targeting moiety; and
the payload is a payload moiety.

46. A conjugate comprising the structure of Formula II: 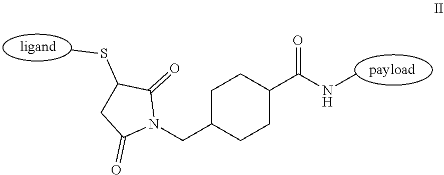
wherein the ligand is a targeting moiety; and
the payload is a payload moiety.

47. A pharmaceutical composition that comprises or delivers the conjugate agent of any one of the preceding claims.

48. A cell with a conjugate agent of any one of claims 1-46 bound thereto.

49. A method of delivering a conjugate agent to a cell, tissue, or subject, the method comprising a step of:
administering to the cell, tissue, or subject, the conjugate agent comprising a targeting moiety directly or indirectly linked with a payload moiety of any one of claims 1-46, or the pharmaceutical composition of claim 47.

50. A method of treating a disease or disorder, the method comprising a step of:
administering to a subject suffering from or susceptible to the disease or disorder, the conjugate agent comprising a targeting moiety directly or indirectly linked with a payload moiety of any one of claims 1-46, or the pharmaceutical composition of claim 47.

51. The method of claim 50, wherein the disease is a disease associated with expression of a cell surface receptor, optionally wherein the disease is a disease comprising a cell in which both a cell surface receptor and a target recognized by the payload moiety are present.

52. A method of improving delivery of an agent to a cell, the method comprising contacting a system or subject comprising at least one cell with a conjugate agent of any one of claims 1-46 or a pharmaceutical composition of claim 47, wherein the cell is a cell that expresses a kidney cell surface factor.

53. The method of any one of claim 49 or 51-52, wherein the cell is chosen from: kidney cells, thyroid cells, parathyroid cells, cells of the inner ear or nervous system cells, or a combination thereof.

54. The method of claim 53, wherein the kidney cell is chosen from a proximal tubular epithelial cell and/or a podocyte.

55. The method of any one of claim 49-51 or 53-54, wherein administering the conjugate agent to the cell, tissue or organism, delivers the payload moiety to at least 5% more target cells compared to:
(a) an otherwise similar cell, tissue or organism delivered an unconjugated payload moiety;
(b) a non-target cell; or
(c) both (a) and (b).

56. The method of claim 55, wherein the target cell is or comprises a kidney cell.

57. The method of claim 55 or 56, wherein the target cell is or comprises a cell that has expression of a kidney cell surface factor chosen from megalin or cubilin.

58. The conjugate agent of any one of claims 1-46, wherein the targeting moiety binds the kidney cell surface factor at the nephron apical membrane.

59. The conjugate agent of any one of claims 1-46, wherein the targeting moiety binds the kidney cell surface factor at the nephron basolateral membrane.


SEQUENCE LISTING (29 sequences)

SEQ ID NO: 1 - protein, 16 aa, synthetic construct
KKEEEKKEEEKKEEEK

SEQ ID NO: 2 - protein, 357 aa, Homo sapiens
MAPRRVRSFLRGLPALLLLLLFLGPWPAASHGGKYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNEL

SEQ ID NO: 3 - protein, 4655 aa, Homo sapiens
MDRGPAAVACTLLLALVACLAPASGQECDSAHFRCGSGHCIPADWRCDGTKDCSDDADEIGCAVVTCQQGYFKCQSEGQCIPNSWVCDQDQDCDDGSDERQDCSQSTCSSHQITCSNGQCIPSEYRCDHVRDCPDGADENDCQYPTCEQLTCDNGACYNTSQKCDWKVDCRDSSDEINCTEICLHNEFSCGNGECIPRAYVCDHDNDCQDGSDEHACNYPTCGGYQFTCPSGRCIYQNWVCDGEDDCKDNGDEDGCESGPHDVHKCSPREWSCPESGRCISIYKVCDGILDCPGREDENNTSTGKYCSMTLCSALNCQYQCHETPYGGACFCPPGYIINHNDSRTCVEFDDCQIWGICDQKCESRPGRHLCHCEEGYILERGQYCKANDSFGEASIIFSNGRDLLIGDIHGRSFRILVESQNRGVAVGVAFHYHLQRVFWTDTVQNKVFSVDINGLNIQEVLNVSVETPENLAVDWVNNKIYLVETKVNRIDMVNLDGSYRVTLITENLGHPRGIAVDPTVGYLFFSDWESLSGEPKLERAFMDGSNRKDLVKTKLGWPAGVTLDMISKRVYWVDSRFDYIETVTYDGIQRKTVVHGGSLIPHPFGVSLFEGQVFFTDWTKMAVLKANKFTETNPQVYYQASLRPYGVTVYHSLRQPYATNPCKDNNGGCEQVCVLSHRTDNDGLGFRCKCTFGFQLDTDERHCIAVQNFLIFSSQVAIRGIPFTLSTQEDVMVPVSGNPSFFVGIDFDAQDSTIFFSDMSKHMIFKQKIDGTGREILAANRVENVESLAFDWISKNLYWTDSHYKSISVMRLADKTRRTVVQYLNNPRSVVVHPFAGYLFFTDWFRPAKIMRAWSDGSHLLPVINTTLGWPNGLAIDWAASRLYWVDAYFDKIEHSTFDGLDRRRLGHIEQMTHPFGLAIFGEHLFFTDWRLGAIIRVRKADGGEMTVIRSGIAYILHLKSYDVNIQTGSNACNQPTHPNGDCSHFCFPVPNFQRVCGCPYGMRLASNHLTCEGDPTNEPPTEQCGLFSFPCKNGRCVPNYYLCDGVDDCHDNSDEQLCGTLNNTCSSSAFTCGHGECIPAHWRCDKRNDCVDGSDEHNCPTHAPASCLDTQYTCDNHQCISKNWVCDTDNDCGDGSDEKNCNSTETCQPSQFNCPNHRCIDLSFVCDGDKDCVDGSDEVGCVLNCTASQFKCASGDKCIGVTNRCDGVFDCSDNSDEAGCPTRPPGMCHSDEFQCQEDGICIPNFWECDGHPDCLYGSDEHNACVPKTCPSSYFHCDNGNCIHRAWLCDRDNDCGDMSDEKDCPTQPFRCPSWQWQCLGHNICVNLSVVCDGIFDCPNGTDESPLCNGNSCSDFNGGCTHECVQEPFGAKCLCPLGFLLANDSKTCEDIDECDILGSCSQHCYNMRGSFRCSCDTGYMLESDGRTCKVTASESLLLLVASQNKIIADSVTSQVHNIYSLVENGSYIVAVDFDSISGRIFWSDATQGKTWSAFQNGTDRRVVFDSSIILTETIAIDWVGRNLYWTDYALETIEVSKIDGSHRTVLISKNLTNPRGLALDPRMNEHLLFWSDWGHHPRIERASMDGSMRTVIVQDKIFWPCGLTIDYPNRLLYFMDSYLDYMDFCDYNGHHRRQVIASDLIIRHPYALTLFEDSVYWTDRATRRVMRANKWHGGNQSVVMYNIQWPLGIVAVHPSKQPNSVNPCAFSRCSHLCLLSSQGPHFYSCVCPSGWSLSPDLLNCLRDDQPFLITVRQHIIFGISLNPEVKSNDAMVPIAGIQNGLDVEFDDAEQYIYWVENPGEIHRVKTDGTNRTVFASISMVGPSMNLALDWISRNLYSTNPRTQSIEVLTLHGDIRYRKTLIANDGTALGVGFPIGITVDPARGKLYWSDQGTDSGVPAKIASANMDGTSVKTLFTGNLEHLECVTLDIEEQKLYWAVTGRGVIERGNVDGTDRMILVHQLSHPWGIAVHDSFLYYTDEQYEVIERVDKATGANKIVLRDNVPNLRGLQVYHRRNAAESSNGCSNNMNACQQICLPVPGGLFSCACATGFKLNPDNRSCSPYNSFIVVSMLSAIRGFSLELSDHSETMVPVAGQGRNALHVDVDVSSGFIYWCDFSSSVASDNAIRRIKPDGSSLMNIVTHGIGENGVRGIAVDWVAGNLYFTNAFVSETLIEVLRINTTYRRVLLKVTVDMPRHIVVDPKNRYLFWADYGQRPKIERSFLDCTNRTVLVSEGIVTPRGLAVDRSDGYVYWVDDSLDIIARIRINGENSEVIRYGSRYPTPYGITVFENSIIWVDRNLKKIFQASKEPENTEPPTVIRDNINWLRDVTIFDKQVQPRSPAEVNNNPCLENNGGCSHLCFALPGLHTPKCDCAFGTLQSDGKNCAISTENFLIFALSNSLRSLHLDPENHSPPFQTINVERTVMSLDYDSVSDRIYFTQNLASGVGQISYATLSSGIHTPTVIASGIGTADGIAFDWITRRIYYSDYLNQMINSMAEDGSNRTVIARVPKPRAIVLDPCQGYLYWADWDTHAKIERATLGGNFRVPIVNSSLVMPSGLTLDYEEDLLYWVDASLQRIERSTLTGVDREVIVNAAVHAFGLTLYGQYIYWTDLYTQRIYRANKYDGSGQIAMTTNLLSQPRGINTVVKNQKQQCNNPCEQFNGGCSHICAPGPNGAECQCPHEGNWYLANNRKHCIVDNGERCGASSFTCSNGRCISEEWKCDNDNDCGDGSDEMESVCALHTCSPTAFTCANGRCVQYSYRCDYYNDCGDGSDEAGCLFRDCNATTEFMCNNRRCIPREFICNGVDNCHDNNTSDEKNCPDRTCQSGYTKCHNSNICIPRVYLCDGDNDCGDNSDENPTYCTTHTCSSSEFQCASGRCIPQHWYCDQETDCFDASDEPASCGHSERTCLADEFKCDGGRCIPSEWICDGDNDCGDMSDEDKRHQCQNQNCSDSEFLCVNDRPPDRRCIPQSWVCDGDVDCTDGYDENQNCTRRTCSENEFTCGYGLCIPKIFRCDRHNDCGDYSDERGCLYQTCQQNQFTCQNGRCISKTFVCDEDNDCGDGSDELMHLCHTPEPTCPPHEFKCDNGRCIEMMKLCNHLDDCLDNSDEKGCGINECHDPSISGCDHNCTDTLTSFYCSCRPGYKLMSDKRTCVDIDECTEMPFVCSQKCENVIGSYICKCAPGYLREPDGKTCRQNSNIEPYLIFSNRYYLRNLTIDGYFYSLILEGLDNVVALDFDRVEKRLYWIDTQRQVIERMFLNKTNKETIINHRLPAAESLAVDWVSRKLYWLDARLDGLFVSDLNGGHRRMLAQHCVDANNTFCFDNPRGLALHPQYGYLYWADWGHRAYIGRVGMDGTNKSVIISTKLEWPNGITIDYTNDLLYWADAHLGYIEYSDLEGHHRHTVYDGALPHPFAITIFEDTIYWTDWNTRTVEKGNKYDGSNRQTLVNTTHRPFDIHVYHPYRQPIVSNPCGTNNGGCSHLCLIKPGGKGFTCECPDDFRTLQLSGSTYCMPMCSSTQFLCANNEKCIPIWWKCDGQKDCSDGSDELALCPQRFCRLGQFQCSDGNCTSPQTLCNAHQNCPDGSDEDRLLCENHHCDSNEWQCANKRCIPESWQCDTFNDCEDNSDEDSSHCASRTCRPGQFRCANGRCIPQAWKCDVDNDCGDHSDEPIEECMSSAHLCDNFTEFSCKTNYRCIPKWAVCNGVDDCRDNSDEQGCEERTCHPVGDFRCKNHHCIPLRWQCDGQNDCGDNSDEENCAPRECTESEFRCVNQQCIPSRWICDHYNDCGDNSDERDCEMRTCHPEYFQCTSGHCVHSELKCDGSADCLDASDEADCPTRFPDGAYCQATMFECKNHVCIPPYWKCDGDDDCGDGSDEELHLCLDVPCNSPNRFRCDNNRCIYSHEVCNGVDDCGDGTDETEEHCRKPTPKPCTEYEYKCGNGHCIPHDNVCDDADDCGDWSDELGCNKGKERTCAENICEQNCTQLNEGGFICSCTAGFETNVFDRTSCLDINECEQFGTCPQHCRNTKGSYECVCADGFTSMSDRPGKRCAAEGSSPLLLLPDNVRIRKYNLSSERFSEYLQDEEYIQAVDYDWDPKDIGLSVVYYTVRGEGSRFGAIKRAYIPNFESGRNNLVQEVDLKLKYVMQPDGIAVDWVGRHIYWSDVKNKRIEVAKLDGRYRKWLISTDLDQPAAIAVNPKLGLMFWTDWGKEPKIESAWMNGEDRNILVFEDLGWPTGLSIDYLNNDRIYWSDFKEDVIETIKYDGTDRRVIAKEAMNPYSLDIFEDQLYWISKEKGEVWKQNKFGQGKKEKTLVVNPWLTQVRIFHQLRYNKSVPNLCKQICSHLCLLRPGGYSCACPQGSSFIEGSTTECDAAIELPINLPPPCRCMHGGNCYFDETDLPKCKCPSGYTGKYCEMAFSKGISPGTTAVAVLLTILLIVVIGALAIAGFFHYRRTGSLLPALPKLPSLSSLVKPSENGNGVTFRSGADLNMDIGVSGFGPETAIDRSMAMSEDFVMEMGKQPIIFENPMYSARDSAVKVVQPIQVTVSENVDNKNYGSPINPSEIVPETNPTSPAADGTQVTKWNLFKRKSKQTTNFENPIYAQMENEQKESVAATPPPSPSLPAKPKPPSRRDPTPTYSATEDTFKDTANLVKEDSEV

SEQ ID NO: 4 - DNA, 15657 bp, Homo sapiens
  source 1..15657: mol_type = other DNA
aggcttttggccactaggagctggcggaggtgcagacctaaaggagcgttcgctagcagaggcgctgccggtgcggtgtgctacgcgcgcccacctcccggggaaggaacggcgaggccggggaccgtcgcggagatggatcgcgggccggcagcagtggcgtgcacgctgctcctggctctcgtcgcctgcctagcgccggccagtggccaagaatgtgacagtgcgcattttcgctgtggaagtgggcattgcatccctgcagactggaggtgtgatgggaccaaagactgttcagatgacgcggatgaaattggctgcgctgttgtgacctgccagcagggctatttcaagtgccagagtgagggacaatgcatccccaactcctgggtgtgtgaccaagatcaagactgtgatgatggctcagatgaacgtcaagattgctcacaaagtacatgctcaagtcatcagataacatgctccaatggtcagtgtatcccaagtgaatacaggtgcgaccacgtcagagactgccccgatggagctgatgagaatgactgccagtacccaacatgtgagcagcttacttgtgacaatggggcctgctataacaccagtcagaagtgtgattggaaagttgattgcagggactcctcagatgaaatcaactgcactgagatatgcttgcacaatgagttttcatgtggcaatggagagtgtatccctcgtgcttatgtctgtgaccatgacaatgattgccaagacggcagtgacgaacatgcttgcaactatccgacctgcggtggttaccagttcacttgccccagtggccgatgcatttatcaaaactgggtttgtgatggagaagatgactgtaaagataatggagatgaagatggatgtgaaagcggtcctcatgatgttcataaatgttccccaagagaatggtcttgcccagagtcgggacgatgcatctccatttataaagtttgtgatgggattttagattgcccaggaagagaagatgaaaacaacactagtaccggaaaatactgtagtatgactctgtgctctgccttgaactgccagtaccagtgccatgagacgccgtatggaggagcgtgtttttgtcccccaggttatatcatcaaccacaatgacagccgtacctgtgttgagtttgatgattgccagatatggggaatttgtgaccagaagtgtgaaagccgacctggccgtcacctgtgccactgtgaagaagggtatatcttggagcgtggacagtattgcaaagctaatgattcctttggcgaggcctccattatcttctccaatggtcgggatttgttaattggtgatattcatggaaggagcttccggatcctagtggagtctcagaatcgtggagtggccgtgggtgtggctttccactatcacctgcaaagagttttttggacagacaccgtgcaaaataaggttttttcagttgacattaatggtttaaatatccaagaggttctcaatgtttctgttgaaaccccagagaacctggctgtggactgggttaataataaaatctatctagtggaaaccaaggtcaaccgcatagatatggtaaatttggatggaagctatcgggttacccttataactgaaaacttggggcatcctagaggaattgccgtggacccaactgttggttatttatttttctcagattgggagagcctttctggggaacctaagctggaaagggcattcatggatggcagcaaccgtaaagacttggtgaaaacaaagctgggatggcctgctggggtaactctggatatgatatcgaagcgtgtttactgggttgactctcggtttgattacattgaaactgtaacttatgatggaattcaaaggaagactgtagttcatggaggctccctcattcctcatccctttggagtaagcttatttgaaggtcaggtgttctttacagattggacaaagatggccgtgctgaaggcaaacaagttcacagagaccaacccacaagtgtactaccaggcttccctgaggccctatggagtgactgtttaccattccctcagacagccctatgctaccaatccgtgtaaagataacaatgggggctgtgagcaggtctgtgtcctcagccacagaacagataatgatggtttgggtttccgttgcaagtgcacattcggcttccaactggatacagatgagcgccactgcattgctgttcagaatttcctcattttttcatcccaagttgctattcgtgggatcccgttcaccttgtctacccaggaagatgtcatggttccagtttcggggaatccttctttctttgtcgggattgattttgacgcccaggacagcactatctttttttcagatatgtcaaaacacatgatttttaagcaaaagattgatggcacaggaagagaaattctcgcagctaacagggtggaaaatgttgaaagtttggcttttgattggatttcaaagaatctctattggacagactctcattacaagagtatcagtgtcatgaggctagctgataaaacgagacgcacagtagttcagtatttaaataacccacggtcggtggtagttcatccttttgccgggtatctattcttcactgattggttccgtcctgctaaaattatgagagcatggagtgacggatctcacctcttgcctgtaataaacactactcttggatggcccaatggcttggccatcgattgggctgcttcacgattgtactgggtagatgcctattttgataaaattgagcacagcacctttgatggtttagacagaagaagactgggccatatagagcagatgacacatccgtttggacttgccatctttggagagcatttattttttactgactggagactgggtgccattattcgagtcaggaaagcagatggtggagaaatgacagttatccgaagtggcattgcttacatactgcatttgaaatcgtatgatgtcaacatccagactggttctaacgcctgtaatcaacccacgcatcctaacggtgactgcagccacttctgcttcccggtgccaaatttccagcgagtgtgtgggtgcccttatggaatgaggctggcttccaatcacttgacatgcgagggggacccaaccaatgaaccacccacagagcagtgtggcttattttccttcccctgtaaaaatggcagatgtgtgcccaattactatctctgtgatggagtcgatgattgtcatgataacagtgatgagcaactatgtggcacacttaataatacctgttcatcttcggcgttcacctgtggccatggggagtgcattcctgcacactggcgctgtgacaaacgcaacgactgtgtggatggcagtgatgagcacaactgccccacccacgcacctgcttcctgccttgacacccaatacacctgtgataatcaccagtgtatctcaaagaactgggtctgtgacacagacaatgattgtggggatggatctgatgaaaagaactgcaattcgacagagacatgccaacctagtcagtttaattgccccaatcatcgatgtattgacctatcgtttgtctgtgatggtgacaaggattgtgttgatggatctgatgaggttggttgtgtattaaactgtactgcttctcaattcaagtgtgccagtggggataaatgtattggcgtcacaaatcgttgtgatggtgtttttgattgcagtgacaactcggatgaagcaggctgtccaaccaggcctcctggtatgtgccactcagatgaatttcagtgccaagaagatggtatctgcatcccgaacttctgggaatgtgatgggcatccagactgcctctatggatctgatgagcacaatgcctgtgtccccaagacttgcccttcatcatatttccactgtgacaacggaaactgcatccacagggcatggctctgtgatcgggacaatgactgcggggatatgagtgatgagaaggactgccctactcagccctttcgctgtcctagttggcaatggcagtgtcttggccataacatctgtgtgaatctgagtgtagtgtgtgatggcatctttgactgccccaatgggacagatgagtccccactttgcaatgggaacagctgctcagatttcaatggtggttgtactcacgagtgtgttcaagagccctttggggctaaatgcctatgtccattgggattcttacttgccaatgattctaagacctgtgaagacatagatgaatgtgatattctaggctcttgtagccagcactgttacaatatgagaggttctttccggtgctcgtgtgatacaggctacatgttagaaagtgatgggaggacttgcaaagttacagcatctgagagtctgctgttacttgtggcaagtcagaacaaaattattgccgacagtgtcacctcccaggtccacaatatctattcattggtcgagaatggttcttacattgtagctgttgattttgattcaattagtggtcgtatcttttggtctgatgcaactcagggtaaaacctggagtgcgtttcaaaatggaacggacagaagagtggtatttgacagtagcatcatcttgactgaaactattgcaatagattgggtaggtcgtaatctttactggacagactatgctctggaaacaattgaagtctccaaaattgatgggagccacaggactgtgctgattagtaaaaacctaacaaatccaagaggactagcattagatcccagaatgaatgagcatctactgttctggtctgactggggccaccaccctcgcatcgagcgagccagcatggacggcagcatgcgcactgtcattgtccaggacaagatcttctggccctgcggcttaactattgactaccccaacagactgctctacttcatggactcctatcttgattacatggacttttgtgattataatggacaccatcggagacaggtgatagccagtgatttgattatacggcacccctatgccctaactctctttgaagactctgtgtactggactgaccgtgctactcgtcgggttatgcgagccaacaagtggcatggagggaaccagtcagttgtaatgtataatattcaatggccccttgggattgttgcggttcatccttcgaaacaaccaaattccgtgaatccatgtgccttttcccgctgcagccatctctgcctgctttcctcacaggggcctcatttttactcctgtgtttgtccttcaggatggagtctgtctcctgatctcctgaattgcttgagagatgatcaacctttcttaataactgtaaggcaacatataatttttggaatctcccttaatcctgaggtgaagagcaatgatgctatggtccccatagcagggatacagaatggtttagatgttgaatttgatgatgctgagcaatacatctattgggttgaaaatccaggtgaaattcacagagtgaagacagatggcaccaacaggacagtatttgcttctatatctatggtggggccttctatgaacctggccttagattggatttcaagaaacctttattctaccaatcctagaactcagtcaatcgaggttttgacactccacggagatatcagatacagaaaaacattgattgccaatgatgggacagctcttggagttggctttccaattggcataactgttgatcctgctcgtgggaagctgtactggtcagaccaaggaactgacagtggggttcctgccaagatcgccagtgctaacatggatggcacatctgtgaaaactctctttactgggaacctcgaacacctggagtgtgtcactcttgacatcgaagagcagaaactctactgggcagtcactggaagaggagtgattgaaagaggaaacgtggatggaacagatcgaatgatcctggtacaccagctttcccacccctggggaattgcagtccatgattctttcctttattatactgatgaacagtatgaggtcattgaaagagttgataaggccactggggccaacaaaatagtcttgagagataatgttccaaatctgaggggtcttcaagtttatcacagacgcaatgccgccgaatcctcaaatggctgtagcaacaacatgaatgcctgtcagcagatttgcctgcctgtaccaggaggattgttttcctgcgcctgtgccactggatttaaactcaatcctgataatcggtcctgctctccatataactctttcattgttgtttcaatgctgtctgcaatcagaggctttagcttggaattgtcagatcattcagaaaccatggtgccggtggcaggccaaggacgaaacgcactgcatgtggatgtggatgtgtcctctggctttatttattggtgtgattttagcagctcagtggcatctgataatgcgatccgtagaattaaaccagatggatcttctctgatgaacattgtgacacatggaataggagaaaatggagtccggggtattgcagtggattgggtagcaggaaatctttatttcaccaatgcctttgtttctgaaacactgatagaagttctgcggatcaatactacttaccgccgtgttcttcttaaagtcacagtggacatgcctaggcatattgttgtagatcccaagaacagatacctcttctgggctgactatgggcagagaccaaagattgagcgttctttccttgactgtaccaatcgaacagtgcttgtgtcagagggcattgtcacaccacggggcttggcagtggaccgaagtgatggctacgtttattgggttgatgattctttagatataattgcaaggattcgtatcaatggagagaactctgaagtgattcgttatggcagtcgttacccaactccttatggcatcactgtttttgaaaattctatcatatgggtagataggaatttgaaaaagatcttccaagccagcaaggaaccagagaacacagagccacccacagtgataagagacaatatcaactggctaagagatgtgaccatctttgacaagcaagtccagccccggtcaccagcagaggtcaacaacaacccttgcttggaaaacaatggtgggtgctctcatctctgctttgctctgcctggattgcacaccccaaaatgtgactgtgcctttgggaccctgcaaagtgatggcaagaattgtgccatttcaacagaaaatttcctcatctttgccttgtctaattccttgagaagcttacacttggaccctgaaaaccatagcccacctttccaaacaataaatgtggaaagaactgtcatgtctctagactatgacagtgtaagtgatagaatctacttcacacaaaatttagcctctggagttggacagatttcctatgccaccctgtcttcagggatccatactccaactgtcattgcttcaggtatagggactgctgatggcattgcctttgactggattactagaagaatttattacagtgactacctcaaccagatgattaattccatggctgaagatgggtctaaccgcactgtgatagcccgcgttccaaaaccaagagcaattgtgttagatccctgccaagggtacctgtactgggctgactgggatacacatgccaaaatcgagagagccacattgggaggaaacttccgcgtacccattgtgaacagcagtctggtcatgcccagtgggctgactctggactatgaagaggaccttctctactgggtggatgctagtctgcagaggattgaacgcagcactctgacgggcgtggatcgtgaagtcattgtcaatgcagccgttcatgcttttggcttgactctctatggccagtatatttactggactgacttgtacacacaaagaatttaccgagctaacaaatatgacgggtcaggtcagattgcaatgaccacaaatttgctctcccagcccaggggaatcaacactgttgtgaagaaccagaaacaacagtgtaacaatccttgtgaacagtttaatgggggctgcagccatatctgtgcaccaggtccaaatggtgccgagtgccagtgtccacatgagggcaactggtatttggccaacaacaggaagcactgcattgtggacaatggtgaacgatgtggtgcatcttccttcacctgctccaatgggcgctgcatctcggaagagtggaagtgtgataatgacaacgactgtggggatggcagtgatgagatggaaagtgtctgtgcacttcacacctgctcaccgacagccttcacctgtgccaatgggcgatgtgtccaatactcttaccgctgtgattactacaatgactgtggtgatggcagtgatgaggcagggtgcctgttcagggactgcaatgccaccacggagtttatgtgcaataacagaaggtgcatacctcgtgagtttatctgcaatggtgtagacaactgccatgataataacacttcagatgagaaaaattgccctgatcgcacttgccagtctggatacacaaaatgtcataattcaaatatttgtattcctcgcgtttatttgtgtgacggagacaatgactgtggagataacagtgatgaaaaccctacttattgcaccactcacacgtgcagcagcagtgagttccaatgcgcatctgggcgctgtattcctcaacattggtattgtgatcaagaaacagattgttttgatgcctctgatgaacctgcctcttgtggtcactctgagcgaacatgcctagctgatgagttcaagtgtgatggtgggaggtgcatcccaagcgaatggatctgtgacggtgataatgactgtggggatatgagtgacgaggataaaaggcaccagtgtcagaatcaaaactgctcggattccgagtttctctgtgtaaatgacagacctccggacaggaggtgcattccccagtcttgggtctgtgatggcgatgtggattgtactgacggctacgatgagaatcagaattgcaccaggagaacttgctctgaaaatgaattcacctgtggttacggactgtgtatcccaaagatattcaggtgtgaccggcacaatgactgtggtgactatagcgacgagaggggctgcttataccagacttgccaacagaatcagtttacctgtcagaacgggcgctgcattagtaaaaccttcgtctgtgatgaggataatgactgtggagacggatctgatgagctgatgcacctgtgccacaccccagaacccacgtgtccacctcacgagttcaagtgtgacaatgggcgctgcatcgagatgatgaaactctgcaaccacctagatgactgtttggacaacagcgatgagaaaggctgtggcattaatgaatgccatgacccttcaatcagtggctgcgatcacaactgcacagacaccttaaccagtttctattgttcctgtcgtcctggttacaagctcatgtctgacaagcggacttgtgttgatattgatgaatgcacagagatgccttttgtctgtagccagaagtgtgagaatgtaataggctcctacatctgtaagtgtgccccaggctacctccgagaaccagatggaaagacctgccggcaaaacagtaacatcgaaccctatctcatttttagcaaccgttactatttgagaaatttaactatagatggctatttttactccctcatcttggaaggactggacaatgttgtggcattagattttgaccgagtagagaagagattgtattggattgatacacagaggcaagtcattgagagaatgtttctgaataagacaaacaaggagacaatcataaaccacagactaccagctgcagaaagtctggctgtagactgggtttccagaaagctctactggttggatgcccgcctggatggcctctttgtctctgacctcaatggtggacaccgccgcatgctggcccagcactgtgtggatgccaacaacaccttctgctttgataatcccagaggacttgcccttcaccctcaatatgggtacctctactgggcagactggggtcaccgcgcatacattgggagagtaggcatggatggaaccaacaagtctgtgataatctccaccaagttagagtggcctaatggcatcaccattgattacaccaatgatctactctactgggcagatgcccacctgggttacatagagtactctgatttggagggccaccatcgacacacggtgtatgatggggcactgcctcaccctttcgctattaccatttttgaagacactatttattggacagattggaatacaaggacagtggaaaagggaaacaaatatgatggatcaaatagacagacactggtgaacacaacacacagaccatttgacatccatgtgtaccatccatataggcagcccattgtgagcaatccctgtggtaccaacaatggtggctgttctcatctctgcctcatcaagccaggaggaaaagggttcacttgcgagtgtccagatgacttccgcacccttcagctgagtggcagcacctactgcatgcccatgtgctccagcacccagttcctgtgcgctaacaatgaaaagtgcattcctatctggtggaaatgtgatggacagaaagactgctcagatggctctgatgaactggccctttgcccgcagcgcttctgccgactgggacagttccagtgcagtgacggcaactgcaccagcccgcagactttatgcaatgctcaccaaaattgccctgatgggtctgatgaagaccgtcttctttgtgagaatcaccactgtgactccaatgaatggcagtgcgccaacaaacgttgcatcccagaatcctggcagtgtgacacatttaacgactgtgaggataactcagatgaagacagttcccactgtgccagcaggacctgccggccgggccagtttcggtgtgctaatggccgctgcatcccgcaggcctggaagtgtgatgtggataatgattgtggagaccactcggatgagcccattgaagaatgcatgagctctgcccatctctgtgacaacttcacagaattcagctgcaaaacaaattaccgctgcatcccaaagtgggccgtgtgcaatggtgtagatgactgcagggacaacagtgatgagcaaggctgtgaggagaggacatgccatcctgtgggggatttccgctgtaaaaatcaccactgcatccctcttcgttggcagtgtgatgggcaaaatgactgtggagataactcagatgaggaaaactgtgctccccgggagtgcacagagagcgagtttcgatgtgtcaatcagcagtgcattccctcgcgatggatctgtgaccattacaacgactgtggggacaactcagatgaacgggactgtgagatgaggacctgccatcctgaatattttcagtgtacaagtggacattgtgtacacagtgaactgaaatgcgatggatccgctgactgtttggatgcgtctgatgaagctgattgtcccacacgctttcctgatggtgcatactgccaggctactatgttcgaatgcaaaaaccatgtttgtatcccgccatattggaaatgtgatggcgatgatgactgtggcgatggttcagatgaagaacttcacctgtgcttggatgttccctgtaattcaccaaaccgtttccggtgtgacaacaatcgctgcatttatagtcatgaggtgtgcaatggtgtggatgactgtggagatggaactgatgagacagaggagcactgtagaaaaccgacccctaaaccttgtacagaatatgaatataagtgtggcaatgggcattgcattccacatgacaatgtgtgtgatgatgccgatgactgtggtgactggtccgatgaactgggttgcaataaaggaaaagaaagaacatgtgctgaaaatatatgcgagcaaaattgtacccaattaaatgaaggaggatttatctgctcctgtacagctgggttcgaaaccaatgtttttgacagaacctcctgtctagatatcaatgaatgtgaacaatttgggacttgtccccagcactgcagaaataccaaaggaagttatgagtgtgtctgtgctgatggcttcacgtctatgagtgaccgccctggaaaacgatgtgcagctgagggtagctctcctttgttgctactgcctgacaatgtccgaattcgaaaatataatctctcatctgagaggttctcagagtatcttcaagatgaggaatatatccaagctgttgattatgattgggatcccaaggacataggcctcagtgttgtgtattacactgtgcgaggggagggctctaggtttggtgctatcaaacgtgcctacatccccaactttgaatccggccgcaataatcttgtgcaggaagttgacctgaaactgaaatacgtaatgcagccagatggaatagcagtggactgggttggaaggcatatttactggtcagatgtcaagaataaacgcattgaggtggctaaacttgatggaaggtacagaaagtggctgatttccactgacctggaccaaccagctgctattgctgtgaatcccaaactagggcttatgttctggactgactggggaaaggaacctaaaatcgagtctgcctggatgaatggagaggaccgcaacatcctggttttcgaggaccttggttggccaactggcctttctatcgattatttgaacaatgaccgaatctactggagtgacttcaaggaggacgttattgaaaccataaaatatgatgggactgataggagagtcattgcaaaggaagcaatgaacccttacagcctggacatctttgaagaccagttatactggatatctaaggaaaagggagaagtatggaaacaaaataaatttgggcaaggaaagaaagagaaaacgctggtagtgaacccttggctcactcaagttcgaatctttcatcaactcagatacaataagtcagtgcccaacctttgcaaacagatctgcagccacctctgccttctgagacctggaggatacagctgtgcctgtccccaaggctccagctttatagaggggagcaccactgagtgtgatgcagccatcgaactgcctatcaacctgccccccccatgcaggtgcatgcacggaggaaattgctattttgatgagactgacctccccaaatgcaagtgtcctagcggctacaccggaaaatattgtgaaatggcgttttcaaaaggcatctctccaggaacaaccgcagtagctgtgctgttgacaatcctcttgatcgtcgtaattggagctctggcaattgcaggattcttccactatagaaggaccggctcccttttgcctgctctgcccaagctgccaagcttaagcagtctcgtcaagccctctgaaaatgggaatggggtgaccttcagatcaggggcagatcttaacatggatattggagtgtctggttttggacctgagactgctattgacaggtcaatggcaatgagtgaagactttgtcatggaaatggggaagcagcccataatatttgaaaacccaatgtactcagccagagacagtgctgtcaaagtggttcagccaatccaggtgactgtatctgaaaatgtggataataagaattatggaagtcccataaacccttctgagatagttccagagacaaacccaacttcaccagctgctgatggaactcaggtgacaaaatggaatctcttcaaacgaaaatctaaacaaactaccaactttgaaaatccaatctatgcacagatggagaacgagcaaaaggaaagtgttgctgcgacaccacctccatcaccttcgctccctgctaagcctaagcctccttcgagaagagacccaactccaacctattctgcaacagaagacacttttaaagacaccgcaaatcttgttaaagaagactctgaagtatagctataccagctatttagggaataattagaaacacacttttgcacatatattttttacaaacagatgaaaaaagttaacattcagtactttatgaaaaaaatatatttttccctgtttgcctatagttggaggtatcctgtgtgtctttttttacttatgccgtctcatatttttacaaataattatcacaatgtactatatgtatatctttgcactgaagttgtctgaaggtaatactataaatatattgtatatttgtaaattttggaaagattatcctgttactgaatttgctaataaagatgtctgctgatttggttggtgatcattatagtaaatgatccaacaagaaaaggaattgactggggacctttagccgtgtctaaagaagaggcaccactcatatttcctataaaattatctaggaaaggaatccaggccccgctcttgggtccatttttacacattagcacttaattaatgttcaatattacatgtcaatttgattaatggctatgttgataggggccactatgtgttgtatagacatctggacttgactgtagactcctcagataatacagaaggtaggaaaagcaattcagtttggcccttctgtgtgttggcattgtctaaccagaactctctgtttcatgtgtgttctctcactagctgccaagacaacatttttatttgtgatgtctatgaggaaatcccatatcattaagtgccagtgtcctgcattgagtttgtggttaattaaatgagctcttctgctgatggaccctggagcaatttctcccctcacctgacattcaaggtggtcacctgccctagtagttggagctcagtagctgaatttctgaaaccaaatctgtgtcttcataaaataaggtgcaaaaaaaaaaaataccagttaagtaaagcctcaactgggtttttgtttctatgaaaatatcattataatcactatttatttcctaagttgaacctgaatagaaagggaaaccattcttattaagctttttattaggccctgtggctaaatgtgtacatttatattagaatgtactgtacagtccagatcttttctttaattcttattggttttttttttttttttttttttagagatggagtcttgctatattgccaaggctgatcttgaagtcctgggctcaagtgatcctcccacctcagcctcctgagtggttggggttacgggcgtgagccactgtgcctggcttccagctctcctcttaaatagtgggtatagtctgcacaacaggaaccatggcaggaatatacactttcccatagcaaatagcatacctgactctctgtgctaatattgcacatttgttaaacaatgaatgaatggatggatggatggatggatgaatgaatgaaacatatactactgattattttattccagagttctcaaaatatttgttgctgatattttgagtgctgactgtaattactttgattagataaacaactggaaataatgctgctgaaaaagttctaataaatgtgtattttatcaga

SEQ ID NO: 5 - protein, 3623 aa, Homo sapiens
MMNMSLPFLWSLLTLLIFAEVNGEAGELELQRQKRSINLQQPRMATERGNLVFLTGSAQNIEFRTGSLGKIKLNDEDLSECLHQIQKNKEDIIELKGSAIGLPQNISSQIYQLNSKLVDLERKFQGLQQTVDKKVCSSNPCQNGGTCLNLHDSFFCICPPQWKGPLCSADVNECEIYSGTPLSCQNGGTCVNTMGSYSCHCPPETYGPQCASKYDDCEGGSVARCVHGICEDLMREQAGEPKYSCVCDAGWMFSPNSPACTLDRDECSFQPGPCSTLVQCFNTQGSFYCGACPTGWQGNGYICEDINECEINNGGCSVAPPVECVNTPGSSHCQACPPGYQGDGRVCTLTDICSVSNGGCHPDASCSSTLGSLPLCTCLPGYTGNGYGPNGCVQLSNICLSHPCLNGQCIDTVSGYFCKCDSGWTGVNCTENINECLSNPCLNGGTCVDGVDSFSCECTRLWTGALCQVPQQVCGESLSGINGSFSYRSPDVGYVHDVNCFWVIKTEMGKVLRITFTFFRLESMDNCPHEFLQVYDGDSSSAFQLGRFCGSSLPHELLSSDNALYFHLYSEHLRNGRGFTVRWETQQPECGGILTGPYGSIKSPGYPGNYPPGRDCVWIVVTSPDLLVTFTFGTLSLEHHDDCNKDYLEIRDGPLYQDPLLGKFCTTFSVPPLQTTGPFARIHFHSDSQISDQGFHITYLTSPSDLRCGGNYTDPEGELFLPELSGPFTHTRQCVYMMKQPQGEQIQINFTHVELQCQSDSSQNYIEVRDGETLLGKVCGNGTISHIKSITNSVWIRFKIDASVEKASFRAVYQVACGDELTGEGVIRSPFFPNVYPGERTCRWTIHQPQSQVILLNFTVFEIGSSAHCETDYVEIGSSSILGSPENKKYCGTDIPSFITSVYNFLYVTFVKSSSTENHGFMAKFSAEDLACGEILTESTGTIQSPGHPNVYPHGINCTWHILVQPNHLIHLMFETFHLEFHYNCTNDYLEVYDTDSETSLGRYCGKSIPPSLTSSGNSLMLVFVTDSDLAYEGFLINYEAISAATACLQDYTDDLGTFTSPNFPNNYPNNWECIYRITVRTGQLIAVHFTNFSLEEAIGNYYTDFLEIRDGGYEKSPLLGIFYGSNLPPTIISHSNKLWLKFKSDQIDTRSGFSAYWDGSSTGCGGNLTTSSGTFISPNYPMPYYHSSECYWWLKSSHGSAFELEFKDFHLEHHPNCTLDYLAVYDGPSSNSHLLTQLCGDEKPPLIRSSGDSMFIKLRTDEGQQGRGFKAEYRQTCENVVIVNQTYGILESIGYPNPYSENQHCNWTIRATTGNTVNYTFLAFDLEHHINCSTDYLELYDGPRQMGRYCGVDLPPPGSTTSSKLQVLLLTDGVGRREKGFQMQWFVYGCGGELSGATGSFSSPGFPNRYPPNKECIWYIRTDPGSSIQLTIHDFDVEYHSRCNFDVLEIYGGPDFHSPRIAQLCTQRSPENPMQVSSTGNELAIRFKTDLSINGRGFNASWQAVTGGCGGIFQAPSGEIHSPNYPSPYRSNTDCSWVIRVDRNHRVLLNFTDFDLEPQDSCIMAYDGLSSTMSRLARTCGREQLANPIVSSGNSLFLRFQSGPSRQNRGFRAQFRQACGGHILTSSFDTVSSPRFPANYPNNQNCSWIIQAQPPLNHITLSFTHFELERSTTCARDFVEILDGGHEDAPLRGRYCGTDMPHPITSFSSALTLRFVSDSSISAGGFHTTVTASVSACGGTFYMAEGIFNSPGYPDIYPPNVECVWNIVSSPGNRLQLSFISFQLEDSQDCSRDFVEIREGNATGHLVGRYCGNSFPLNYSSIVGHTLWVRFISDGSGSGTGFQATFMKIFGNDNIVGTHGKVASPFWPENYPHNSNYQWTVNVNASHVVHGRILEMDIEEIQNCYYDKLRIYDGPSIHARLIGAYCGTQTESFSSTGNSLTFHFYSDSSISGKGFLLEWFAVDAPDGVLPTIAPGACGGFLRTGDAPVFLFSPGWPDSYSNRVDCTWLIQAPDSTVELNILSLDIESHRTCAYDSLVIRDGDNNLAQQLAVLCGREIPGPIRSTGEYMFIRFTSDSSVTRAGFNASFHKSCGGYLHADRGIITSPKYPETYPSNLNCSWHVLVQSGLTIAVHFEQPFQIPNGDSSCNQGDYLVLRNGPDICSPPLGPPGGNGHFCGSHASSTLFTSDNQMFVQFISDHSNEGQGFKIKYEAKSLACGGNVYIHDADSAGYVTSPNHPHNYPPHADCIWILAAPPETRIQLQFEDRFDIEVTPNCTSNYLELRDGVDSDAPILSKFCGTSLPSSQWSSGEVMYLRFRSDNSPTHVGFKAKYSIAQCGGRVPGQSGVVESIGHPTLPYRDNLFCEWHLQGLSGHYLTISFEDFNLQNSSGCEKDFVEIWDNHTSGNILGRYCGNTIPDSIDTSSNTAVVRFVTDGSVTASGFRLRFESSMEECGGDLQGSIGTFTSPNYPNPNPHGRICEWRITAPEGRRITLMFNNLRLATHPSCNNEHVIVFNGIRSNSPQLEKLCSSVNVSNEIKSSGNTMKVIFFTDGSRPYGGFTASYTSSEDAVCGGSLPNTPEGNFTSPGYDGVRNYSRNLNCEWTLSNPNQGNSSISIHFEDFYLESHQDCQFDVLEFRVGDADGPLMWRLCGPSKPTLPLVIPYSQVWIHFVTNERVEHIGFHAKYSFTDCGGIQIGDSGVITSPNYPNAYDSLTHCSSLLEAPQGHTITLTFSDFDIEPHTTCAWDSVTVRNGGSPESPIIGQYCGNSNPRTIQSGSNQLVVTFNSDHSLQGGGFYATWNTQTLGCGGIFHSDNGTIRSPHWPQNFPENSRCSWTAITHKSKHLEISFDNNFLIPSGDGQCQNSFVKVWAGTEEVDKALLATGCGNVAPGPVITPSNTFTAVFQSQEAPAQGFSASFVSRCGSNFTGPSGYIISPNYPKQYDNNMNCTYVIEANPLSVVLLTFVSFHLEARSAVTGSCVNDGVHIIRGYSVMSTPFATVCGDEMPAPLTIAGPVLLNFYSNEQITDFGFKFSYRIISCGGVFNFSSGIITSPAYSYADYPNDMHCLYTITVSDDKVIELKFSDFDVVPSTSCSHDYLAIYDGANTSDPLLGKFCGSKRPPNVKSSNNSMLLVFKTDSFQTAKGWKMSFRQTLGPQQGCGGYLTGSNNTFASPDSDSNGMYDKNLNCVWIIIAPVNKVIHLTFNTFALEAASTRQRCLYDYVKLYDGDSENANLAGTFCGSTVPAPFISSGNFLTVQFISDLTLEREGFNATYTIMDMPCGGTYNATWTPQNISSPNSSDPDVPFSICTWVIDSPPHQQVKITVWALQLTSQDCTQNYLQLQDSPQGHGNSRFQFCGRNASAVPVFYSSMSTAMVIFKSGVVNRNSRMSFTYQIADCNRDYHKAFGNLRSPGWPDNYDNDKDCTVTLTAPQNHTISLFFHSLGIENSVECRNDFLEVRNGSNSNSPLLGKYCGTLLPNPVFSQNNELYLRFKSDSVTSDRGYEIIWTSSPSGCGGTLYGDRGSFTSPGYPGTYPNNTYCEWVLVAPAGRLVTINFYFISIDDPGDCVQNYLTLYDGPNASSPSSGPYCGGDTSIAPFVASSNQVFIKFHADYARRPSAFRLTWDS

SEQ ID NO: 6 - DNA, 11933 bp, Homo sapiens
  source 1..11933: mol_type = other DNA
atgctcagttggttggagtggcctcactcttacctgccaacctgggaggttgatgatgaacatgtctttaccttttctttggagtttgcttaccttattaatatttgctgaagtaaatggcgaagctggagaacttgagctgcagagacaaaaaagaagcatcaatctccaacagcctcgaatggctacagagagaggaaatttggtgtttcttacggggtctgctcaaaacattgagtttagaaccggatccctgggaaaaattaaattaaatgatgaagatctcagtgagtgtttacatcagatccagaaaaacaaagaagatattatagagttaaaagggagtgcaattggtctgcctcaaaatatatctagtcaaatctatcagcttaattccaagctggtggatcttgagagaaaattccaaggcttgcagcagactgttgacaaaaaggtttgcagcagcaatccttgccagaatggtggaacctgcctcaatctgcatgattcctttttttgtatctgtcccccacagtggaagggtcctctctgctcagctgatgttaacgaatgtgagatttactcaggaacacccttgagctgccagaatggaggcacatgtgttaatacaatgggaagttacagttgtcactgcccacctgagacgtacggaccccagtgtgcatccaaatatgacgactgtgaagggggttctgtggcacgctgtgtccatggcatctgtgaggatttaatgcgagagcaagctggagagcccaagtacagctgcgtctgtgatgctgggtggatgttttcacccaacagccctgcctgcacgctggacagagacgagtgcagcttccagcccgggccttgctccacacttgtgcagtgtttcaacactcaaggctctttctactgtggggcctgtccaacaggctggcaaggcaatggatatatttgcgaagatatcaatgaatgtgagataaataacggcggctgttctgtggctccacccgttgagtgtgtgaatacacctgggtcttcccactgccaggcctgtccaccagggtaccagggtgacggaagagtgtgcacactcacagacatctgctcagtcagtaatggaggctgccacccagatgcctcatgctcctcaactctaggttccttacctctctgcacgtgtctcccgggttatactggaaatggttatgggccaaatggatgtgtgcagctcagtaatatttgcctaagtcacccctgtctaaatggacaatgcatcgacactgtctctggttatttttgtaagtgtgactcaggttggacaggtgtcaactgtacagaaaacatcaatgagtgtttgagcaacccctgtttgaatggaggaacttgtgttgatggcgttgattctttcagttgtgaatgcacacgtctctggactggagctctctgtcaggttcctcagcaagtttgtggagagtccctctcaggaataaatggaagcttcagctacaggagcccggatgttggttatgttcatgatgttaactgcttctgggttatcaaaactgaaatgggaaaggtcctgcgtatcactttcacttttttccggttagaatccatggacaactgtccacacgagtttcttcaggtttatgatggagattcctcttctgcttttcaacttggaagattttgtggctccagcctccctcatgaactcctcagcagtgacaatgctctctattttcatctctattctgaacatttaagaaatgggagaggctttacagtaagatgggaaacacagcaaccagagtgtggaggtatcctgactggtccttacggttctattaagtctccggggtatcctggaaactatcccccaggaagagattgtgtctggattgttgtaactagtcctgacctcctggtaacatttacttttgggaccttgagcctcgagcaccatgatgactgcaacaaagattaccttgagattcgagatggtcctttgtatcaggacccccttcttgggaagttctgcaccactttctctgtcccaccgctccagactactggcccctttgccagaattcacttccattcagactcccagattagtgaccaaggcttccatatcacctacttaacatcaccttcggatctgcgttgtggtgggaactacacggacccagagggtgaactcttcttgcctgagttgtctgggcctttcactcacaccaggcaatgcgtctatatgatgaagcagccccagggagaacaaatacaaatcaacttcacccacgtggagctgcaatgccagagtgacagttctcagaattacattgaggttcgagatggtgaaaccttacttggaaaagtctgtggcaacggaaccatctctcacattaaatccattactaatagtgtctggatcaggtttaaaatagatgcttctgttgaaaaagctagtttcagagctgtttatcaagtcgcttgcggggatgaattaactggagaaggggtcattcgctcgcctttttttcctaacgtgtatcctggagaaagaacctgtaggtggaccatccaccagccccaaagccaagtcattctcctcaacttcactgtctttgaaattggaagttctgcccactgtgaaacagattatgttgagattggtagcagttccattttgggttctcctgaaaataaaaagtattgcggtacagacataccttcatttataacatctgtgtacaattttctttatgtcacattcgtgaaaagttcttctactgaaaaccatggtttcatggctaagttcagtgctgaggatttggcatgtggagaaattcttacagaatcaacagggaccattcaaagtcctggccatccaaatgtctacccccacggtatcaactgtacttggcatatattagtccaacctaatcacctgattcatttaatgttcgaaacatttcatctggagtttcattacaattgcacaaacgactacttggaagtttatgacaccgactctgagacatcccttggaagatactgtggaaagtcgatcccgccatctctcacaagcagtggtaactcattgatgctggtgtttgtgactgactccgacctcgcttatgaaggcttcttaataaactatgaagcaatcagtgcagcaacagcatgtttgcaagactacacagatgatttggggacattcacttctccaaacttccccaataattatcccaacaactgggaatgcatttatcggatcacagtgagaactggccaactgattgcagtgcacttcacaaacttctccttggaggaagccattggaaactattatacagattttctggaaatcagagatggaggctatgaaaaatcaccattgctgggaatattctatggctcaaatctacccccaacaatcatctctcatagtaacaaactatggttaaaatttaagagtgaccaaatagacacaaggtctggattctcagcttactgggatgggtcatcaacaggttgcgggggtaatctcaccacttcaagcggcacgttcatatctcccaactacccgatgccctattaccacagctctgaatgctactggtggttgaaatctagccacggcagcgcatttgaactggaattcaaagactttcacttggagcatcatccaaactgcactttagattacctggctgtatatgatggcccaagtagcaactctcatctgctaactcagctttgtggggatgagaaaccccctcttattcgttctagtggagacagcatgtttataaaactgaggacagatgaaggtcagcaaggacgtggcttcaaggctgaataccggcagacatgtgagaatgtggtaatagtcaatcaaacctatggcatcttagagagtatagggtatccgaatccttattctgaaaatcagcattgcaactggaccatccgggcaacaacaggcaacactgtgaactacacatttttagcatttgacttggaacatcacataaactgctccacagattatttagagctctatgatggaccacggcagatgggacgctactgtggagtagacctgccccctccagggagtactacaagctccaagcttcaagtgctgctccttacagatggggttggccgccgtgagaaaggatttcagatgcagtggtttgtttacggttgtggtggagagctgtctggggccacaggctccttcagcagccccgggttccccaacaggtatccaccaaacaaggagtgtatctggtacattaggacggaccccgggagtagcattcagctcaccatccatgacttcgatgtggagtatcattcaaggtgcaactttgatgtcttggagatctatggaggccccgatttccactctcccagaatagcccaactgtgtacccagagatcacctgagaaccccatgcaggtctccagcactggaaatgagctagcaattcgattcaagaccgacttgtccataaatgggagaggcttcaatgcgtcatggcaagcagtcactggaggttgtggtgggattttccaggctcccagtggagagattcattctccaaattaccccagtccttataggagcaacacagactgttcttgggtcattcgggttgacagaaatcatcgtgttctcttgaacttcactgactttgatcttgaaccacaagactcttgtattatggcatacgatggcttaagctccacaatgtcccgccttgccaggacgtgtggaagggagcagctggctaaccccatcgtctcctcaggaaacagcctcttcttgagatttcagtctggcccttccagacagaacagaggcttccgagctcaattcaggcaagcctgcggaggccacatcctcaccagctcatttgatactgtttcctctccacggttccctgccaattatccaaacaatcagaactgcagctggatcattcaagcgcaacctccattaaatcatatcaccctctcttttacccactttgaacttgaaagaagcacaacgtgtgcacgtgactttgtagaaattttggatggcggccacgaagacgcgcccctccgaggccgttactgtggcaccgacatgccccatcctatcacatccttcagcagcgccctgacgctgagattcgtctctgattctagcatcagtgctgggggtttccacaccacggtcaccgcatcagtgtcggcttgtggtggaacgttctacatggctgaaggcatcttcaacagccctggctacccagacatttatccccctaatgtggaatgtgtctggaacatcgtcagttcccctggcaaccggctccagctgtcttttatatctttccagttggaagactctcaggactgcagcagagattttgtggagatccgtgaaggaaatgccacgggtcacttggtgggacgatactgtggaaactccttccctctcaattattcttccatcgttggacataccctgtgggtcagatttatctcagatggttctggcagcggcacgggcttccaggccacatttatgaagatatttggcaatgataatattgtgggaactcatgggaaagtcgcctctcctttctggcctgaaaactacccacataactccaattaccaatggacagtaaatgtgaatgcatctcacgttgtccatggtagaatcttggagatggacatagaagaaatacaaaactgctattatgacaaattaaggatctatgatgggcctagcattcacgcccgcctaattggagcttactgtggtacccagactgaatctttcagctccactggaaattctttgacatttcatttttactccgactcttcaatctcagggaagggattccttctggagtggtttgcagtggatgcacctgatggtgttttacctaccattgctccaggtgcttgtggtggcttcctgaggacgggagatgcacccgtgtttctcttctccccgggctggcctgacagttacagtaatagagtggactgtacgtggctcatccaggctcccgactctaccgtggaactcaacattctttccctggacattgaatctcaccgaacgtgtgcctatgatagccttgtgatacgagatggagataataacttggcccagcagctagcagttctctgtggcagagagatccctgggcccatccggtctactggagagtacatgttcatccgcttcacctcggactccagtgtaaccagggcaggcttcaatgcatcctttcacaagagctgcggtggatatttgcatgcagacagagggatcatcacgtcccccaagtatccagagacttacccatccaacctcaactgttcttggcacgtcctggtccaaagtggcctgaccattgctgtccattttgaacagcctttccagattccaaatggagattcttcttgcaaccagggggattacttggtgctaagaaatggtcctgatatctgttctccacccttgggaccccctggaggaaatggtcatttttgtggcagtcatgcttcatcaactctgttcacctcggataatcaaatgtttgttcagtttatttctgatcacagtaatgaagggcaaggatttaaaatcaaatatgaggcaaagagtttagcctgtgggggcaacgtctacatccatgatgctgattctgctgggtatgtgacctcccccaaccaccctcataattatcccccgcacgctgattgcatttggatcttagcggctccaccggaaacacgcatacagctgcaatttgaagatcgattcgatattgaagtaacacccaactgtacttccaactaccttgagttgcgggatggagtggattcggatgcaccaatactttccaaattttgtgggacatctttgcccagcagtcagtggtcctcaggagaggttatgtatttgagatttcgatctgacaacagccccacacatgtgggattcaaggccaagtattctatagctcagtgtgggggaagagtaccagggcaaagtggtgttgttgaaagcattggacatccaacacttccatacagagacaacttattctgtgagtggcatctccaggggctctctggacactatctcaccatctcttttgaagactttaaccttcagaattcttctggctgtgaaaaagacttcgtggagatctgggacaatcatacctctggaaacatcttgggcagatactgtggaaacaccattcctgacagcatagacacttctagcaatactgctgtggtcaggtttgtcacagacggctctgtgactgcctcaggattcagactgcgatttgaatccagtatggaagagtgtggtggggatcttcagggctctattggaacatttacttctcccaactacccgaacccaaatcctcatggccggatctgcgagtggagaatcactgccccggagggaaggcggatcaccctaatgtttaacaacctgaggctggccacgcatccgtcctgcaacaatgagcatgtgatagtattcaatggcattagaagtaactcaccccagctagagaaactgtgtagtagtgtgaatgtaagcaatgagattaaatcttcaggaaacacaatgaaagtcatttttttcacggatggatccaggccatatggcggcttcactgcttcctatacctccagtgaagatgcagtgtgtggtgggtctcttccaaatactcctgaaggaaactttacttctcctggctatgacggagtcaggaattactcaagaaacctgaactgcgaatggactctcagcaatccaaatcagggaaattcatccatttccattcactttgaagatttttacctagaaagtcaccaagactgtcaatttgatgtcctcgagtttcgagtgggtgatgctgatgggcccctgatgtggagactttgtggtccttcaaagcctacattgccattggttataccttattctcaggtatggattcactttgtcaccaacgaacgtgtagaacacattggattccatgcaaagtattcctttacagattgtggcggaatacagataggtgacagtggagtgatcacaagccccaactatccaaatgcttatgacagcctgacccactgctcttcgctgttggaggccccacaagggcacaccatcactctcacatttagtgactttgatattgaaccccatacaacttgtgcttgggactctgtcactgtcaggaatggtgggtcccctgaatcacccatcataggacaatactgtggaaattcaaaccccaggacaatacagtcaggttccaatcagctggtcgtgacttttaactcagaccattcattgcaaggtggtggattttatgctacgtggaacacacaaactttaggttgtggtggaatatttcattctgataatggtacaatcagatcccctcactggcctcagaattttcccgaaaacagcagatgttcctggacggccattactcacaaaagtaaacacttggagatcagctttgacaacaacttcctaatccccagcggtgatggacaatgtcagaatagcttcgtgaaggtgtgggcaggaactgaggaggtggacaaagccctgctagccactggctgtgggaacgtggctccgggtcccgttatcacaccaagtaacacattcactgccgtcttccagtctcaggaggcaccagctcagggcttctccgcgtcctttgttagccgatgtggaagtaatttcactggcccttcaggttacatcatttctccaaattacccaaaacaatatgacaacaacatgaattgcacctatgtcatagaggctaatcctctgtcagtggtcctcttgacttttgtgtccttccacttagaagctcgttccgctgtgacgggaagctgtgtcaacgatggcgtgcacattatcagaggttacagcgtcatgtccaccccatttgctactgtgtgtggggatgagatgccagctcccctcaccatcgctgggccggttctgcttaacttctactccaacgagcaaatcacagacttcggattcaagttttcctataggataatctcctgtggtggtgtgttcaatttctcttctggaatcatcacaagtcctgcctattcatacgcagactacccaaatgatatgcactgtctgtataccatcaccgttagtgacgacaaggtgatcgagctcaagttcagtgattttgatgtggttccctccacctcctgctcccatgactacctggcaatttacgatggtgccaataccagcgatccccttcttggcaaattctgcggttccaagcgcccaccaaatgtgaagagcagcaataatagtatgctcctggtgttcaagacagattcatttcagacagcaaaaggctggaagatgtctttccggcagacattggggcctcagcaaggatgtggtggttatctgacaggctcgaataatacctttgcctctcctgattctgattcgaatggaatgtatgacaagaatttaaactgtgtatggatcataattgcacctgtaaacaaagtaattcacctcaccttcaatacatttgctctggaggcagcaagtactaggcaaagatgcctttatgattatgtaaagttatatgatggggatagtgaaaatgcgaacttggctggaacgttttgtggttccacagtacctgctccttttatctcttctggtaacttccttacggttcaattcatcagtgacttaacattagagagggaaggatttaatgctacatacaccatcatggacatgccttgtggtggaacatacaatgcaacttggaccccacaaaatatttcatcacccaattcatcagacccagatgtcccattttccatctgtacttgggtcattgattcccctccgcatcagcaggtcaagataactgtgtgggcattacagctgacctcgcaagactgcacgcagaattacttacagcttcaggactcaccgcagggtcacggaaattcaagatttcagttctgtggcagaaatgcttcggctgtgccagtgttttattcttctatgagtactgcaatggtcattttcaaatctggagttgtaaacagaaactctagaatgagtttcacctatcagattgcagattgcaacagagactatcacaaggcatttggcaacctgagaagccctggatggccagataactacgacaatgacaaggattgcaccgttactctcacagccccccagaaccacaccatttccctcttttttcattcacttggcatcgagaactcagttgaatgcagaaacgatttcttggaggtgagaaatggaagtaacagcaattcaccattactgggcaagtactgtggaactctgctgccaaaccctgtcttctctcaaaataatgaactatacctacgatttaagagtgatagtgtaacttctgatcgtggatatgaaatcatctggacttcatcaccctctggatgtggtggaactctttatggagacagaggctcattcaccagccccggctatccaggcacatacccaaacaacacgtactgcgagtgggtccttgttgctcctgctggaaggcttgtcaccatcaacttctacttcatcagcattgacgatccaggagactgtgtccagaactatctcacactctatgatgggcccaacgccagctctccatcctctggaccatactgcggaggcgacaccagcatagctcccttcgtggcttcctcaaatcaggtcttcataaaatttcatgctgattatgcacggcgtccatccgcattccgattaacttgggacagctaagtgggtaacaactcgtgttcactcagcactttccctctgcagcacgctggacagcactctgccatcctgatacatgacccctgctgatgccacagagaataagctgaacttgtatggtttttcaccaaaccatggatagaatcaatatttgtaggccaggcgtggtggctcacccccctgtattctcagcactttgggaggccgaggcaggttgatcacctgaggtcaggagtttgagactagcctggccagcatggtgaaacctcatctctctaacaatataataattagccaggcgtggtggtgggtgcctgtaattccagccactcgagaggctgaggcaggagaattgcttgaacccaggaggcagaggttgcagtgagctaagatcacaccactacactccagcctgggcgagacggcaagactccatctcaaaaaaaaaagaaacaaaaaaaaccagaatcaatatttgtacattttctcgaacatagaatatagcttctttagtcttgagtgtgcatttcattctaatattttgagctgaaatttaaaaaaactttgaaagagttggaaatgattatggcatatgtgacatacatttttaaaagttaataataatagccaggggcagtggctcatacccataatcccagcacgctgggaggccatgatgggaggattgcttgaacctaggagtttgagaccagcctgggcaacaaagtgagacctgatttttacaaaaaatcaaaaaattagccaggcatggtggcatgcacccgtggttccagctacacaggaggttgaagcaggaggatcacttgagcccagtaggttaaggctgcagtgaaaccctgtgaattaaccactgtactccagcctgggtgacagactgagaccctatctcaaaaatgacaacaagaacaacaaaagttaatgataatatagaagcataaatttcctgtgaatgttcaattacacataataaacattattgaattgtacacaa

SEQ ID NO: 7 - RNA, 20 nt, synthetic construct
  source 1..20: mol_type = other RNA
  ncRNA 1..20: ncRNA_class = sgRNA
tctcgtcgcctgcctagcgc

SEQ ID NO: 8 - RNA, 20 nt, synthetic construct
  source 1..20: mol_type = other RNA
  ncRNA 1..20: ncRNA_class = sgRNA
atcctcttgatcgtcgtaat

SEQ ID NO: 9 - RNA, 20 nt, synthetic construct
  source 1..20: mol_type = other RNA
  ncRNA 1..20: ncRNA_class = sgRNA
ccaatggcttggccatcgat

SEQ ID NO: 10 - protein, 16 aa, synthetic construct
CKKEEEKKEEEKKEEE

SEQ ID NO: 11 - protein, 17 aa, synthetic construct
CANTPCGPYTHDCPVKR

SEQ ID NO: 12 - DNA, 25 bp, Mus musculus
  source 1..25: mol_type = other DNA
tcgaggtcttactaacctgtccata

SEQ ID NO: 13 - DNA, 25 bp, Homo sapiens
  source 1..25: mol_type = other DNA
aaatgtcatacataccttgcgacct

SEQ ID NO: 14 - DNA, 25 bp, Mus musculus
  source 1..25: mol_type = other DNA
cccttcacctccatactcactgcac

SEQ ID NO: 15 - protein, 16 aa, synthetic construct
  MOD_RES 1: note = L-lysine-e-HyNic
XKKEEEKKEEEKKEEE

SEQ ID NO: 16 - protein, 17 aa, synthetic construct
  MOD_RES 1: note = L-lysine-e-HyNic
XANTPCGPYTHDCPVKR

SEQ ID NO: 17 - protein, 15 aa, synthetic construct
  MOD_RES 2: note = 6-amino-hexanoic acid
  MOD_RES 5: note = 6-amino-hexanoic acid
  MOD_RES 8: note = 6-amino-hexanoic acid
  MOD_RES 11: note = 6-amino-hexanoic acid
  MOD_RES 13: note = 6-amino-hexanoic acid
  SITE 1: note = D-Arginine
  SITE 3..4: note = D-Arginine
  SITE 6..7: note = D-Arginine
  SITE 9..10: note = D-Arginine
  SITE 12: note = D-Arginine
  MOD_RES 14: note = Beta-Alanine
  MOD_RES 15: note = L-lysine-e-HyNic
RXRRXRRXRRXRXBX

SEQ ID NO: 18 - RNA, 21 nt, Mus musculus
  source 1..21: mol_type = other RNA
  modified_base order(1..6,8,12..21): mod_base = OTHER; note = 2-Prime-methyl nucleotides
  modified_base order(7,9..11): mod_base = OTHER; note = 2-Prime-fluoro nucleotides
  modified_base 1^2: mod_base = OTHER; note = Phosphorothioate internucleotide linkage
  modified_base 2^3: mod_base = OTHER; note = Phosphorothioate internucleotide linkage
  modified_base 19^20: mod_base = OTHER; note = Phosphorothioate internucleotide linkage
  modified_base 20^21: mod_base = OTHER; note = Phosphorothioate internucleotide linkage
  ncRNA 1..21: ncRNA_class = siRNA
ttttcacacccaatatcacaa

SEQ ID NO: 19 - RNA, 23 nt, Mus musculus
  source 1..23: mol_type = other RNA
  modified_base order(1,3..5,7..13,15,17..23): mod_base = OTHER; note = 2-Prime-methyl nucleotides
  modified_base order(2,6,14,16): mod_base = OTHER; note = 2-Prime-fluoro nucleotides
  modified_base 1^2: mod_base = OTHER; note = Phosphorothioate internucleotide linkage
  modified_base 2^3: mod_base = OTHER; note = Phosphorothioate internucleotide linkage
  modified_base 21^22: mod_base = OTHER; note = Phosphorothioate internucleotide linkage
  modified_base 22^23: mod_base = OTHER; note = Phosphorothioate internucleotide linkage
  ncRNA 1..23: ncRNA_class = siRNA
ttgtgatattgggtgtgaaaagg

SEQ ID NO: 20 - RNA, 21 nt, Homo sapiens
  source 1..21: mol_type = other RNA
  modified_base order(1..6,8,12..21): mod_base = OTHER; note = 2-Prime-methyl nucleotides
  modified_base order(7,9..11): mod_base = OTHER; note = 2-Prime-fluoro nucleotides
  modified_base 1^2: mod_base = OTHER; note = Phosphorothioate internucleotide linkage
  modified_base 2^3: mod_base = OTHER; note = Phosphorothioate internucleotide linkage
  modified_base 19^20: mod_base = OTHER; note = Phosphorothioate internucleotide linkage
  modified_base 20^21: mod_base = OTHER; note = Phosphorothioate internucleotide linkage
  ncRNA 1..21: ncRNA_class = siRNA
tcctatgactgtagattttaa

SEQ ID NO: 21 - RNA, 23 nt, Homo sapiens
  source 1..23: mol_type = other RNA
  modified_base order(1,3..5,7..13,15,17..23): mod_base = OTHER; note = 2-Prime-methyl nucleotides
  modified_base order(2,6,14,16): mod_base = OTHER; note = 2-Prime-fluoro nucleotides
  modified_base 1^2: mod_base = OTHER; note = Phosphorothioate internucleotide linkage
  modified_base 2^3: mod_base = OTHER; note = Phosphorothioate internucleotide linkage
  modified_base 21^22: mod_base = OTHER; note = Phosphorothioate internucleotide linkage
  modified_base 22^23: mod_base = OTHER; note = Phosphorothioate internucleotide linkage
  ncRNA 1..23: ncRNA_class = siRNA
ttaaaatctacagtcataggaat

SEQ ID NO: 22 - protein, 5 aa, synthetic construct
CKKEE

SEQ ID NO: 23 - protein, 5 aa, synthetic construct
KKEEE

SEQ ID NO: 24 - protein, 15 aa, Homo sapiens
KKEEEKKEEEKKEEE

SEQ ID NO: 25 - protein, 6 aa, synthetic construct
  REGION 2..6: note = The amino acids 2-6 can be repeated two and three times.
KEEEKK

SEQ ID NO: 26 - protein, 4 aa, synthetic construct
YWTD

SEQ ID NO: 27 - protein, 4 aa, synthetic construct
KKEE

SEQ ID NO: 28 - protein, 16 aa, synthetic construct
CKKEEEKKEEEKKEEE

SEQ ID NO: 29 - protein, 8 aa, synthetic construct
KKEEEEEK